(19) United States
(12) Patent Application Publication
(10) Pub. No.: US 2026/0274928 A1
(43) Pub. Date: Sep. 17, 2026

(54) ANTIBODIES THAT BIND TO MULTIPLE SARBECOVIRUSES

(71) Applicant: Vir Biotechnology, Inc., San Francisco, CA (US)

(72) Inventors: Paul M. Chang, Pasadena, CA (US); Justin W. Chartron, Pasadena, CA (US); Davide Corti, Bellinzona (CH); Anna De Marco, Bellinzona (CH); Frederick S. Lee, Pasadena, CA (US); Barry D. Olafson, Pasadena, CA (US); Dora Pinto, Bellinzona (CH); Laura Rosen, San Francisco, CA (US); Gyorgy Snell, San Francisco, CA (US)

(21) Appl. No.: 19/473,428

(22) PCT Filed: Apr. 10, 2024
(86) PCT No.: PCT/US2024/023878

§ 371 Date: Oct. 7, 2025

Related U.S. Application Data
(60) Provisional application No. 63/495,260, filed on Apr. 10, 2023.
(60) Provisional application No. 63/626,876, filed on Jan. 30, 2024.
(60) Provisional application No. 63/557,275, filed on Feb. 23, 2024.

(51) Int. Cl. C07K 16/104; A61K 47/68; A61P 31/14

(52) U.S. Cl. CPC C07K 16/104; A61K 47/6803; A61K 47/6841; A61P 31/14; C07K 2317/33; C07K 2317/55; C07K 2317/56; C07K 2317/732; C07K 2317/76; C07K 2317/92

(57) ABSTRACT

The instant disclosure provides antibodies and antigen-binding fragments thereof that can bind to S proteins of sarbecoviruses (including, in some embodiments, multiple sarbecoviruses) and, in certain embodiments, are capable of neutralizing infection by multiple sarbecoviruses.

### REFERENCE TO AN ELECTRONIC SEQUENCE LISTING

This application contains a Sequence Listing, which has been submitted electronically in xml format and is hereby incorporated by reference in its entirety. Said xml copy, created on Oct. 6, 2025, is named SeqList-368564-45001.xml and is 312,786 bytes in size.

### BACKGROUND

Sarbecoviruses are divided into four clades: 1a, 1b, 2, and 3. SARS-CoV is a member of clade 1a, while SARS-CoV-2 is a member of clade 1b. Therapies for preventing or treating sarbecovirus infections, and diagnostic reagents for diagnosing sarbecovirus infections, are needed.

### BRIEF DESCRIPTION OF THE SEVERAL VIEWS OF THE DRAWINGS

FIG. 1 shows a phylogenetic tree of sarbecoviruses, divided into clade 1a (also referred to as clade 1, which includes SARS-CoV viral lineages), clade 1b (also referred to as clade 1/2, which includes SARS-CoV-2 lineages), clade 2 (also referred to as Bat SE Asian), and clade 3 (also referred to as Bat non-Asian). GenBank or NCBI accession numbers are indicated.

FIG. 2 illustrates the SARS-CoV-2 RBD antigen-binding sites of comparison antibodies S2H97 (site V), S309 (sotrovimab, site IV), S2X324 (site I/IIa), S2K146 (site Ia), and S2X259 (site IIa).

FIG. 3 shows ELISA binding results for S2V29-v1.1 to various sarbecoviruses representing different viral clades.

FIG. 4 shows neutralization by S2V29-v1.1 against SARS-CoV-2 variant lineages.

FIG. 5 shows neutralizing activity against SARS-CoV-2 Omicron variant lineages for S2V29-v1.1 and comparison antibodies S2K146, S2X259, S2X259v50, sotrovimab (which has the same RBD binding location as S309), S2X324, S3L17, S312, and S3013.

FIG. 6A shows inhibition of ACE2 to the SARS-CoV-2 (Clade 1b) RBD by S2V29-v1.1 and comparison antibody binding.

FIG. 6B shows inhibition of ACE2 to the SARS-CoV (Clade 1a) RBD by S2V29-v1.1 and comparison antibody binding.

FIG. 7A shows the results of evolutionary breadth binding testing for comparison antibody S2V29-v1.2.

FIG. 7B shows the results of evolutionary breadth binding testing for S2V29-v37.2.

FIG. 8 shows likely SARS-CoV-2 RBD epitopes for S2V29-v1.1 and comparison antibody S3L17 and binding of both antibodies to the SARS-CoV-2 RBD.

FIG. 9 shows how binding by S2V29-v1.2 and S2V29-v37.2 compares to ACE2 binding to the SARS-CoV-2 BQ.1.1 variant lineage RBD and highlights key RBD ACE-2 contact residues; resolution is 1.67 Angstroms.

FIG. 10 shows SARS-CoV-2 neutralization data for S2V29 variant antibodies, including S2V29-v37.2, comparison antibody S2V29-v1.2 and other S2V29 comparison variant antibodies with VL.2 as indicated by labels above each graph.

FIG. 11 shows further SARS-CoV-2 and SARS-Co-V neutralization data for S2V29 variant antibodies, including S2V29-v37.2, comparison antibody S2V29-v1.2, and other comparison variant antibodies comprising VL.2. Neutralization data includes IC50 (FIG. 11A), fold change (FC) as compared to S2V29-v1.2 (FIG. 11B), and FC of neutralization of SARS-CoV-2 BQ. 1.1 variant lineage versus SARS-CoV-2BQ.1.1-F456L variant lineage (FIG. 11C). The legend shown in FIG. 11C is also applicable to FIG. 11A and FIG. 11B.

FIG. 12 summarizes neutralization data for S2V29 variant antibodies, including S2V29-v37.2 and comparison antibody S2V29-v1.2 against specific SARS-CoV-2 BQ.1.1 variant lineage mutations.

FIG. 13 shows the neutralization data summarized in FIG. 12. The specific SARS-CoV-2 BQ1.1 variant lineage is noted on the top of each graph.

FIG. 14 shows additional SARS-CoV-2 variant lineage (as indicated at the top of each graph) and SARS-CoV neutralization data for S2V29-v37.2.

FIG. 15 shows the effects on SARS-CoV-2 RBD binding of changes at amino acids 50 and 57 of the VH of S2V29-v1.2 in S2V29-v.37.2.

FIG. 16 shows results for S2V29-v.37.2 neutralization and comparison antibody S2V29-v1.2 neutralization of SARS-CoV (SARS1) and SARS-CoV-2 (SARS2) partial escape variant lineages as determined by pseudovirus assay.

FIG. 17 shows results for S2V29-v37.2 and comparison antibodies REGEN-CoV (casirivimab/imdevimab, as sold by Regeneron, NY, US) and EVUSHIELD (AZD7442, tixagevimab/cilgavimab, Astra Zeneca, Cambridge, UK) neutralization of a variety of SARS-CoV-2 variant lineages as determined by pseudovirus assay.

FIG. 18 shows results of a comparison of pseudovirus neutralization assay results and authentic virus neutralization assay results for various SARS-CoV-2 variant lineages for S2V29-v37.2 and comparison antibody S2V29-v1.2.

FIG. 19 summarizes binding affinity results for S2V29-v37.2 and comparison antibody S2V29-v1.2 to various sarbecoviruses representing Clade 1a, Clade 1b, Clade 2, and Clade 3 (FIG. 19A) and RBD escape mutations (FIG. 19B) in SARS-CoV-2 BQ.1.1 variant lineages as determined by surface plasmon resonance (SPR).

FIG. 20 shows pharmacokinetic testing data in Tg32 mice for S2V29-v37.2 and comparison antibody S2V29-v1.2 Time is day post-injection.

FIG. 21A shows S2V29-v37.2-mediated pseudovirus neutralization assay results for SARS-CoV-2 variant lineages.

FIG. 21B shows S2V29-v37.2-mediated neutralization of authentic live virus SARS-CoV-2 variant lineages. Wild-type virus isolate has the same S haplotype as Wuhan-Hu-1.

FIG. 22 shows S2V29-v37.2 Fab binding affinity measured by SPR. Bar shade denotes sarbecovirus clade. In particular SARS-CoV-2 variant lineages Wuhan-Hu-1, BA.1, BA.2, BA.2.75.2, BA.5, BQ.1.1, XBB.1.5, CH.1.1, EG.5, HK.3, BA.2.68, and JN. 1 are all Clade 1b SARS-CoV-2 variant lineages; SARS-CoV-1 and WIV-1 are both Clade 1a lineages (SARS-CoV-1 RBD is the Urbani strain); BM48-31, BtKY72, and Khosta-2 are all Clade 3 lineages; and Anlong-112, SC2018, Shaanxi2011, and YN2013 are all Clade 2 lineages.

FIG. 23 summarizes pseudovirus neutralization by S2V29-v37.2, comparison antibody S2V29-v1.2, and other comparison antibodies, shaded by sarbecovirus clade as indicated. SVB designates SOTROVIMAB (Vir, San Francisco, CA). SA55 designates an antibody in development by Sinovac, Beijing, CN. Omi-42 designates an antibody developed by University of Oxford (Oxford, UK). Data points within the gray shaded bar at the top of the graph indicate IC50>10,000 ng/ml.

FIG. 24 shows pseudovirus neutralization assay results for S2V29-v37.2 and comparison antibody S2V29-v1.2, for Wuhan-Hu-1 and SARS-CoV-1 (SARS-CoV-1, Clade 1a).

FIG. 25 shows bio-layer interferometry (BLI) results for S2V29-v37.2 Fab fragments or comparison antibody Fab fragments (comparison antibodies all bind SARS-CoV-2 RBD) competing with monomeric ACE2 for binding to Wuhan-Hu-1 RBD.

FIG. 26 shows results for S2V29-v37.2 from a S1 shedding assay using Wuhan-Hu-1 or SARS-CoV-2 XBB.1.5 variant lineage S1 protein transiently expressed on the surface of Expi-CHO cells. Control antibody S2M28 is an anti-SARS-CoV-2 S protein N-terminal domain (NTD) control mAb.

FIG. 27 shows activation of human FcγRIIa by the indicated antibodies. Target cells were CHO cells stably expressing Wuhan-Hu-1 SARS-CoV-2 S protein. Effector cells were Jurkat cells expressing the indicated FcγR and engineered with a NFAT-mediated luciferase reporter to reflect activation of human FcγRs. Data points show means±SD of duplicates. Results for S2M28 anti-NTD control antibody and anti-SARS-CoV-2 RBD comparison antibody VIR-8000 (VIR) are also presented.

FIG. 28 shows results from a human FcγRIIIa (D) activation assay using the indicated antibodies. Target cells were CHO stably expressing SARS-CoV-2 spike protein. Effector cells were Jurkats expressing the indicated FcγR and engineered with a NFAT-mediated luciferase reporter to reflect activation of human FcγRs. Data points show means±SD of duplicates. Results for S2M28 anti-NTD control antibody and anti-SARS-CoV-2 RBD antibody VIR-8000 (VIR) are also presented.

FIG. 29 shows results from NK-cell mediated antibody-dependent cell cytotoxicity (ADCC) studies using the indicated donor antibodies and donor cells expressing FcγRIIIa-genotypes: heterozygous (F/V158; FIG. 29A); homozygous high-affinity (V/V158; FIG. 29B). Results for S2M28 anti-NTD control antibody and anti-SARS-CoV-2 RBD antibody VIR-8000 (VIR) are also presented.

FIG. 30 shows S2V29-v37.2-mediated pseudovirus neutralization of the SARS-CoV-2 XBB.1.5 variant lineage with the indicated RBD epitope mutations. Mutations with >0.005% frequency in the Global Initiative on Sharing All Influenza Data (GISAID) database as of June 2023 were tested.

FIG. 31 show the results of serial passaging of SARS-CoV-2 Wuhan-Hu-1 and XBB.1.5 variant lineage rVSV in the presence of S2V29-v.37.2 and comparison antibodies SA55 and Omi-42. Variant lineages and S protein mutations that were observed as having deep mutational scanning (DMS) binding escape mutations, alone or plus F456, were also tested. Two independent replicates were performed for each experiment.

FIG. 32 shows S2V29-v37.2-mediated pseudovirus neutralization of SARS-CoV-2 variant lineages also having the S protein mutations indicated on the x axis. ND indicates not determined.

FIG. 33 shows S2V29-v37.2-mediated pseudovirus neutralization of SARS-CoV-2 variant lineages also carrying the S protein mutations indicated on the x axis.

FIG. 34 shows S2V29-v37.2-mediated pseudovirus neutralization of SARS-CoV-2 variant lineages BQ.1.1 or XBB.1.5 carrying the indicated S protein mutations, which were all below 0.005% frequency in the GISAID database as of June 2023, but were all accessible by a single nucleotide change from wild-type sequences.

FIG. 35 shows results indicating the impact of mutations at SARS-CoV-2 RBD position 455 on S2V29-v37.2 Fab fragment binding affinity measured by SPR (top) and on S2V29-v37.2-mediated pseudovirus neutralization (bottom). S proteins also including the F456L mutation, or the L455S mutation are indicated by bars. EG.5+L455F also carried the non-RBD Q52H S mutation (i.e. HK3 strain haplotype).

FIG. 36 shows results indicating the impact of mutations at SARS-CoV-2 RBD position 455 on ACE2 affinity as measured by SPR. S proteins also including the F456L mutation, or the L455S mutation are indicated by bars. EG.5+L455F also carried the non-RBD Q52H S mutation (i.e. HK3 strain haplotype).

FIG. 37 shows the results of cell-cell fusion assays between VeroE6/TMPRSS2 and SARS-CoV-2 S-expressing cells for the XBB.1.5 and EG.5 variant lineages, normalized by SARS-CoV-2 S expression.

### DETAILED DESCRIPTION

Provided herein are antibodies and antigen-binding fragments that are capable of binding to a sarbecovirus (e.g. SARS-CoV-2). In some embodiments, an antibody or antigen-binding fragment is capable of binding to multiple sarbecoviruses (e.g., binding to a surface glycoprotein, as described herein, of one or more (e.g., one, two, three, four, five, six, or more) different sarbecoviruses, optionally comprised on a virion and/or expressed on the surface of a cell infected by two or more sarbecoviruses). In certain embodiments, the multiple sarbecoviruses comprise one or more Clade 1b sarbecoviruses. In certain embodiments, the multiple sarbecoviruses comprise one or more Clade 1a sarbecovirus. In certain embodiments, the multiple sarbecoviruses comprise one or more Clade 1a viruses and one or more Clade 1b viruses. In still other embodiments, the multiple sarbecoviruses comprise one or more sarbecoviruses from each of Clades 1a, 1b, 2, and 3. Antibodies antigen-binding fragments may be monospecific or multispecific, such as bispecific. In certain embodiments, presently disclosed antibodies and antigen-binding fragments can neutralize infection by one or more sarbecovirus (e.g., one, two, three, four, or more sarbecoviruses) in an in vitro model of infection and/or in an animal model and/or in a human subject. In the case of multispecific antibodies or antigen-binding fragments, in some embodiments, a single first antigen binding domain formed by a single VH/VL combination, out of the multiple antigen binding domains in the multispecific antibody or antigen-binding fragment, may exhibit binding to and/or neutralization of one or more viruses representing these various clades. The other antigen binding domain(s) may bind the same viruses, viral lineages or epitopes as the first antigen binding domain, or different viruses, viral lineages, or epitopes than the first antigen binding domain. Combinations of two or more different antibodies or antigen-binding fragments, e.g. for use in viral neutralization, therapy and/or prophylaxis, are also provided. In addition, antibody drug conjugates (ADCs) including antibodies or antigen-binding fragments of the present disclosure, and in some embodiments conjugated small molecules, or administration of antibodies or antigen-binding fragments of the present disclosure with small molecules is also provided.

Also provided are polynucleotides, vectors, DNA therapeutics, RNA therapeutics, and host cells able to produce antibodies or antigen-binding fragments of the present disclosure, and related compositions.

The disclosure further provides methods of using the antibodies, antigen-binding fragments, polynucleotides, vectors, DNA therapeutics, RNA therapeutics, host cells, and related compositions to treat (e.g., reduce, delay, eliminate, or prevent) infection by two or more sarbecoviruses in a subject and/or in the manufacture of a medicament for treating infection in a subject by one or more sarbecovirus (e.g. one, two, three, four, or more sarbecoviruses). The disclosure also provides antibodies, antigen-binding fragments, polynucleotides, vectors, DNA therapeutics, RNA therapeutics, host cells, and related compositions for use in treating (e.g., reducing, delaying, eliminating, or preventing) infection by two or more sarbecoviruses in a subject and/or in the manufacture of a medicament for treating infection in a subject by one or more sarbecovirus (e.g. one, two, three, four, or more sarbecoviruses).

Prior to setting forth this disclosure in more detail, it may be helpful to understand definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components, provided, however, that “an amino acid sequence” or “a polypeptide sequence” should not be interpreted as referring to only a portion (e.g. one or two amino acids or polypeptides) of the sequence. The use of the alternative (e.g., “or”) should be understood to mean either one, both, or any combination thereof of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not. In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain) or a protein “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, an “anti-sarbecovirus antibody or antigen-binding fragment” specifically binds one or more sarbecovirus and may, in some embodiments, bind two or more, three or more, four or more, or five or more sarbecoviruses. These sarbecoviruses may be from the same or different clades. In some embodiments, variant lineages of a particular sarbecovirus are not considered to be different sarbecoviruses (e.g. SARS-CoV-2 BA.5 may not be a different virus than SARS-CoV-2 XBB.1).

As used herein, “sarbecovirus” refers to any betacoronavirus within lineage B, and includes lineage B viruses in clade 1a, clade 1b, clade 2, and clade 3.

Examples of clade 1a sarbecoviruses are SARS-CoV and Bat SARS-like coronavirus WIV1 (WIV1).

Examples of clade 1b sarbecoviruses are SARS-CoV-2, RatG13, Pangolin-Guanxi-2017 (PANG/GX) and Pangolin-Guangdon-2019 (PANG/GD).

Examples of clade 1b also include SARS-CoV-2 variant lineages, for example variant lineages with any of the mutations: Δ67V, A69-70, T95I, G142D, 137-145de, 143-145de, Y145H, N211I, Δ212, V213G, ins214TDR, ins215EPE, A222V, G339D, R346K, R346S, V367F, S371L, S373P, S375F, T376A, P384L, N394S, D405N, R408S, Q414K, K417N, K417V, K417T, N439K, N440K, G446S, Y449H, Y449N, L452R, L452Q, L452X (where X is any amino acid), Y453F, S477N, T478K, V483A, E484A, E484Q, E484K, E484X (where X is any amino acid), F490R, F486V, F490S, R493Q, Q493R, S494P, G496S, Q498R, N501Y, N501T, Y505H, E516Q, T547K, Q613H, D614G, A653V, H655Y, G669S, Q677H, N679K, ins679GIAL, P681H, P681R, A701V, N764K, D796Y, N856K, Q954H, N969K, L981F, or the variants in B.1.1.7 and Q lineages and descendant lineages (Alpha); B.1.351 and descendant lineages (Beta); B.1.429 and B.1.427 and descendant lineages (Epsilon); P.1 and descendant lineages (Gamma); B.1.1.222; C.37; B.1.617.2; AY.1, AY.2, other AY lineages, and descendant lineages (Delta); B.1.525 and descendant lineages (Eta); B.1.526 and descendant lineages (Iota); B.1.617.1 and descendant lineages (Kappa); 1.617.3; B.1.621 and B.1.621.1 and descendant lineages (Mu); P.2 (Zeta); and B.1.1.529.1, BA.1, BA.2, BA.2.12, BA.2.75.2, BA.2.86, BA.3, BA.4, BA.5 and descendant lineages (Omicron); as well as BQ.1.1, XBB.1, XBB.1.5, CH.1.1, XBB.2.3, EG.5, EG.5.1, XBB.1.16.1, XBB.1.16.6, FL. 1.5.1, HK.3, HV.1, JD.1.1, and JN. 1 and descendant lineages.

Examples of clade 2 sarbecoviruses are Bat ZC45 (ZC45), Bat ZXC21 (ZXC21), YN2013, RmYN02, Anlong112, SC2018, SX2011.

Examples of clade 3 sarbecoviruses are BtkY72 and BGR2008.

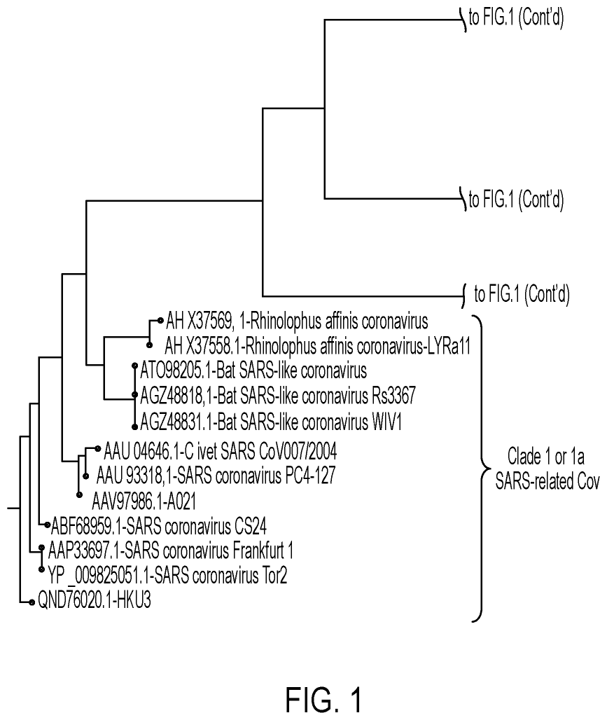

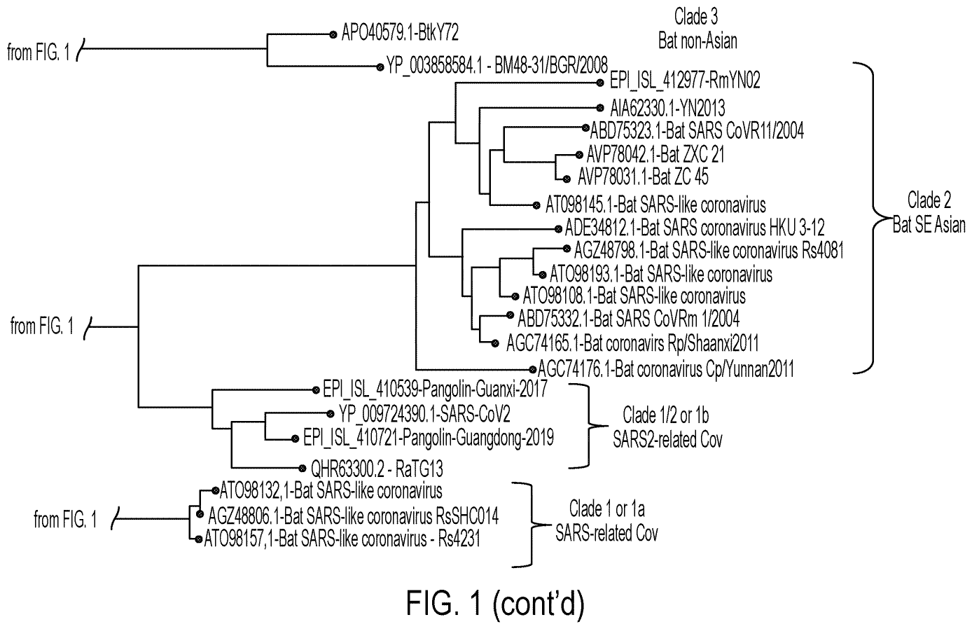

Sarbecovirus clades as of June 2023 are also illustrated in FIG. 1. Further clades or members of the clades indicated in FIG. 1 may be identifies as sarbecoviruses continue to evolve.

In some embodiments, an antibody or antigen-binding fragment is capable of binding to a sarbecovirus of clade 1b, such as SARS-CoV-2 (including all variant lineages described herein), RatG13, Pangolin-Guanxi-2017 (PANG/GX), Pangolin-Guangdon-209, or any combination thereof. Such an antibody or antigen-binding fragment may be referred to as “anti-clade 1b” or “anti-sarbecovirus clade 1b.”

In certain further embodiments, an antibody or antigen-binding fragment is capable of binding to a SARS-CoV-2 variant lineage; e.g. examples of clade 1b also include SARS-CoV-2 variant lineages, SARS-CoV-2 variant lineages, for example variant lineages with any of the mutations: A67V, Δ69-70, T95I, G142D, 137-145de, 143-145de, Y145H, N211I, Δ212, V213G, ins214TDR, ins215EPE, A222V, G339D, R346K, R346S, V367F, S371L, S373P, S375F, T376A, P384L, N394S, D405N, R408S, Q414K, K417N, K417V, K417T, N439K, N440K, G446S, Y449H, Y449N, L452R, L452Q, L452X (where X is any amino acid except L), Y453F, S477N, T478K, V483A, E484A, E484Q, E484K, E484X (where X is any amino acid except E), F490R, F486V, F490S, R493Q, Q493R, S494P, G496S, Q498R, N501Y, N501T, Y505H, E516Q, T547K, Q613H, D614G, A653V, H655Y, G669S, Q677H, N679K, ins679GIAL, P681H, P681R, A701V, N764K, D796Y, N856K, Q954H, N969K, L981F, D614, E340A, or the variants in B.1.1.7 and Q lineages and descendant lineages (Alpha); B.1.351 and descendant lineages (Beta); B.1.429 and B.1.427 and descendant lineages (Epsilon); P.1 and descendant lineages (Gamma); B.1.1.222; C.37; B.1.617.2; AY.1, AY.2, other AY lineages, and descendant lineages (Delta); B.1.525 and descendant lineages (Eta); B.1.526 and descendant lineages (Iota); B.1.617.1 and descendant lineages (Kappa); 1.617.3; B.1.621 and B.1.621.1 and descendant lineages (Mu); P.2 (Zeta); and B.1.1.529.1, BA.1, BA.2, BA.2.12, BA.2.75.2, BA.2.86, BA.3, BA.4, BA.5 and descendant lineages (Omicron); as well as BQ.1.1, XBB.1, XBB.1.5, CH.1.1, XBB.2.3, EG.5, EG.5.1, XBB.1.16.1, XBB.1.16.6, FL. 1.5.1, HK.3, HV.1, JD. 1.1, and JN. 1 and descendant lineages, or any combination thereof. Such an antibody or antigen-binding fragment may be referred to as “anti-SARS-CoV-2.” Such an antibody may also be referred to as “anti-[SARS-CoV-2 lineage],” or, if applicable, an “anti-[SARS-CoV-2 lineage] variants.”

In general, any antibody or antigen-binding fragment disclosed herein may be referred to as “anti” the virus type, clade, specific virus, or lineage, or variant lineage, any of which may be carrying a specified mutation, to which the antibody or antigen-binding fragment binds. A single antibody or antigen-binding fragment may, therefore, be referred to as “anti” multiple other terms. For example an S2V29 antibody or antigen-binding fragments thereof may be appropriately referred to as any of “anti-sarbecovirus,” “anti-clade 1,” anti-clade 1/2,” “anti-clades 1 and 1/2,” anti-sarbecovirus clade 1,” “anti-sarbecovirus clades 1/2,” “anti-sarbecovirus clades 1 and 1/2,” “anti-SARS-CoV,” “anti-SARS-CoV-1,” “anti-SARS-CoV-2,” “anti-SARS-CoV and SARS-CoV-2,” “anti-BQ.1.1,” “anti-BQ.1.1 variants,” “anti-BQ.1.1 and BQ.1.1 variants,” “anti-SARS-CoV-2 BQ.1.1,” “anti-SARS-CoV-2 BQ.1.1 variants,” and “anti-SARS-CoV-2 BQ.1.1 and BQ.1.1 variants.” The preceding list is for exemplification only and does S2V29 and antigen-binding fragments thereof may also be appropriately referred to as “anti” a number of other viruses, lineages, etc. to which they bind.

“S2V29” as used herein includes any of the VH and VL variants disclosed herein, each of which may be referred to as “a S2V29 antibody.” S2V29 variant antibodies are labeled by VH and VL combination. For example, a S2V29 antibody having VH.22 and VL.2 is designated S2V29-v22.2. S2V29-v.1.1, discussed specifically herein, has VH.1 and VL.1 (also referred to as VL11a). S2V29-v1.2, discussed specifically herein, has VH.1 and VL.2. S2V29-v37.2, discussed specifically herein, has VH.37 and VL.2. S2V29-v37.2 variant antibodies, discussed specifically herein, have variations of VH.37 and/or VL.2

In some embodiments, an antibody is (or an antigen-binding fragment is from) an S2V29 antibody, or the antibody or antigen-binding fragment comprises the (e.g. six) CDRs and optionally one or more framework regions or the whole or partial VH and VL of an S2V29 antibody, particularly a S2V29-v37.2 or variant antibody or antigen-binding fragment thereof. In some embodiments, an antibody or a fragment thereof, comprises sufficient CDR, VH, and/or VL identity to an S2V29 antibody disclosed herein, particularly S2V39-v37.2, to confer specific binding to the same sarbecoviruses as the S2V29 antibody, and is capable of inhibiting a binding interaction between human ACE2 and a sarbecovirus (e.g., SARS-CoV-2) receptor binding domain (RBD) with an IC50 in a range between about 0.5 ng/ml to about 100 ng/ml, about 1 ng/ml to about 100 ng/ml, about 2.0 ng/ml to about 100 ng/ml, about 2.5 ng/ml to about 100 ng/ml, about 5.0 ng/ml to about 100 ng/ml, about 7.5 ng/mL to about 100 ng/ml, about 8.0 ng/ml to about 100 ng/mL, about 9.0 ng/ml to about 100 ng/mL, about 10.0 ng/ml to about 100 ng/mL, about 12.5 ng/mL to about 100 ng/mL, about 15.0 ng/ml to about 100 ng/ml, about 17.5 ng/ml to about 100 ng/mL, about 20 ng/ml to about 100 ng/ml, about 25.0 ng/ml to about 100 ng/mL, about 27.5 ng/ml to about 100 ng/mL, about 30 ng/mL to about 100 ng/mL, about 0.5 ng/ml to about 50 ng/mL, about 1 ng/mL to about 50 ng/mL, about 2.0 ng/ml to about 50 ng/mL, about 2.5 ng/mL to about 50 ng/ml, about 5.0 ng/ml to about 50 ng/mL, about 7.5 ng/mL to about 50 ng/mL, about 8.0 ng/mL to about 50 ng/ml, about 9.0 ng/ml to about 50 ng/mL, about 10.0 ng/ml to about 50 ng/mL, about 12.5 ng/ml to about 50 ng/mL, about 15.0 ng/ml to about 50 ng/mL, about 17.5 ng/mL to about 50 ng/ml, about 20 ng/ml to about 50 ng/ml, about 25.0 ng/mL to about 50 ng/mL, about 27.5 ng/ml to 50 ng/ml, or about 30 ng/ml to about 50 ng/mL, about 0.5 ng/mL, about 0.9 ng/mL, about 1.0 ng/ml, about 1.25 ng/ml, about 1.5 ng/mL, about 1.75 ng/ml, about 2.0 ng/ml, about 2.25 ng/ml, about 2.5 ng/ml, about 3.0 ng/mL, about 4.0 ng/ml, about 5.0 ng/ml, about 7.5 ng/ml, about 8.0 ng/ml, about 9.0 ng/ml, about 10.0 ng/ml, about 12.5 ng/mL, about 15.0 ng/mL, about 17.5 ng/ml, about 20.0 ng/mL, about 22.5 ng/ml, about 25.0 ng/mL, about 27.5 ng/mL, or about 30 ng/ml, or at least about 30 ng/mL to about 50 ng/ml, about 0.5 ng/ml, about 0.9 ng/ml, about 1.0 ng/ml, about 1.25 ng/ml, about 1.5 ng/ml, about 1.75 ng/mL, about 2.0 ng/mL, about 2.25 ng/ml, about 2.5 ng/ml, about 3.0 ng/mL, about 4.0 ng/mL, about 5.0 ng/ml, about 7.5 ng/mL, about 8.0 ng/ml, about 9.0 ng/ml, about 10.0 ng/ml, about 12.5 ng/ml, about 15.0 ng/ml, about 17.5 ng/ml, about 20.0 ng/ml, about 22.5 ng/mL, about 25.0 ng/mL, about 27.5 ng/ml, or about 30 ng/mL.

In a particular embodiment in which the antibody or antigen-binding fragment is a S2V29-v37.2 or variant antibody or antigen-binding fragment thereof, or comprises the CDRs. and optionally one or more framework regions or the whole or partial VH and VL of S2V29-v37.2 or a variant antibody thereof, the antibody or antigen-binding fragment may be capable of inhibiting a binding interaction between human ACE2 and a sarbecovirus (e.g., SARS-CoV-2) receptor binding domain (RBD) with an IC50 in a range between about 100 and 103 ng/ml, about 1×10¹ and 2×10² ng/ml, or about 1×10¹ and 102 ng/ml.

In some embodiments, the S2V29 antibody, particularly S2V29-v37.2 or variant antibody or antigen-binding fragment thereof is capable of binding two or more (e.g., two, three, four, etc.) sarbecoviruses, such as sarbecoviruses from different clades or subclades, or both SARS-CoV and SARS-CoV-2 with an IC50 for each virus in a range as indicated above.

As used herein, “SARS-CoV-2”, also originally referred to as “Wuhan coronavirus”, “Wuhan seafood market pneumonia virus”, or “Wuhan CoV”, “novel CoV”, or “nCoV”, or “2019 nCoV”, or “Wuhan nCoV”, or a variant lineage thereof, is a betacoronavirus of lineage B (sarbecovirus). SARS-CoV-2 was first identified in Wuhan, Hubei province, China, in late 2019 and spread within China and to other parts of the world by early 2020. SARS CoV-2 infection can result in a disease known as CoVID-19; symptoms of CoVID-19 include fever or chills, dry cough, dyspnea, fatigue, body aches, headache, new loss of taste or smell, sore throat, congestions or runny nose, nausea or vomiting, diarrhea, persistent pressure or pain in the chest, new confusion, inability to wake or stay awake, and bluish lips or face.

The genomic sequence of SARS-CoV-2 isolate Wuhan-Hu-1 is provided at GenBank MN908947.3, Jan. 23, 2020, and the amino acid translation of the genome is at GenBank QHD43416.1, Jan. 23, 2020. These GenBank sequences and GenBank sequences for all SARS-CoV-2 variant lineages are incorporated by reference herein as describing a sarbecovirus or a SARS-CoV-2 that an antibody or antigen-binding fragment according to the present disclosure specifically binds.

The genomic sequences of variant lineages and of other sarbecoviruses, such as SARS-CoV, and variant lineages thereof are also provided in GenBank and other publicly available sources, and should be referred to as they existed on Apr. 10, 2023. S proteins and RBDs of these other sarbecoviruses are also identified in GenBank.

Like other sarbecoviruses, and particularly coronaviruses (e.g., SARS-CoV), SARS-CoV-2 comprises a surface (“S”) type I transmembrane glycoprotein (also referred to as a “surface glycoprotein,” “S protein,” “spike,” or “spike protein”) containing a receptor binding domain (RBD). RBD is believed to mediate entry of the Clade 1b SARS coronavirus to respiratory epithelial cells by binding to the cell surface receptor angiotensin-converting enzyme 2 (ACE2).

In particular, a receptor binding motif (RBM) in the virus RBD is believed to interact with ACE2. Other sarbecoviruses have an S protein containing an RBDs that further comprise an RBM that interacts with a target protein required for the sarbecovirus to infect a mammalian cell.

The amino acid sequence of the Wuhan-Hu-1 surface glycoprotein is provided in SEQ ID NO: 1. The amino acid sequence of the Wuhan-Hu-1 RBD is provided in SEQ ID NO:2. Wuhan-Hu-1 S protein has approximately 73% amino acid sequence identity with that of SARS-CoV. The amino acid sequence of Wuhan-Hu-1 RBM is provided in SEQ ID NO:3.

There have been a number of emerging SARS-CoV-2 variant lineages, which may differ in genomic and amino acid sequences, particularly of the surface glycoprotein or the RBD. Variants may also be referred to as different strains or lineages or as having mutations as compared to a reference variant or strain. Some SARS-CoV-2 variant lineages have mutations that increase affinity to the ACE receptor and/or infectiveness of the virus. Significant variant lineages of SARS-CoV-2 include for example variants with any of the mutations: A67V, Δ69-70, T95I, G142D, 137-145de, 143-145de, Y145H, N211I, Δ212, V213G, ins214TDR, ins215EPE, A222V, G339D, R346K, R346S, V367F, S371L, S373P, S375F, T376A, P384L, N394S, D405N, R408S, Q414K, K417N, K417V, K417T, N439K, N440K, G446S, Y449H, Y449N, L452R, L452Q, L452X (where X is any amino acid except L), Y453F, S477N, T478K, V483A, E484A, E484Q, E484K, E484X (where X is any amino acid except E), F490R, F486V, F490S, R493Q, Q493R, S494P, G496S, Q498R, N501Y, N501T, Y505H, E516Q, T547K, Q613H, D614G, A653V, H655Y, G669S, Q677H, N679K, ins679GIAL, P681H, P681R, A701V, N764K, D796Y, N856K, Q954H, N969K, L981F, D614, E340A, or the variants in B. 1.1.7 and Q lineages and descendant lineages (Alpha); B.1.351 and descendant lineages (Beta); B.1.429 and B.1.427 and descendant lineages (Epsilon); P.1 and descendant lineages (Gamma); B.1.1.222; C.37; B.1.617.2; AY.1, AY.2, other AY lineages, and descendant lineages (Delta); B.1.525 and descendant lineages (Eta); B.1.526 and descendant lineages (Iota); B.1.617.1 and descendant lineages (Kappa); 1.617.3; B.1.621 and B.1.621.1 and descendant lineages (Mu); P.2 (Zeta); B.1.1.529.1, BA.1, BA.2, BA.2.12, BA.2.75.2, BA.2.86, BA.3, BA.4, BA.5 and descendant lineages (Omicron); as well as BQ.1.1, XBB.1, XBB.1.5, CH.1.1, XBB.2.3, EG.5, EG.5.1, XBB.1.16.1, XBB.1.16.6, FL.1.5.1, HK.3, HV.1, JD.1.1, and JN.1 and descendant lineages, or any combination thereof. Variant lineages of SARS-CoV-2 circulating in the United States are classified as variants of concern by the U.S. Centers for Disease Control and Prevention (see cdc.gov/coronavirus/2019-ncov/variants/variant-info).

In some embodiments, an antibody or antigen-binding fragment is provided for treating a sarbecovirus infection. In certain embodiments, the sarbecovirus infection comprises a SARS-CoV-2 infection. Treating a SARS CoV-2 infection in accordance with the present disclosure includes treating infection by any one or more of the aforementioned SARS-CoV-2 viruses. In certain embodiments, treating a SARS-CoV-2 infection comprises treating infection by any one or more of SARS CoV-2 Wuhan-Hu-1, the variant lineages identified above, or other variant lineages identified herein, optionally with further mutations identified herein. Treating may further comprise treating infection by new variant lineages and mutations.

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid, and non-naturally occurring amino acid polymers. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90%, 91%, 92%, 93%, 94%, 95%, 96%, 97%, 98%, 99%, or 99.9% identical to an amino acid sequence of a defined or reference amino acid sequence as described herein.

Any polypeptide of this disclosure (e.g., VH, VL, Fab, Fd, antibody heavy chain, antibody light chain) can, as encoded by a polynucleotide sequence, comprise a “signal peptide” (also known as a leader sequence, leader peptide, or transit peptide). Signal peptides target newly synthesized polypeptides to their appropriate location inside or outside the cell. A signal peptide may be removed in whole or in part from the polypeptide during or once localization or secretion is completed. Polypeptides that have a (n, e.g., full-length) signal peptide can be referred to as a “pre-protein” and polypeptides having their signal peptide removed—at least in part—can be referred to as “mature” proteins or polypeptides. In certain embodiments, an antibody or antigen-binding fragment is a mature protein or a pre-protein.

In some embodiments, the antibody or antigen-binding fragment may include a signal peptide that causes secretion of the antibody or antigen-binding fragment from the host cell. The present disclosure contemplates the addition of a signal peptide to any protein having a sequence specifically set forth therein, or any variations thereof with amounts of identity as set forth herein.

“Nucleic acid molecule” or “polynucleotide” or “polynucleic acid” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), which includes mRNA, microRNA, siRNA, self-assembling RNA, self-amplifying RNA (sa RNA), trans-amplifying RNA (taRNA), viral genomic RNA, circular RNA (circRNA), and synthetic RNA, and polydeoxyribonucleic acid (DNA), which includes cDNA, genomic DNA, and synthetic DNA, either of which may be single or double stranded. If single-stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a binding domain thereof having a functionality described herein, such as binding a target molecule.

“mRNA” refers to any form of messenger RNA having a protein-coding region able to be translated in a host cell to produce a produce the protein coded, in particular an antibody or antigen-binding fragment.

In some embodiments, each distinct mRNA may encode only one protein molecule, such as a heavy chain (HC), a heavy chain variable domain (VH)-containing fragment thereof, a CDRH1-H3-containing fragment thereof, or a light chain (LC), a light chain variable domain (VL)-containing fragment thereof, or a CDRL1-L3-containing fragment thereof. In such embodiments, a RNA therapeutic containing such mRNA may include at least two distinct mRNAs, one encoding a HC or a VH-containing fragment thereof, and the other encoding a LC or a VL-containing fragment thereof, so that, upon expression of the mRNA in the cell, the resulting VH and VL domains may combine to form an antigen-binding domain.

In other embodiments, multiple protein subunits of an antibody or and antigen-binding fragment, or at least a protein comprising a VH and a protein comprising a VL, may be encoded in a single mRNA. In some such embodiments, the VH and VL may be expressed as separate proteins due to regulatory elements or self-cleaving elements disposed between sequences of the VH or VL, such as signal peptides, spacers, and protein-regulatory sequences, such as internal ribosome entry site (IRES) sequences. In certain embodiments, the order in which the VH and VL and/or other expressed proteins occur in the mRNA may be optimized. In particular, RNA therapeutics containing saRNA or circRNA may be optimized in this manner, in some embodiments by placing VH-encoding sequences 5′ to VL-encoding sequences.

In some embodiments, the antibody or antigen-binding fragment may include a signal peptide that causes secretion of the antibody or antigen-binding fragment from a host cell expressing the same.

mRNA of the present disclosure, in particular circRNA, taRNA, or saRNA, may further contain one or more modified nucleosides. In particular embodiments, the modified nucleosides may stabilize and reduce degradation of the mRNA in vitro, such as during production, storage, or after reconstitution and prior to administration of the mRNA, a vector comprising or consisting of the mRNA, an mRNA therapeutic construct, or a composition containing and of the preceding. In particular embodiments, the modified nucleosides may stabilize and reduce degradation of the mRNA in vivo, such as in the skin, muscle, blood, interstitial fluid, other extracellular environment, or intracellular environment after administration of the mRNA, a vector or construct containing the mRNA, an mRNA therapeutic construct, or a composition containing and of the preceding. In particular embodiments, the modified nucleosides may reduce or avoid a cellular immune response to the mRNA. In particular embodiments, the modified nucleosides may enhance amplification of the mRNA (particularly in the case of taRNA or saRNA) in the host cell and/or expression of a protein or peptide encoded by the mRNA. Modified nucleosides may include pseudouridine, such as N¹-methylpseudouridine, 5-methylcytidine, 2-thiouridine, N6-methyladenonsine. Nucleoside modifications suitable for use with RNA are further described in Zhang et al. Front. Immunol., DOI: 10.3389/fimmu. 2019.00594 (2019); Eyler et al. PNAS 116(46):23068-23071; DOI: 10.1073/pnas.1821754116 (2019); Nance and Meier, ACS Cent. Sci. 2021, 7, 5, 748-756; doi. org/10.1021/acscentsci. 1c00197 (2021), and van Hoecke and Roose, J. Translational Med 17:54 (2019); doi. org/10.1186/s12967-019-1804-8, which modified nucleosides and mRNA features are incorporated herein by reference. mRNA may include more than two or more types of modified nucleosides.

In some embodiments, methylation of a naturally occurring (unmodified) nucleotides may be used alone or in combination with a modified nucleotide to achieve any of the above effects achievable using a modified nucleotide. In some embodiments, the proportion of nucleotides having a specific base or located in a specific sequence that are methylated may be used to achieve such effects. For example, methylation may be used to reduce recognition of the mRNA (or, in the case of DNA therapeutics, the DNA) as foreign in a mammalian host cell. Types of RNA methylation and their effects are further described in Yujia Z., et al., “Principles of RNA methylation and their implications for biology and medicine,” Biomedicine & Pharmacotherapy (131) 110731 (2020), which is incorporated by reference in its entirety herein.

In some embodiments, the mRNA, particularly aRNA or taRNA, may include a cap or a cap analog, more specifically a 7-methylguanosine moiety linked via a phosphate, particularly a trisphosphate to an end nucleotide. The including of a cap or cap analog may help prevent exonuclease cleavage of the mRNA, particularly saRNA or taRNA, and/or initiate translation of the mRNA, particularly saRNA or taRNA, in a mammalian host cell. In specific embodiments, the cap or cap analog may initiate translation of the replication protein or peptide. circRNA may lack a suitable location for a cap or cap analog because of the absence of an otherwise unbound 5′ end. However, linear mRNA, prior to circularization to form circRNA, may contain a cap or cap analog to, for example, increase stability and/or amplification of the linear mRNA prior to circularization.

In some embodiments, the mRNA may include one or more untranslated region (UTR) sequence that regulates expression of an encoded protein or peptide. In some embodiments the UTR may be endogenous to the host cell. In some embodiments, the UTR may be exogenous to the host cell or artificially designed. In some embodiments, the mRNA may include two UTRs, one 5′ and one 3′ of the nucleic acid sequence encoding the protein or peptide. For mRNAs encoding more than one protein or peptide, UTRs may be 5′ and 3′ of the sequence encoding one or more, typically each, protein or peptide, or 5′ and 3′ of the sequence encoding at least two proteins or peptides, all proteins or peptides that function together once expressed (e.g. the VH and VL-containing sequences), or all proteins and peptides encoded by the mRNA.

In some embodiments, the mRNA, particularly saRNA or taRNA, may include a poly A tail or a sequence that results in a poly A tail in mRNA produced from saRNA or taRNA (e.g. by reverse transcription) in a mammalian host cell. In some embodiments, the poly A tail, prior to initial translation of mRNA, particularly saRNA or taRNA, particularly taRNA sequences encoding amplification proteins, containing the poly A tail, may have a length of at least or approximately 250, 200, 100, 50, 20, or 10 nucleotides, or a length in a range between 10 and 250, 10 and 200, 10 and 100, 10 and 50, 10 and 20, 20 and 250, 20 and 200, 20 and 100, 20 and 50, 50 and 250, 50 and 200, 50 and 100, 100 and 250, 100 and 200, or 200 and 250 nucleotides. The length of the poly A tail may be varied to affect the lifespan of the mRNA in the host cell, with a longer poly A tail resulting in a longer lifespan and typically also a greater number of times the mRNA is expressed prior to degradation. circRNA may lack a suitable location for a polyA tail because of the absence of an unbound end. However, linear mRNA, prior to circularization to form circRNA, may contain a poly A tail, for example, increase stability and/or amplification of the linear mRNA prior to circularization.

In certain embodiments, the mRNA, including circRNA, taRNA, or saRNA, may be produced in a production host cell, which is a type of host cell. In other embodiments, the mRNA, including circRNA, taRNA, or saRNA, may be produced in a cell-free system, such as a system using a DNA template and enzymes.

“Circular RNA” (“circRNA”) is a type of single-stranded mRNA that forms a covalently-closed continuous loop. In some embodiments, the 3′ and 5′ ends of a corresponding linear protein-coding mRNA are covalently bound in circRNA. In some embodiments circRNA may be resistant to exonuclease degradation as compared to corresponding linear mRNA. In specific embodiments, the circRNA includes one or more nucleotide sequences encoding one or more antibodies or antigen-binding fragments that are expressed when the circRNA is present in the host cell. In a specific embodiment of the present disclosure, circRNA may include one or more nucleotide sequences encoding an antibody or antigen-binding fragment according to the present disclosure that are expressed and produce the antibody or antigen-binding fragment in a human host cell in vivo.

In some embodiments circRNA may be formed from mature linear mRNA, particularly by self-splicing. In some embodiments, circRNA may self-splice by back-splicing, in which the 3′ end, after a protein coding region, binds to a 5′ end before the protein-coding region. In some embodiments, circRNA may self-splice by intron-pairing driven circularization, in which introns in the protein-coding region bind to one another, for example using Alu repeats. In some embodiments, circRNA may form a debranching-resistant lariat in an intron of the protein-coding region. In some embodiments, circRNA may for an exon-skipping lariat within the protecin-coding region.

In all of these embodiments, the circRNA or linear mRNA from which it is formed may contain circularization sequences, which facilitate circularization of linear mRNA. In some embodiments, circRNA may be formed from linear mRNA via action of a spliceosome in an in vitro production host cell.

In some embodiments, circRNA may be introduced to the human host cell via a circRNA therapeutic construct. This introduction may occur in vivo in a human subject and, in particular in an in vivo human host cell. Once in the cytosol, the circRNA is translated, thereby expressing the encoded protein(s).

“Self-amplifying RNA” (“sa RNA”) is also sometimes referred to as “self-replicating RNA” or a “replicon.” saRNA is a type of mRNA that contains one or more nucleotide sequences that, when the saRNA is present in a host cell, cause replication of the saRNA and that also includes one or more nucleotide sequences encoding one or more antibodies or antigen-binding fragments that are expressed when the saRNA is present in the host cell. In a specific embodiment of the present disclosure, a saRNA may include i) one or more nucleotide sequences encoding an antibody or antigen-binding fragment according to the present disclosure that are expressed and produce the antibody or antigen-binding fragment in a human host cell in vivo, and ii) one or more nucleotide sequences that cause replication of the saRNA in the human host cell in vivo. In some embodiments, the one or more nucleotide sequences that cause replication of the saRNA in a host cell may encode one or more replication proteins or peptides.

In some embodiments, the saRNA further includes one or more promoters that cause expression of the replication protein or peptide and the antibody or antigen-binding fragment in the host cell. In specific embodiments, the promoter has a sequence comprising, consisting essentially of, or consisting of the following sequence UAACCUGAAUGGACUACGACAUAGUCUAGUCCGCCAAGUCUAGCAUAUGGCCACC AUG (SEQ ID NO: 205). In embodiments in which a form is DNA is used to amplify or otherwise construct the saRNA, U will be replaced with T in this promoter sequence.

In specific embodiments, the one of more nucleotide sequences that cause replication of the saRNA in a human host cell include sequences encoding one or more replication proteins or peptides that replicate the saRNA. In a more specific embodiment, the replication protein or peptide may be an RNA polymerase, such as an RNA-dependent RNA polymerase (RDRP). In some embodiments, the RDRP may be an alphavirus RDRP or a variant thereof, more particularly a Venezuelan Equine Encephalitis Virus (VEEV), particularly the VEE TC-83 virus, Semliki Forest Virus (SFV), or Sindbis Virus RDRP or variant thereof. In more particular embodiments, the replication proteins may comprise, consist essentially of, or consist of non-structural proteins 1-4 (nsP1-4), in which nsP1 is a protein that causes mRNA capping, nsP2 is a NTPase/helicase/protease, nsP3 is a macrodomain that mediates interactions between viral and host proteins, and nsP4 is RDRP.

In some embodiments, the following sequence includes the amino acid sequence for nsP1-4:

(SEQ ID NO: 206)
MEKVHVDIEEDSPFLRALQRSFPQFEVEAKQVTDNDHANARAFSHLASKLIETEVDPSDT
ILDIGSAPARRMYSKHKYHCICPMRCAEDPDRLYKYATKLKKNCKEITDKELDKKMKEL
AAVMSDPDLETETMCLHDDESCRYEGQVAVYQDVYAVDGPTSLYHQANKGVRVAYW
IGFDTTPFMFKNLAGAYPSYSTNWADETVLTARNIGLCSSDVMERSRRGMSILRKKYLK
PSNNVLFSVGSTIYHEKRDLLRSWHLPSVFHLRGKQNYTCRCETIVSCDGYVVKRIAISP
GLYGKPSGYAATMHREGFLCCKVTDTLNGERVSFPVCTYVPATLCDQMTGILATDVSA
DDAQKLLVGLNQRIVVNGRTQRNTNTMKNYLLPVVAQAFARWAKEYKEDQEDERPLG
LRDRQLVMGCCWAFRRHKITSIYKRPDTQTIIKVNSDFHSFVLPRIGSNTLEIGLRTRIRK
MLEEHKEPSPLITAEDVQEAKCAADEAKEVREAEELRAALPPLAADVEEPTLEADVDLM
LQEAGAGSVETPRGLIKVTSYDGEDKIGSYAVLSPQAVLKSEKLSCIHPLAEQVIVITHSG
RKGRYAVEPYHGKVVVPEGHAIPVQDFQALSESATIVYNEREFVNRYLHHIATHGGALN
TDEEYYKTVKPSEHDGEYLYDIDRKQCVKKELVTGLGLTGELVDPPFHEFAYESLRTRP
AAPYQVPTIGVYGVPGSGKSGIIKSAVTKKDLVVSAKKENCAEIIRDVKKMKGLDVNAR
TVDSVLLNGCKHPVETLYIDEAFACHAGTLRALIAIIRPKKAVLCGDPKQCGFFNMMCL
KVHFNHEICTQVFHKSISRRCTKSVTSVVSTLFYDKKMRTTNPKETKIVIDTTGSTKPKQD
DLILTCFRGWVKQLQIDYKGNEIMTAAASQGLTRKGVYAVRYKVNENPLYAPTSEHVN
VLLTRTEDRIVWKTLAGDPWIKTLTAKYPGNFTATIEEWQAEHDAIMRHILERPDPTDVF
QNKANVCWAKALVPVLKTAGIDMTTEQWNTVDYFETDKAHSAEIVLNQLCVRFFGLD
LDSGLFSAPTVPLSIRNNHWDNSPSPNMYGLNKEVVRQLSRRYPQLPRAVATGRVYDM
NTGTLRNYDPRINLVPVNRRLPHALVLHHNEHPQSDFSSFVSKLKGRTVLVVGEKLSVP
GKMVDWLSDRPEATFRARLDLGIPGDVPKYDIIFVNVRTPYKYHHYQQCEDHAIKLSML
TKKACLHLNPGGTCVSIGYGYADRASESIIGAIARQFKFSRVCKPKSSLEETEVLFVFIGY
DRKARTHNSYKLSSTLTNIYTGSRLHEAGCAPSYHVVRGDIATATEGVIINAANSKGQPG
GGVCGALYKKFPESFDLQPIEVGKARLVKGAAKHIIHAVGPNFNKVSEVEGDKQLAEAY
ESIAKIVNDNNYKSVAIPLLSTGIFSGNKDRLTQSLNHLLTALDTTDADVAIYCRDKKWE
MTLKEAVARREAVEEICISDDSSVTEPDAELVRVHPKSSLAGRKGYSTSDGKTFSYLEGT
KFHQAAKDIAEINAMWPVATEANEQVCMYILGESMSSIRSKCPVEESEASTPPSTLPCLCI
HAMTPERVQRLKASRPEQITVCSSFPLPKYRITGVQKIQCSQPILFSPKVPAYIHPRKYLVE
TPPVDETPEPSAENQSTEGTPEQPPLITEDETRTRTPEPIIIEEEEEDSISLLSDGPTHQVLQV
EADIHGPPSVSSSSWSIPHASDFDVDSLSILDTLEGASVTSGATSAETNSYFAKSMEFLARP
VPAPRTVFRNPPHPAPRTRTPSLAPSRACSRTSLVSTPPGVNRVITREELEALTPSRTPSRS
VSRTSLVSNPPGVNRVITREEFEAFVAQQQ*RFDAGAYIFSSDTGQGHLQQKSVRQTVLS
EVVLERTELEISYAPRLDQEKEELLRKKLQLNPTPANRSRYQSRKVENMKAITARRILQG
LGHYLKAEGKVECYRTLHPVPLYSSSVNRAFSSPKVAVEACNAMLKENFPTVASYCIIPE
YDAYLDMVDGASCCLDTASFCPAKLRSFPKKHSYLEPTIRSAVPSAIQNTLONVLAAAT
KRNCNVTQMRELPVLDSAAFNVECFKKYACNNEYWETFKENPIRLTEENVVNYITKLK
GPKAAALFAKTHNLNMLQDIPMDRFVMDLKRDVKVTPGTKHTEERPKVQVIQAADPLA
TAYLCGIHRELVRRLNAVLLPNIHTLFDMSAEDFDAIIAEHFQPGDCVLETDIASFDKSED
DAMALTALMILEDLGVDAELLTLIEAAFGEISSIHLPTKTKFKFGAMMKSGMFLTLFVNT
VINIVIASRVLRERLTGSPCAAFIGDDNIVKGVKSDKLMADRCATWLNMEVKIIDAVVGE
KAPYFCGGFILCDSVTGTACRVADPLKRLFKLGKPLAADDEHDDDRRRALHEESTRWN
RVGILSELCKAVESRYETVGTSIIVMAMTTLASSVKSFSYLRGAPITLYG.

In some embodiments, the saRNA encodes one or more replication protein having a sequence with at least 85% (e.g. 85%, 86, 87, 88, 89, 90, 91, 92, 93, 94, 95, 96, 97, 98, 99, or 100%) identity to SEQ ID NO: 206.

Modifications and production methods described above for saRNA may, in some embodiments, be present or used with other types of mRNA.

In some embodiments, the saRNA may be introduced to the human host cell via an saRNA therapeutic construct. This introduction may occur in vivo in a human subject and, in particular in an in vivo human host cell. Once in the cytosol, ribosomes may translate the saRNA, thereby expressing the one or more replication proteins. The replication proteins or peptides may then produce a negative saRNA strand (a strand complementary to the saRNA as delivered to the host cell), which is then used as a template by the replication proteins or peptides to produce replicated positive saRNA strands. In some embodiments, the replicated positive saRNA strands may be identical in sequence to the saRNA delivered to the host cell or may otherwise comprise both the sequence encoding the replication proteins or peptides and the sequence encoding the antibody or antigen-binding fragment, and may be referred to as “complete replicated saRNA.” In other embodiments, the replicated positive saRNA strands may comprise the sequence encoding the antibody or antigen-binding fragment, but lack the sequence encoding the replication proteins or peptides, and may be referred to as “antibody-restricted replicated saRNA.” In some embodiments, both complete replicated saRNA and antibody-restricted replicated saRNA are produced in the human host cell.

The complete replicated saRNA or the antibody-restricted replicated saRNA, or both, may then be translated by cellular ribosomes to express the antibody or antigen-binding fragment.

“Trans-amplifying RNA” or “taRNA” refers to a collection of mRNAs in a taRNA therapeutic construct, that may function similarly to an saRNA therapeutic construct. Such a taRNA therapeutic construct may comprise multiple distinct mRNA molecules that collectively encode the replication proteins or peptides and the antibody or antigen-binding fragment that may be found in an saRNA therapeutic construct. For example, the taRNA therapeutic construct may comprise a first mRNA encoding the antibody or antigen-binding fragment and a second or additional mRNA encoding one or more replication proteins or peptides. For example, the taRNA therapeutic construct may comprise a distinct mRNA for each antibody or antigen-binding fragment and replication protein or peptide that might otherwise be found in an saRNA therapeutic construct.

In some embodiments, one or more taRNA in a taRNA therapeutic construct encodes one or more replication protein having a sequence with at least 85% (e.g. 85%, 86, 87, 88, 89, 90, 91, 92, 93, 94, 95, 96, 97, 98, 99, or 100%) identity to SEQ ID NO: 206.

In further specific embodiments, the saRNA or the taRNA may be incapable of genomic insertion in the host cell, particularly in a human host cell in vivo.

In some embodiments, saRNA or taRNA may also be circRNA.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

Other examples include Clustal W, MAFFT, Clustal Omega, AlignMe, Praline, GAP, BESTFIT, Needle (EMBOSS), Stretcher (EMBOSS), GGEARCH2SEQ, Water (EMBOSS), Matcher (EMBOSS), LALIGN, and SSEARCH2SEQ. A global alignment algorithm, such as a Needleman and Wunsch algorithm, can be used to align two sequences over their entire length, maximizing the number of matches and minimizes the number of gaps. Default values can be used.

To generate similarity scores for two amino acid sequences, scoring matrices can be used that assign positive scores for some non-identical amino acids (e.g., conservative amino acid substitutions, amino acids with similar physio-chemical properties, and/or amino acids that exhibit frequent substitutions in orthologs, homologs, or paralogs). Non-limiting examples of scoring matrices include PAM30, PAM70, PAM250, BLOSUM45, BLOSUM50, BLOUM62, BLOSUM80, and BLOSUM90.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector, DNA therapeutic, RNA therapeutic and/or such nucleic acid or polypeptide could be part of a composition (e.g., a cell lysate), and still be isolated in that such vector, DNA therapeutic, RNA therapeutic, or composition is not part of the natural environment for the nucleic acid or polypeptide.

The term “gene” means the segment of DNA or RNA involved in producing a polypeptide chain; in certain contexts, it includes regions preceding and following the coding region (e.g., 5′ untranslated region (UTR) and 3′ UTR) as well as intervening sequences (introns) between individual coding segments (exons).

A “functional variant” refers to a polypeptide or polynucleotide that is structurally similar or substantially structurally similar to a parent or reference compound of this disclosure, but differs slightly in composition (e.g., one base, atom or functional group is different, added, or removed), such that the polypeptide or encoded polypeptide is capable of performing one or more function of the parent polypeptide with at least 50% efficiency, preferably at least 55%, 60%, 70%, 75%, 80%, 85%, 90%, 95%, 96%, 97%, 98%, 99%, 99.9%, or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (K_{D}) constant).

As used herein, a “functional portion” or “functional fragment” refers to a polypeptide or polynucleotide comprising only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity).

As used herein, the term “engineered,” “recombinant,” or “non-natural” refers to an organism, microorganism, cell, nucleic acid molecule, vector, DNA therapeutic, or RNA therapeutic that includes one or more genetic alteration or has been modified by introduction of an exogenous or heterologous nucleic acid molecule, wherein such alterations or modifications are introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

In certain embodiments, a nucleic acid molecule or portion thereof native to a host cell will be considered heterologous to the host cell if it has been altered or mutated, or a nucleic acid molecule native to a host cell may be considered heterologous if it has been altered with a heterologous expression control sequence or has been altered with an endogenous expression control sequence not normally associated with the nucleic acid molecule native to a host cell. In addition, the term “heterologous” can refer to a biological activity that is different, altered, or not endogenous to a host cell. As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other.

For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a protein (e.g., a heavy chain of an antibody), or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule (or, when the context clearly indicates, a fusion protein of the present disclosure). A (polynucleotide) construct may be present in a vector (e.g., a bacterial vector, a viral vector) or may be integrated into a genome.

A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mol. Ther. 8:108, 2003: Mátés et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, “expression vector” or “vector” refers to a DNA or RNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself or deliver the polynucleotide contained in the vector into the genome without the vector sequence. In the present specification, “plasmid,” “expression plasmid,” “virus,” and “vector” are often used interchangeably.

In some embodiments, the “vector” may comprise or consist of mRNA, such as saRNA, taRNA, or circRNA.

The term “introduced” in the context of inserting a nucleic acid molecule into a cell, means “transfection”, “transformation,” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon (e.g. a replicon formed by saRNA), or transiently expressed (e.g., transfected mRNA, such as circRNA, taRNA, or saRNA).

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox, and canarypox). Other viruses include, for example, Norwalk virus, togavirus, flavivirus, reoviruses, papovavirus, hepadnavirus, and hepatitis virus. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vectors” include HIV-based lentiviral vectors for gene delivery, which can be integrative or non-integrative, have relatively large packaging capacity, and can transduce a range of different cell types. Lentiviral vectors are usually generated following transient transfection of three (packaging, envelope, and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors that can be used with the compositions and methods of this disclosure include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Plasmid vectors, including DNA-based antibody or antigen-binding fragment-encoding plasmid vectors for direct administration to a subject, are described further herein.

As used herein, a “carrier” or “vehicle” comprises a molecule able to introduce a polynucleotide into a host cell. In specific embodiments, the carrier may comprise a lipid, a lipid-derived delivery vehicle, such as a liposome, a solid lipid nanoparticle, an oily suspension, a submicron lipid emulsion, a lipid microbubble, an inverse lipid micelle, a cochlear liposome, a lipid microtubule, a lipid microcylinder, lipid nanoparticle (LNP), a liopolyplex (LPP), a cationic polypeptide, a polymeric nanoparticle, or a nanoscale platform, such as a nanoemulsion. (see, e.g., Li et al. Wilery Interdiscip Rev. Nanomed Nanobiotechnol. 11(2):e1530 (2019), incorporated by reference herein with respect to its description of carriers and vehicles and methods of making and using such carriers and vehicles to form therapeutics).

Principles, reagents, and techniques for designing appropriate mRNA and formulating mRNA-LNP and delivering the same are described in, for example, Pardi et al. (J Control Release 217345-351 (2015)); Thess et al. (Mol Ther 23:1456-1464 (2015)); Thran et al. (EMBO Mol Med 9(10):1434-1448 (2017); Kose et al. (Sci. Immunol. 4 eaaw6647 (2019); and Sabnis et al. (Mol. Ther. 26:1509-1519 (2018)), which techniques, include capping, codon optimization, nucleoside modification, purification of mRNA, incorporation of the mRNA into stable lipid nanoparticles (e.g., ionizable cationic lipid/phosphatidylcholine/cholesterol/PEG-lipid; ionizable lipid: distearoyl PC: cholesterol: polyethylene glycol lipid), and subcutaneous, intramuscular, intradermal, intravenous, intraperitoneal, and intratracheal administration of the same, are incorporated herein by reference.

In some embodiments, LNPs may be formed from a cationic lipid, in particular an ionizable lipid. In other embodiments, LNPs may be formed from lipidoids, which are molecules derived from ionizable lipids and dendrimers. LNPs, in some embodiments, may further include structural lipids, such as a phospholipid, cholesterol, or both. In some embodiments, the LNPs may comprise i) an ionizable lipid having a positive charge able to bind to the negatively charged RNA backbone, ii) a PEGylated lipid, iii) a cholesterol, and iv) a phospholipid.

As used herein, an “RNA therapeutic” is an RNA therapeutic construct or naked RNA, including naked circRNA, taRNA, or saRNA. An “RNA therapeutic construct” comprises an RNA molecule and a carrier.

In some embodiments, the RNA therapeutic construct may “self assemble” from a mixture comprising the RNA and the carrier or carrier precursors, such as smaller lipid vesicles. Self-assembly may particularly occur with LNP carriers.

A “circRNA therapeutic construct” comprises circRNA and a carrier. circRNA may present different behaviors in the formation and function of a circRNA therapeutic construct than are observed with respect to corresponding linear mRNA in the presence of the same carrier. For example, due to the circular nature of circRNA, the number of copies that can be contained in a carrier may be different than the number of copies possible using corresponding linear mRNA. As another example, the conditions that result in encapsulation of circRNA in a carrier, or its release in vivo from the carrier, may also be different than the conditions applicable to corresponding linear mRNA.

A “taRNA therapeutic construct” comprises at least two distinct taRNAs, a first taRNA encoding a replication protein and a second taRNA encoding a protein or peptide of interest, typically an antibody or antigen-binding fragment. In some embodiments, the second taRNA may encode a VH-containing peptide and the taRNA therapeutic construct may further include a third taRNA that encodes a VL-containing peptide.

An “saRNA therapeutic construct” comprises an saRNA and a carrier. saRNA, due to the inclusion of sequences encoding replication proteins or peptides, is often larger than mRNA encoding only a protein or peptide of interest, and saRNA therapeutic constructs may therefore benefit from the use of carriers functional with longer RNA molecules, such as carriers, particularly LNPs, comprising polyethylenimine (PEI).

In some embodiments, the saRNA therapeutic construct may include at least 10, 50, or 100, or between 10 and 50, between 10 and 100, or between 50 and 100 copies of the saRNA per carrier.

As used herein a “DNA therapeutic” is an DNA therapeutic construct, or naked DNA, including naked DNA vectors. A “DNA therapeutic construct” comprises a DNA molecule and a carrier.

As used herein, the term “host” refers to a cell or microorganism targeted for genetic modification with a heterologous nucleic acid molecule to produce a polypeptide of interest (e.g., an antibody of the present disclosure).

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

In embodiments relating to RNA therapeutics, including circRNA therapeutics, taRNA therapeutics, or saRNA therapeutics, and DNA therapeutics the host cell may be human cell. In some more specific embodiments, the host cell may be a cell able to produce antibodies prior to introduction of the RNA or DNA, such as a B cell. In other more specific embodiments, the host cell may be a cell not otherwise able to produce antibodies prior to introduction of the RNA or DNA, such as a muscle cell.

In embodiments relating to RNA therapeutics or DNA therapeutics that are not produced using a cell-free system, at least two types of host cells may exist, a “production host cell” used to produce RNA or DNA for the DNA therapeutic, and an “in vivo human host cell” into which the RNA or DNA is introduced.

“Antigen” or “Ag”, as used herein, refers to an immunogenic molecule that provokes an immune response. This immune response may involve antibody production, activation of specific immunologically competent cells, activation of complement, antibody dependent cytotoxicity, or any combination thereof. An antigen (immunogenic molecule) may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, stool samples, cells, biological fluids, or combinations thereof. Antigens can be produced by cells that have been modified or genetically engineered to express an antigen. Antigens can also be present in a sarbecovirus (e.g., a surface glycoprotein or portion thereof), such as present in a virion, or expressed or presented on the surface of a cell infected by a sarbecovirus. Antibodies or antigen-binding fragments thereof may be referred to as “anti” an antigen to which they bind.

The term “epitope” or “antigenic epitope” includes any molecule, structure, amino acid sequence, or protein determinant that is recognized and specifically bound by a cognate binding molecule, such as an immunoglobulin, or other binding molecule, domain, or protein. Epitopic determinants generally contain chemically active surface groupings of molecules, such as amino acids or sugar side chains, and can have specific three-dimensional structural characteristics, as well as specific charge characteristics. Where an antigen is or comprises a peptide or protein, the epitope can be comprised of consecutive amino acids (e.g., a linear epitope), or can be comprised of amino acids from different parts or regions of the protein that are brought into proximity by protein folding (e.g., a discontinuous or conformational epitope), or non-contiguous amino acids that are in close proximity irrespective of protein folding.

### Antibodies, Antigen-Binding Fragments, and Compositions

In one aspect, the present disclosure provides an isolated antibody, or an antigen-binding fragment, that is comprises a S2V29 antibody, particularly S2V29-v37.2 or variant antibody or antigen-binding fragment thereof, capable of binding to a surface glycoprotein of a sarbecoviruses (e.g. of a SARS-CoV-2). In some embodiments, the antibody or antigen-binding fragment is capable of binding to a surface glycoprotein of two or more sarbecoviruses, three or more sarbecoviruses, four or more sarbecoviruses, or five or more sarbecoviruses. In some embodiments, the antibody or antigen-binding fragment comprises a heavy chain variable domain (VH) comprising a CDRH1, a CDRH2, and a CDRH3, and a light chain variable domain (VL) comprising a CDRL1, a CDRL2, and a CDRL3. In some embodiments, the antibody or antigen-binding fragment comprises a heavy chain (HC) comprising a VH (and, in some embodiments, a heavy chain constant domain (CH)) and a light chain (LC) comprising a VL (and, in some embodiments, a light chain constant domain (CL)).

In some embodiments, the two or more, three or more, four or more, or five or more sarbecoviruses comprise or one or more, or are selected from, clade 1b sarbecoviruses or naturally occurring variant lineages thereof, and any combination thereof. In certain embodiments, the antibody or antigen-binding fragment is capable of binding to a surface glycoprotein of two or more, three or more, four or more, or five or more sarbecoviruses; e.g. capable of binding when a sarbecovirus surface glycoprotein is expressed on a cell surface of a host cell and/or on a sarbecovirus virion. In certain embodiments, the two or more, three or more, four or more, or five or more sarbecoviruses are selected from SARS-CoV-2, PANG/GD, PANG/GX, RatG13, and naturally occurring variant lineages thereof. In some embodiments, the two or more, three or more, four or more, or five or more sarbecoviruses include one or more of SARS-CoV-2 variant lineages. In certain further embodiments, an antibody or antigen-binding fragment is capable of binding to one or more SARS-CoV-2 variant lineage. Examples of clade 1b also include SARS-CoV-2 variant lineages, for example variant lineages with any of the mutations: A67V, Δ69-70, T95I, G142D, 137-145de, 143-145de, Y145H, N211I, Δ212, V213G, ins214TDR, ins215EPE, A222V, G339D, R346K, R346S, V367F, S371L, S373P, S375F, T376A, P384L, N394S, D405N, R408S, Q414K, K417N, K417V, K417T, N439K, N440K, G446S, Y449H, Y449N, L452R, L452Q, L452X (where X is any amino acid except L), Y453F, S477N, T478K, V483A, E484A, E484Q, E484K, E484X (where X is any amino acid except E), F490R, F486V, F490S, R493Q, Q493R, S494P, G496S, Q498R, N501Y, N501T, Y505H, E516Q, T547K, Q613H, D614G, A653V, H655Y, G669S, Q677H, N679K, ins679GIAL, P681H, P681R, A701V, N764K, D796Y, N856K, Q954H, N969K, L981F, D614, E340A, or the B.1.1.7 and Q lineages and descendant lineages (Alpha); B.1.351 and descendant lineages (Beta); B.1.429 and B.1.427 and descendant lineages (Epsilon); P.1 and descendant lineages (Gamma); B.1.1.222; C.37; B.1.617.2; AY.1, AY.2, other AY lineages, and descendant lineages (Delta); B.1.525 and descendant lineages (Eta); B.1.526 and descendant lineages (Iota); B.1.617.1 and descendant lineages (Kappa); 1.617.3; B.1.621 and B.1.621.1 and descendant lineages (Mu); P.2 (Zeta); B.1.1.529.1, BA.1, BA.2, BA.2.12, BA.2.75.2, BA.2.86, BA.3, BA.4, BA.5 and descendant lineages (Omicrom); as well as BQ.1.1, XBB.1, XBB.1.5, CH.1.1, XBB.2.3, EG.5, EG.5.1, XBB.1.16.1, XBB.1.16.6, FL. 1.5.1, HK.3, HV.1, JD. 1.1, and JN. 1 and descendant lineages.

In some embodiments, the two or more, three or more, four or more, or five or more sarbecoviruses include one or more SARS-CoV-2 variant lineages having S protein mutations D614G, Q493R, G496S, Q498R, N501Y, Y453F, N439K, K417V, E484K, or any combination thereof, or mutations found in the BQ.1.1 or XBB.1 variant lineages and descendant lineages thereof. In certain embodiments, two or more sarbecoviruses include one or more SARS-CoV-2 variant lineages having S protein mutations K417N, Q493K, G496S, or any combination thereof.

In certain embodiments, an antibody or antigen-binding fragment of the present disclosure associates with or unites with a sarbecovirus surface glycoprotein epitope or antigen comprising the epitope, while not significantly associating or uniting with any other molecules or components in a sample. In some embodiments, the epitope is comprised in a S1 subunit of a S protein. In further embodiments, the epitope is comprised in a RBD of a S protein. In some embodiments, the epitope is a conformational epitope or a linear epitope.

In certain embodiments, an antibody or antigen-binding fragment of the present disclosure associates with or unites (e.g., binds) to a first sarbecovirus surface glycoprotein epitope, and can also associate with or unite with an epitope from another sarbecovirus present in the sample, but not significantly associating or uniting with any other molecules or components in the sample. In other words, in certain embodiments, an antibody or antigen-binding fragment of the present disclosure is cross-reactive against and specifically binds to two or more sarbecoviruses (e.g. against SARS-CoV-2 Wuhan-Hu-1 and one or more variant lineages thereof or against SARS-CoV and SARS-CoV-2).

In certain embodiments, an antibody or antigen-binding fragment of the present disclosure specifically binds to a sarbecovirus surface glycoprotein. As used herein, “specifically binds” refers to an association or union of an antibody or antigen-binding fragment to an antigen with an affinity or Kₐ (i.e., an equilibrium association constant of a particular binding interaction with units of 1/M) equal to or greater than 10⁵ M⁻¹ (which equals the ratio of the on-rate [Kₒₙ] to the off rate [K_{off}] for this association reaction), while not significantly associating or uniting with any other molecules or components in a sample. Alternatively, affinity may be defined as an equilibrium dissociation constant (K_{d}) of a particular binding interaction with units of M (e.g., 10⁻⁵ M to 10⁻¹³ M). Antibodies may be classified as “high-affinity” antibodies or as “low-affinity” antibodies. “High-affinity” antibodies refer to those antibodies having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low-affinity” antibodies refer to those antibodies having a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M).

A variety of assays are known for identifying antibodies of the present disclosure that bind a particular target, as well as determining binding domain or binding protein affinities, such as Western blot, ELISA (e.g., direct, indirect, or sandwich), analytical ultracentrifugation, biolayer interferometry, isothermal titration calorimetry, spectroscopy, and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). Assays for assessing affinity or apparent affinity or relative affinity are also known.

In certain embodiments, an antibody or antigen-binding fragment of the present disclosure binds one or more, or two or more, sarbecoviruses of Clade 1b or SARS-CoV and SARS-CoV-2 and, optionally, one or more additional sarbecoviruses with an EC50 in a range between about 0.5 ng/mL to about 100 ng/mL, about 1 ng/ml to about 100 ng/ml, about 2.0 ng/ml to about 100 ng/mL, about 2.5 ng/mL to about 100 ng/ml, about 5.0 ng/mL to about 100 ng/ml, about 7.5 ng/mL to about 100 ng/ml, about 8.0 ng/ml to about 100 ng/ml, about 9.0 ng/ml to about 100 ng/ml, about 10.0 ng/ml to about 100 ng/ml, about 12.5 ng/mL to about 100 ng/mL, about 15.0 ng/ml to about 100 ng/ml, about 17.5 ng/ml to about 100 ng/ml, about 20 ng/ml to about 100 ng/ml, about 25.0 ng/ml to about 100 ng/mL, about 27.5 ng/mL to about 100 ng/ml, about 30 ng/ml to about 100 ng/ml, about 0.5 ng/ml to about 50 ng/mL, about 1 ng/ml to about 50 ng/ml, about 2.0 ng/ml to about 50 ng/ml, about 2.5 ng/ml to about 50 ng/mL, about 5.0 ng/ml to about 50 ng/mL, about 7.5 ng/mL to about 50 ng/mL, about 8.0 ng/ml to about 50 ng/ml, about 9.0 ng/mL to about 50 ng/mL, about 10.0 ng/mL to about 50 ng/ml, about 12.5 ng/ml to about 50 ng/ml, about 15.0 ng/ml to about 50 ng/ml, about 17.5 ng/ml to about 50 ng/mL, about 20 ng/mL to about 50 ng/mL, about 25.0 ng/ml to about 50 ng/mL, about 27.5 ng/ml to 50 ng/mL, or about 30 ng/ml to about 50 ng/mL, about 0.5 ng/ml, about 0.9 ng/mL, about 1.0 ng/mL, about 1.25 ng/ml, about 1.5 ng/mL, about 1.75 ng/mL, about 2.0 ng/mL, about 2.25 ng/ml, about 2.5 ng/ml, about 3.0 ng/ml, about 4.0 ng/ml, about 5.0 ng/ml, about 7.5 ng/mL, about 8.0 ng/ml, about 9.0 ng/mL, about 10.0 ng/mL, about 12.5 ng/mL, about 15.0 ng/mL, about 17.5 ng/mL, about 20.0 ng/ml, about 22.5 ng/ml, about 25.0 ng/ml, about 27.5 ng/mL, or about 30 ng/mL, or at least about 0.5 ng/ml, about 0.9 ng/ml, about 1.0 ng/ml, about 1.25 ng/ml, about 1.5 ng/ml, about 1.75 ng/ml, about 2.0 ng/ml, about 2.25 ng/ml, about 2.5 ng/mL, about 3.0 ng/mL, about 4.0 ng/ml, about 5.0 ng/mL, about 7.5 ng/ml, about 8.0 ng/mL, about 9.0 ng/mL, about 10.0 ng/ml, about 12.5 ng/ml, about 15.0 ng/ml, about 17.5 ng/mL, about 20.0 ng/mL, about 22.5 ng/mL, about 25.0 ng/ml, about 27.5 ng/mL, or about 30 ng/mL, which may be as determined by ELISA. In some embodiments, the antibody or antigen-binding fragment binds to a spike(S) protein RBD from one, two, three, four, or five sarbecoviruses, in particular embodiments SARS-CoV and SARS-CoV-2.

In some embodiments, the antibody or antigen-binding fragment is capable of binding to a first and a second sarbecovirus each independently selected from Clade 1b or SARS-CoV and SARS-CoV-2, wherein the antibody or antigen-binding fragment and is capable of binding to the first sarbecovirus with an EC50 of about 0.5 ng/ml, about 0.9 ng/ml, about 1.0 ng/ml, about 1.25 ng/mL, about 1.5 ng/ml, about 1.75 ng/ml, about 2.0 ng/ml, about 2.25 ng/ml, about 2.5 ng/mL, about 3.0 ng/ml, about 4.0 ng/mL, about 5.0 ng/ml, about 7.5 ng/ml, about 8.0 ng/ml, about 9.0 ng/mL, about 10.0 ng/mL, about 12.5 ng/mL, about 15.0 ng/mL, about 17.5 ng/ml, about 20.0 ng/ml, about 22.5 ng/ml, about 25.0 ng/mL, about 27.5 ng/mL, or about 30 ng/ml, and is capable of binding to the second sarbecovirus with an EC50 of about 7.5 ng/ml, about 8.0 ng/mL, about 9.0 ng/mL, about 10.0 ng/mL, about 12.5 ng/mL, about 15.0 ng/ml, about 17.5 ng/ml, about 20.0 ng/mL, about 22.5 ng/mL, about 25.0 ng/ml, about 27.5 ng/mL, or about 30 ng/mL.

In certain examples, binding can be determined by recombinantly expressing a sarbecovirus antigen in a host cell (e.g., by transfection) and immunostaining the (e.g., fixed, or fixed and permeabilized) host cell with antibody and analyzing binding by flow cytometry (e.g., using a ZE5 Cell Analyzer (BioRad®) and FlowJo software (TreeStar). In some embodiments, positive binding can be defined by differential staining by antibody of sarbecovirus-expressing cells versus control (e.g., mock) cells.

In some embodiments, an antibody or antigen-binding fragment of the present disclosure binds to a sarbecovirus spike protein (i.e., from two or more, three or more, four or more, or five or more sarbecoviruses) expressed on the surface of a host cell (e.g., an Expi-CHO cell), as determined by flow cytometry.

In some embodiments an antibody or antigen-binding fragment of the present disclosure binds to a sarbecovirus S protein, as measured using biolayer interferometry.

In certain embodiments, an antibody of the present disclosure is capable of neutralizing infection by one or more, or by two or more, sarbecoviruses. As used herein, a “neutralizing antibody” is one that can neutralize, i.e., prevent, inhibit, reduce, impede, or interfere with, the ability of a pathogen to initiate and/or perpetuate an infection in a host. The terms “neutralizing antibody” and “an antibody that neutralizes” or “antibodies that neutralize” are used interchangeably herein. In any of the presently disclosed embodiments, the antibody or antigen-binding fragment is capable of preventing and/or neutralizing infection by one or more, or by two or more, sarbecoviruses in an in vitro model of infection and/or in an in vivo animal model of infection and/or in a human.

In some embodiments, the antibody is (or the antigen-binding fragment is from) S2V29-v37.2 or variant antibody or antigen-binding fragment thereof that comprises the CDRs and optionally at least a portion of the VH and VL of S2V29-v37.2 or a variant antibody thereof disclosed herein. In some embodiments, the S2V29 antibody or an antigen-binding fragment comprises sufficient CDRH1-H3, CDRL1-L3, VH, and/or VL identity to S2V29-v.37.2, including, in some embodiments, exact identity, to confer similar specific binding to SARS-CoV-2 in a range between about 0.5 ng/mL to about 100 ng/mL. Similar specific binding to another sarbecovirus, such as SARS-CoV, may also be present. In some embodiments, an antibody or an antigen-binding fragment comprises sufficient CDRH1-H3, CDRL1-L3, VH, and/or VL identity to S2V29-v.37.2, including, in some embodiments, exact identity, to be capable of neutralizing infection by SARS-CoV-2 in a pseudovirus system (e.g., MLV-pp-based or VSV-pp-based), or in a live virus assay in a range between about 0.5 ng/ml to about 500 ng/ml, 0.5 ng/ml to about 100 ng/mL, about 50 ng/mL to about 500 ng/ml, or about 50 ng/ml to about 100 ng/ml. Similar specific binding to another sarbecovirus, such as SARS-CoV, may also be present.

In certain embodiments, the antibody or antigen-binding fragment (i) recognizes an epitope in the Spike protein of two or more sarbecoviruses; (ii) is capable of blocking an interaction between the Spike protein of one or more sarbecoviruses and a cell surface receptor; (iii) recognizes an epitope that is conserved in the Spike protein of two or more sarbecoviruses; (iv) is cross-reactive against two or more sarbecoviruses; or (v) any combination of (i)-(iv).

Terms understood by those in the art of antibody technology are each given the meaning acquired in the art, unless expressly defined differently herein. For example, the term “antibody” refers to an intact antibody comprising at least two heavy (H) chains and two light (L) chains inter-connected by disulfide bonds, as well as any antigen-binding portion or fragment of an intact antibody that has or retains the ability to bind to the antigen target molecule recognized by the intact antibody, such as an scFv, Fab, or Fab′2 fragment. Thus, the term “antibody” herein is used in the broadest sense and includes polyclonal and monoclonal antibodies, including intact antibodies and functional (antigen-binding) antibody fragments thereof, including fragment antigen-binding (Fab) fragments, F(ab′) 2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, tetrabodies, tandem di-scFv, and tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof (IgG1, IgG2, IgG3, IgG4), IgM, IgE, IgA, and IgD.

The terms “V_{L}” or “VL” and “V_{H}” or “VH” refer to the variable binding region (also referred to as a variable domain) from an antibody light chain and an antibody heavy chain, respectively. In certain embodiments, a VL is a kappa (κ) class (also “VK” herein). In certain embodiments, a VL is a lambda (λ) class. In certain embodiments, the VL of S2V29 antibodies, particularly S2V29-v37.2, is a lambda (λ) class. The variable binding regions comprise discrete, well-defined sub-regions known as “complementarity determining regions” (CDRs) and “framework regions” (FRs). The terms “complementarity determining region,” and “CDR,” are synonymous with “hypervariable region” or “HVR,” and refer to sequences of amino acids within antibody variable regions, which, in general, together confer the antigen specificity and/or binding affinity of the antibody, wherein consecutive CDRs (i.e., CDR1 and CDR2, CDR2 and CDR3) are separated from one another in primary structure by a framework region. There are three CDRs in each variable region (HCDR1, HCDR2, HCDR3; LCDR1, LCDR2, LCDR3; also referred to as CDRHs and CDRLs, or CDRH1-3 and CDRL1-3, respectively). In certain embodiments, an antibody VH comprises four FRs and three CDRs as follows: FR1-HCDR1-FR2-HCDR2-FR3-HCDR3-FR4, and an antibody VL comprises four FRs and three CDRs as follows: FR1-LCDR1-FR2-LCDR2-FR3-LCDR3-FR4. In general, the VH and the VL together form the antigen-binding site through their respective CDRs.

As used herein, a “variant” of a CDR refers to a functional variant of a CDR sequence having up to 1-3 amino acid substitutions (e.g., conservative or non-conservative substitutions), deletions, or combinations thereof.

Numbering of CDR and framework regions may be according to any known method, system, or scheme, such as the Kabat, Chothia, EU, IMGT, and AHo numbering schemes (see, e.g., Kabat et al., “Sequences of Proteins of Immunological Interest, US Dept. Health and Human Services, Public Health Service National Institutes of Health, 1991, 5^{th} ed.; Chothia and Lesk, J. Mol. Biol. 196:901-917 (1987)); Lefranc et al., Dev. Comp. Immunol. 27:55, 2003; Honegger and Plückthun, J. Mol. Bio. 309:657-670 (2001)). Equivalent residue positions can be annotated and for different molecules to be compared using Antigen receptor Numbering And Receptor Classification (ANARCI) software tool (2016, Bioinformatics 15:298-300). Accordingly, identification of CDRs of an exemplary variable domain (VH or VL) sequence as provided herein according to one numbering scheme is not exclusive of an antibody comprising CDRs of the same variable domain as determined using a different numbering scheme.

In any of the presently disclosed embodiments, the antibody or antigen-binding fragment is capable of preventing and/or neutralizing infection by one or more, or by two or more sarbecoviruses in an in vitro model of infection and/or in an in vivo animal model of infection and/or in a human.

In certain embodiments, the antibody or antigen-binding fragment comprises a S2V29 VH.22, VH.24, VH.25, VH. 30, VH. 31, VH.32, VH.37, or VH.37 and any S2V29 VL disclosed herein, such as the S2V29 parental VL (also referred to as VL.1) or VL.2.

In certain embodiments, the antibody or antigen-binding fragment comprises a VH comprising, consisting essentially of, or consisting of an amino acid sequence according to any one of SEQ ID NOs: 166, 170, 174, 179, 182, or 184.

In certain embodiments, the antibody or antigen-binding fragment comprises a VH comprising, consisting essentially of, or consisting of an amino acid sequence according to any one of SEQ ID NOs: 184 and 187-202. In more specific embodiments the substitutions in SEQ ID NOs: 188 and 193 are conservative substitutions. In further embodiments, the antibody or antigen-binding fragment may comprise a VL comprising, consisting essentially of, or consisting of an amino acid sequence according to SEQ ID NO: 113.

In certain embodiments, the antibody or antigen-binding fragment is provided comprising a VH comprising, consisting essentially of, or consisting of an amino acid sequence according to any one of SEQ ID NOs: 67, 81, 84, 87, 90, 93, 97, 101, 105, 109, 137, 139, 142, 166, 170, 174, 179, 182, and 184, and a VL comprising, consisting essentially of, or consisting of an amino acid sequence according to any one of SEQ ID NOs: 71, 77, 113, 116, 119, 122, 125, 128, 131, 133, 145, 150, 152, 155, 159, and 163. In some embodiments, the antibody may not include both a VH consisting of an amino acid sequence according to SEQ ID NO: 67 and a VL consisting of an amino acid sequence according to SEQ ID NO: 71.

In certain embodiments, the antibody or antigen-binding fragment is provided comprising a VH comprising, consisting essentially of, or consisting of an amino acid sequence according to any one of SEQ ID NOs: 166, 170, 174, 179, 182, and 184, and a VL comprising, consisting essentially of, or consisting of an amino acid sequence according to any one of SEQ ID NOs: 71, 77, 113, 116, 119, 122, 125, 128, 131, 133, 145, 150, 152, 155, 159, and 163.

In certain embodiments, the antibody or antigen-binding fragment is provided comprising a VH and a VL that comprise, consist essentially of, or consist of the amino acid sequences according to SEQ ID NOs: 1) 166 and 113, 2) 170 and 113, 3) 174 and 113, 4) 177 and 113, 5) 179 and 113, 6) 182 and 113, or 7) 184 and 113, respectively.

In certain embodiments, the antibody or antigen-binding fragment is an S2V29-v37.2 or variant antibody or antigen-binding fragment thereof and the VH comprises an amino acid sequence according to SEQ ID NO: 184 and the VL comprises an amino acid sequence according SEQ ID NO: 113.

In certain embodiments, the antibody or antigen-binding fragment is an S2V29-v37.2 or variant antibody or antigen-binding fragment thereof and the VH comprises an amino acid sequence according SEQ ID NO: 184 and the VL consists essentially of an amino acid sequence according SEQ ID NO: 113.

In certain embodiments, the antibody or antigen-binding fragment is an S2V29-v37.2 or variant antibody or antigen-binding fragment thereof and the VH comprises an amino acid sequence according SEQ ID NO: 184 and the VL consists of an amino acid sequence according SEQ ID NO: 113.

In certain embodiments, the antibody or antigen-binding fragment is S2V29-v37.2 or variant antibody or antigen-binding fragment thereof and the VH consists essentially of an amino acid sequence according SEQ ID NO: 184 and the VL comprises an amino acid sequence according SEQ ID NO: 113.

In certain embodiments, the antibody or antigen-binding fragment is an S2V29-v37.2 or variant antibody or antigen-binding fragment thereof and the VH consists essentially of an amino acid sequence according SEQ ID NO: 184 and the VL consists essentially of an amino acid sequence according SEQ ID NO: 113.

In certain embodiments, the antibody or antigen-binding fragment is an S2V29-v37.2 or variant antibody or antigen-binding fragment thereof and the VH consists essentially of an amino acid sequence according SEQ ID NO: 184 and the VL consists of an amino acid sequence according SEQ ID NO: 113.

In certain embodiments, the antibody or antigen-binding fragment is an S2V29-v37.2 or variant antibody or antigen-binding fragment thereof and the VH consists of an amino acid sequence according SEQ ID NO: 184 and the VL comprises an amino acid sequence according SEQ ID NO: 113.

In certain embodiments, the antibody or antigen-binding fragment is an S2V29-v37.2 or variant antibody or antigen-binding fragment thereof and the VH consists of an amino acid sequence according SEQ ID NO: 184 and the VL consists essentially of an amino acid sequence according SEQ ID NO: 113.

In certain embodiments, the antibody or antigen-binding fragment is an S2V29-v37.2 antibody or antigen-binding fragment and the VH consists of an amino acid sequence according SEQ ID NO: 184 and the VL consists of an amino acid sequence according SEQ ID NO: 113. In certain embodiments, the antibody or antigen-binding fragment is an S2V29-v37.2 or variant antibody or antigen-binding fragment thereof and the VH comprises, consists essentially of, or consists of an amino acid sequence according to one of SEQ ID NOs: 187-202 and the VL comprises, consists essentially of, or consists of an amino acid sequence according to SEQ ID NO: 113.

In certain embodiments, an antibody or antigen-binding fragment is provided comprising CDRs identified in a VH sequence according to any one of SEQ ID NOs: 67, 81, 84, 87, 90, 93, 97, 101, 105, 109, 137, 139, 142, 166, 170, 174, 179, 182, and 184, and in a VL sequence according to any one of SEQ ID NOs: 71, 77, 113, 116, 119, 122, 125, 128, 131, 133, 145, 150, 152, 155, 159, and 163, as determined using any known CDR numbering method, including the Kabat, Chothia, EU, IMGT, Martin (Enhanced Chothia), Contact, and AHo numbering methods, or a combination of two or more of these. In certain embodiments, CDRs are according to the antibody numbering method developed by the Chemical Computing Group (CCG); e.g., using Molecular Operating Environment (MOE) software (www.chemcomp.com). In some embodiments, CDRs are in accordance with the IMGT numbering method.

In certain embodiments, an antibody or antigen-binding fragment is provided comprising CDRs identified in a VH sequence according to any one of SEQ ID NOs: 166, 170, 174, 179, 182, and 184 and in a VL sequence according to any one of SEQ ID NOs: 71, 77, 113, 116, 119, 122, 125, 128, 131, 133, 145, 150, 152, 155, 159, and 163, as determined using any known CDR numbering method, including the Kabat, Chothia, EU, IMGT, Martin (Enhanced Chothia), Contact, and AHo numbering methods, or a combination of two or more of these. In certain embodiments, CDRs are according to the antibody numbering method developed by the Chemical Computing Group (CCG); e.g., using Molecular Operating Environment (MOE) software (www.chemcomp.com). In some embodiments, CDRs are in accordance with the IMGT numbering method. In some embodiments, the antibody may not include both a VH consisting of an amino acid sequence according to SEQ ID NO: 67 and a VL consisting of an amino acid sequence according to SEQ ID NO: 71.

In certain embodiments, an antibody or antigen-binding fragment is S2V29-v37.2 or variant antibody or antigen-binding fragment thereof and comprises (e.g. three) CDRs identified in a VH sequence according to SEQ ID NO: 184 and (e.g. three CDRS identified) in a VL sequence according to SEQ ID NO: 113, as determined using any known CDR numbering method, including the Kabat, Chothia, EU, IMGT, Martin (Enhanced Chothia), Contact, and AHo numbering methods, or a combination of two or more of these. In certain embodiments, CDRs are according to the antibody numbering method developed by the Chemical Computing Group (CCG); e.g., using Molecular Operating Environment (MOE) software (www.chemcomp.com). In some embodiments, CDRs are in accordance with the IMGT numbering method. In some embodiments, the antibody may not include both a VH consisting of an amino acid sequence according to SEQ ID NO: 67 and a VL consisting of an amino acid sequence according to SEQ ID NO: 71.

In certain embodiments, an antibody or an antigen-binding fragment is provided comprising a heavy chain variable domain (VH) comprising a CDRH1, a CDRH2, and a CDRH3, and a light chain variable domain (VL) comprising a CDRL1, a CDRL2, and a CDRL3, wherein: (i) the CDRH1 comprises or consists of the amino acid sequence according to SEQ ID NO: 68 or a sequence variant thereof comprising one, two, or three acid substitutions, one or more of which substitutions is optionally a conservative substitution and/or is a substitution to a germline-encoded amino acid; (ii) the CDRH2 comprises or consists of the amino acid sequence according to any one of SEQ ID NOs: 69, 82, 85, 88, 91, 94, 98, 102, 106, 110, 140, 167, 171, 175, and 180, or a sequence variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence according to any one of SEQ ID NOs: 70, 95, 99, 103, 111, 168, and 172, or a sequence variant thereof comprising one, two, or three amino acid substitutions, one or more of which substitutions is optionally a conservative substitution and/or is a substitution to a germline-encoded amino acid; (iv) the CDRL1 comprises or consists of the amino acid sequence according to any one of SEQ ID NOs: 72, 146, and 156, or a sequence variant thereof comprising one, two, or three amino acid substitutions, one or more of which substitutions is optionally a conservative substitution and/or is a substitution to a germline-encoded amino acid; (v) the CDRL2 comprises or consists of the amino acid sequence according to any one of SEQ ID NOs: 73, 147, and 160, or a sequence variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence according to any one of SEQ ID NOs: 74, 78, 114, 117, 120, 123, 126, 148, 153, 157, 161, and 164, or a sequence variant thereof comprising having one, two, or three amino acid substitutions, one or more of which substitutions is optionally a conservative substitution and/or is a substitution to a germline-encoded amino acid, wherein the antibody or antigen-binding fragment is capable of binding to a surface glycoprotein of one or more or two or more sarbecoviruses expressed on a cell surface of a host cell. In some embodiments, the antibody may not include both a VH consisting of an amino acid sequence according to SEQ ID NO: 67 and a VL consisting of an amino acid sequence according to SEQ ID NO: 71.

In certain embodiments, an antibody or an antigen-binding fragment is provided comprising a heavy chain variable domain (VH) comprising a CDRH1, a CDRH2, and a CDRH3, and a light chain variable domain (VL) comprising a CDRL1, a CDRL2, and a CDRL3, wherein: (i) the CDRH1 comprises or consists of the amino acid sequence according to SEQ ID NO: 68 or a sequence variant thereof comprising one, two, or three acid substitutions, one or more of which substitutions is optionally a conservative substitution and/or is a substitution to a germline-encoded amino acid; (ii) the CDRH2 comprises or consists of the amino acid sequence according to any one of SEQ ID NOs: 167, 171, 175, and 180, or a sequence variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence according to any one of SEQ ID NOs: 70, 168, and 172, or a sequence variant thereof comprising one, two, or three amino acid substitutions, one or more of which substitutions is optionally a conservative substitution and/or is a substitution to a germline-encoded amino acid; (iv) the CDRL1 comprises or consists of the amino acid sequence according to any one of SEQ ID NOs: 72, 146, and 156, or a sequence variant thereof comprising one, two, or three amino acid substitutions, one or more of which substitutions is optionally a conservative substitution and/or is a substitution to a germline-encoded amino acid; (v) the CDRL2 comprises or consists of the amino acid sequence according to any one of SEQ ID NOs: 73, 147, and 160, or a sequence variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence according to any one of SEQ ID NOs: 74, 78, 114, 117, 120, 123, 126, 148, 153, 157, 161, and 164, or a sequence variant thereof comprising having one, two, or three amino acid substitutions, one or more of which substitutions is optionally a conservative substitution and/or is a substitution to a germline-encoded amino acid, wherein the antibody or antigen-binding fragment is capable of binding to a surface glycoprotein of one or more or two or more sarbecoviruses expressed on a cell surface of a host cell. In some embodiments, the antibody may not include both a VH consisting of an amino acid sequence according to SEQ ID NO: 67 and a VL consisting of an amino acid sequence according to SEQ ID NO: 71.

In certain embodiments, an antibody or an antigen-binding fragment is a S2V29-v37.2 or variant antibody or antigen-binding fragment thereof and comprises a heavy chain variable domain (VH) comprising a CDRH1, a CDRH2, and a CDRH3, and a light chain variable domain (VL) comprising a CDRL1, a CDRL2, and a CDRL3, wherein: (i) the CDRH1 comprises, consists essentially of, or consists of the amino acid sequence according to SEQ ID NO: 68 or a sequence variant thereof comprising one, two, or three acid substitutions, one or more of which substitutions is optionally a conservative substitution and/or is a substitution to a germline-encoded amino acid; (ii) the CDRH2 comprises, consists essentially of, or consists of the amino acid sequence according to SEQ ID NO: 171, or a sequence variant thereof comprising one, two, or three amino acid substitutions, one or more of which substitutions is optionally a conservative substitution and/or is a substitution to a germline-encoded amino acid; (iii) the CDRH3 comprises, consists essentially of or consists of the amino acid sequence according to SEQ ID NO: 70, or a sequence variant thereof comprising one, two, or three amino acid substitutions, one or more of which substitutions is optionally a conservative substitution and/or is a substitution to a germline-encoded amino acid; (iv) the CDRL1 comprises, consists essentially of, or consists of the amino acid sequence according to SEQ ID NO: 72, or a sequence variant thereof comprising one, two, or three amino acid substitutions, one or more of which substitutions is optionally a conservative substitution and/or is a substitution to a germline-encoded amino acid; (v) the CDRL2 comprises, consists essentially of, or consists of the amino acid sequence according to SEQ ID NO: 73, or a sequence variant thereof comprising one, two, or three amino acid substitutions, one or more of which substitutions is optionally a conservative substitution and/or is a substitution to a germline-encoded amino acid; and/or (vi) the CDRL3 comprises, consists essentially of, or consists of the amino acid sequence according to SEQ ID NO: 114, or a sequence variant thereof comprising having one, two, or three amino acid substitutions, one or more of which substitutions is optionally a conservative substitution and/or is a substitution to a germline-encoded amino acid, wherein the antibody or antigen-binding fragment is capable of binding to a surface glycoprotein of one or more or two or more sarbecoviruses expressed on a cell surface of a host cell. In some embodiments, the antibody may not include both a VH consisting of an amino acid sequence according to SEQ ID NO: 67 and a VL consisting of an amino acid sequence according to SEQ ID NO: 71.

In certain embodiments, an antibody or an antigen-binding fragment is a S2V29-v37.2 or variant antibody or antigen-binding fragment thereof and comprises a heavy chain variable domain (VH) comprising a CDRH1, a CDRH2, and a CDRH3, and a light chain variable domain (VL) comprising a CDRL1, a CDRL2, and a CDRL3, wherein: (i) the CDRH1 comprises or consists of the amino acid sequence according to SEQ ID NO: 68 or a sequence variant thereof comprising one, two, or three acid substitutions, one or more of which substitutions is optionally a conservative substitution and/or is a substitution to a germline-encoded amino acid; (ii) the CDRH2 comprises or consists of the amino acid sequence according to SEQ ID NO: 171, or a sequence variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence according to SEQ ID NO: 70, or a sequence variant thereof comprising one, two, or three amino acid substitutions, one or more of which substitutions is optionally a conservative substitution and/or is a substitution to a germline-encoded amino acid; (iv) the CDRL1 comprises or consists of the amino acid sequence according to SEQ ID NO: 72, or a sequence variant thereof comprising one, two, or three amino acid substitutions, one or more of which substitutions is optionally a conservative substitution and/or is a substitution to a germline-encoded amino acid; (v) the CDRL2 comprises or consists of the amino acid sequence according to SEQ ID NO: 73, or a sequence variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence according to SEQ ID NO: 114, or a sequence variant thereof comprising having one, two, or three amino acid substitutions, one or more of which substitutions is optionally a conservative substitution and/or is a substitution to a germline-encoded amino acid, wherein the antibody or antigen-binding fragment is capable of binding to a surface glycoprotein of one or more or two or more sarbecoviruses expressed on a cell surface of a host cell. In some embodiments, the antibody may not include both a VH consisting of an amino acid sequence according to SEQ ID NO: 67 and a VL consisting of an amino acid sequence according to SEQ ID NO: 71.

In certain embodiments, an antibody or an antigen-binding fragment is a S2V29-v37.2 or variant antibody or antigen-binding fragment thereof and comprises a heavy chain variable domain (VH) comprising a CDRH1, a CDRH2, and a CDRH3, and a light chain variable domain (VL) comprising a CDRL1, a CDRL2, and a CDRL3, wherein: (i) the CDRH1 comprises the amino acid sequence according to SEQ ID NO: 68 or a sequence variant thereof comprising one, two, or three acid substitutions, one or more of which substitutions is optionally a conservative substitution and/or is a substitution to a germline-encoded amino acid; (ii) the CDRH2 comprises the amino acid sequence according to SEQ ID NO: 171, or a sequence variant thereof comprising one, two, or three amino acid substitutions, one or more of which substitutions is optionally a conservative substitution and/or is a substitution to a germline-encoded amino acid; (iii) the CDRH3 comprises the amino acid sequence according to SEQ ID NO: 70, or a sequence variant thereof comprising one, two, or three amino acid substitutions, one or more of which substitutions is optionally a conservative substitution and/or is a substitution to a germline-encoded amino acid; (iv) the CDRL1 comprises the amino acid sequence according to SEQ ID NO: 72, or a sequence variant thereof comprising one, two, or three amino acid substitutions, one or more of which substitutions is optionally a conservative substitution and/or is a substitution to a germline-encoded amino acid; (v) the CDRL2 comprises the amino acid sequence according to SEQ ID NO: 73, or a sequence variant thereof comprising one, two, or three amino acid substitutions, one or more of which substitutions is optionally a conservative substitution and/or is a substitution to a germline-encoded amino acid; and/or (vi) the CDRL3 comprises the amino acid sequence according to SEQ ID NO: 114, or a sequence variant thereof comprising having one, two, or three amino acid substitutions, one or more of which substitutions is optionally a conservative substitution and/or is a substitution to a germline-encoded amino acid, wherein the antibody or antigen-binding fragment is capable of binding to a surface glycoprotein of one or more or two or more sarbecoviruses expressed on a cell surface of a host cell. In some embodiments, the antibody may not include both a VH consisting of an amino acid sequence according to SEQ ID NO: 67 and a VL consisting of an amino acid sequence according to SEQ ID NO: 71.

In certain embodiments, an antibody or an antigen-binding fragment is a S2V29-v37.2 or variant antibody or antigen-binding fragment thereof and comprises a heavy chain variable domain (VH) comprising a CDRH1, a CDRH2, and a CDRH3, and a light chain variable domain (VL) comprising a CDRL1, a CDRL2, and a CDRL3, wherein: (i) the CDRH1 consists essentially of the amino acid sequence according to SEQ ID NO: 68 or a sequence variant thereof comprising one, two, or three acid substitutions, one or more of which substitutions is optionally a conservative substitution and/or is a substitution to a germline-encoded amino acid; (ii) the CDRH2 consists essentially of the amino acid sequence according to SEQ ID NO: 171, or a sequence variant thereof comprising one, two, or three amino acid substitutions, one or more of which substitutions is optionally a conservative substitution and/or is a substitution to a germline-encoded amino acid; (iii) the CDRH3 consists essentially of the amino acid sequence according to SEQ ID NO: 70, or a sequence variant thereof comprising one, two, or three amino acid substitutions, one or more of which substitutions is optionally a conservative substitution and/or is a substitution to a germline-encoded amino acid; (iv) the CDRL1 consists essentially of the amino acid sequence according to SEQ ID NO: 72, or a sequence variant thereof comprising one, two, or three amino acid substitutions, one or more of which substitutions is optionally a conservative substitution and/or is a substitution to a germline-encoded amino acid; (v) the CDRL2 consists essentially of the amino acid sequence according to SEQ ID NO: 73, or a sequence variant thereof comprising one, two, or three amino acid substitutions, one or more of which substitutions is optionally a conservative substitution and/or is a substitution to a germline-encoded amino acid; and/or (vi) the CDRL3 consists essentially of the amino acid sequence according to SEQ ID NO: 114, or a sequence variant thereof comprising having one, two, or three amino acid substitutions, one or more of which substitutions is optionally a conservative substitution and/or is a substitution to a germline-encoded amino acid, wherein the antibody or antigen-binding fragment is capable of binding to a surface glycoprotein of one or more or two or more sarbecoviruses expressed on a cell surface of a host cell. In some embodiments, the antibody may not include both a VH consisting of an amino acid sequence according to SEQ ID NO: 67 and a VL consisting of an amino acid sequence according to SEQ ID NO: 71.

In certain embodiments, an antibody or an antigen-binding fragment is a S2V29-v37.2 or variant antibody or antigen-binding fragment thereof and comprises a heavy chain variable domain (VH) comprising a CDRH1, a CDRH2, and a CDRH3, and a light chain variable domain (VL) comprising a CDRL1, a CDRL2, and a CDRL3, wherein: (i) the CDRH1 consists of the amino acid sequence according to SEQ ID NO: 68 or a sequence variant thereof comprising one, two, or three acid substitutions, one or more of which substitutions is optionally a conservative substitution and/or is a substitution to a germline-encoded amino acid; (ii) the CDRH2 consists of the amino acid sequence according to SEQ ID NO: 171, or a sequence variant thereof comprising one, two, or three amino acid substitutions, one or more of which substitutions is optionally a conservative substitution and/or is a substitution to a germline-encoded amino acid; (iii) the CDRH3 consists of the amino acid sequence according to SEQ ID NO: 70, or a sequence variant thereof comprising one, two, or three amino acid substitutions, one or more of which substitutions is optionally a conservative substitution and/or is a substitution to a germline-encoded amino acid; (iv) the CDRL1 consists of the amino acid sequence according to SEQ ID NO: 72, or a sequence variant thereof comprising one, two, or three amino acid substitutions, one or more of which substitutions is optionally a conservative substitution and/or is a substitution to a germline-encoded amino acid; (v) the CDRL2 consists of the amino acid sequence according to SEQ ID NO: 73, or a sequence variant thereof comprising one, two, or three amino acid substitutions, one or more of which substitutions is optionally a conservative substitution and/or is a substitution to a germline-encoded amino acid; and/or (vi) the CDRL3 consists of the amino acid sequence according to SEQ ID NO: 114, or a sequence variant thereof comprising having one, two, or three amino acid substitutions, one or more of which substitutions is optionally a conservative substitution and/or is a substitution to a germline-encoded amino acid, wherein the antibody or antigen-binding fragment is capable of binding to a surface glycoprotein of one or more or two or more sarbecoviruses expressed on a cell surface of a host cell. In some embodiments, the antibody may not include both a VH consisting of an amino acid sequence according to SEQ ID NO: 67 and a VL consisting of an amino acid sequence according to SEQ ID NO: 71. In some embodiments, the antibody or antigen-binding fragment VH may comprise an amino acid sequence according to one or more of SEQ ID NOs: 207-210, and 215-221. In some embodiments, the antibody or antigen-binding fragment VL may comprise an amino acid sequence according to one or more of SEQ ID NOs: 211-214 and 222-224.

In certain embodiments, an antibody or an antigen-binding fragment is S2V29-v37.2 or variant antibody or antigen-binding fragment thereof and comprises a heavy chain variable domain (VH) comprising a CDRH1, a CDRH2 region, and a CDRH3, and a light chain variable domain (VL) comprising a CDRL1, a CDRL2, and a CDRL3, wherein: (i) the CDRH1 consists of the amino acid sequence according to SEQ ID NO: 68 or a sequence variant thereof comprising one, two, or three acid substitutions, one or more of which substitutions is optionally a conservative substitution and/or is a substitution to a germline-encoded amino acid; (ii) the CDRH2 region consists of the amino acid sequence according to SEQ ID NO: 215, or a sequence variant thereof comprising one, two, or three amino acid substitutions, one or more of which substitutions is optionally a conservative substitution and/or is a substitution to a germline-encoded amino acid; (iii) the CDRH3 consists of the amino acid sequence according to SEQ ID NO: 70, or a sequence variant thereof comprising one, two, or three amino acid substitutions, one or more of which substitutions is optionally a conservative substitution and/or is a substitution to a germline-encoded amino acid; (iv) the CDRL1 consists of the amino acid sequence according to SEQ ID NO: 72, or a sequence variant thereof comprising one, two, or three amino acid substitutions, one or more of which substitutions is optionally a conservative substitution and/or is a substitution to a germline-encoded amino acid; (v) the CDRL2 consists of the amino acid sequence according to SEQ ID NO: 73, or a sequence variant thereof comprising one, two, or three amino acid substitutions, one or more of which substitutions is optionally a conservative substitution and/or is a substitution to a germline-encoded amino acid; and/or (vi) the CDRL3 consists of the amino acid sequence according to SEQ ID NO: 114, or a sequence variant thereof comprising having one, two, or three amino acid substitutions, one or more of which substitutions is optionally a conservative substitution and/or is a substitution to a germline-encoded amino acid, wherein the antibody or antigen-binding fragment is capable of binding to a surface glycoprotein of one or more or two or more sarbecoviruses expressed on a cell surface of a host cell. In some embodiments, the antibody may not include both a VH consisting of an amino acid sequence according to SEQ ID NO: 67 and a VL consisting of an amino acid sequence according to SEQ ID NO: 71. In some embodiments, the antibody or antigen-binding fragment VH may comprise an amino acid sequence according to one or more of SEQ ID NOs: 207-210, and 216-221. In some embodiments, the antibody or antigen-binding fragment VL may comprise an amino acid sequence according to one or more of SEQ ID NOs: 211-214 and 222-224.

In certain embodiments, an antibody or an antigen-binding fragment is a S2V29-v37.2 or variant antibody or antigen-binding fragment thereof and comprises a heavy chain variable domain (VH) comprising a CDRH1, a CDRH2 region, and a CDRH3, and a light chain variable domain (VL) comprising a CDRL1, a CDRL2, and a CDRL3, wherein: (i) the CDRH1 consists of the amino acid sequence according to SEQ ID NO: 68 or a sequence variant thereof comprising one, two, or three acid substitutions, one or more of which substitutions is optionally a conservative substitution and/or is a substitution to a germline-encoded amino acid; (ii) the CDRH2 region consists of the amino acid sequence according to SEQ ID NO: 216, or a sequence variant thereof comprising one, two, or three amino acid substitutions, one or more of which substitutions is optionally a conservative substitution and/or is a substitution to a germline-encoded amino acid; (iii) the CDRH3 consists of the amino acid sequence according to SEQ ID NO: 70, or a sequence variant thereof comprising one, two, or three amino acid substitutions, one or more of which substitutions is optionally a conservative substitution and/or is a substitution to a germline-encoded amino acid; (iv) the CDRL1 consists of the amino acid sequence according to SEQ ID NO: 72, or a sequence variant thereof comprising one, two, or three amino acid substitutions, one or more of which substitutions is optionally a conservative substitution and/or is a substitution to a germline-encoded amino acid; (v) the CDRL2 consists of the amino acid sequence according to SEQ ID NO: 73, or a sequence variant thereof comprising one, two, or three amino acid substitutions, one or more of which substitutions is optionally a conservative substitution and/or is a substitution to a germline-encoded amino acid; and/or (vi) the CDRL3 consists of the amino acid sequence according to SEQ ID NO: 114, or a sequence variant thereof comprising having one, two, or three amino acid substitutions, one or more of which substitutions is optionally a conservative substitution and/or is a substitution to a germline-encoded amino acid, wherein the antibody or antigen-binding fragment is capable of binding to a surface glycoprotein of one or more or two or more sarbecoviruses expressed on a cell surface of a host cell. In some embodiments, the antibody may not include both a VH consisting of an amino acid sequence according to SEQ ID NO: 67 and a VL consisting of an amino acid sequence according to SEQ ID NO: 71. In some embodiments, the antibody or antigen-binding fragment VH may comprise an amino acid sequence according to one or more of SEQ ID NOs: 207-210, and 217-221. In some embodiments, the antibody or antigen-binding fragment VL may comprise an amino acid sequence according to one or more of SEQ ID NOs: 211-214 and 222-224.

In certain embodiments, the antibody or antigen-binding fragment comprises CDRH1, CDRH2, CDRH3, CDRL1, CDRL2, and CDRL3 amino acid sequences according to SEQ ID NOs: 1) 68, 167, 168, 72, 73, and 114; 2) 68, 171, 172, 72 73, and 114; 3) 68, 175, 172, 72, 73, and 114; 4) 68, 180, 172, 72, 73, and 114; 5) 68, 180, 70, 72, 73, and 114; or 6) 68, 171, 70, 72, 73, and 114, respectively.

In certain embodiments, the antibody or antigen-binding fragment is a S2V29-v37.2 or variant antibody or antigen-binding fragment thereof and comprises CDRH1, CDRH2, CDRH3, CDRL1, CDRL2, and CDRL3 amino acid sequences according to SEQ ID NOs: 68, 171, 70, 72, 73, and 114, respectively. In some embodiments, the antibody or antigen-binding fragment VH may comprise an amino acid sequence according to one or more of SEQ ID NOs: 207-210, and 215-221. In some embodiments, the antibody or antigen-binding fragment VL may comprise an amino acid sequence according to one or more of SEQ ID NOs: 211-214 and 222-224.

In certain embodiments, the antibody or antigen-binding fragment is a S2V29-v37.2 or variant antibody or antigen-binding fragment thereof and comprises a VH or VL comprising SEQ ID NOs: 184 and 113, respectively, or at having at least 85%, 90%, or 95% identity to these sequences. In some embodiments, the VH having at least 85%, 90%, or 95% identity to SEQ ID NO: 184 may comprise an amino acid sequence according to any one of SEQ ID NOs: 184-202. In some embodiments, the VH having at least 85%, 90%, or 95% identity to SEQ ID NO: 184 may consist essentially of an amino acid sequence according to any one of SEQ ID NOs: 184-202. In some embodiments, the VH having at least 85%, 90%, or 95% identity to SEQ ID NO: 184 may consist of an amino acid sequence according to any one of SEQ ID NOs: 184-202. In some embodiments, the VH having at least 85%, 90%, or 95% identity to SEQ ID NO: 184 may comprise an amino acid sequence according to one or more of SEQ ID NOs: 215-221. In some embodiments, the VH having at least 85%, 90%, or 95% identity to SEQ ID NO: 184 may comprise an amino acid sequence according to one or more of SEQ ID NOs: 207-210. In some embodiments, the VH having at least 85%, 90%, or 95% identity to SEQ ID NO: 184 may comprise an amino acid sequence according to one or more of SEQ ID NOs: 215-221 and further comprise an amino acid sequence according to SEQ ID NOs: 207-210, which may, in some embodiments, be comprised with SEQ ID NOs: 215-221. In some embodiments, the VH having at least 85%, 90%, or 95% identity to SEQ ID NO: 184 may comprise CDRH1, CDRH2, CDRH3 amino acid sequences according to SEQ ID NOs: 68, 171, and 70, and further comprise an amino acid sequence according to one or more of SEQ ID NOs: 207-210. In some embodiments, the VH having at least 85%, 90%, or 95% identity to SEQ ID NO: 184 may comprise CDRH1, CDRH2, CDRH3 amino acid sequences according to SEQ ID NOs: 68, 171, and 70, and further comprise an amino acid sequence according to one or more of SEQ ID NOs: 215-221. In some embodiments, the VH having at least 85%, 90%, or 95% identity to SEQ ID NO: 184 may comprise CDRH1, CDRH2, CDRH3 amino acid sequences according to SEQ ID NOs: 68, 171, and 70, and further comprise an amino acid sequence according to one or more of SEQ ID NOS: 207-210, and still further comprise an amino acid sequence according to one or more of SEQ ID NOs: 215-221. In some embodiments, the VL having at least 85%, 90%, or 95% identity to SEQ ID NO: 113 may comprise an amino acid sequence according to one or more of SEQ ID NOs: 222-224. In some embodiments, the VL having at least 85%, 90%, or 95% identity to SEQ ID NO: 113 may comprise an amino acid sequence according to one or more of SEQ ID NOs: 211-214. In some embodiments, the VL having at least 85%, 90%, or 95% identity to SEQ ID NO: 113 may comprise an amino acid sequence according to one or more of SEQ ID NOs: 222-224, and further comprise an amino acid sequence according to SEQ ID NOs: 211-214, which may, in some embodiments, be comprised with SEQ ID NOs: 222-224. In some embodiments, the VL having at least 85%, 90%, or 95% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 211-214. In some embodiments, the VL having at least 85%, 90%, or 95% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 222-224. In some embodiments, the VL having at least 85%, 90%, or 95% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 211-214, and still further comprise an amino acid sequence according to one or more of SEQ ID NOs: 222-224. In some embodiments, the antibody may not include both a VH consisting of an amino acid sequence according to SEQ ID NO: 67 and a VL consisting of an amino acid sequence according to SEQ ID NO: 71.

In certain embodiments, the antibody or antigen-binding fragment is a S2V29-v37.2 or variant antibody or antigen-binding fragment thereof and comprises a VH or VL consisting essentially of SEQ ID NOs: 184 and 113, respectively, or at having at least 85%, 90%, or 95% identity to these sequences. In some embodiments, the VH having at least 85%, 90%, or 95% identity to SEQ ID NO: 184 may comprise an amino acid sequence according to any one of SEQ ID NOs: 184-202. In some embodiments, the VH having at least 85%, 90%, or 95% identity to SEQ ID NO: 184 may consist essentially of an amino acid sequence according to any one of SEQ ID NOs: 184-202. In some embodiments, the VH having at least 85%, 90%, or 95% identity to SEQ ID NO: 184 may consist of an amino acid sequence according to any one of SEQ ID NOs: 184-202. In some embodiments, the VH having at least 85%, 90%, or 95% identity to SEQ ID NO: 184 may comprise an amino acid sequence according to one or more of SEQ ID NOs: 215-221. In some embodiments, the VH having at least 85%, 90%, or 95% identity to SEQ ID NO: 184 may comprise an amino acid sequence according to one or more of SEQ ID NOs: 207-210. In some embodiments, the VH having at least 85%, 90%, or 95% identity to SEQ ID NO: 184 may comprise an amino acid sequence according to one or more of SEQ ID NOs: 215-221 and further comprise an amino acid sequence according to SEQ ID NOs: 207-210, which may, in some embodiments, be comprised with SEQ ID NOs: 215-221. In some embodiments, the VH having at least 85%, 90%, or 95% identity to SEQ ID NO: 184 may comprise CDRH1, CDRH2, CDRH3 amino acid sequences according to SEQ ID NOs: 68, 171, and 70, and further comprise an amino acid sequence according to one or more of SEQ ID NOs: 207-210. In some embodiments, the VH having at least 85%, 90%, or 95% identity to SEQ ID NO: 184 may comprise CDRH1, CDRH2, CDRH3 amino acid sequences according to SEQ ID NOs: 68, 171, and 70, and further comprise an amino acid sequence according to one or more of SEQ ID NOS: 215-221. In some embodiments, the VH having at least 85%, 90%, or 95% identity to SEQ ID NO: 184 may comprise CDRH1, CDRH2, CDRH3 amino acid sequences according to SEQ ID NOs: 68, 171, and 70, and further comprise an amino acid sequence according to one or more of SEQ ID NOs: 207-210, and still further comprise an amino acid sequence according to one or more of SEQ ID NOs: 215-221. In some embodiments, the VL having at least 85%, 90%, or 95% identity to SEQ ID NO: 113 may comprise an amino acid sequence according to one or more of SEQ ID NOs: 222-224. In some embodiments, the VL having at least 85%, 90%, or 95% identity to SEQ ID NO: 113 may comprise an amino acid sequence according to one or more of SEQ ID NOs: 211-214. In some embodiments, the VL having at least 85%, 90%, or 95% identity to SEQ ID NO: 113 may comprise an amino acid sequence according to one or more of SEQ ID NOs: 222-224, and further comprise an amino acid sequence according to SEQ ID NOs: 211-214, which may, in some embodiments, be comprised with SEQ ID NOs: 222-224. In some embodiments, the VL having at least 85%, 90%, or 95% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 211-214. In some embodiments, the VL having at least 85%, 90%, or 95% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 222-224. In some embodiments, the VL having at least 85%, 90%, or 95% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 211-214, and still further comprise an amino acid sequence according to one or more of SEQ ID NOs: 222-224. In some embodiments, the antibody may not include both a VH consisting of an amino acid sequence according to SEQ ID NO: 67 and a VL consisting of an amino acid sequence according to SEQ ID NO: 71.

In certain embodiments, the antibody or antigen-binding fragment is a S2V29-v37.2 or variant antibody or antigen-binding fragment thereof and comprises a VH or VL consisting of SEQ ID NOs: 184 and 113, respectively, or at having at least 85%, 90%, or 95% identity to these sequences. In some embodiments, the VH having at least 85%, 90%, or 95% identity to SEQ ID NO: 184 may comprise an amino acid sequence according to any one of SEQ ID NOs: 184-202. In some embodiments, the VH having at least 85%, 90%, or 95% identity to SEQ ID NO: 184 may consist essentially of an amino acid sequence according to any one of SEQ ID NOs: 184-202. In some embodiments, the VH having at least 85%, 90%, or 95% identity to SEQ ID NO: 184 may consist of an amino acid sequence according to any one of SEQ ID NOs: 184-202. In some embodiments, the VH having at least 85%, 90%, or 95% identity to SEQ ID NO: 184 may comprise an amino acid sequence according to one or more of SEQ ID NOs: 215-221. In some embodiments, the VH having at least 85%, 90%, or 95% identity to SEQ ID NO: 184 may comprise an amino acid sequence according to one or more of SEQ ID NOs: 207-210. In some embodiments, the VH having at least 85%, 90%, or 95% identity to SEQ ID NO: 184 may comprise an amino acid sequence according to one or more of SEQ ID NOs: 215-221 and further comprise an amino acid sequence according to SEQ ID NOs: 207-210, which may, in some embodiments, be comprised with SEQ ID NOs: 215-221. In some embodiments, the VH having at least 85%, 90%, or 95% identity to SEQ ID NO: 184 may comprise CDRH1, CDRH2, CDRH3 amino acid sequences according to SEQ ID NOs: 68, 171, and 70, and further comprise an amino acid sequence according to one or more of SEQ ID NOs: 207-210. In some embodiments, the VH having at least 85%, 90%, or 95% identity to SEQ ID NO: 184 may comprise CDRH1, CDRH2, CDRH3 amino acid sequences according to SEQ ID NOs: 68, 171, and 70, and further comprise an amino acid sequence according to one or more of SEQ ID NOs: 215-221. In some embodiments, the VH having at least 85%, 90%, or 95% identity to SEQ ID NO: 184 may comprise CDRH1, CDRH2, CDRH3 amino acid sequences according to SEQ ID NOs: 68, 171, and 70, and further comprise an amino acid sequence according to one or more of SEQ ID NOs: 207-210, and still further comprise an amino acid sequence according to one or more of SEQ ID NOs: 215-221. In some embodiments, the VL having at least 85%, 90%, or 95% identity to SEQ ID NO: 113 may comprise an amino acid sequence according to one or more of SEQ ID NOs: 222-224. In some embodiments, the VL having at least 85%, 90%, or 95% identity to SEQ ID NO: 113 may comprise an amino acid sequence according to one or more of SEQ ID NOs: 211-214. In some embodiments, the VL having at least 85%, 90%, or 95% identity to SEQ ID NO: 113 may comprise an amino acid sequence according to one or more of SEQ ID NOs: 222-224, and further comprise an amino acid sequence according to SEQ ID NOs: 211-214, which may, in some embodiments, be comprised with SEQ ID NOs: 222-224. In some embodiments, the VL having at least 85%, 90%, or 95% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 211-214. In some embodiments, the VL having at least 85%, 90%, or 95% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 222-224. In some embodiments, the VL having at least 85%, 90%, or 95% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 211-214, and still further comprise an amino acid sequence according to one or more of SEQ ID NOs: 222-224. In some embodiments, the antibody may not include both a VH consisting of an amino acid sequence according to SEQ ID NO: 67 and a VL consisting of an amino acid sequence according to SEQ ID NO: 71.

In certain embodiments, the antibody or antigen-binding fragment is a S2V29-v37.2 or variant antibody or antigen-binding fragment thereof and comprises CDRH1, CDRH2, CDRH3, CDRL1, CDRL2, and CDRL3 amino acid sequences according to SEQ ID NOs: 68, 171, 70, 72, 73, and 114, respectively and a VH comprising, consisting essentially of, or consisting of an amino acid sequence according to any one of SEQ ID NOs: 184-202. The antibody or antigen-binding fragment may further comprise a VL, which may comprise, consist essentially of, or consist of an amino acid sequence according to SEQ ID NO: 113, or at having at least 85%, 90%, or 95% identity to SEQ ID NO: 113. In some embodiments, the VL having at least 85%, 90%, or 95% identity to SEQ ID NO: 113 may comprise an amino acid sequence according to one or more of SEQ ID NOs: 222-224. In some embodiments, the VL having at least 85%, 90%, or 95% identity to SEQ ID NO: 113 may comprise an amino acid sequence according to one or more of SEQ ID NOs: 211-214. In some embodiments, the VL having at least 85%, 90%, or 95% identity to SEQ ID NO: 113 may comprise an amino acid sequence according to one or more of SEQ ID NOs: 222-224, and further comprise an amino acid sequence according to SEQ ID NOs: 211-214, which may, in some embodiments, be comprised with SEQ ID NOs: 222-224. In some embodiments, the VL having at least 85%, 90%, or 95% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 211-214. In some embodiments, the VL having at least 85%, 90%, or 95% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 222-224. In some embodiments, the VL having at least 85%, 90%, or 95% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 211-214, and still further comprise an amino acid sequence according to one or more of SEQ ID NOs: 222-224. In some embodiments, the antibody may not include both a VH consisting of an amino acid sequence according to SEQ ID NO: 67 and a VL consisting of an amino acid sequence according to SEQ ID NO: 71.

In certain embodiments, the antibody or antigen-binding fragment is a S2V29-v37.2 or variant antibody or antigen-binding fragment thereof and comprises CDRH1, CDRH2, CDRH3, CDRL1, CDRL2, and CDRL3 amino acid sequences according to SEQ ID NOs: 68, 171, 70, 72, 73, and 114, respectively and a VH comprising an amino acid sequence according to any one of SEQ ID NOs: 214-221. The VH may further comprise an amino acid sequence according to one or more of SEQ ID NOs: 207-210, which may, in some embodiments, be comprised with SEQ ID NOs: 222-224. The antibody or antigen-binding fragment may further comprise a VL, which may comprise, consist essentially of, or consist of an amino acid sequence according to SEQ ID NO: 113, or at having at least 85%, 90%, or 95% identity to SEQ ID NO: 113. In some embodiments, the VL having at least 85%, 90%, or 95% identity to SEQ ID NO: 113 may comprise an amino acid sequence according to one or more of SEQ ID NOs: 222-224. In some embodiments, the VL having at least 85%, 90%, or 95% identity to SEQ ID NO: 113 may comprise an amino acid sequence according to one or more of SEQ ID NOs: 211-214. In some embodiments, the VL having at least 85%, 90%, or 95% identity to SEQ ID NO: 113 may comprise an amino acid sequence according to one or more of SEQ ID NOs: 222-224, and further comprise an amino acid sequence according to SEQ ID NOs: 211-214, which may, in some embodiments, be comprised with SEQ ID NOs: 222-224. In some embodiments, the VL having at least 85%, 90%, or 95% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 211-214. In some embodiments, the VL having at least 85%, 90%, or 95% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOS: 222-224. In some embodiments, the VL having at least 85%, 90%, or 95% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 211-214, and still further comprise an amino acid sequence according to one or more of SEQ ID NOs: 222-224. In some embodiments, the antibody may not include both a VH consisting of an amino acid sequence according to SEQ ID NO: 67 and a VL consisting of an amino acid sequence according to SEQ ID NO: 71.

It will be understood that, for example, production in a mammalian cell line can remove one or more C-terminal lysine of an antibody heavy chain (see, e.g., Liu et al. mAbs 6 (5): 1145-1154 (2014)). Accordingly, an antibody or antigen-binding fragment of the present disclosure can comprise a heavy chain, a CH1-CH3, a CH3, or an Fc polypeptide wherein a C-terminal lysine residue is present or is absent; in other words, encompassed are embodiments where the C-terminal residue of a heavy chain, a CH1-CH3, or an Fc polypeptide is not a lysine, and embodiments where a lysine is the C-terminal residue. In certain embodiments, a composition comprises a plurality of an antibody and/or an antigen-binding fragment of the present disclosure, wherein one or more antibody or antigen-binding fragment does not comprise a lysine residue at the C-terminal end of the heavy chain, CH1-CH3, or Fc polypeptide, and wherein one or more antibody or antigen-binding fragment comprises a lysine residue at the C-terminal end of the heavy chain, CH1-CH3, or Fc polypeptide.

A “Fab” (fragment antigen-binding) is the part of an antibody that binds to antigens and includes the variable region and CH1 of the heavy chain linked to the light chain via an inter-chain disulfide bond. Each Fab fragment is monovalent with respect to antigen-binding, i.e., it has a single antigen-binding site. Pepsin treatment of an antibody yields a single large F(ab′)2 fragment that roughly corresponds to two disulfide linked Fab fragments having divalent antigen-binding activity and is still capable of cross-linking antigen. Both the Fab and F(ab′)2 are examples of “antigen-binding fragments.” Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

Fab fragments may be joined, e.g., by a peptide linker, to form a single chain Fab, also referred to herein as “scFab.” In these embodiments, an inter-chain disulfide bond that is present in a native Fab may not be present, and the linker serves in full or in part to link or connect the Fab fragments in a single polypeptide chain. A heavy chain-derived Fab fragment (e.g., comprising, consisting of, or consisting essentially of VH+CH1, or “Fd”) and a light chain-derived Fab fragment (e.g., comprising, consisting of, or consisting essentially of VL+CL) may be linked in any arrangement to form a scFab. For example, a scFab may be arranged, in N-terminal to C-terminal direction, according to (heavy chain Fab fragment-linker-light chain Fab fragment) or (light chain Fab fragment-linker-heavy chain Fab fragment). Peptide linkers and exemplary linker sequences for use in scFabs are discussed in further detail herein.

“Fv” is a small antibody fragment that contains a complete antigen-recognition and antigen-binding site. This fragment generally consists of a dimer of one heavy- and one light-chain variable region domain in tight, non-covalent association. However, even a single variable domain (or half of an Fv comprising only three CDRs specific for an antigen) has the ability to recognize and bind antigen, although typically at a lower affinity than the entire binding site.

“Single-chain Fv” also abbreviated as “sFv” or “scFv”, are antibody fragments that comprise the V_{H} and V_{L} antibody domains connected into a single polypeptide chain. In some embodiments, the scFv polypeptide comprises a polypeptide linker disposed between and linking the VH and VL domains that enables the scFv to retain or form the desired structure for antigen-binding. Such a peptide linker can be incorporated into a fusion polypeptide using standard techniques well known in the art. For a review of scFv, see Pluckthun in The Pharmacology of Monoclonal Antibodies, vol. 113, Rosenburg and Moore eds., Springer-Verlag, New York, pp. 269-315 (1994); Borrebaeck 1995, infra. In certain embodiments, the antibody or antigen-binding fragment comprises a scFv comprising a VH domain, a VL domain, and a peptide linker linking the VH domain to the VL domain. In particular embodiments, a scFv comprises a VH domain linked to a VL domain by a peptide linker, which can be in a VH-linker-VL orientation or in a VL-linker-VH orientation. Any scFv of the present disclosure may be engineered so that the C-terminal end of the VL domain is linked by a short peptide sequence to the N-terminal end of the VH domain, or vice versa (i.e., (N)VL(C)-linker-(N)VH(C) or (N)VH(C)-linker-(N)VL(C). Alternatively, in some embodiments, a linker may be linked to an N-terminal portion or end of the VH domain, the VL domain, or both.

Peptide linker sequences may be chosen, for example, based on: (1) their ability to adopt a flexible extended conformation; (2) their inability or lack of ability to adopt a secondary structure that could interact with functional epitopes on the first and second polypeptides and/or on a target molecule; and/or (3) the lack or relative lack of hydrophobic or charged residues that might react with the polypeptides and/or target molecule. Other considerations regarding linker design (e.g., length) can include the conformation or range of conformations in which the VH and VL can form a functional antigen-binding site. In certain embodiments, peptide linker sequences contain, for example, Gly, Asn and Ser residues. Other near neutral amino acids, such as Thr and Ala, may also be included in a linker sequence. Other amino acid sequences which may be usefully employed as linker include those disclosed in Maratea et al., Gene 40:39 46 (1985); Murphy et al., Proc. Natl. Acad. Sci. USA 83:8258 8262 (1986); U.S. Pat. Nos. 4,935,233, and 4,751,180. Other illustrative and non-limiting examples of linkers may include, for example, Glu-Gly-Lys-Ser-Ser-Gly-Ser-Gly-Ser-Glu-Ser-Lys-Val-Asp (SEQ ID NO: 19) (Chaudhary et al., Proc. Natl. Acad. Sci. USA 87:1066-1070 (1990)) and Lys-Glu-Ser-Gly-Ser-Val-Ser-Ser-Glu-Gln-Leu-Ala-Gln-Phe-Arg-Ser-Leu-Asp (SEQ ID NO: 20) (Bird et al., Science 242:423-426 (1988)) and the pentamer Gly-Gly-Gly-Gly-Ser (SEQ ID NO: 21) when present in a single iteration or repeated 1 to 5 or more times, or more. Any suitable linker may be used, and in general can be about 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100 amino acids in length, or less than about 200 amino acids in length, and will preferably comprise a flexible structure (can provide flexibility and room for conformational movement between two regions, domains, motifs, fragments, or modules connected by the linker), and will preferably be biologically inert and/or have a low risk of immunogenicity in a human. Exemplary linkers include those comprising or consisting of the amino acid sequence according to any one or more of SEQ ID NOs: 4-13 and 251-252. In certain embodiments, the linker comprises or consists of an amino acid sequence having at least 75% (e.g. 75%, 80%, 85%, 90%, 91%, 92%, 93%, 94%, 95%, 96%, 97%, 98%, 99%, or more) identity to the amino acid sequence according to any one of SEQ ID NOs: 4-13 and 251-252.

scFv can be constructed using any combination of the VH and VL sequences or any combination of the CDRH1, CDRH2 or CDRH2 regions, CDRH3, CDRL1, CDRL2, and CDRL3 sequences disclosed herein.

In some embodiments, linker sequences are not required; for example, when the first and second polypeptides have non-essential N-terminal (or C-terminal) amino acid regions that can be used to separate the functional domains and prevent steric interference.

During antibody development, DNA in the germline variable (V), joining (J), and diversity (D) gene loci may be rearranged and insertions and/or deletions of nucleotides in the coding sequence may occur. Somatic mutations may be encoded by the resultant sequence, and can be identified by reference to a corresponding known germline sequence. In some contexts, somatic mutations that are not critical to a desired property of the antibody (e.g., binding to a SARS-CoV-2 antigen), or that confer an undesirable property upon the antibody (e.g., an increased risk of immunogenicity in a subject administered the antibody), or both, may be replaced by the corresponding germline-encoded amino acid, or by a different amino acid, so that a desirable property of the antibody is improved or maintained and the undesirable property of the antibody is reduced or abrogated. Thus, in some embodiments, the antibody or antigen-binding fragment of the present disclosure comprises one or more more germline-encoded amino acid in a variable region as compared to a parent antibody or antigen-binding fragment, provided that the parent antibody or antigen-binding fragment comprises one or more somatic mutations.

Variable region and CDR amino acid sequences of exemplary anti-sarbecovirus antibodies of the present disclosure are provided in Table 2, Table 3, and Table 4 herein.

In certain embodiments, an antibody or antigen-binding fragment comprises an amino acid modification (e.g., a substitution mutation) to remove an undesired risk of oxidation, deamination, and/or isomerization.

Provided herein are variant antibodies and antigen-binding fragments that comprise one or more amino acid alterations in a variable region (e.g., VH, VL, framework or CDR) as compared to the presently disclosed S2V29 antibodies, particularly a S2V29-v37.2 antibody, wherein the variant antibody or antigen-binding fragment is capable of binding to a SARS-CoV-2 antigen.

In certain embodiments, (i) the VH comprises, consists essentially of, or consists of an amino acid sequence having at least 85% (e.g. 85%, 86, 87, 88, 89, 90, 91, 92, 93, 94, 95, 96, 97, 98, 99, or 100%) identity to the amino acid sequence according to SEQ ID NO: 67, 81, 84, 87, 90, 93, 97, 101, 105, 109, 137, 139, 142, 166, 170, 174, 179, 182, or 184, wherein the variation is optionally limited to one or more framework regions and/or the variation comprises one or more substitution to a germline-encoded amino acid; and/or (ii) the VL comprises, consists essentially of, or consists of an amino acid sequence having at least 85% (e.g. 85%, 86, 87, 88, 89, 90, 91, 92, 93, 94, 95, 96, 97, 98, 99, or 100%) identity to the amino acid sequence according to SEQ ID NO: 71, 77, 113, 116, 119, 122, 125, 128, 131, 133, 145, 150, 152, 155, 159, or 163, wherein the variation is optionally limited to one or more framework regions and/or the variation comprises one or more substitution to a germline-encoded amino acid. In some embodiments, the antibody may not include both a VH consisting of an amino acid sequence according to SEQ ID NO: 67 and a VL consisting of an amino acid sequence according to SEQ ID NO: 71.

In certain embodiments, (i) the VH comprises, consists essentially of, or consists of an amino acid sequence having at least 85% (e.g. 85%, 86, 87, 88, 89, 90, 91, 92, 93, 94, 95, 96, 97, 98, 99, or 100%) identity to the amino acid sequence according to SEQ ID NO: 166, 170, 174, 179, 182, or 184, wherein the variation is optionally limited to one or more framework regions and/or the variation comprises one or more substitution to a germline-encoded amino acid; and/or (ii) the VL comprises, consists essentially of, or consists of an amino acid sequence having at least 85% (e.g. 85%, 86, 87, 88, 89, 90, 91, 92, 93, 94, 95, 96, 97, 98, 99, or 100%) identity to the amino acid sequence according to SEQ ID NO: 71, 77, 113, 116, 119, 122, 125, 128, 131, 133, 145, 150, 152, 155, 159, or 163, wherein the variation is optionally limited to one or more framework regions and/or the variation comprises one or more substitution to a germline-encoded amino acid. In some embodiments, the antibody may not include both a VH consisting of an amino acid sequence according to SEQ ID NO: 67 and a VL consisting of an amino acid sequence according to SEQ ID NO: 71.

In certain embodiments, (i) the VH comprises, consists essentially of, or consists of an amino acid sequence having at least 85% (e.g. 85%, 86, 87, 88, 89, 90, 91, 92, 93, 94, 95, 96, 97, 98, 99, or 100%) identity to the amino acid sequence according to SEQ ID NO: 166, 170, 174, 179, 182, or 184, wherein the variation is optionally limited to one or more framework regions and/or the variation comprises one or more substitution to a germline-encoded amino acid; and/or (ii) the VL comprises, consists essentially of, or consists of an amino acid sequence having at least 85% (e.g. 85%, 86, 87, 88, 89, 90, 91, 92, 93, 94, 95, 96, 97, 98, 99, or 100%) identity to the amino acid sequence according to SEQ ID NO: 113, wherein the variation is optionally limited to one or more framework regions and/or the variation comprises one or more substitution to a germline-encoded amino acid. In some embodiments, the antibody may not include both a VH consisting of an amino acid sequence according to SEQ ID NO: 67 and a VL consisting of an amino acid sequence according to SEQ ID NO: 71.

In certain embodiments, the antibody or antigen-binding fragment is a S2V29-v37.2 or variant antibody or antigen-binding fragment thereof and (i) the VH comprises, consists essentially of, or consists of an amino acid sequence having at least 85% (e.g. 85%, 86, 87, 88, 89, 90, 91, 92, 93, 94, 95, 96, 97, 98, 99, or 100%) identity to the amino acid sequence according to SEQ ID NO: 184, wherein the variation is optionally limited to one or more framework regions and/or the variation comprises one or more substitution to a germline-encoded amino acid; and/or (ii) the VL comprises, consists essentially of, or consists of an amino acid sequence having at least 85% (e.g. 85%, 86, 87, 88, 89, 90, 91, 92, 93, 94, 95, 96, 97, 98, 99, or 100%) identity to the amino acid sequence according to SEQ ID NO: 113, wherein the variation is optionally limited to one or more framework regions and/or the variation comprises one or more substitution to a germline-encoded amino acid. In some embodiments, the antibody may not include both a VH consisting of an amino acid sequence according to SEQ ID NO: 67 and a VL consisting of an amino acid sequence according to SEQ ID NO: 71.

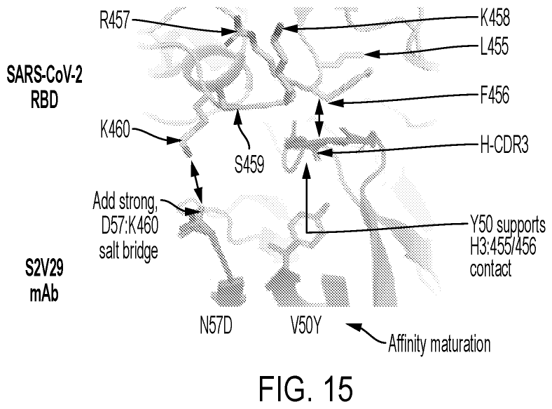

In some embodiments, the VH comprises an amino acid sequence according to any one of SEQ ID NOs: 187-201, particularly SEQ ID NO: 188 or 193. In some embodiments, the variation in the VH comprises a substitution of N57 that maintains a salt bridge to K460 of SARS-CoV-2 S protein, as illustrated in FIG. 15, in particular an acidic amino acid substitution according to SEQ ID NO: 194 or 188. In some embodiments, the variation in the VH comprises a N57D substitution and a substitution of V50 according to any one of SEQ ID NOs: 199-202. In some embodiments, the variation in the VH comprises a substitution of V50 that maintains the ability of the amino acid to support contact between amino acids 455 and 456 of the S protein, as illustrated in FIG. 15, in particular an aromatic amino acid substitution according to SEQ ID NO: 189 or 200. In some embodiments, the variation in the VH comprises a V50Y substation and a substitution of N57 according to any one of SEQ ID NOs: 196-198.

The term “CL” refers to an “immunoglobulin light chain constant region,” a “light chain constant region,” or a “light chain constant domain,” i.e., a constant region from an antibody light chain. The term “CH” refers to an “immunoglobulin heavy chain constant region,” a “heavy chain constant region,” or a “heavy chain constant domain,” which is further divisible, depending on the antibody isotype into CH1, CH2, and CH3 (IgA, IgD, IgG), or CH1, CH2, CH3, and CH4 domains (IgE, IgM). The Fc moiety of an antibody heavy chain is described further herein. In any of the presently disclosed embodiments, an antibody or antigen-binding fragment of the present disclosure comprises any one or more of CL, a CH1, a CH2, and a CH3.

In some embodiments, an antibody or antigen-binding fragment comprises an (e.g. human) IgG (e.g., IgG1, IgG2, IgG3, or IgG4), IgA, IgD, IgE, or IgM isotype, or comprises amino acid sequences from two or more of these isotypes. In some embodiments, an antibody or antigen-binding fragment comprises an IgG1 isotype; it will be understood that such an antibody or antigen-binding fragment may comprise one or more amino acid substitutions in a heavy chain constant domain and still be considered an “IgG1” isotype. In some embodiments, an IgG1 may comprise or consist of amino acid sequences having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to, or comprising or consisting of, the amino acid sequences according to SEQ ID NOs: 30-66.

Any of these antibodies or antigen-binding fragments may include an Fc modification as described herein, or may have an unmodified human IgG1 constant region.

Specifically, the antibody or antigen-binding fragment may be an S2V29-v37.2 or variant antibody or antigen-binding fragment thereof and have a VH comprising, consisting essentially of, or consisting of the amino acid sequence according to SEQ ID NO: 184 or having at least 85%, 90%, or 95% identity to SEQ ID NO: 184, a CH comprising, consisting essentially of, or consisting of an amino acid sequence according to any one of SEQ ID NOs: 30-66, particularly SEQ ID NO: 49, a VL comprising, consisting essentially of, or consisting of an amino acid sequence according to SEQ ID NO: 113, or having at least 85%, 90%, or 95% identity to SEQ ID NO: 113, and a CL comprising, consisting essentially of, or consisting of an amino acid sequence according to SEQ ID NO: 186. In some embodiments, the VH having at least 85%, 90%, or 95% identity to SEQ ID NO: 184 may comprise an amino acid sequence according to any one of SEQ ID NOs: 184-202. In some embodiments, the VH having at least 85%, 90%, or 95% identity to SEQ ID NO: 184 may consist essentially of an amino acid sequence according to any one of SEQ ID NOs: 184-202. In some embodiments, the VH having at least 85%, 90%, or 95% identity to SEQ ID NO: 184 may consist of an amino acid sequence according to any one of SEQ ID NOS: 184-202. In some embodiments, the VH having at least 85%, 90%, or 95% identity to SEQ ID NO: 184 may comprise an amino acid sequence according to one or more of SEQ ID NOs: 215-221. In some embodiments, the VH having at least 85%, 90%, or 95% identity to SEQ ID NO: 184 may comprise an amino acid sequence according to one or more of SEQ ID NOs: 207-210. In some embodiments, the VH having at least 85%, 90%, or 95% identity to SEQ ID NO: 184 may comprise an amino acid sequence according to one or more of SEQ ID NOs: 215-221 and further comprise an amino acid sequence according to SEQ ID NOs: 207-210, which may, in some embodiments, be comprised with SEQ ID NOs: 215-221. In some embodiments, the VH having at least 85%, 90%, or 95% identity to SEQ ID NO: 184 may comprise CDRH1, CDRH2, CDRH3 amino acid sequences according to SEQ ID NOs: 68, 171, and 70, and further comprise an amino acid sequence according to one or more of SEQ ID NOs: 207-210. In some embodiments, the VH having at least 85%, 90%, or 95% identity to SEQ ID NO: 184 may comprise CDRH1, CDRH2, CDRH3 amino acid sequences according to SEQ ID NOs: 68, 171, and 70, and further comprise an amino acid sequence according to one or more of SEQ ID NOs: 215-221. In some embodiments, the VH having at least 85%, 90%, or 95% identity to SEQ ID NO: 184 may comprise CDRH1, CDRH2, CDRH3 amino acid sequences according to SEQ ID NOs: 68, 171, and 70, and further comprise an amino acid sequence according to one or more of SEQ ID NOs: 207-210, and still further comprise an amino acid sequence according to one or more of SEQ ID NOs: 215-221. In some embodiments, the VL having at least 85%, 90%, or 95% identity to SEQ ID NO: 113 may comprise an amino acid sequence according to one or more of SEQ ID NOs: 222-224. In some embodiments, the VL having at least 85%, 90%, or 95% identity to SEQ ID NO: 113 may comprise an amino acid sequence according to one or more of SEQ ID NOs: 211-214. In some embodiments, the VL having at least 85%, 90%, or 95% identity to SEQ ID NO: 113 may comprise an amino acid sequence according to one or more of SEQ ID NOs: 222-224, and further comprise an amino acid sequence according to SEQ ID NOs: 211-214, which may, in some embodiments, be comprised with SEQ ID NOs: 222-224. In some embodiments, the VL having at least 85%, 90%, or 95% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 211-214. In some embodiments, the VL having at least 85%, 90%, or 95% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 222-224. In some embodiments, the VL having at least 85%, 90%, or 95% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 211-214, and still further comprise an amino acid sequence according to one or more of SEQ ID NOs: 222-224. In some embodiments, the antibody may not include both a VH consisting of an amino acid sequence according to SEQ ID NO: 67 and a VL consisting of an amino acid sequence according to SEQ ID NO: 71.

In some embodiments, the S2V29-v37.2 or variant antibody or antigen-binding fragment thereof may comprise, consist essentially of, or consist of two identical polypeptides comprising a VH and CH as set forth above. In some embodiments, the S2V29-v37.2 or variant antibody or antigen-binding fragment thereof may comprise, consist essentially of, or consist of two identical polypeptides comprising a VL and CL as set forth above. In some embodiments, the S2V29-v37.2 or variant antibody or antigen-binding fragment thereof may comprise, consist essentially of, or consist of two identical polypeptides comprising, consisting essentially of, or consisting of a VH and CH as set forth above, and two identical polypeptides comprising, consisting essentially of, or consisting of a VL and CL as set forth above. In some embodiments, the amino acid sequences of the VH, CH, CL, and/or VL may have at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequences identified above, respectively.

In a more specific embodiment, antibody or antigen-binding fragment may be an S2V29-v37.2 or variant antibody or antigen-binding fragment thereof and have a VH comprising the amino acid sequence according to SEQ ID NO: 184, a CH comprising an amino acid sequence according to any one of SEQ ID NOs: 44-80, particularly SEQ ID NO: 49, a VL comprising an amino acid sequence according to SEQ ID NO: 113, and a CL comprising an amino acid sequence according to SEQ ID NO: 186. In some embodiments, the S2V29-v37.2 or variant antibody or antigen-binding fragment thereof may comprise two identical polypeptides comprising a VH and CH as set forth above. In some embodiments, the S2V29-v37.2 or variant antibody or antigen-binding fragment thereof may comprise two identical polypeptides comprising a VL and CL as set forth above. In some embodiments, the S S2V29-v37.2 or variant antibody or antigen-binding fragment thereof may comprise two identical polypeptides comprising a VH and CH as set forth above, and two identical polypeptides comprising a VL and CL as set forth above. In some embodiments, the amino acid sequences of the VH, CH, CL, and/or VL may have at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequences identified above, respectively.

In some embodiments, the antibody or antigen-binding fragment comprises a HC that consists of an amino acid sequence according to SEQ ID NO: 203 or SEQ ID NO: 203 without the C-terminal lysine and a LC comprising, consists essentially of, or consists of an amino acid sequence according to SEQ ID NO: 204.

In some embodiments, the antibody or antigen-binding fragment comprises a HC that comprises or consists essentially of an amino acid sequence according to SEQ ID NO: 203 or SEQ ID NO: 203 without the C-terminal lysine and a LC comprising, consists essentially of, or consists of an amino acid sequence according to SEQ ID NO: 204. In some embodiments, the antibody or antigen-binding fragment comprises a HC that consists of an amino acid sequence according to SEQ ID NO: 203 or SEQ ID NO: 203 without the C-terminal lysine and a LC comprising, consists essentially of, or consists of an amino acid sequence according to SEQ ID NO: 204. In some embodiments, the HC having at least 85%, 90%, or 95% identity to SEQ ID NO: 203 may comprise an amino acid sequence according to any one of SEQ ID NOs: 184-202. In some embodiments, the HC having at least 85%, 90%, or 95% identity to SEQ ID NO: 203 may consist essentially of an amino acid sequence according to any one of SEQ ID NOs: 184-202. In some embodiments, the HC having at least 85%, 90%, or 95% identity to SEQ ID NO: 203 may consist of an amino acid sequence according to any one of SEQ ID NOs: 184-202. In some embodiments, the HC having at least 85%, 90%, or 95% identity to SEQ ID NO: 203 may comprise an amino acid sequence according to one or more of SEQ ID NOs: 215-221. In some embodiments, the HC having at least 85%, 90%, or 95% identity to SEQ ID NO: 203 may comprise an amino acid sequence according to one or more of SEQ ID NOs: 207-210. In some embodiments, the HC having at least 85%, 90%, or 95% identity to SEQ ID NO: 203 may comprise an amino acid sequence according to one or more of SEQ ID NOs: 215-221 and further comprise an amino acid sequence according to SEQ ID NOs: 207-210, which may, in some embodiments, be comprised with SEQ ID NOs: 215-221. In some embodiments, the HC having at least 85%, 90%, or 95% identity to SEQ ID NO: 203 may comprise CDRH1, CDRH2, CDRH3 amino acid sequences according to SEQ ID NOs: 68, 171, and 70, and further comprise an amino acid sequence according to one or more of SEQ ID NOs: 207-210. In some embodiments, the HC having at least 85%, 90%, or 95% identity to SEQ ID NO: 203 may comprise CDRH1, CDRH2, CDRH3 amino acid sequences according to SEQ ID NOs: 68, 171, and 70, and further comprise an amino acid sequence according to one or more of SEQ ID NOs: 215-221. In some embodiments, the HC having at least 85%, 90%, or 95% identity to SEQ ID NO: 203 may comprise CDRH1, CDRH2, CDRH3 amino acid sequences according to SEQ ID NOs: 68, 171, and 70, and further comprise an amino acid sequence according to one or more of SEQ ID NOs: 207-210, and still further comprise an amino acid sequence according to one or more of SEQ ID NOs: 215-221. In some embodiments, the LC having at least 85%, 90%, or 95% identity to SEQ ID NO: 204 may comprise an amino acid sequence according to one or more of SEQ ID NOs: 222-224. In some embodiments, the LC having at least 85%, 90%, or 95% identity to SEQ ID NO: 204 may comprise an amino acid sequence according to one or more of SEQ ID NOs: 211-214. In some embodiments, the LC having at least 85%, 90%, or 95% identity to SEQ ID NO: 204 may comprise an amino acid sequence according to one or more of SEQ ID NOs: 222-224, and further comprise an amino acid sequence according to SEQ ID NOs: 211-214, which may, in some embodiments, be comprised with SEQ ID NOs: 222-224. In some embodiments, the LC having at least 85%, 90%, or 95% identity to SEQ ID NO: 204 may comprise CDRL1, CDRL2, CDRL3 amino acid sequences according to SEQ ID NOs: 72, 73, and 114, and further comprise an amino acid sequence according to one or more of SEQ ID NOs: 211-214. In some embodiments, the LC having at least 85%, 90%, or 95% identity to SEQ ID NO: 204 may comprise CDRL1, CDRL2, CDRL3 amino acid sequences according to SEQ ID NOs: 72, 73, and 114, and further comprise an amino acid sequence according to one or more of SEQ ID NOs: 222-224. In some embodiments, the LC having at least 85%, 90%, or 95% identity to SEQ ID NO: 204 may comprise CDRL1, CDRL2, CDRL3 amino acid sequences according to SEQ ID NOs: 72, 73, and 114, and further comprise an amino acid sequence according to one or more of SEQ ID NOs: 211-214, and still further comprise an amino acid sequence according to one or more of SEQ ID NOs: 222-224. In some embodiments, the antibody may not include both a VH consisting of an amino acid sequence according to SEQ ID NO: 67 and a VL consisting of an amino acid sequence according to SEQ ID NO: 71.

In some embodiments, the antibody or antigen-binding fragment comprises a HC that comprises an amino acid sequence according to SEQ ID NO: 203 or SEQ ID NO: 203 without the C-terminal lysine and a LC comprising, consists essentially of, or consists of an amino acid sequence according to SEQ ID NO: 204.

In some embodiments, the antibody or antigen-binding fragment comprises a HC that consists essentially of an amino acid sequence according to SEQ ID NO: 203 or SEQ ID NO: 203 without the C-terminal lysine and a LC comprising, consists essentially of, or consists of an amino acid sequence according to SEQ ID NO: 204.

In some embodiments, the antibody or antigen-binding fragment comprises a HC that consists of an amino acid sequence according to SEQ ID NO: 203 or SEQ ID NO: 203 without the C-terminal lysine and a LC comprising, consists essentially of, or consists of an amino acid sequence according to SEQ ID NO: 204.

In some embodiments, the antibody or antigen-binding fragment comprises a HC that comprises, consists essentially of, or consists of an amino acid sequence according to SEQ ID NO: 203 or SEQ ID NO: 203 without the C-terminal lysine and a LC comprising an amino acid sequence according to SEQ ID NO: 204.

In some embodiments, the antibody or antigen-binding fragment comprises a HC that comprises, consists essentially of, or consists of an amino acid sequence according to SEQ ID NO: 203 or SEQ ID NO: 203 without the C-terminal lysine and a LC consisting essentially of an amino acid sequence according to SEQ ID NO: 204.

In some embodiments, the antibody or antigen-binding fragment comprises a HC that comprises, consists essentially of, or consists of an amino acid sequence according to SEQ ID NO: 203 or SEQ ID NO: 203 without the C-terminal lysine and a LC consisting of an amino acid sequence according to SEQ ID NO: 204.

In some embodiments, the antibody or antigen-binding fragment comprises a HC that comprises an amino acid sequence according to SEQ ID NO: 203 or SEQ ID NO: 203 without the C-terminal lysine and a LC comprising an amino acid sequence according to SEQ ID NO: 204.

In some embodiments, the antibody or antigen-binding fragment comprises a HC that consists essentially of an amino acid sequence according to SEQ ID NO: 203 or SEQ ID NO: 203 without the C-terminal lysine and a LC consisting essentially of an amino acid sequence according to SEQ ID NO: 204.

In some embodiments, the antibody or antigen-binding fragment comprises a HC that consists of an amino acid sequence according to SEQ ID NO: 203 or SEQ ID NO: 203 without the C-terminal lysine and a LC consisting of an amino acid sequence according to SEQ ID NO: 204.

In some embodiments, the antibody or antigen-binding fragment comprises a HC that comprises, consists essentially of, or consists of an amino acid sequence having at least 85%, 90%, 95%, or 99% identity to SEQ ID NO: 203 or SEQ ID NO: 203 without the C-terminal lysine and a LC comprising, consists essentially of, or consists of an amino acid sequence having at least 85%, 90%, 95%, or 99% identity to SEQ ID NO: 204. In some embodiments, the antibody or antigen-binding fragment comprises a HC that consists of an amino acid sequence according to SEQ ID NO: 203 or SEQ ID NO: 203 without the C-terminal lysine and a LC comprising, consists essentially of, or consists of an amino acid sequence according to SEQ ID NO: 204. In some embodiments, the HC having at least 85%, 90%, or 95% identity to SEQ ID NO: 203 may comprise an amino acid sequence according to any one of SEQ ID NOs: 184-202. In some embodiments, the HC having at least 85%, 90%, or 95% identity to SEQ ID NO: 203 may consist essentially of an amino acid sequence according to any one of SEQ ID NOs: 184-202. In some embodiments, the HC having at least 85%, 90%, or 95% identity to SEQ ID NO: 203 may consist of an amino acid sequence according to any one of SEQ ID NOs: 184-202. In some embodiments, the HC having at least 85%, 90%, or 95% identity to SEQ ID NO: 203 may comprise an amino acid sequence according to one or more of SEQ ID NOs: 215-221. In some embodiments, the HC having at least 85%, 90%, or 95% identity to SEQ ID NO: 203 may comprise an amino acid sequence according to one or more of SEQ ID NOs: 207-210. In some embodiments, the HC having at least 85%, 90%, or 95% identity to SEQ ID NO: 203 may comprise an amino acid sequence according to one or more of SEQ ID NOs: 215-221 and further comprise an amino acid sequence according to SEQ ID NOs: 207-210, which may, in some embodiments, be comprised with SEQ ID NOs: 215-221. In some embodiments, the HC having at least 85%, 90%, or 95% identity to SEQ ID NO: 203 may comprise CDRH1, CDRH2, CDRH3 amino acid sequences according to SEQ ID NOs: 68, 171, and 70, and further comprise an amino acid sequence according to one or more of SEQ ID NOs: 207-210. In some embodiments, the HC having at least 85%, 90%, or 95% identity to SEQ ID NO: 203 may comprise CDRH1, CDRH2, CDRH3 amino acid sequences according to SEQ ID NOs: 68, 171, and 70, and further comprise an amino acid sequence according to one or more of SEQ ID NOs: 215-221. In some embodiments, the HC having at least 85%, 90%, or 95% identity to SEQ ID NO: 203 may comprise CDRH1, CDRH2, CDRH3 amino acid sequences according to SEQ ID NOs: 68, 171, and 70, and further comprise an amino acid sequence according to one or more of SEQ ID NOs: 207-210, and still further comprise an amino acid sequence according to one or more of SEQ ID NOs: 215-221. In some embodiments, the LC having at least 85%, 90%, or 95% identity to SEQ ID NO: 204 may comprise an amino acid sequence according to one or more of SEQ ID NOs: 222-224. In some embodiments, the LC having at least 85%, 90%, or 95% identity to SEQ ID NO: 204 may comprise an amino acid sequence according to one or more of SEQ ID NOs: 211-214. In some embodiments, the LC having at least 85%, 90%, or 95% identity to SEQ ID NO: 204 may comprise an amino acid sequence according to one or more of SEQ ID NOs: 222-224, and further comprise an amino acid sequence according to SEQ ID NOs: 211-214, which may, in some embodiments, be comprised with SEQ ID NOs: 222-224. In some embodiments, the LC having at least 85%, 90%, or 95% identity to SEQ ID NO: 204 may comprise CDRL1, CDRL2, CDRL3 amino acid sequences according to SEQ ID NOs: 72, 73, and 114, and further comprise an amino acid sequence according to one or more of SEQ ID NOs: 211-214. In some embodiments, the LC having at least 85%, 90%, or 95% identity to SEQ ID NO: 204 may comprise CDRL1, CDRL2, CDRL3 amino acid sequences according to SEQ ID NOs: 72, 73, and 114, and further comprise an amino acid sequence according to one or more of SEQ ID NOs: 222-224. In some embodiments, the LC having at least 85%, 90%, or 95% identity to SEQ ID NO: 204 may comprise CDRL1, CDRL2, CDRL3 amino acid sequences according to SEQ ID NOs: 72, 73, and 114, and further comprise an amino acid sequence according to one or more of SEQ ID NOs: 211-214, and still further comprise an amino acid sequence according to one or more of SEQ ID NOs: 222-224. In some embodiments, the antibody may not include both a VH consisting of an amino acid sequence according to SEQ ID NO: 67 and a VL consisting of an amino acid sequence according to SEQ ID NO: 71.

In some embodiments, antibody or antigen-binding fragment comprises two heavy chains that each comprise the amino acid sequence according to SEQ ID NO:203 or SEQ ID NO:203 without the C-terminal lysine and two light chains that each comprise the amino acid sequence according to SEQ ID NO:204, wherein, further optionally, the antibody or antigen-binding fragment comprises two heavy chains that each consist of the amino acid sequence according to SEQ ID NO:203 or SEQ ID NO:203 without the C-terminal lysine and two light chains that each consist of the amino acid sequence according to SEQ ID NO:204.

Monospecific or multispecific antibodies or antigen-binding fragments of the present disclosure constructed comprise any combination of the VH and VL sequences and/or any combination of the CDRH1, CDRH2, CDRH3, CDRL1, CDRL2, and CDRL3 sequences disclosed herein. A bispecific or multispecific antibody or antigen-binding fragment may, in some embodiments, comprise one, two, or more antigen-binding domains (e.g., a VH and a VL) of the instant disclosure. Two or more binding domains may be present that bind to the same or a different SARS-CoV-2 epitope, and a bispecific or multispecific antibody or antigen-binding fragment as provided herein can, in some embodiments, comprise a further SARS-CoV-2 binding domain, and/or can comprise a binding domain that binds to a different antigen or pathogen altogether.

In any of the presently disclosed embodiments, the antibody or antigen-binding fragment can be multispecific; e.g., bispecific, trispecific, or the like.

In certain embodiments, the antibody or antigen-binding fragment comprises: (i) a first VH and a first VL; and (ii) a second VH and a second VL, wherein the first VH and the second VH are different.

In some embodiments, the first VH and VL comprise an amino acid sequence having at least 85% (e.g. 85%, 86%, 87%, 88%, 89%, 90%, 91%, 92%, 93%, 94%, 95%, 96%, 97%, 98%, 99%, or 100%) identity to the amino acid sequence according to any one of SEQ ID NOs: 67, 81, 84, 87, 90, 93, 97, 101, 105, 109, 137, 139, 142, 166, 170, 174, 179, 182, and 184, and an amino acid sequence having at least 85% (e.g. 85%, 86%, 87%, 88%, 89%, 90%, 91%, 92%, 93%, 94%, 95%, 96%, 97%, 98%, 99%, or 100%) identity to the amino acid sequence according to any one of SEQ ID NOs: 71, 77, 113, 116, 119, 122, 125, 128, 131, 133, 145, 150, 152, 155, 159, and 163, respectively; and wherein the second VH and VL comprise an amino acid sequence having at least 85% (e.g. 85%, 86%, 87%, 88%, 89%, 90%, 91%, 92%, 93%, 94%, 95%, 96%, 97%, 98%, 99%, or 100%) identity to the amino acid sequence according to any one of SEQ ID NOS: 67, 81, 84, 87, 90, 93, 97, 101, 105, 109, 137, 139, 142, 166, 170, 174, 179, 182, and 184, and an amino acid sequence having at least 85% (e.g. 85%, 86%, 87%, 88%, 89%, 90%, 91%, 92%, 93%, 94%, 95%, 96%, 97%, 98%, 99%, or 100%) identity to the amino acid sequence according to any one of SEQ ID NOs: 71, 77, 113, 116, 119, 122, 125, 128, 131, 133, 145, 150, 152, 155, 159, and 163, respectively; wherein the first VH and the second VL are different from the second VH and VL, and wherein the first VH and the first VL together form a first antigen-binding site, and wherein the second VH and the second VL together form a second antigen-binding site.

In some embodiments, the first VH and VL comprise an amino acid sequence having at least 85% (e.g. 85%, 86%, 87%, 88%, 89%, 90%, 91%, 92%, 93%, 94%, 95%, 96%, 97%, 98%, 99%, or 100%) identity to the amino acid sequence according to any one of SEQ ID NOs: 166, 170, 174, 179, 182, and 184, and an amino acid sequence having at least 85% (e.g. 85%, 86%, 87%, 88%, 89%, 90%, 91%, 92%, 93%, 94%, 95%, 96%, 97%, 98%, 99%, or 100%) identity to the amino acid sequence according to any one of SEQ ID NOs: 71, 77, 113, 116, 119, 122, 125, 128, 131, 133, 145, 150, 152, 155, 159, and 163, respectively; and wherein the second VH and VL comprise an amino acid sequence having at least 85% (e.g. 85%, 86%, 87%, 88%, 89%, 90%, 91%, 92%, 93%, 94%, 95%, 96%, 97%, 98%, 99%, or 100%) identity to the amino acid sequence according to any one of SEQ ID NOs: 67, 81, 84, 87, 90, 93, 97, 101, 105, 109, 137, 139, 142, 166, 170, 174, 179, 182, and 184, and an amino acid sequence having at least 85% (e.g. 85%, 86%, 87%, 88%, 89%, 90%, 91%, 92%, 93%, 94%, 95%, 96%, 97%, 98%, 99%, or 100%) identity to the amino acid sequence according to any one of SEQ ID NOs: 71, 77, 113, 116, 119, 122, 125, 128, 131, 133, 145, 150, 152, 155, 159, and 163, respectively; wherein the first VH and the second VL are different from the second VH and VL, and wherein the first VH and the first VL together form a first antigen-binding site, and wherein the second VH and the second VL together form a second antigen-binding site.

In some embodiments, the first VH and VL comprise an amino acid sequence having at least 85% (e.g. 85%, 86%, 87%, 88%, 89%, 90%, 91%, 92%, 93%, 94%, 95%, 96%, 97%, 98%, 99%, or 100%) identity to the amino acid sequence according to any one of SEQ ID NOs: 166, 170, 174, 179, 182, and 184, and an amino acid sequence having at least 85% (e.g. 85%, 86%, 87%, 88%, 89%, 90%, 91%, 92%, 93%, 94%, 95%, 96%, 97%, 98%, 99%, or 100%) identity to the amino acid sequence according to any one of SEQ ID NOs: 71, 77, 113, 116, 119, 122, 125, 128, 131, 133, 145, 150, 152, 155, 159, and 163, respectively; and wherein the second VH and VL comprise an amino acid sequence having at least 85% (e.g. 85%, 86%, 87%, 88%, 89%, 90%, 91%, 92%, 93%, 94%, 95%, 96%, 97%, 98%, 99%, or 100%) identity to the amino acid sequence according to any one of SEQ ID NOs: 166, 170, 174, 179, 182, and 184, and an amino acid sequence having at least 85% (e.g. 85%, 86%, 87%, 88%, 89%, 90%, 91%, 92%, 93%, 94%, 95%, 96%, 97%, 98%, 99%, or 100%) identity to the amino acid sequence according to any one of SEQ ID NOs: 71, 77, 113, 116, 119, 122, 125, 128, 131, 133, 145, 150, 152, 155, 159, and 163, respectively; wherein the first VH and the second VL are different from the second VH and VL, and wherein the first VH and the first VL together form a first antigen-binding site, and wherein the second VH and the second VL together form a second antigen-binding site.

In some embodiments, the antibody or antigen-binding fragment comprises an S2V29-v37.2 or variant antibody or antigen-binding fragment thereof and the first VH and VL comprise an amino acid sequence having at least 85% (e.g. 85%, 86%, 87%, 88%, 89%, 90%, 91%, 92%, 93%, 94%, 95%, 96%, 97%, 98%, 99%, or 100%) identity to the amino acid sequence according to SEQ ID NO: 184, and an amino acid sequence having at least 85% (e.g. 85%, 86%, 87%, 88%, 89%, 90%, 91%, 92%, 93%, 94%, 95%, 96%, 97%, 98%, 99%, or 100%) identity to the amino acid sequence according to SEQ ID NO: 113, respectively; and wherein the second VH and VL comprise an amino acid sequence for a second antibody or antigen-binding fragment; wherein the first VH and the second VL are different from the second VH and VL, and wherein the first VH and the first VL together form a first antigen-binding site, and wherein the second VH and the second VL together form a second antigen-binding site. In some embodiments, the VH having at least 85% identity to SEQ ID NO: 184 may comprise an amino acid sequence according to any one of SEQ ID NOs: 184-202. In some embodiments, the VH having at least 85% identity to SEQ ID NO: 184 may consist essentially of an amino acid sequence according to any one of SEQ ID NOs: 184-202. In some embodiments, the VH having at least 85% identity to SEQ ID NO: 184 may consist of an amino acid sequence according to any one of SEQ ID NOs: 184-202. In some embodiments, the VH having at least 85% identity to SEQ ID NO: 184 may comprise an amino acid sequence according to one or more of SEQ ID NOs: 215-221. In some embodiments, the VH having at least 85% identity to SEQ ID NO: 184 may comprise an amino acid sequence according to one or more of SEQ ID NOs: 207-210. In some embodiments, the VH having at least 85% identity to SEQ ID NO: 184 may comprise an amino acid sequence according to one or more of SEQ ID NOs: 215-221 and further comprise an amino acid sequence according to SEQ ID NOs: 207-210, which may, in some embodiments, be comprised with SEQ ID NOs: 215-221. In some embodiments, the VH having at least 85% identity to SEQ ID NO: 184 may comprise CDRH1, CDRH2, CDRH3 amino acid sequences according to SEQ ID NOS: 68, 171, and 70, and further comprise an amino acid sequence according to one or more of SEQ ID NOs: 207-210. In some embodiments, the VH having at least 85% identity to SEQ ID NO: 184 may comprise CDRH1, CDRH2, CDRH3 amino acid sequences according to SEQ ID NOs: 68, 171, and 70, and further comprise an amino acid sequence according to one or more of SEQ ID NOs: 215-221. In some embodiments, the VH having at least 85% identity to SEQ ID NO: 184 may comprise CDRH1, CDRH2, CDRH3 amino acid sequences according to SEQ ID NOs: 68, 171, and 70, and further comprise an amino acid sequence according to one or more of SEQ ID NOs: 207-210, and still further comprise an amino acid sequence according to one or more of SEQ ID NOs: 215-221. In some embodiments, the VL having at least 85% identity to SEQ ID NO: 113 may comprise an amino acid sequence according to one or more of SEQ ID NOs: 222-224. In some embodiments, the VL having at least 85% identity to SEQ ID NO: 113 may comprise an amino acid sequence according to one or more of SEQ ID NOs: 211-214. In some embodiments, the VL having at least 85% identity to SEQ ID NO: 113 may comprise an amino acid sequence according to one or more of SEQ ID NOs: 222-224, and further comprise an amino acid sequence according to SEQ ID NOs: 211-214, which may, in some embodiments, be comprised with SEQ ID NOs: 222-224. In some embodiments, the VL having at least 85% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 211-214. In some embodiments, the VL having at least 85% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 222-224. In some embodiments, the VL having at least 85% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 211-214, and still further comprise an amino acid sequence according to one or more of SEQ ID NOs: 222-224.

In some embodiments, the antibody or antigen-binding fragment comprises an S2V29-v37.2 or variant antibody or antigen-binding fragment thereof and the first VH and VL comprise an amino acid sequence having at least 85% (e.g. 85%, 86%, 87%, 88%, 89%, 90%, 91%, 92%, 93%, 94%, 95%, 96%, 97%, 98%, 99%, or 100%) identity to the amino acid sequence according to SEQ ID NO: 184, and an amino acid sequence having at least 85% (e.g. 85%, 86%, 87%, 88%, 89%, 90%, 91%, 92%, 93%, 94%, 95%, 96%, 97%, 98%, 99%, or 100%) identity to the amino acid sequence according to SEQ ID NO: 113, respectively; and wherein the second VH and VL comprise an amino acid sequence having at least 85% (e.g. 85%, 86%, 87%, 88%, 89%, 90%, 91%, 92%, 93%, 94%, 95%, 96%, 97%, 98%, 99%, or 100%) identity to the amino acid sequence according to any one of SEQ ID NOs: 67, 81, 84, 87, 90, 93, 97, 101, 105, 109, 137, 139, 142, 166, 170, 174, 179, 182, and 184, and an amino acid sequence having at least 85% (e.g. 85%, 86%, 87%, 88%, 89%, 90%, 91%, 92%, 93%, 94%, 95%, 96%, 97%, 98%, 99%, or 100%) identity to the amino acid sequence according to any one of SEQ ID NOs: 71, 77, 113, 116, 119, 122, 125, 128, 131, 133, 145, 150, 152, 155, 159, and 163, respectively; wherein the first VH and the second VL are different from the second VH and VL, and wherein the first VH and the first VL together form a first antigen-binding site, and wherein the second VH and the second VL together form a second antigen-binding site. In some embodiments, the VH having at least 85% identity to SEQ ID NO: 184 may comprise an amino acid sequence according to any one of SEQ ID NOs: 184-202. In some embodiments, the VH having at least 85% identity to SEQ ID NO: 184 may consist essentially of an amino acid sequence according to any one of SEQ ID NOs: 184-202. In some embodiments, the VH having at least 85% identity to SEQ ID NO: 184 may consist of an amino acid sequence according to any one of SEQ ID NOs: 184-202. In some embodiments, the VH having at least 85% identity to SEQ ID NO: 184 may comprise an amino acid sequence according to one or more of SEQ ID NOs: 215-221. In some embodiments, the VH having at least 85% identity to SEQ ID NO: 184 may comprise an amino acid sequence according to one or more of SEQ ID NOs: 207-210. In some embodiments, the VH having at least 85% identity to SEQ ID NO: 184 may comprise an amino acid sequence according to one or more of SEQ ID NOs: 215-221 and further comprise an amino acid sequence according to SEQ ID NOs: 207-210, which may, in some embodiments, be comprised with SEQ ID NOs: 215-221. In some embodiments, the VH having at least 85% identity to SEQ ID NO: 184 may comprise CDRH1, CDRH2, CDRH3 amino acid sequences according to SEQ ID NOs: 68, 171, and 70, and further comprise an amino acid sequence according to one or more of SEQ ID NOs: 207-210. In some embodiments, the VH having at least 85% identity to SEQ ID NO: 184 may comprise CDRH1, CDRH2, CDRH3 amino acid sequences according to SEQ ID NOs: 68, 171, and 70, and further comprise an amino acid sequence according to one or more of SEQ ID NOS: 215-221. In some embodiments, the VH having at least 85% identity to SEQ ID NO: 184 may comprise CDRH1, CDRH2, CDRH3 amino acid sequences according to SEQ ID NOs: 68, 171, and 70, and further comprise an amino acid sequence according to one or more of SEQ ID NOS: 207-210, and still further comprise an amino acid sequence according to one or more of SEQ ID NOs: 215-221. In some embodiments, the VL having at least 85% identity to SEQ ID NO: 113 may comprise an amino acid sequence according to one or more of SEQ ID NOs: 222-224. In some embodiments, the VL having at least 85% identity to SEQ ID NO: 113 may comprise an amino acid sequence according to one or more of SEQ ID NOs: 211-214. In some embodiments, the VL having at least 85% identity to SEQ ID NO: 113 may comprise an amino acid sequence according to one or more of SEQ ID NOs: 222-224, and further comprise an amino acid sequence according to SEQ ID NOs: 211-214, which may, in some embodiments, be comprised with SEQ ID NOs: 222-224. In some embodiments, the VL having at least 85% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 211-214. In some embodiments, the VL having at least 85% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 222-224. In some embodiments, the VL having at least 85% identity to SEQ ID NO: 113 may comprise CDRL1, CDRL2, CDRL3 amino acid sequences according to SEQ ID NOs: 72, 73, and 114, and further comprise an amino acid sequence according to one or more of SEQ ID NOs: 211-214, and still further comprise an amino acid sequence according to one or more of SEQ ID NOs: 222-224.

In some embodiments, the antibody or antigen-binding fragment is an IgG (e.g., IgG1, IgG2, IgG3, or IgG4), IgA, IgM, IgE, or IgD isotype, or comprises amino acid sequences from two or more of these. In some embodiments, the antibody or antigen-binding fragment is human, humanized, or chimeric.

An antibody or antigen-binding fragment, may be of any allotype or combination of allotypes. “Allotype” refers to the allelic variation found among the IgG subclasses. For example, an allotype may comprise G1m1 (or G1m(a)), G1m2 (or G1m(x)), G1m3 (or G1m(f)), G1m17 (or Gm(z))m), G1m27, and/or G1m28 (G1m27 and G1m28 have been described as “alloallotypes”).

The G1m3 and G1m17 allotypes are located at the same position in the CH1 domain (position 214 according to EU numbering). G1m3 comprises R214 (EU), while G1m17 comprises K214 (EU). The G1m1 allotype is located in the CH3 domain (at positions 356 and 358 (EU)) and refers to the replacements E356D and M358L. The G1m2 allotype refers to a replacement of the alanine in position 431 (EU) by a glycine. G1m allotypes, alloallotypes, and features thereof are known in the art and described at, for example, www.imgt.org/IMGTrepertoire/Proteins/allotypes/human/IGH/IGHC/G1m_allotypes.html and Lefranc, M.-P. and Lefranc, G. Human Gm, Km and Am allotypes and their molecular characterization: a remarkable demonstration of polymorphism In: B. Tait, F. Christiansen (Eds.), Immunogenetics, chap. 34, Humana Press, Springer, New York, USA. Methods Mol. Biol. 2012; 882, 635-680. PMID: 22665258, LIGM: 406, the contents and allotypes and allotype information of which are incorporated herein by reference.

The G1m1 allotype may be combined, for example, with the G1m3, G1m17, G1m27, G1m2, and/or G1m28 allotype. In some embodiments, an allotype is G1m3 with no G1m1 (G1m3,-1). In some embodiments, an allotype is G1m17,1 allotype. In some embodiments, an allotype is G1m3,1. In some embodiments, an allotype is G1m17 with no G1m1 (G1m17,-1). Optionally, these allotypes may be combined (or not combined) with the G1m2, G1m27 or G1m28 allotype. For example, an allotype may be G1m17,1,2.

In some embodiments, an antibody or antigen-binding fragment of the present disclosure comprises a G1m3 allotype or a G1m3,1 allotype. In some embodiments, an antibody or antigen-binding fragment of the present disclosure comprises a G1m3 allotype and comprises M428L and N434S or M428L and N434A mutations or any other mutation(s) that enhance binding to a human FcRn, such as those described herein. In some embodiments, an antibody or antigen-binding fragment of the present disclosure comprises a G1m3,1 allotype and comprises M428L and N434S or M428L and N434A mutations or any other mutation(s) that enhance binding to a human FcRn, such as those described herein. In some embodiments, an antibody or antigen-binding fragment of the present disclosure comprises a G1m17, 1 allotype. In some embodiments, an antibody or antigen-binding fragment of the present disclosure comprises a G1m17, 1 allotype and comprises M428L and N434S or M428L and N434A mutations or any other mutation(s) that enhance binding to a human FcRn, as described further herein.

In some embodiments, the antibody, or the antigen-binding fragment, comprises a human antibody, a monoclonal antibody, a purified antibody, a single chain antibody, a Fab, a Fab′, a F(ab′)2, a Fv, a scFv, or a scFab.

In certain embodiments, an antibody or antigen-binding fragment of the present disclosure is monospecific (e.g., binds to a single epitope) or is multispecific (e.g., binds to multiple epitopes and/or target molecules). Antibodies and antigen-binding fragments may be constructed in various formats. Exemplary antibody formats disclosed in Spiess et al., Mol. Immunol. 67(2):95 (2015), and in Brinkmann and Kontermann, mAbs 9(2):182-212 (2017), which formats and methods of making the same are incorporated herein by reference and include, for example, Bispecific T cell Engagers (BiTEs), DARTs, Knobs-Into-Holes (KIH) assemblies, scFv-CH3-KIH assemblies, KIH Common Light-Chain antibodies, TandAbs, Triple Bodies, TriBi Minibodies, Fab-scFv, scFv-CH-CL-scFv, F(ab′)2-scFv2, tetravalent HCabs, Intrabodies, CrossMabs, Dual Action Fabs (DAFs) (two-in-one or four-in-one), DutaMabs, DT-IgG, Charge Pairs, Fab-arm Exchange, SEEDbodies, Triomabs, LUZ-Y assemblies, Fcabs, κλ-bodies, orthogonal Fabs, DVD-Igs (e.g., U.S. Pat. No. 8,258,268, which formats are incorporated herein by reference in their entirety), IgG (H)-scFv, scFv-(H) IgG, IgG (L)-scFv, scFv-(L) IgG, IgG (L,H)-Fv, IgG (H)-V, V(H)-IgG, IgG (L)-V, V(L)-IgG, KIH IgG-scFab, 2scFv-IgG, IgG-2scFv, scFv4-Ig, Zybody, and DVI-IgG (four-in-one), as well as so-called FIT-Ig (e.g., PCT Publication No. WO 2015/103072, which formats are incorporated herein by reference in their entirety), so-called WuxiBody formats (e.g., PCT Publication No. WO 2019/057122, which formats are incorporated herein by reference in their entirety), and so-called In-Elbow-Insert Ig formats (IEI-Ig; e.g., PCT Publication Nos. WO 2019/024979 and WO 2019/025391, which formats are incorporated herein by reference in their entirety).

In certain embodiments, the antibody or antigen-binding fragment comprises two or more VH domains, two or more VL domains, or both (i.e., two or more VH domains and two or more VL domains). In particular embodiments, an antigen-binding fragment comprises the format (N-terminal to C-terminal direction) VH-linker-VL-linker-VH-linker-VL, wherein the two VH sequences can be the same or different and the two VL sequences can be the same or different. Such linked scFvs can include any combination of VH and VL domains arranged to bind to a given target, and in formats comprising two or more VH and/or two or more VL, one, two, or more different epitopes or antigens may be bound. It will be appreciated that formats incorporating multiple antigen-binding domains may include VH and/or VL sequences in any combination or orientation. For example, the antigen-binding fragment can comprise the format VL-linker-VH-linker-VL-linker-VH, VH-linker-VL-linker-VL-linker-VH, or VL-linker-VH-linker-VH-linker-VL.

In certain embodiments, the antibody or antigen-binding fragment comprises a Fc polypeptide, or a fragment thereof, which may be present in an “Fc moiety” or a fragment thereof. The “Fc” fragment or Fc polypeptide comprises the carboxy-terminal portions (i.e., the CH2 and CH3 domains of IgG) of both antibody H chains held together by disulfides. An Fc may comprise a dimer comprised of two Fc polypeptides (i.e., two CH2-CH3 polypeptides).

In some embodiments, the antibody or antigen-binding fragment or multispecific antibody may further comprise an Fc moiety comprising or consisting of a polypeptide or fragment thereof comprising or consists of amino acid sequences having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to, or comprises or consists of, the amino acid sequences according to any one of SEQ ID NOs: 30-66.

In a further specific embodiment, the antibody or antigen-binding fragment may further comprise a Fc moiety comprising or consisting of a polypeptide or fragment thereof comprising or consists of amino acid sequences having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to, or comprises or consists of, the amino acid sequences according to SEQ ID NO: 49.

In some embodiments, the antibody or antigen-binding fragment may further comprise a LC comprising or consists of amino acid sequences having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to, or comprises or consists of, the amino acid sequences according to SEQ ID NO: 186.

Antibody “effector functions” refer to those biological activities attributable to the Fc moiety (a native sequence Fc moiety or amino acid sequence variant Fc moiety) of an antibody, and vary with the antibody isotype. Examples of antibody effector functions include: Clq binding and complement dependent cytotoxicity; Fc receptor binding; antibody-dependent cell-mediated cytotoxicity (ADCC); phagocytosis; down regulation of cell surface receptors (e.g., B cell receptor); and B cell activation. As discussed herein, modifications (e.g., amino acid substitutions) may be made to an Fc domain in order to modify (e.g., improve, reduce, or ablate) one or more functionality of an Fc-containing polypeptide (e.g., an antibody of the present disclosure). Such functions include, for example, Fc receptor (FcR) binding, antibody half-life modulation (e.g., by binding to FcRn), ADCC function, protein A binding, protein G binding, and complement binding. Amino acid modifications that modify (e.g., improve, reduce, or ablate) Fc functionalities include, for example, the T250Q/M428L, M252Y/S254T/T256E, H433K/N434F, M428L/N434S, M428L/N434A, E233P/L234V/L235A/G236+A327G/A330S/P331S, E333A, S239D/A330L/1332E, P257I/Q311, K326W/E333S, S239D/1332E/G236A, N297Q, K322A, S228P, L235E+E318A/K320A/K322A, L234A/L235A (also referred to herein as “LALA”), and L234A/L235A/P329G mutations, which mutations are summarized and annotated in “Engineered Fc moietys”, published by InvivoGen (2011) and available online at invivogen.com/PDF/review/review-Engineered-Fc-Regions-invivogen.pdf?utm_source=review&utm_medium=pdf&utm_campaign=review&utm_content=E ngineered-Fc-Regions, and are incorporated herein by reference.

For example, to activate the complement cascade, the C1q protein complex can bind to at least two molecules of IgG1 or one molecule of IgM when the immunoglobulin molecule(s) is attached to the antigenic target (Ward, E. S., and Ghetie, V., Ther. Immunol. 2 (1995) 77-94). Burton, D. R., described (Mol. Immunol. 22 (1985) 161-206) that the heavy chain region comprising amino acid residues 318 to 337 is involved in complement fixation. Duncan, A. R., and Winter, G. (Nature 332 (1988) 738-740), using site directed mutagenesis, reported that Glu318, Lys320 and Lys322 form the binding site to C1q. The role of Glu318, Lys320 and Lys 322 residues in the binding of C1q was confirmed by the ability of a short synthetic peptide containing these residues to inhibit complement mediated lysis.

For example, FcR binding can be mediated by the interaction of the Fc moiety (of an antibody) with Fc receptors (FcRs), which are specialized cell surface receptors on cells including hematopoietic cells. Fc receptors belong to the immunoglobulin superfamily, and shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC; Van de Winkel, J. G., and Anderson, C. L., J. Leukoc. Biol. 49 (1991) 511-524). FcRs are defined by their specificity for immunoglobulin classes; Fc receptors for IgG antibodies are referred to as FcγR, for IgE as FcεR, for IgA as FcαR and so on and neonatal Fc receptors are referred to as FcRn. Fc receptor binding is described for example in Ravetch, J. V., and Kinet, J. P., Annu. Rev. Immunol. 9 (1991) 457-492; Capel, P. J., et al., Immunomethods 4 (1994) 25-34; de Haas, M., et al., J Lab. Clin. Med. 126 (1995) 330-341; and Gessner, J. E., et al., Ann. Hematol. 76 (1998) 231-248.

Cross-linking of receptors by the Fc domain of native IgG antibodies (FcγR) triggers a wide variety of effector functions including phagocytosis, antibody-dependent cellular cytotoxicity, and release of inflammatory mediators, as well as immune complex clearance and regulation of antibody production. Fc moieties providing cross-linking of receptors (e.g., FcγR) are contemplated herein. In humans, three classes of FcγR have been characterized to-date, which are: (i) FcγRI (CD64), which binds monomeric IgG with high affinity and is expressed on macrophages, monocytes, neutrophils and eosinophils; (ii) FcγRII (CD32), which binds complexed IgG with medium to low affinity, is widely expressed, in particular on leukocytes, is believed to be a central player in antibody-mediated immunity, and which can be divided into FcγRIIA, FcγRIIB and FcγRIIC, which perform different functions in the immune system, but bind with similar low affinity to the IgG-Fc, and the ectodomains of these receptors are highly homologous; and (iii) FcγRIII (CD16), which binds IgG with medium to low affinity and has been found in two forms: FcγRIIIA, which has been found on NK cells, macrophages, eosinophils, and some monocytes and T cells, and is believed to mediate ADCC; and FcγRIIIB, which is highly expressed on neutrophils.

FcγRIIA is found on many cells involved in killing (e.g. macrophages, monocytes, neutrophils) and seems able to activate the killing process. FcγRIIB seems to play a role in inhibitory processes and is found on B-cells, macrophages and on mast cells and eosinophils. Importantly, it has been shown that 75% of all FcγRIIB is found in the liver (Ganesan, L. P. et al., 2012: “FcγRIIb on liver sinusoidal endothelium clears small immune complexes,” Journal of Immunology 189:4981-4988). FcγRIIB is abundantly expressed on Liver Sinusoidal Endothelium, called LSEC, and in Kupffer cells in the liver and LSEC are the major site of small immune complexes clearance (Ganesan, L. P. et al., 2012: FcγRIIb on liver sinusoidal endothelium clears small immune complexes. Journal of Immunology 189:4981-4988).

In some embodiments, the antibodies disclosed herein and the antigen-binding fragments thereof comprise an Fc polypeptide or fragment thereof for binding to FcγRIIb, in particular an Fc moiety, such as, for example IgG-type antibodies. Moreover, it is possible to engineer the Fc moiety to enhance FcγRIIB binding by introducing the mutations S267E and L328F as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933. Thereby, the clearance of immune complexes can be enhanced (Chu, S., et al., 2014: Accelerated Clearance of IgE In Chimpanzees Is Mediated By Xmab7195, An Fc-Engineered Antibody With Enhanced Affinity For Inhibitory Receptor FcγRIIb. Am J Respir Crit, American Thoracic Society International Conference Abstracts). In some embodiments, the antibodies of the present disclosure, or the antigen-binding fragments thereof, comprise an engineered Fc moiety with the mutations S267E and L328F, in particular as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933.

On B cells, FcγRIIB may function to suppress further immunoglobulin production and isotype switching to, for example, the IgE class. On macrophages, FcγRIIB is thought to inhibit phagocytosis as mediated through FcγRIIA. On eosinophils and mast cells, the B form may help to suppress activation of these cells through IgE binding to its separate receptor.

Regarding FcγRI binding, modification in native IgG of one or more of E233-G236, P238, D265, N297, A327 and P329 reduces binding to FcγRI. IgG2 residues at positions 233-236, substituted into corresponding positions IgG1 and IgG4, reduces binding of IgG1 and IgG4 to FcγRI by 103-fold and eliminated the human monocyte response to antibody-sensitized red blood cells (Armour, K. L., et al. Eur. J. Immunol. 29 (1999) 2613-2624).

Regarding FcγRII binding, reduced binding for FcγRIIA is found, e.g., for IgG mutation of one or more of E233-G236, P238, D265, N297, A327, P329, D270, Q295, A327, R292 and K414.

Two allelic forms of human FcγRIIA are the “H131” variant, which binds to IgG1 Fc with higher affinity, and the “R131” variant, which binds to IgG1 Fc with low affinityer. See, e.g., Bruhns et al., Blood 113:3716-3725 (2009).

Regarding FcγRIII binding, reduced binding to FcγRIIIA is found, e.g., for mutation of one or more of E233-G236, P238, D265, N297, A327, P329, D270, Q295, A327, S239, E269, E293, Y296, V303, A327, K338 and D376. Mapping of the binding sites on human IgG1 for Fc receptors, the above-mentioned mutation sites, and methods for measuring binding to FcγRI and FcγRIIA, are described in Shields, R. L., et al., J. Biol. Chem. 276 (2001) 6591-6604.

Two allelic forms of human FcγRIIIA are the “F158” variant, which binds to IgG1 Fc with lower affinity, and the “V158” variant, which binds to IgG1 Fc with higher affinity. See, e.g., Bruhns et al., Blood 113:3716-3725 (2009).

Regarding binding to FcγRII, two regions of native IgG Fc appear to be involved in interactions between FcγRIIs and IgGs, namely (i) the lower hinge site of IgG Fc, in particular amino acid residues L, L, G, G (234-237, EU numbering), and (ii) the adjacent region of the CH2 domain of IgG Fc, in particular a loop and strands in the upper CH2 domain adjacent to the lower hinge region, e.g. in a region of P331 (Wines, B. D., et al., J. Immunol. 2000; 164:5313-5318). Moreover, FcγRI appears to bind to the same site on IgG Fc, whereas FcRn and Protein A bind to a different site on IgG Fc, which appears to be at the CH2-CH3 interface (Wines, B. D., et al., J. Immunol. 2000; 164:5313-5318).

Also contemplated are mutations that increase binding affinity of an Fc polypeptide or fragment thereof of the present disclosure to a (i.e., one or more) Fcγ receptor (e.g., as compared to a reference Fc polypeptide or fragment thereof or containing the same that does not comprise the mutation(s)). See, e.g., Delillo and Ravetch, Cell 161(5):1035-1045 (2015) and Ahmed et al., J. Struc. Biol. 194(1):78 (2016), the Fc mutations and techniques of which are incorporated herein by reference.

In any of the herein disclosed embodiments, an S2V29 antibody or antigen-binding fragment, particularly an S2V29-v37.2 or variant antibody or antigen-binding fragment thereof, can comprise a Fc polypeptide or fragment thereof comprising a mutation selected from G236A; S239D; A330L; and I332E; or a combination comprising any two or more of the same; e.g., S239D/1332E; S239D/A330L/1332E; G236A/S239D/1332E; G236A/A330L/1332E (also referred to herein as “GAALIE”); or G236A/S239D/A330L/1332E. In some embodiments, the Fc polypeptide or fragment thereof does not comprise S239D. In some embodiments, the Fc polypeptide or fragment thereof comprises S at position 239 (EU numbering). In some embodiments, the Fc polypeptide or fragment thereof comprises the amino acid sequences according to any one of SEQ ID NOs: 38-44 and 57-66.

In certain embodiments, the Fc polypeptide or fragment thereof may comprise or consist of at least a portion of an Fc polypeptide or fragment thereof that is involved in FcRn binding. In certain embodiments, the Fc polypeptide or fragment thereof comprises one or more amino acid modifications that improve binding affinity for (e.g., enhance binding to) FcRn (e.g., at a pH of about 6.0) and, in some embodiments, thereby extend in vivo half-life of a molecule comprising the Fc polypeptide or fragment thereof (e.g., as compared to a reference Fc polypeptide or fragment thereof or antibody that is otherwise the same but does not comprise the modification(s)). In certain embodiments, the Fc polypeptide or fragment thereof comprises or is derived from a IgG Fc and a half-life-extending mutation comprises any one or more of: M428L; N434S; N434H; N434A; N434S; M252Y; S254T; T256E; T250Q; P257I; Q311I; D376V; T307A; E380A (EU numbering). In certain embodiments, a half-life-extending mutation comprises M428L/N434S (also referred to herein as “MLNS”, “LS”, “_LS”, and “-LS”). In certain embodiments, the half-life extending mutation is in a Fc polypeptide or fragment thereof comprising or consisting of the amino acid sequences according to any one of SEQ ID NOs: 45-50 and 57-61. In certain embodiments, a half-life-extending mutation comprises M252Y/S254T/T256E. In certain embodiments, a half-life-extending mutation comprises T250Q/M428L. In certain embodiments, a half-life-extending mutation comprises P257I/Q311I. In certain embodiments, a half-life-extending mutation comprises P257I/N434H. In certain embodiments, a half-life-extending mutation comprises D376V/N434H. In certain embodiments, a half-life-extending mutation comprises T307A/E380A/N434A. In certain embodiments, a half-life-extending mutation comprises M428L/N434A (also referred to herein as “MLNA”, “LA”, _LA″, and “-LA”). In certain embodiments, the half-life extending mutation is in a Fc polypeptide or fragment thereof comprising or consisting of the amino acid sequences according to any one of SEQ ID NOs: 51-56 and 62-66.

In certain embodiments, a half-life-extending mutation comprises M252Y/S254T/T256E (also referred to as “YTE”). Although YTE is known to reduce effector functions, the extension in half-life provided by this mutation may prove sufficiently beneficial to outweigh any effector function reduction.

In some embodiments, an S2V29 antibody or antigen-binding fragment, particularly an S2V29-v37.2 or variant antibody or antigen-binding fragment thereof, includes a Fc moiety comprising the substitution mutations M428L/N434S or M428L/N434A. In some embodiments, an antibody or antigen-binding fragment includes a Fc polypeptide or fragment thereof comprising the substitution mutations G236A/A330L/1332E. In certain embodiments, an antibody or antigen-binding fragment includes a (e.g., IgG) Fc moiety comprising a G236A mutation, an A330L mutation, and a 1332E mutation (GAALIE), and does not comprise a S239D mutation (e.g., comprises a native S at position 239). In particular embodiments, an antibody or antigen-binding fragment includes an Fc polypeptide or fragment thereof comprising the substitution mutation: M428L/N434S and G236A/A330L/1332E, (and may comprises or consist of the amino acid sequences according to any one of SEQ ID NOs: 57-61) and, optionally does not comprise S239D (e.g., comprises S at 239). In particular embodiments, an antibody or antigen-binding fragment includes an Fc polypeptide or fragment thereof comprising the substitution mutation: M428L/N434A and G236A/A330L/1332E, (and may comprises or consist of the amino acid sequences according to any one of SEQ ID NOs: 62-66) and, optionally does not comprise S239D (e.g., comprises S at 239). In certain embodiments, an antibody or antigen-binding fragment includes a Fc polypeptide or fragment thereof comprising the substitution mutations: M428L/N434S (or M428L/N434A) and G236A/S239D/A330L/1332E. Any such antibody or antigen-binding fragment may further comprise a YTE mutation.

In some embodiments, an S2V29 antibody or antigen-binding fragment, particularly an S2V29-v37.2 or variant antibody or antigen-binding fragment thereof, is provided comprising, in a (n e.g. human) IgG1 heavy chain, the amino acid mutation(s) set forth in any one of (i)-(xix): (i) G236A, L328V, and Q295E; (ii) G236A, P230A, and Q295E; (iii) G236A, R292P, and I377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, S267E; (xviii) G236A, S239D, and H268E, and (xix) M252Y/S254T/T256E, wherein the numbering of amino acid residues is according to the EU index as set forth in Kabat. In certain embodiments, the antibody or antigen-binding fragment is afucosylated. In some embodiments, the antibody or antigen-binding fragment further comprises one or more mutation that enhances binding to a human FcRn, such as M428L and N434S mutations or M428L and N434A mutations (EU numbering) or any other mutation(s) that enhance binding to a human FcRn, such as those described herein.

In certain embodiments, the S2V29 antibody or antigen-binding fragment, particularly an S2V29-v37.2 or variant antibody or antigen-binding fragment thereof, is afucosylated. In certain embodiments, the antibody or antigen-binding fragment is provided comprising a VH and a VL that comprise or consist of the amino acid sequences according to SEQ ID NOs: 1) 166 and 113, 2) 170 and 113, 3) 174 and 113, 4) 177 and 113, 5) 179 and 113, 6) 182 and 113, or 7) 184 and 113, respectively, in a more specific embodiment relating to an S2V29-v37.2 or variant antibody or antigen-binding fragment thereof, SEQ ID NOs: 184 and 113, and (A) a Fc moiety comprising the substitution mutations: (i) G236A, L328V, and Q295E; (ii) G236A, P230A, and Q295E; (iii) G236A, R292P, and I377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, and S267E; (xviii) G236A, S239D, and H268E; (xix) M428L/N434S; (xx) M428L/N434A; (xxi) G236A/A330L/1332E/M428L/N434S; (xxii) G236A/A330L/1332E/M428L/N434A; (xxiii) M252Y/S254T/T256E, or (xxiv) any two or more of (i)-(xxiii); or (B) an Fc moiety comprising or consisting of a Fc polypeptide or fragment thereof comprising or consisting of amino acid sequences having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 30-66, optionally other than naturally occurring variants thereof, or comprising or consists of, the amino acid sequence according to SEQ ID NO: 49. In certain embodiments, the antibody may optionally further comprise an light chain polypeptide having a sequence at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 186.

In certain embodiments, the S2V29 antibody or antigen-binding fragment is provided comprising a VH and a VL that comprise or consist of the amino acid sequences according to SEQ ID NOs: 1) 166 and 113, 2) 170 and 113, 3) 174 and 113, 4) 177 and 113, 5) 179 and 113, 6) 182 and 113, or 7) 184 and 113, respectively, a light chain lambda constant region (SEQ ID NO: 186), and an IgG1 G1m17,1 IGHG1*01, LS heavy chain constant region (SEQ ID NO: 49).

In certain embodiments, the antibody or antigen-binding fragment is an S2V29-v37.2 or variant antibody or antigen-binding fragment thereof and comprises a VH and a VL that comprise, consist essentially of, or consist of the amino acid sequences according to SEQ ID NOs: 184 and 113, respectively, a light chain lambda constant region (SEQ ID NO: 186), and an IgG1 G1m17,1 IGHG1*01, LS heavy chain constant region (SEQ ID NO: 49).

In certain embodiments, the antibody or antigen-binding fragment comprises a mutation that alters glycosylation, wherein the mutation that alters glycosylation comprises N297A, N297Q, or N297G, and/or the antibody or antigen-binding fragment is partially or fully aglycosylated and/or is partially or fully afucosylated. Host cell lines and methods of making partially or fully aglycosylated or partially or fully afucosylated antibodies and antigen-binding fragments are known (see, e.g., PCT Publication No. WO 2016/181357; Suzuki et al. Clin. Cancer Res. 13 (6): 1875-82 (2007); Huang et al. MAbs 6:1-12 (2018)).

An antibody or antigen-binding fragment of the present disclosure can be fucosylated (e.g., comprising one or more fucosyl moiety, and typically comprising a native (wild-type) fucosylation pattern or a fucosylation pattern that includes one or more additional, or fewer, fucosyl moieties as compared to native), or can be afucosylated. In particular, native IgG1 antibodies carry a glycan site at N297, and this is typically the only site where a core fucose moiety may be found in the antibody, though some glycan sites may arise through mutation (e.g. in the variable domains) during antibody development. Fucosylation of an Fc polypeptide or fragment thereof, or of an antibody, can be effected by introducing amino acid mutations to introduce or disrupt a fucosylation site (e.g. a mutation at N297, such as N297Q or N297A, to disrupt formation of a glycan that can include a core fucose moiety), though typically it is preferred to maintain N297 and the glycan thereof, such as by expressing the polypeptide in a host cell which has been genetically engineered to lack the ability (or have an inhibited or compromised ability) to fucosylate the polypeptide; by expressing the polypeptide under conditions in which a host cell is impaired in its ability to fucosylate the polypeptide (e.g., in the presence of 2-fluoro-L-fucose (2FF)), or the like. An afucosylated polypeptide can comprise no fucose moieties, or substantially no fucose moieties, and/or can be expressed by a host cell that is genetically engineered to lack the ability (or have an inhibited or compromised ability) to fucosylate the polypeptide and/or can be expressed under conditions in which a host cell is impaired in its ability to fucosylate the polypeptide (e.g., in the presence of 2-fluoro-L-fucose (2FF)). In some embodiments, a polypeptide does not comprise a core fucose moiety at Asn297. In some embodiments, afucosylated polypeptides have increased binding to FcγRIIIA. In some contexts, addition of 2FF to a culture media comprising host cells expressing an antibody results in about 85% or more of the antibodies not carrying a fucose moiety. Accordingly, a plurality of antibodies may be described as “afucosylated” when the plurality was produced in the presence of 2FF or like reagent. In some contexts, a plurality of polypeptides or antibodies may be described as, for example, afucosylated, meaning that about 85% or more of the single polypeptide or antibody molecules of the plurality do not comprise a fucose moiety. In certain preferred embodiments, an afucosylated antibody or polypeptide or a population or a plurality thereof comprises an asparagine (N) at EU position 297. Fucosylation or lack thereof can be assessed using, for example, mass spectrometry (e.g. Electrospray mass spectrometry (ESI-MS)). In some embodiments, compositions are provided that comprise a plurality of any one or more of the presently disclosed polypeptides, wherein the composition comprises afucosylated polypeptides.

In certain embodiments, the antibody or antigen-binding fragment is capable of eliciting continued protection in vivo in a subject even once no detectable levels of the antibody or antigen-binding fragment can be found in the subject (i.e., when the antibody or antigen-binding fragment has been cleared from the subject following administration). Such protection is referred to herein as a vaccinal effect. Without wishing to be bound by theory, it is believed that dendritic cells can internalize complexes of antibody and antigen and thereafter induce or contribute to an endogenous immune response against antigen. In certain embodiments, an antibody or antigen-binding fragment comprises one or more modifications, such as, for example, mutations in the Fc comprising G236A, A330L, and I332E, that are capable of activating dendritic cells that may induce, e.g., T cell immunity to the antigen.

In certain embodiments, an antibody or antigen-binding fragment of the present disclosure comprises an Fc variant selected from the Fc variants summarized in Table 1 (see also PCT Publication No. WO 2022/251119). In certain embodiments, the Fc variant, or the antibody or antigen-binding fragment, is fucosylated. In other embodiments, the Fc variant, or the antibody or antigen-binding fragment, is afucosylated.

TABLE 1
Fc Variants (fucosylated unless otherwise indicated) and
Properties Thereof

| Variant | Certain properties of the indicated |
|---|---|
| (substitution mutation(s) vs. | variant(s), as compared to |
| wild-type human IgG1 Fc) | fucosylated wild-type human IgG1 |
| G236A_L328V_Q295E | Increased binding to human FcγRIIa |
| G236A_P230A_Q295E | (H131 allele and R131 allele); |
| G236A_R292P_I377N | comparable or decreased binding |
| G236A_K334A_Q295E | to human FcγRIIb (e.g. by MSD |
| G236S_R292P_Y300L | assay and/or surface plasmon |
| | resonance); increased ratio of: |
| | binding to human FcγRIIa (H131 |
| | allele or R131 allele) versus binding |
| | to human FcγRIIb; comparable |
| | binding to human FcRn; comparable |
| | production titer; increased signaling |
| | in a host cell via FcγRIIa and/or |
| | decreased signaling in a host cell |
| | via FcγRIIb; Tm within 12° C. or |
| | less of wild-type; |
| | G236S_R292P_Y300L has |
| | improved binding to |
| | C1q |
| G236A_Y300L | Increased binding to human FcγRIIa |
| | (H131 (over 18-fold) and R131 |
| | (over 4-fold)); similar binding to |
| | human FcγRIIb or reduced binding |
| | to human FcγRIIb (e.g. as measured |
| | by surface plasmon resonance); |
| | increased ratio of: binding to human |
| | FcγRIIa (H131 or R131) versus |
| | binding to human FcγRIIb; |
| | comparable binding to human FcRn; |
| | comparable production titer; |
| | increased signaling in a host cell via |
| | FcγRIIa and/or decreased signaling |
| | in a host cell via FcγRIIb; Tm |
| | within 4.5° C. of wild-type |
| G236A_R292P_Y300L | Increased binding to human FcγRIIa |
| | (H131 (over 14-fold) and R131 |
| | (over 2.7-fold)); similar binding to |
| | human FcγRIIb; increased ratio |
| | of: binding to human FcγRIIa |
| | (H131 or R131) versus binding to |
| | human FcγRIIb; increased binding |
| | to human FcγRIIIa (V158 allele |
| | and F158 allele); comparable |
| | binding to human FcRn; comparable |
| | production titer; increased signaling |
| | in a host cell via FcγRIIa and/or |
| | FcγRIIIa, and/or decreased signaling |
| | in a host cell via FcγRIIb; |
| | increased signaling in a host cell |
| | via FcγRIIa and/or decreased |
| | signaling in a host cell via FcγRIIb; |
| | Tm within 4° C. of wild-type; |
| | comparable binding to human C1q |
| G236S_G420V_G446E_L309T | Increased binding to human FcγRIIa; |
| G236A R292P | decreased binding to human |
| | FcγRIIb (less than 0.5-fold); |
| | increased ratio of: binding to human |
| | FcγRIIa (H131 or R131) versus |
| | binding to human FcγRIIb; |
| | comparable binding to human |
| | FcRn; comparable production titer; |
| | increased signaling in a host cell |
| | via FcγRIIa and/or FcγRIIIa, and/or |
| | decreased signaling in a host cell |
| | via FcγRIIb; Tm within 4° C. or less |
| | of wild-type |
| R292P_Y300L | Increased binding to human FcγRIIIa |
| | (V158 and F158); increased |
| | binding to human Clq; Tm |
| | within 4° C. of wild-type |
| Y300L | Increased binding to human C1q |
| E345K_G236S_L235Y_S267E | |
| E272R_L309T_S219Y_S267E | |
| G236Y | |
| G236W | |
| F243L_G446E_P396L_S267E | |
| G236A (afucosylated) | Increased binding to human |
| | FcγRIIa (H131) and mouse FcγRIIa |
| | (R131), decreased binding human |
| | FcγRIIb, increased binding to |
| | human FcγRIIIa (V158) and mouse |
| | FcγRIIIa (F158), increased |
| | binding to human FcγRIIIb, somewhat |
| | decreased binding to human |
| | FcRn, Tm within 0.15° C. of wild- |
| | type or within 0.9° C. of wild-type or |
| | within 0.8° C. of wild-type or |
| | within 0.7° C. of wild-type |
| S239D_H268E_G236A | Increased binding to and signaling |
| | via all human FcγRs tested: |
| | FcγRIIA (H131); FcγRIIA (R131); |
| | FcγRIIB; FcγRIIIA (V158); |
| | FcγRIIIA (F158); FcγRIIIB; |
| | additionally, when anti-HBV |
| | antibody bearing S239D_H268E_ |
| | G236A_M428L_N434S was |
| | combined with hBsAg, the immune |
| | complexes formed thereby |
| | were incubated with |
| | MoDCs; subsequent incubation of |
| | the MoDCs with donor CD4+ T |
| | cells resulted in an increased |
| | percentage of NFAT+CD69+ |
| | CD3+CD4+ T cells as compared |
| | to antibodies bearing |
| | M428L_N434S only. |

In some embodiments, an anti-sarbecovirus antibody or antigen-binding fragment is provided comprising, in a human IgG1 heavy chain the amino acid mutation(s) set forth in any one of (i)-(xix): (i) G236A, L328V, and Q295E; (ii) G236A, P230A, and Q295E; (iii) G236A, R292P, and I377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, and S267E; (xviii) G236A, S239D, and H268E, and (xix) M252Y/S254T/T256E, wherein the numbering of amino acid residues is according to the EU index as set forth in Kabat. In certain embodiments, the antibody or antigen-binding fragment is afucosylated. In some embodiments, the antibody or antigen-binding fragment further comprises one or more mutation that enhances binding to a human FcRn, such as M428L and N434S mutations or M428L and N434A mutations (EU numbering) or any other mutation(s) that enhance binding to a human FcRn, such as those described herein. In certain embodiments, the antibody or antigen-binding fragment is afucosylated.

In any of the presently disclosed embodiments, the antibody or antigen-binding fragment comprises a Fc polypeptide or a fragment thereof, including a CH2 (or a fragment thereof, a CH3 (or a fragment thereof), or a CH2 and a CH3, wherein the CH2, the CH3, or both can be of any isotype and may contain amino acid substitutions or other modifications as compared to a corresponding wild-type CH2 or CH3, respectively. In certain embodiments, a Fc of the present disclosure comprises two CH2-CH3 polypeptides that associate to form a dimer.

In any of the presently disclosed embodiments, the antibody or antigen-binding fragment can be monoclonal. The term “monoclonal antibody” (mAb) as used herein refers to an antibody obtained from a population of substantially homogeneous antibodies, i.e., individual antibodies comprising the population are identical except for possible naturally occurring mutations that may be present, in some cases in minor amounts. Monoclonal antibodies are highly specific, being directed against a single antigenic site. Furthermore, in contrast to polyclonal antibody preparations that include different antibodies directed against different epitopes, each monoclonal antibody is directed against a single epitope of the antigen. In addition to their specificity, the monoclonal antibodies are advantageous in that they may be synthesized uncontaminated by other antibodies. The term “monoclonal” is not to be construed as requiring production of the antibody by any particular method. For example, monoclonal antibodies useful in the present invention may be prepared by the hybridoma methodology first described by Kohler et al., Nature 256:495 (1975), or may be made using recombinant DNA methods in bacterial, eukaryotic animal, or plant cells (see, e.g., U.S. Pat. No. 4,816,567). Monoclonal antibodies may also be isolated from phage antibody libraries using the techniques described in Clackson et al., Nature, 352:624-628 (1991) and Marks et al., J. Mol. Biol., 222:581-597 (1991), for example. Monoclonal antibodies may also be obtained using methods disclosed in PCT Publication No. WO 2004/076677A2.

Antibodies and antigen-binding fragments of the present disclosure include “chimeric antibodies” in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, U.S. Pat. Nos. 4,816,567; 5,530,101 and 7,498,415; and Morrison et al., Proc. Natl. Acad. Sci. USA, 81:6851-6855 (1984)). For example, chimeric antibodies may comprise human and non-human residues. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). Chimeric antibodies also include primatized and humanized antibodies.

A “humanized antibody” is generally considered to be a human antibody that has one or more amino acid residues introduced into it from a source that is non-human. These non-human amino acid residues are typically taken from a variable domain. Humanization may be performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting non-human variable sequences for the corresponding sequences of a human antibody. Accordingly, such “humanized” antibodies are chimeric antibodies (U.S. Pat. Nos. 4,816,567; 5,530,101 and 7,498,415) wherein substantially less than an intact human variable domain has been substituted by the corresponding sequence from a non-human species. In some instances, a “humanized” antibody is one which is produced by a non-human cell or animal and comprises human sequences, e.g., Hc domains.

A “human antibody” is an antibody containing only sequences that are present in an antibody that is produced by a human. However, as used herein, human antibodies may comprise residues or modifications not found in a naturally occurring human antibody (e.g., an antibody that is isolated from a human), including those modifications and variant sequences described herein. These are typically made to further refine or enhance antibody performance. In some instances, human antibodies are produced by transgenic animals. For example, see U.S. Pat. Nos. 5,770,429; 6,596,541 and 7,049,426.

In certain embodiments, an antibody or antigen-binding fragment of the present disclosure is chimeric, humanized, or human.

In some embodiments, the antibody or antigen-binding fragment may be present in an antibody drug conjugate (“ADC”), which comprises the antibody or antigen-binding fragment and a compound comprising or consisting of a small molecule active component (the “drug”).

In specific embodiments, the drug may be an anti-viral agent able to treat sarbecovirus infection, particularly a drug able to treat SARS-CoV-2 infection, such as nucleotide analogs or nucleotide analog prodrugs such as, for example, remdesivir, sofosbuvir, acyclovir, and zidovudine, favipiravir, nirmatrelvir (PF-07321332), lufotrelvir, atazanavir, ebselen, lopinavir, ritonavir, danoprevir, boceprevir, PBI-0451, EDP-235, S-217622, 13b, GC-376, GRL-0920, GRL-1720, IPA-3, JX-06, LN5535, S-217622, EB2-7, EB2-19, GC-14, ML-300, ML-188, PF-07321332, PF-00835231, PF-07304814, N3, UAWJ9d-36-3, MI-09, MI-30, SH-5, YH-53, YH-71, and variations thereof described in Kronenberger T, Laufer S A, Pillaiyar T. CoVID-19 therapeutics: Small-molecule drug development targeting SARS-CoV-2 main protease. Drug Discov Today. 2023 June; 28(6):103579, and any combinations thereof.

In specific embodiments, the drug may be an anti-inflammatory agent, such as dexamethasone, prednisone, or the like.

In some embodiments, the ADC may include both an anti-viral agent and an anti-inflammatory agent.

In some embodiments, the antibody or antigen-binding fragment may comprise, consist essentially of, or consist of any S2V29-v37.2 or variant antibody or antigen-binding fragment thereof specifically described herein.

In some embodiments, the ADC may further comprise a conjugating linker that may be able to bond to both the antibody or antigen-binding fragment and the small molecule.

As described herein, ADCs that comprise an antibody or antigen-binding fragment may comprise various drug-to-antibody ratios (DARs) (i.e., the ratio of the total number of active small molecules attached to the antibody or antigen-binding fragment; DAR is typically reported as an average across the conjugate molecules within a sample), various linker-to-antibody ratios (LARs) (i.e., the total number of conjugations-to-linker sites per antibody or antigen-binding fragment; LAR may be reported as an average across the conjugate molecules within a sample), or both. In certain embodiments, two ADC molecules may possess different DARs but the same LAR. In other embodiments, two ADC molecules possess different LARs but the same—or different—DAR. In some embodiments, a DAR can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 21, 32, or greater. In some embodiments, DAR is 1-5, 6-10, 11-15, 16-20, 21-25, 26-30, 31-35, or greater than 35. In some embodiments, DAR is 1-8, 9-12, 13-18, 19-22, 23-28, 29-32, 33-38, or greater than 38.

In some embodiments, a LAR can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 21, 32, or greater. In some embodiments, LAR is 1-5, 6-10, 11-15, 16-20, 21-25, 26-30, 31-35, or greater than 35. In some embodiments, LAR is 1-8, 9-12, 13-18, 19-22, 23-28, 29-32, 33-38, or greater than 38.

In some embodiments, a ratio of drug molecules: conjugating linker molecules in an ADC is 1:1, 2:1, 4:1, or greater. In certain embodiments, an ADC may comprise: a LAR of 7 and a DAR of 7; a LAR of 4 and a DAR of 8; a LAR of 5 and a DAR of 10; a LAR of 6 and a DAR of 12; a LAR of 16 and a DAR of 32; or a LAR of 8 and a DAR of 32. In some embodiments, the ADC has a LAR of 5 and a DAR of 10. In some embodiments, the ADC has a LAR of 6 and a DAR of 12. A comparison of DAR to LAR in an ADC may be expressed as DAR/LAR, for example, an ADC comprising DAR 3 and LAR 3 has a DAR/LAR of 1. As another example, an ADC comprising DAR 6 and LAR 3 has a DAR/LAR of 2. As yet another example, an ADC comprising DAR 12 and LAR 3 has a DAR/LAR of 4.

DAR or LAR may be determined, for example, by intact mass spectrometry analysis of a de-glycosylated ADC. The molecular weight of the small molecule and the antibody components are used to match each peak in the spectra to a DAR or LAR. In some embodiments, for the purposes of defining each batch of prepared ADC, the DAR or LAR reported refers to the highest intensity peak of the mass distribution. In some embodiments, the DAR or LAR is reported as an average across ADC molecules within a sample.

In some embodiments, an ADC retains, or substantially retains, one or more antibody effector function of the parent antibody or antigen-binding fragment (i.e., of the antibody or antigen-binding fragment without conjugation to the drug). In some embodiments, an ADC has an in vivo half-life in a subject that is substantially the same as, or is greater than, the half-life of the unconjugated antibody or antigen-binding fragment.

In some embodiments, the conjugating linker may be a bivalent linker, a trivalent linker, or a tetravalent linker. “Bivalent linker” refers to a contiguous chain of atoms that includes connection points via single bonds to two portions of the same molecule. “Trivalent linker” refers to a contiguous chain of atoms that includes connection points via single bonds to three portions of the same molecule. “Tetravalent linker” refers to a contiguous chain of atoms that includes connection points via single bonds to four portions of the same molecule.

In some embodiments, the conjugating linker may comprise a chemical linker such as an alkylene linker, a heteroalkylene linker (e.g., a polyethylene glycol (PEG) linker), or combinations thereof. In some embodiments, a conjugating linker is disposed between and connects the antibody or antigen-binding fragment and an active portion (i.e., having or derived from a compound having antiviral activity) of the drug. A conjugating linker can comprise one or a plurality of PEG units (i.e., —(CH₂CH₂O)ₙ— wherein n is denotes the number of PEG units). In some embodiments, a conjugating linker comprises 5, 6, 7, 8, 9, or 10 PEG units, or comprises more than 10 PEG units.

In some embodiments, the conjugating linker is a non-cleavable linker. The term “non-cleavable linker” refers to a linker that is covalently bound to both the antibody or antigen-binding fragment and the drug under normal physiological conditions. Generally, “normal physiological conditions” include a temperature ranging from about 20 to 40° C., an atmospheric pressure of about 1 atm (101 kPa or 14.7 psi), a pH of about 6 to 8, a glucose concentration of about 1 to 20 mM, atmospheric oxygen concentration, and earth gravity. In some embodiments, physiological conditions include the presence of enzymes (i.e., proteases, or nucleases).

In some embodiments, the conjugating linker is a cleavable linker. The term “cleavable linker” refers to a linker that is no longer covalently bound to both the antibody or antigen-binding fragment and the drug after exposure to normal physiological conditions for a period of time, such as 1 minute, 5 minutes, 10 minutes, or 30 minutes.

In specific embodiments, the conjugating linker may be bound to the antibody or antigen-binding fragment in a location other than the VH or VL.

### Polynucleotides, Vectors, DNA Therapeutics, RNA Therapeutics, and Host Cells

In another aspect, the present disclosure provides isolated polynucleotides that encode any of the presently disclosed antibodies or an antigen-binding fragment, or a portion thereof (e.g., a CDR, a VH, a VL, a heavy chain, or a light chain). In certain embodiments, the polynucleotide is codon-optimized for expression in a host cell. Once a coding sequence is known or identified, codon optimization can be performed using known techniques and tools, e.g., using the GenScript® OptimiumGene™ tool; see also Scholten et al., Clin. Immunol. 119:135, 2006). Codon-optimized sequences include sequences that are partially codon-optimized (i.e., one or more codon is optimized for expression in the host cell) and those that are fully codon-optimized.

It will also be appreciated that polynucleotides encoding antibodies and antigen-binding fragments of the present disclosure may possess different nucleotide sequences while still encoding a same antibody or antigen-binding fragment due to, for example, the degeneracy of the genetic code, splicing, and the like.

In any of the presently disclosed embodiments, the polynucleotide can comprise deoxyribonucleic acid (DNA) or ribonucleic acid (RNA). In some embodiments, the RNA comprises messenger RNA (mRNA), more specifically circRNA, taRNA, or saRNA. In some embodiments the polynucleotide may be optimized for expression in a host cell. In embodiments in which the polynucleotide comprises taRNA or saRNA, one or more sequences encoding a replication protein or peptide, one or more sequences encoding an antibody or antigen-binding fragment, or combinations thereof may be optimized for expression in human cells.

In some embodiments, the polynucleotide comprises, consists essentially of or consists of a nucleic acid sequence having at least 85% (e.g. 85%, 86, 87, 88, 89, 90, 91, 92, 93, 94, 95, 96, 97, 98, 99, or 100%) identity to the nucleic acid sequence according to any one of SEQ ID NOs: 75, 79, 135, 83, 86, 89, 92, 96, 100, 104, 108, 112, 136, 138, 141, 165, 169, 173, 176, 178, 181, 183, 76, 80, 115, 118, 121, 124, 127, 130, 132, 134, 144, 149, 151, 154, 158, and 162. In a more specific embodiment, the polynucleotide comprises, consists essentially of or consists of a nucleic acid sequence having at least 85% (e.g. 85%, 86, 87, 88, 89, 90, 91, 92, 93, 94, 95, 96, 97, 98, 99, or 100%) identity to the nucleic acid sequence according to any one of SEQ ID NOs: 165, 169, 173, 176, 178, 181, and 183. In further embodiments, in which the polynucleotide is an saRNA, all T bases in the preceding sequences may be replaced with U bases and the first polynucleotide may further comprise a nucleic acid sequence encoding a replication protein or peptide, as in SEQ ID NOs: 249-250. In other further embodiments in which the polynucleotide is a taRNA, a second polynucleotide is provided comprising a nucleic acid sequence encoding a replication protein or peptide suitable to cause replication of the first polynucleotide.

In other more specific embodiments, at least two polynucleotides are provided, in which the first polynucleotide comprises, consists essentially of or consists of a nucleic acid sequence having at least 85% (e.g. 85%, 86, 87, 88, 89, 90, 91, 92, 93, 94, 95, 96, 97, 98, 99, or 100%) identity to the nucleic acid sequence according to any one of SEQ ID NOs: 75, 79, 135, 83, 86, 89, 92, 96, 100, 104, 108, 112, 136, 138, 141, 165, 169, 173, 176, 178, 181, and 183 and the second polynucleotide comprises, consists essentially of or consists of a nucleic acid sequence having at least 85% (e.g. 85%, 86, 87, 88, 89, 90, 91, 92, 93, 94, 95, 96, 97, 98, 99, or 100%) identity to the nucleic acid sequence according to any one of SEQ ID NOs: 76, 80, 115, 118, 121, 124, 127, 130, 132, 134, 144, 149, 151, 154, 158, and 162. In further embodiments, in which the polynucleotide is an saRNA, all T bases in the preceding sequences may be replaced with U bases and the first polynucleotide may further comprise a nucleic acid sequence encoding a replication protein or peptide, as in SEQ ID NOs: 249-250. In other further embodiments in which the polynucleotides are taRNAs, a third polynucleotide is provided comprising a nucleic acid sequence encoding a replication protein or peptide suitable to cause replication of the first polynucleotide and second polynucleotide.

In a more specific embodiment, at least two polynucleotides are provided, in which the first polynucleotide comprises, consists essentially of or consists of a nucleic acid sequence having at least 85% (e.g. 85%, 86, 87, 88, 89, 90, 91, 92, 93, 94, 95, 96, 97, 98, 99, or 100%) identity to the nucleic acid sequence according to any one of SEQ ID NOs: 165, 169, 173, 176, 178, 181, and 183 and the second polynucleotide comprises, consists essentially of or consists of a nucleic acid sequence having at least 85% (e.g. 85%, 86, 87, 88, 89, 90, 91, 92, 93, 94, 95, 96, 97, 98, 99, or 100%) identity to the nucleic acid sequence according to any one of SEQ ID NOs: 76, 80, 115, 118, 121, 124, 127, 130, 132, 134, 144, 149, 151, 154, 158, and 162.

In some embodiments in which the antibody or antigen-binding fragment is an S2V29-v37.2 or variant antibody or antigen-binding fragment thereof, the polynucleotide comprises, consists essentially of or consists of a nucleic acid sequence having at least 85% (e.g. 85%, 86, 87, 88, 89, 90, 91, 92, 93, 94, 95, 96, 97, 98, 99, or 100%) identity to the nucleic acid sequence according to one or more of SEQ ID NOs: 115 and 183. In some embodiments the polynucleotide having at least 85% identity to the nucleic acid sequence according to SEQ ID NO: 183 may comprise a nucleic acid sequence according to one or more of SEQ ID NOs: 225-227, 231-234, and 239-245. In some embodiments the polynucleotide having at least 85% identity to the nucleic acid sequence according to SEQ ID NO: 115 may comprise a nucleic acid sequence according to one or more of SEQ ID NOs: 228-230, 235-238, and 246-248.

In some embodiments the polynucleotide comprises, consists essentially of or consists of a nucleic acid sequence having at least 85% (e.g. 85%, 86, 87, 88, 89, 90, 91, 92, 93, 94, 95, 96, 97, 98, 99, or 100%) identity to the nucleic acid sequence according to SEQ ID NO: 183. In some embodiments the polynucleotide having at least 85% identity to the nucleic acid sequence according to SEQ ID NO: 183 may comprise a nucleic acid sequence according to one or more of SEQ ID NOs: 225-227, 231-234, and 239-245.

In a more specific embodiment, at least two polynucleotides are provided, in which the first polynucleotide comprises, consists essentially of or consists of a nucleic acid sequence having at least 85% (e.g. 85%, 86, 87, 88, 89, 90, 91, 92, 93, 94, 95, 96, 97, 98, 99, or 100%) identity to the nucleic acid sequence according to SEQ ID NO: 83 and the second polynucleotide comprises, consists essentially of or consists of a nucleic acid sequence having at least 85% (e.g. 85%, 86, 87, 88, 89, 90, 91, 92, 93, 94, 95, 96, 97, 98, 99, or 100%) identity to the nucleic acid sequence according to SEQ ID NO: 115. In some embodiments the polynucleotide having at least 85% identity to the nucleic acid sequence according to SEQ ID NO: 183 may comprise a nucleic acid sequence according to one or more of SEQ ID NOs: 225-227, 231-234, and 239-245. In some embodiments the polynucleotide having at least 85% identity to the nucleic acid sequence according to SEQ ID NO: 115 may comprise a nucleic acid sequence according to one or more of SEQ ID NOs: 228-230, 235-238, and 246-248.

In further embodiments, in which the polynucleotides are mRNAs one or more and optionally both of the first and second polynucleotide is an saRNA, and further comprise a nucleic acid sequence encoding a replication protein or peptide. In other further embodiments in which the polynucleotides are mRNAs, a third polynucleotide is provided comprising a nucleic acid sequence encoding a replication protein or peptide suitable to cause replication of the first polynucleotide and second polynucleotide.

Vectors are also provided, wherein the vectors comprise or contain a polynucleotide as disclosed herein (e.g., a polynucleotide that encodes an antibody or antigen-binding fragment that binds to two or more sarbecoviruses).

A vector can comprise any one or more of the vectors disclosed herein. In particular embodiments, a vector is provided comprising a DNA plasmid construct encoding the antibody or antigen-binding fragment, or a portion thereof (e.g., so-called “DMAb”; see, e.g., Muthumani et al., J Infect Dis. 214 (3): 369-378 (2016); Muthumani et al., Hum Vaccin Immunother 9:2253-2262 (2013)); Flingai et al., Sci Rep. 5:12616 (2015); and Elliott et al., NPJ Vaccines 18 (2017), which antibody-coding DNA constructs and related methods of use, including administration of the same, are incorporated herein by reference). In certain embodiments, a DNA plasmid construct comprises a single open reading frame encoding a heavy chain and a light chain (or a VH and a VL) of the antibody or antigen-binding fragment, wherein the sequence encoding the heavy chain and the sequence encoding the light chain are optionally separated by polynucleotide encoding a protease cleavage site and/or by a polynucleotide encoding a self-cleaving peptide. In some embodiments, the substituent components of the antibody or antigen-binding fragment are encoded by a polynucleotide comprised in a single plasmid. In other embodiments, the substituent components of the antibody or antigen-binding fragment are encoded by a polynucleotide comprised in two or more plasmids (e.g., a first plasmid comprises a polynucleotide encoding a heavy chain, VH, or VH+CH, and a second plasmid comprises a polynucleotide encoding the cognate light chain, VL, or VL+CL). In certain embodiments, a single plasmid comprises a polynucleotide encoding a heavy chain and/or a light chain from two or more antibodies or antigen-binding fragments of the present disclosure. An exemplary expression vector is pVax1, available from Invitrogen®. A DNA plasmid of the present disclosure can be delivered to a subject by, for example, electroporation (e.g., intramuscular electroporation), or with an appropriate formulation (e.g., hyaluronidase).

In some embodiments, the vector may comprise or consist of circRNA, taRNA, or saRNA. In embodiments where the RNA therapeutic consists of a naked circRNA, taRNA, or saRNA vector, the cirRNA, taRNA, or saRNA may, in particular, contain modified nucleosides or other modifications disclosed herein to facilitate delivery to and expression in a human host cell in vivo.

Similarly, naked DNA vectors or other naked DNA therapeutics may contain modifications to facilitate delivery to and expression in a human host cell in vivo.

DNA therapeutics and RNA therapeutics, including circRNA therapeutics, taRNA therapeutics, or saRNA therapeutics, are also provided, wherein the DNA therapeutics or RNA therapeutics comprise or contain a polynucleotide as disclosed herein (e.g., a polynucleotide that encodes an antibody or antigen-binding fragment that binds to two or more sarbecoviruses).

In a further aspect, the present disclosure also provides a host cell expressing an antibody or antigen-binding fragment according to the present disclosure; or comprising or containing a DNA therapeutic, RNA therapeutic, vector, or polynucleotide according to the present disclosure.

Examples of such cells include but are not limited to, eukaryotic cells, e.g., yeast cells, animal cells, insect cells, plant cells; and prokaryotic cells, including E. coli. In some embodiments, the cells are mammalian cells. In certain such embodiments, the cells are a mammalian cell line such as CHO cells (e.g., DHFR-CHO cells (Urlaub et al., PNAS 77:4216 (1980)), human embryonic kidney cells (e.g., HEK293T cells), PER.C6 cells, Y0 cells, Sp2/0 cells. NS0 cells, human liver cells, e.g. Hepa RG cells, myeloma cells or hybridoma cells. Other examples of mammalian host cell lines include mouse sertoli cells (e.g., TM4 cells); monkey kidney CV1 line transformed by SV40 (COS-7); baby hamster kidney cells (BHK); African green monkey kidney cells (VERO-76); monkey kidney cells (CV1); human cervical carcinoma cells (HELA); human lung cells (W138); human liver cells (Hep G2); canine kidney cells (MDCK; buffalo rat liver cells (BRL 3A); mouse mammary tumor (MMT 060562); TRI cells; MRC 5 cells; and FS4 cells. Mammalian host cell lines suitable for antibody production also include those described in, for example, Yazaki and Wu, Methods in Molecular Biology, Vol. 248 (B. K. C. Lo, ed., Humana Press, Totowa, N.J.), pp. 255-268 (2003).

In certain embodiments, a host cell is a prokaryotic cell, such as an E. coli. The expression of peptides in prokaryotic cells such as E. coli is well established (see, e.g., Pluckthun, A. Bio/Technology 9:545-551 (1991). For example, antibodies may be produced in bacteria, in particular when glycosylation and Fc effector function are not needed. For expression of antibody fragments and polypeptides in bacteria, see, e.g., U.S. Pat. Nos. 5,648,237; 5,789,199; and 5,840,523.

In particular embodiments, the cell may be transfected with a vector, DNA therapeutic, or RNA therapeutic according to the present description with an expression vector. The term “transfection” refers to the introduction of nucleic acid molecules, such as DNA or RNA (e.g. mRNA) molecules, into cells, such as into eukaryotic cells. In the context of the present description, the term “transfection” encompasses any method known to the skilled person for introducing nucleic acid molecules into cells, such as into eukaryotic cells, including into mammalian cells. Such methods encompass, for example, electroporation, lipofection, e.g., based on cationic lipids and/or liposomes, calcium phosphate precipitation, nanoparticle based transfection, virus based transfection, or transfection based on cationic polymers, such as DEAE-dextran or polyethylenimine, etc. In certain embodiments, the introduction is non-viral.

Moreover, host cells of the present disclosure may be transfected stably or transiently with a vector, DNA therapeutic, or RNA therapeutic according to the present disclosure, e.g. for expressing an antibody, or an antigen-binding fragment, according to the present disclosure. In such embodiments, the cells may be stably transfected with the vector or DNA therapeutic as described herein. Alternatively, cells may be transiently transfected with a vector, DNA therapeutic, or RNA therapeutic according to the present disclosure encoding an antibody or antigen-binding fragment as disclosed herein. In any of the presently disclosed embodiments, a polynucleotide may be heterologous to the host cell.

Accordingly, the present disclosure also provides recombinant host cells that heterologously express an antibody or antigen-binding fragment of the present disclosure. For example, the cell may be of a species that is different to the species from which the antibody was fully or partially obtained (e.g., CHO cells expressing a human antibody or an engineered human antibody). In some embodiments, the cell type of the host cell does not express the antibody or antigen-binding fragment in nature. Moreover, the host cell may impart a post-translational modification (PTM; e.g., glycosylation or fucosylation) on the antibody or antigen-binding fragment that is not present in a native state of the antibody or antigen-binding fragment (or in a native state of a parent antibody from which the antibody or antigen-binding fragment was engineered or derived). Such a PTM may result in a functional difference (e.g., reduced immunogenicity). Accordingly, an antibody or antigen-binding fragment of the present disclosure that is produced by a host cell as disclosed herein may include one or more post-translational modification that is distinct from the antibody (or parent antibody) in its native state (e.g., a human antibody produced by a CHO cell can comprise a more post-translational modification that is distinct from the antibody when isolated from the human and/or produced by the native human B cell or plasma cell).

Insect cells useful expressing a binding protein of the present disclosure are known in the art and include, for example, Spodoptera frugipera Sf9 cells, Trichoplusia in BTI-TN5B1-4 cells, and Spodoptera frugipera SfSWT01 “Mimic™” cells. See, e.g., Palmberger et al., J. Biotechnol. 152(3-4):160-166 (2011). Numerous baculoviral strains have been identified which may be used in conjunction with insect cells, particularly for transfection of Spodoptera frugiperda cells.

Eukaryotic microbes such as filamentous fungi or yeast are also suitable hosts for cloning or expressing protein-encoding vectors, DNA therapeutics, or RNA therapeutics and include fungi and yeast strains with “humanized” glycosylation pathways, resulting in the production of an antibody with a partially or fully human glycosylation pattern. See Gerngross, Nat. Biotech. 22:1409-1414 (2004); Li et al., Nat. Biotech. 24:210-215 (2006).

Plant cells can also be utilized as hosts for expressing a binding protein of the present disclosure. For example, PLANTIBODIES™ technology (described in, for example, U.S. Pat. Nos. 5,959,177; 6,040,498; 6,420,548; 7,125,978; and 6,417,429) employs transgenic plants to produce antibodies.

In certain embodiments, the host cell comprises a mammalian cell. In particular embodiments, the host cell is a CHO cell, a HEK293 cell, a PER. C6 cell, a Y0 cell, a Sp2/0 cell, a NS0 cell, a human liver cell, a myeloma cell, or a hybridoma cell.

In some embodiments relating to an RNA therapeutic, particularly a circRNA therapeutic, taRNA therapeutic, or saRNA therapeutic, or a DNA therapeutic, the production host cell may be any host cell described above or otherwise disclosed herein. The in vivo human host cell may be any human cell that is a host cell as described above or otherwise disclosed herein.

In a related aspect, the present disclosure provides methods for producing an antibody, or antigen-binding fragment, wherein the methods comprise culturing a host cell of the present disclosure under conditions and for a time sufficient to produce the antibody, or the antigen-binding fragment. Methods useful for isolating and purifying recombinantly produced antibodies, by way of example, may include obtaining supernatants from suitable host cell/vector systems that secrete the recombinant antibody into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant antibody described herein include batch cell culture, which is monitored and controlled to maintain appropriate culture conditions. Purification of soluble antibodies may be performed according to methods described herein and known in the art and that comport with laws and guidelines of domestic and foreign regulatory agencies.

### Compositions

Also provided herein are compositions that comprise any one or more of the presently disclosed antibodies, antigen-binding fragments, ADCs, polynucleotides, vectors, DNA therapeutics, RNA therapeutics, including circ RNA therapeutics, taRNA therapeutics, and saRNA therapeutics, or host cells, singly or in any combination, and can further comprise a pharmaceutically acceptable carrier, excipient, or diluent. Carriers, excipients, and diluents are discussed in further detail herein.

In some embodiments, a composition comprises an S2V29-v37.2 or variant antibody or antigen-binding fragment thereof and a VL, or CDRS according to the VH and VL comprising amino acid sequences according to SEQ ID NOs: 184 and 113, respectively, or a variant thereof specifically described herein in the description of a S2V29-v37.2 variant antibody.

In certain embodiments, a composition comprises two or more different antibodies or antigen-binding fragments according to the present disclosure. In certain embodiments, antibodies or antigen-binding fragments to be used in a combination each independently have one or more of the following characteristics: neutralize one, two, three, four, five, or more naturally occurring sarbecovirus variant lineages; do not compete with one another for Spike protein binding; bind distinct sarbecovirus Spike protein epitopes; have a reduced formation of resistance to sarbecovirus; when in a combination, have a reduced formation of resistance to sarbecovirus; potently neutralize one, two, three, four, five or more live sarbecoviruses; exhibit additive or synergistic effects on neutralization of one, two, three, four, five or more or more live sarbecoviruses when used in combination; exhibit effector functions; are protective in relevant animal model(s) of infection; are capable of being produced in sufficient quantities for large-scale production. In a specific embodiment, one or more of the sarbecoviruses may be SARS-CoV-2 or SARS-CoV. In another specific embodiment, at least two the sarbecoviruses may be SARS-CoV-2 and SARS-CoV.

In some embodiments: a composition comprises a first antibody or antigen-binding fragment and a second antibody or antigen-binding fragment; a multispecific (e.g. bispecific) antibody or antigen-binding fragment is provided; or a combination therapy comprises a first antibody or antigen-binding fragment and a second antibody or antigen-binding fragment, wherein the first antibody or antigen-binding fragment comprise(s) CDRH1, CDRH2, CDRH3, CDRL1, CDRL2, and CDRL3, and optionally VH and VL, according to any S2V29 antibody described herein and any antibody having a VH and VL both having at least 85% (e.g. 85%, 86%, 87%, 88%, 89%, 90%, 91%, 92%, 93%, 94%, 95%, 96%, 97%, 98%, 99%, or 100%) identity to the amino acid sequences according to SEQ ID NOs: 14 and 15; 16 and 17; 18 and 19; 20 and 21; 22 an 23; 24 and 25; 26 and 27; or 28 and 29, respectively.

In some embodiments comprising an S2V29-v37.2 or variant antibody or antigen-binding fragment thereof, the first antibody or antigen-binding fragment comprise(s) CDRH1, CDRH2, CDRH3, CDRL1, CDRL2, and CDRL3, and optionally VH and VL, according to, and optionally any antibody having a VH and VL both having at least 85% (e.g. 85%, 86%, 87%, 88%, 89%, 90%, 91%, 92%, 93%, 94%, 95%, 96%, 97%, 98%, 99%, or 100%) identity to the amino acid sequences according to SEQ ID NOs: 184 and 113, respectively. More specifically, the first antibody or antigen-binding fragment may be a S2V29-v37.2 or variant antibody or antigen-binding fragment thereof specifically described herein.

In certain embodiments, the antibodies or antigen-binding fragments as described above are present in ADCs.

In certain embodiments, a composition comprises polynucleotide, a vector, a RNA therapeutic, or an DNA therapeutic.

In some embodiments, the composition comprises a single polynucleotide encoding i) a heavy chain, VH, or VH+CH; and ii) a cognate light chain, VL, or VL+CL, of the antibody or antigen-binding fragment. In some such embodiments, the polynucleotide is an saRNA and further encodes a replication protein or peptide. In some embodiments, the polynucleotide is a circRNA.

In some embodiments, the composition comprises a first taRNA encoding i) a heavy chain, VH, or VH+CH; and ii) a cognate light chain, VL, or VL+CL, of the antibody or antigen-binding fragment, and a second taRNA that encodes a replication protein or peptide. In some embodiments, one or more, or both the first and second taRNA is a circRNA.

In some embodiments, the composition comprises a first polynucleotide encoding a heavy chain, VH, or VH+CH, and a second polynucleotide encoding a cognate light chain, VL, or VL+CL, of the antibody or antigen-binding fragment. In some such embodiments in which the polynucleotides are mRNAs, the first polynucleotide, the second polynucleotide, or both are saRNAs or taRNAs an further encode a replication protein or peptide. In some embodiments, one or more, at least two, or all of the polynucleotides is a circRNA.

In some embodiments, the composition comprises a first mRNA encoding a heavy chain, VH, or VH+CH, and a second mRNA encoding a cognate light chain, VL, or VL+CL, of the antibody or antigen-binding fragment. In some such embodiments in which the polynucleotides are mRNAs, the composition is a taRNA composition comprising a third taRNA that encodes a replication protein or peptide. In some embodiments, one or more, at least two, or all of the polynucleotides is a circRNA.

In specific embodiments, the compositions may be formulated for intramuscular administration.

### Methods and Uses

Also provided herein are methods for use of an antibody or antigen-binding fragment, ADC, nucleic acid, vector, cell, or composition of the present disclosure in the diagnosis of a sarbecovirus infection (e.g., in a human subject, or in a sample obtained from a human subject).

Methods of diagnosis (e.g., in vitro, ex vivo) may include contacting an antibody, antibody fragment (e.g., antigen-binding fragment) with a sample. Such samples may be isolated from a subject, for example an isolated tissue sample taken from, for example, nasal passages, sinus cavities, salivary glands, lung, liver, pancreas, kidney, ear, eye, placenta, alimentary tract, heart, ovaries, pituitary, adrenals, thyroid, brain, skin or blood. The methods of diagnosis may also include the detection of an antigen/antibody complex, in particular following the contacting of an antibody or antibody fragment with a sample. Such a detection step can be performed at the bench, i.e. without any contact to the human or animal body. Examples of detection methods are well-known to the person skilled in the art and include, e.g., ELISA (enzyme-linked immunosorbent assay), including direct, indirect, and sandwich ELISA.

Also provided herein are methods of treating a subject using an antibody or antigen-binding fragment or ADC of the present disclosure, or a composition comprising the same, wherein the subject has, is believed to have, or is at risk for having an infection by a sarbecovirus. “Treat,” “treatment,” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising an antibody or composition of the present disclosure is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay or prevention of disease progression; remission; survival; prolonged survival; or any combination thereof. In certain embodiments, therapeutic or prophylactic/preventive benefit includes reduction or prevention of hospitalization for treatment of a sarbecovirus infection (i.e., in a statistically significant manner). In certain embodiments, therapeutic or prophylactic/preventive benefit includes a reduced duration of hospitalization for treatment of a sarbecovirus infection (i.e., in a statistically significant manner). In certain embodiments, therapeutic or prophylactic/preventive benefit includes a reduced or abrogated need for respiratory intervention, such as intubation and/or the use of a respirator device. In certain embodiments, therapeutic or prophylactic/preventive benefit includes reversing a late-stage disease pathology and/or reducing mortality.

A “therapeutically effective amount” or “effective amount” of an antibody, antigen-binding fragment, ADC, polynucleotide, vector, DNA therapeutic, RNA therapeutic, host cells, or related composition of the present disclosure refers to an amount of the composition or molecule sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially, sequentially, or simultaneously. A combination may comprise, for example, two different antibodies that specifically bind sarbecovirus antigens, which in certain embodiments, may be the same or different sarbecovirus antigens, and/or can comprise the same or different epitopes.

Accordingly, in certain embodiments, methods are provided for treating a sarbecovirus infection in a subject, wherein the methods comprise administering to the subject an effective amount of an antibody, antigen-binding fragment, polynucleotide, vector, DNA therapeutic, RNA therapeutic, host cell, or composition as disclosed herein.

Subjects that can be treated by the present disclosure are, in general, human and other primate subjects, such as monkeys and apes for veterinary medicine purposes. Other model organisms, such as mice and rats, may also be treated according to the present disclosure. In any of the aforementioned embodiments, the subject may be a human subject. The subjects can be male or female and can be any suitable age, including infant, juvenile, adolescent, adult, and geriatric subjects.

A number of criteria are believed to contribute to high risk for severe symptoms or death associated with a sarbecovirus infection. These include, but are not limited to, age, occupation, general health, pre-existing health conditions, and lifestyle habits. In some embodiments, a subject treated according to the present disclosure comprises one or more risk factors.

In certain embodiments, a human subject treated according to the present disclosure is an infant, a child, a young adult, an adult of middle age, or an elderly person. In certain embodiments, a human subject treated according to the present disclosure is less than 1 year old, or is 1 to 5 years old, or is between 5 and 125 years old (e.g., 5, 10, 15, 20, 25, 30, 35, 40, 45, 50, 55, 60, 65, 70, 75, 80, 85, 90, 95, 100, 105, 110, 115, or 125 years old, including any and all ages therein or therebetween). In certain embodiments, a human subject treated according to the present disclosure is 0-19 years old, 20-44 years old, 45-54 years old, 55-64 years old, 65-74 years old, 75-84 years old, or 85 years old, or older. Persons of middle, and especially of elderly age are believed to be at particular risk. In particular embodiments, the human subject is 45-54 years old, 55-64 years old, 65-74 years old, 75-84 years old, or 85 years old, or older. In some embodiments, the human subject is male. In some embodiments, the human subject is female.

In certain embodiments, a human subject treated according to the present disclosure is a resident of a nursing home or a long-term care facility, is a hospice care worker, is a healthcare provider or healthcare worker, is a first responder, is a family member or other close contact of a subject diagnosed with or suspected of having a sarbecovirus infection, is overweight or clinically obese, is or has been a smoker, has or had chronic obstructive pulmonary disease (COPD), is asthmatic (e.g., having moderate to severe asthma), has an autoimmune disease or condition (e.g., diabetes), and/or has a compromised or depleted immune system (e.g., due to AIDS/HIV infection, a cancer such as a blood cancer, a lymphodepleting therapy such as a chemotherapy, a bone marrow or organ transplantation, or a genetic immune condition), has chronic liver disease, has cardiovascular disease, has a pulmonary or heart defect, works or otherwise spends time in close proximity with others, such as in a factory, shipping center, hospital setting, or the like.

In certain embodiments, a subject treated according to the present disclosure has received a vaccine for a sarbecovirus and the vaccine is determined to be ineffective, e.g., by post-vaccine infection or symptoms in the subject, by clinical diagnosis or scientific or regulatory criteria.

In certain embodiments, treatment is administered as peri-exposure prophylaxis. In certain embodiments, treatment is administered to a subject with mild-to-moderate disease, which may be in an outpatient setting. In certain embodiments, treatment is administered to a subject with moderate-to-severe disease, such as requiring hospitalization.

Typical routes of administering the presently disclosed compositions thus include, without limitation, oral, topical, transdermal, inhalation, parenteral, sublingual, buccal, rectal, vaginal, and intranasal. The term “parenteral”, as used herein, includes subcutaneous injections, intravenous, intramuscular, intrasternal injection or infusion techniques. In certain embodiments, administering comprises administering by a route that is selected from oral, intravenous, parenteral, intragastric, intrapleural, intrapulmonary, intrarectal, intradermal, intraperitoneal, intratumoral, subcutaneous, topical, transdermal, intracisternal, intrathecal, intranasal, and intramuscular. In particular embodiments, a method comprises orally administering the antibody, antigen-binding fragment, polynucleotide, vector, DNA therapeutic, RNA therapeutic, host cell, or composition to the subject.

Administration may, in specific embodiments, be intramuscular. Administration in other embodiments may be intravenous.

Pharmaceutical compositions according to certain embodiments of the present invention are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described an antibody or antigen-binding in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain an effective amount of an antibody or antigen-binding fragment, polynucleotide, vector, DNA therapeutic, RNA therapeutic, host cell, or composition of the present disclosure, for treatment of a disease or condition of interest in accordance with teachings herein.

A composition may be in the form of a solid or liquid. In some embodiments, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi solid, semi liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the composition is in the form of a capsule, for example, a gelatin capsule, it may contain, in addition to materials of the above type, a liquid carrier such as polyethylene glycol or oil.

The composition may be in the form of a liquid, for example, an elixir, syrup, solution, emulsion or suspension. The liquid may be for oral administration or for delivery by injection, as two examples. When intended for oral administration, preferred compositions contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

Liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid composition intended for either parenteral or oral administration should contain an amount of an antibody or antigen-binding fragment as herein disclosed such that a suitable dosage will be obtained. Typically, this amount is at least 0.01% of the antibody or antigen-binding fragment in the composition. When intended for oral administration, this amount may be varied to be between about 0.1 and about 70% of the weight of the composition. Certain oral pharmaceutical compositions contain between about 4% and about 75% of the antibody or antigen-binding fragment. In certain embodiments, pharmaceutical compositions and preparations according to the present invention are prepared so that a parenteral dosage unit contains between 0.01 to 10% by weight of antibody or antigen-binding fragment prior to dilution.

The composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

A composition may include various materials which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The composition in solid or liquid form may include an agent that binds to the antibody or antigen-binding fragment of the disclosure and thereby assists in the delivery of the compound. Suitable agents that may act in this capacity include monoclonal or polyclonal antibodies, one or more proteins or a liposome. The composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi phasic, or tri phasic systems in order to deliver the active ingredient(s). Delivery of the aerosol includes the necessary container, activators, valves, subcontainers, and the like, which together may form a kit. One of ordinary skill in the art, without undue experimentation, may determine preferred aerosols.

The pharmaceutical compositions may be prepared by methodology well known in the pharmaceutical art. For example, a composition intended to be administered by injection can be prepared by combining a composition comprising an antibody, antigen-binding fragment, or ADC as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the peptide composition so as to facilitate dissolution or homogeneous suspension of the antibody, antigen-binding fragment, or ADC in the aqueous delivery system.

In general, an appropriate dose and treatment regimen provide the composition(s) in an amount sufficient to provide therapeutic and/or prophylactic benefit (such as described herein, including an improved clinical outcome (e.g., a decrease in frequency, duration, or severity of diarrhea or associated dehydration, or inflammation, or longer disease-free and/or overall survival, or a lessening of symptom severity). For prophylactic use, a dose should be sufficient to prevent, delay the onset of, or diminish the severity of a disease associated with disease or disorder. Prophylactic benefit of the compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Compositions are administered in an effective amount (e.g., to treat a sarbecovirus infection), which will vary depending upon a variety of factors including the activity of the specific compound employed; the metabolic stability and length of action of the compound; the age, body weight, general health, sex, and diet of the subject; the mode and time of administration; the rate of excretion; the drug combination; the severity of the particular disorder or condition; and the subject undergoing therapy. In certain embodiments, following administration of therapies according to the formulations and methods of this disclosure, test subjects will exhibit about a 10% up to about a 99% reduction in one or more symptoms associated with the disease or disorder being treated as compared to placebo-treated or other suitable control subjects.

Generally, a therapeutically effective daily dose of an antibody or antigen-binding fragment is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g). ADC may be therapeutically effective in similar doses.

For polynucleotides, vectors, DNA therapeutics, RNA therapeutics, host cells, and related compositions of the present disclosure, a therapeutically effective dose may be different than for an antibody or antigen-binding fragment. In such cases, the dose may be sufficient to cause endogenous production of the antibody or antigen-binding fragment in a human patient that results, within a specified period of time, in serum concentrations equivalent to that of a therapeutically effective daily dose of the antibody or antigen-binding fragment. In some embodiments, the specified period of time may be 1, 2, 5, 7, 14, or 30 days from the time of administration.

In some embodiments, the serum concentration equivalent to that of a therapeutically effective daily dose of the antibody or antigen-binding fragment may be maintained for at least 2 weeks, 1 month, 2 months, 3 months, or 6 months, or for at least a time between 2 weeks and 6 months, 2 weeks and 3 months, 2 weeks and 2 months, 2 weeks and 1 month, 1 and 6 months, 1 and 3 months, 1 and 2 months, 2 and 6 months, 2 and 3 months, or 3 and 6 months from the time of administration.

In some embodiments, the polynucleotide may not integrate into the human host cell genome or persist in the human host, such that the antibody or antigen-binding fragment may be undetectable in serum by at least 1, 2, 3, 6, or 12 months from the time of administration.

In some embodiments in which administration is repeated, the time of administration may be the time of first administration.

In some embodiments, particularly those in which RNA therapeutics are used, serum concentrations may typically exhibit an initial increase, followed by a time of steady concentration, then a decrease.

The period of time to reach a therapeutically effective daily hose of the antibody or antigen-binding fragment in the serum, the period of time for which such dose is maintained, and the period of time required for the antibody or antigen-binding fragment to become undetectable in serum, and the doses required to achieve these results are influenced by the nature of the polynucleotide delivered. For example, in some embodiments, taRNA therapeutics or saRNA therapeutics may achieve results similar to those of non-self-amplifying mRNAs at much smaller doses because the RNA encoding the antibody or antigen-binding fragment is amplified in the in vivo human host cell.

In certain embodiments, a method comprises administering the antibody, antigen-binding fragment, polynucleotide, vector, DNA therapeutic, RNA therapeutic, host cell, or composition to the subject a plurality of times, such as 2, 3, 4, 5, 6, 7, 8, 9, or 10 times, wherein a second or successive administration is performed at about 6, about 7, about 8, about 9, about 10, about 11, about 12, about 24, about 48, about 74, about 96 hours, or more, following a first or prior administration, respectively.

In certain embodiments, a method comprises administering the antibody, antigen-binding fragment, polynucleotide, vector, DNA therapeutic, RNA therapeutic, host cell, or composition one or more time prior to the subject being infected by a sarbecovirus.

Compositions comprising an antibody, antigen-binding fragment, ADC, polynucleotide, vector, DNA therapeutic, RNA therapeutic, host cell, or composition of the present disclosure may also be administered simultaneously with, prior to, or after administration of one or more other therapeutic agents. Such combination therapy may include administration of a single pharmaceutical dosage formulation which contains a compound of the invention and one or more additional active agents, as well as administration of compositions comprising an antibody, antigen-binding fragment, or ADC of the disclosure and each active agent in its own separate dosage formulation. For example, an antibody, antigen-binding fragment, or ADC as described herein and the other active agent can be administered to the patient together in a single oral dosage composition such as a tablet or capsule, or each agent administered in separate oral dosage formulations. Similarly, an antibody, antigen-binding fragment, or ADC as described herein and the other active agent can be administered to the subject together in a single parenteral dosage composition such as in a saline solution or other physiologically acceptable solution, or each agent administered in separate parenteral dosage formulations. Where separate dosage formulations are used, the compositions comprising an antibody, antigen-binding fragment, or ADC and one or more additional active agents can be administered at essentially the same time, i.e., concurrently, or at separately staggered times, i.e., sequentially and in any order; combination therapy is understood to include all these regimens.

In certain embodiments, a combination therapy is provided comprising one or more anti-sarbecovirus antibody (or one or more nucleic acid, host cell, vector, DNA therapeutic, RNA therapeutic, or composition) of the present disclosure and one or more anti-inflammatory agent and/or one or more anti-viral agent. In particular embodiments, the one or more anti-inflammatory agent comprises a corticosteroid such as, for example, dexamethasone, prednisone, or the like. In some embodiments, the one or more anti-inflammatory agents comprise a cytokine antagonist such as, for example, an antibody that binds to IL6 (such as siltuximab), or to IL-6R (such as tocilizumab), or to IL-1B, IL-7, IL-8, IL-9, IL-10, FGF, G-CSF, GM-CSF, IFN—Y, IP-10, MCP-1, MIP-1A, MIP1-B, PDGR, TNF-α, or VEGF. In some embodiments, anti-inflammatory agents such as leronlimab, ruxolitinib and/or anakinra are used. In some embodiments, the one or more anti-viral agents comprise nucleotide analogs or nucleotide analog prodrugs such as, for example, remdesivir, sofosbuvir, acyclovir, and zidovudine, or other anti-viral agents described above as also suitable for inclusion in ADCs. In particular embodiments, an anti-viral agent comprises lopinavir, ritonavir, favipiravir, or any combination thereof. Other anti-inflammatory agents for use in a combination therapy of the present disclosure include non-steroidal anti-inflammatory drugs (NSAIDS). It will be appreciated that in such a combination therapy, the one or more antibody (or one or more nucleic acid, host cell, vector, DNA therapeutic, RNA therapeutic, or composition) and the one or more anti-inflammatory agent and/or one or the more antiviral agent can be administered in any order and any sequence, or together.

In some embodiments, an antibody (or one or more nucleic acid, host cell, vector, DNA therapeutic, RNA therapeutic, or composition) is administered to a subject who has previously received one or more anti-inflammatory agent and/or one or more antiviral agent. In some embodiments, one or more anti-inflammatory agent and/or one or more antiviral agent is administered to a subject who has previously received an antibody (or one or more nucleic acid, host cell, vector, DNA therapeutic, RNA therapeutic, or composition).

In certain embodiments, a combination therapy is provided comprising two or more anti-sarbecovirus antibodies, antigen-binding fragments, or ADCs of the present disclosure. A method can comprise administering a first antibody, antigen-binding fragment, or ADC to a subject who has received a second antibody, or can comprise administering two or more antibodies together. For example, in particular embodiments, a method is provided comprising administering to the subject (a) a first antibody, antigen-binding fragment, or ADC when the subject has received a second antibody or antigen-binding fragment; (b) the second antibody, antigen-binding fragment, or ADC when the subject has received the first antibody, antigen-binding fragment, or ADC; or (c) the first antibody, antigen-binding fragment, or ADC and the second antibody, antigen-binding fragment, or ADC.

In a related aspect, uses of the presently disclosed antibodies, antigen-binding fragments, ADCs, vectors, DNA therapeutics, RNA therapeutics, host cells, and compositions are provided.

The present disclosure further provides a kit comprising one or more of any antibodies, antigen-binding fragments, ADCs, polynucleotides, nucleic acids, vectors, DNA therapeutics, RNA therapeutics, or other compositions disclosed herein. The kit may further include one or more of a container, such as a tube, vial, or syringe, an activator, a valve, a subcontainer, or instructions for use, such as for administering to a subject.

In certain embodiments, an antibody, antigen-binding fragment, ADC, polynucleotide, vector, DNA therapeutic, RNA therapeutic, host cell, or composition is provided for use in a method of treating a sarbecovirus infection in a subject.

In certain embodiments, an antibody, antigen-binding fragment, or composition is provided for use in a method of manufacturing or preparing a medicament for treating a sarbecovirus infection in a subject.

The disclosure provides the following specific embodiments:

Embodiment 1. An antibody, or an antigen-binding fragment, comprising a heavy chain variable domain (VH) comprising a complementarity determining region (CDR) H1, CDRH2, and a CDRH3, and a light chain variable domain (VL) comprising a CDRL1, CDRL2, CDRL3, wherein the CDRH1, CDRH2, CDRH3, CDRL1, CDRL2, and CDRL3 are according to SEQ ID NOs: 68, 171, 70, 72, 73, and 114, respectively.

Embodiment 2. An antibody, or an antigen-binding fragment, comprising a heavy chain variable domain (VH) comprising a complementarity determining region (CDR) H1, CDRH2, and a CDRH3, and a light chain variable domain (VL) comprising a CDRL1, CDRL2, CDRL3, comprising amino acid sequences according to SEQ ID NOs: 68, 171, 70, 72, 73, and 114, respectively.

Embodiment 3. An antibody, or an antigen-binding fragment, comprising a heavy chain variable domain (VH) comprising a CDRH1, CDRH2, and a CDRH3, and a light chain variable domain (VL) comprising a CDRL1, CDRL2, CDRL3, consisting of amino acid sequences according to SEQ ID NOs: 68, 171, 70, 72, 73, and 114, respectively.

Embodiment 4. An antibody, or an antigen-binding fragment, comprising a heavy chain variable domain (VH) comprising a complementarity determining region (CDR) H1, CDRH2 region, and a CDRH3, and a light chain variable domain (VL) comprising a CDRL1, CDRL2, CDRL3, wherein the CDRH1, CDRH2, CDRH3, CDRL1, CDRL2, and CDRL3 are according to SEQ ID NOs: 68, 215 or 216, 70, 72, 77, and 114, respectively.

Embodiment 5. An antibody, or an antigen-binding fragment, comprising a heavy chain variable domain (VH) comprising a complementarity determining region (CDR) H1, CDRH2 region, and a CDRH3, and a light chain variable domain (VL) comprising a CDRL1, CDRL2, CDRL3, comprising amino acid sequences according to SEQ ID NOs: 68, 215 or 216, 70, 72, 73, and 114, respectively.

Embodiment 6. An antibody, or an antigen-binding fragment, comprising a heavy chain variable domain (VH) comprising a CDRH1, CDRH2 region, and a CDRH3, and a light chain variable domain (VL) comprising a CDRL1, CDRL2, CDRL3, consisting of amino acid sequences according to SEQ ID NOs: 68, 215 or 216, 70, 72, 73, and 114, respectively.

Embodiment 7. An antibody, or an antigen-binding fragment, comprising a heavy chain variable domain (VH) and a light chain variable domain (VH), wherein the VH and VL are according to SEQ ID NOs: 184 and 113, respectively.

Embodiment 8. An antibody, or an antigen-binding fragment, comprising a heavy chain variable domain (VH) comprising an amino acid sequence according to SEQ ID NO: 184 and a light chain variable domain (VL) comprising an amino acid sequence according to SEQ ID NO: 113.

Embodiment 9. An antibody, or an antigen-binding fragment, comprising a heavy chain variable domain (VH) consisting of an amino acid sequence according to SEQ ID NO: 184 and a light chain variable domain (VL) consisting of an amino acid sequence according to SEQ ID NO: 113.

Embodiment 10. An antibody, or an antigen-binding fragment, comprising a heavy chain variable domain (VH) and a light chain variable domain (VL), wherein the VH is according to any one of SEQ ID NOs: 184 and 187-202, and the VL is according to SEQ ID NO: 113.

Embodiment 11. An antibody, or an antigen-binding fragment, comprising a heavy chain variable domain (VH) comprising an amino acid sequence according to any one of SEQ ID NOs: 184 and 187-202 and a light chain variable domain (VL) comprising an amino acid sequence according to SEQ ID NO: 113.

Embodiment 12. An antibody, or an antigen-binding fragment, comprising a heavy chain variable domain (VH) consisting of an amino acid sequence according to any one of SEQ ID NOs: 184 and 187-202 and a light chain variable domain (VL) consisting of an amino acid sequence according to SEQ ID NO: 113.

Embodiment 13. An antibody, or an antigen-binding fragment, comprising a heavy chain variable domain (VH) comprising an amino acid sequence according to one or more of SEQ ID NOs: 215-221, and a light chain variable domain (VL) comprising an amino acid sequence according to any one of SEQ ID NOs: 113 and 222-224.

Embodiment 14. An antibody, or an antigen-binding fragment, comprising a heavy chain variable domain (VH) comprising an amino acid sequence according to any one of SEQ ID NOs: 184 and 215-221, and a light chain variable domain (VL) comprising an amino acid sequence according to one or more of SEQ ID NOs: 222-224.

Embodiment 15. The antibody, or an antigen-binding fragment according to Embodiment 13 or Embodiment 14, wherein the VH further comprises an amino acid sequence according to one or more of SEQ ID NOs: 207-210.

Embodiment 16. The antibody, or an antigen-binding fragment according to any one of Embodiments 13-15, wherein the VL further comprises an amino acid sequence according to one or more of SEQ ID NOs: 211-214.

Embodiment 17. An antibody, or an antigen-binding fragment comprising a heavy chain variable domain (VH) comprising an amino acid sequence having at least 85%, 90%, or 95% identity to SEQ ID NO: 184.

Embodiment 18. The antibody or antigen-binding fragment according to Embodiment 17, wherein the VH further comprises an amino acid sequence according to any one of SEQ ID NOs: 184-202, and, optionally, a complementarity determining region (CDR) H1, CDRH2, and CDRH3 according to SEQ ID NOs: 68, 171, and 70.

Embodiment 19. The antibody or antigen-binding fragment according to Embodiment 17, wherein the VH further comprises an amino acid sequence according to one or more of SEQ ID NOs: 215-221, and, optionally a complementarity determining region (CDR) H1, CDRH2, and CDRH3 according to SEQ ID NOs: 68, 171, and 70.

Embodiment 20. The antibody or antigen-binding fragment according to Embodiment 19, wherein the VH further comprises an amino acid sequence according to one or more of SEQ ID NOs: 207-210.

Embodiment 21. The antibody or antigen-binding fragment according to any one of Embodiments 17-20, further comprising a light chain variable domain (VL) comprising an amino acid sequence having at least 85%, 90%, or 95% identity to SEQ ID NO: 113, and, optionally a complementarity determining region (CDR) L1, CDRL2, and CDRL3 according to SEQ ID NOs: 72, 73, and 114.

Embodiment 22. The antibody or antigen-binding fragment according to Embodiment 21, wherein the VL further comprises an amino acid sequence according to one or more of SEQ ID NOs: 222-224, and, optionally a complementarity determining region (CDR) L1, CDRL2, and CDRL3 according to SEQ ID NOs: 72, 73, and 114.

Embodiment 23. The antibody or antigen-binding fragment according to Embodiment 22, wherein the VL further comprises an amino acid sequence according to one or more of SEQ ID NOs: 211-214.

Embodiment 24. The antibody or antigen-binding fragment according to any one of Embodiments 17-23, wherein the VH comprises a complementarity determining region (CDR) H1, CDRH2, and CDRH3 according to SEQ ID NOs: 68, 171, and 70, respectively.

Embodiment 25. The antibody or antigen-binding fragment according to any one of Embodiments 17-23, wherein the VH comprises a complementarity determining region (CDR) H1, a CDRH2 region, CDRH3 according to SEQ ID NOs: 68, 215 or 216, and 70, respectively.

Embodiment 26. The antibody or antigen-binding fragment according to any one of Embodiments 21-25, wherein the VL comprises a complementarity determining region (CDR) L1, CDRL2, and CDRL3 according to SEQ ID NOs: 72, 73, and 114, respectively.

Embodiment 27. The antibody or antigen-binding fragment according to any one of Embodiments 17-26, wherein the antibody may not include both a VH consisting of an amino acid sequence according to SEQ ID NO: 67 and a VL consisting of an amino acid sequence according to SEQ ID NO: 71.

Embodiment 28. An antibody, or an antigen-binding fragment, comprising a heavy chain variable domain (VH) comprising an amino acid sequence according to SEQ ID NO: 67, 81, 84, 87, 90, 93, 97, 101, 105, 109, 137, 139, 142, 166, 170, 174, 179, 182, or 184, and a light chain variable domain (VL) comprising an amino acid sequence according to SEQ ID NO: 71, 77, 113, 116, 119, 122, 125, 128, 131, 133, 145, 150, 152, 155, 159, or 163, wherein the antibody may not include both a VH consisting of an amino acid sequence according to SEQ ID NO: 67 and a VL consisting of an amino acid sequence according to SEQ ID NO: 71.

Embodiment 29. The antibody or antigen-binding fragment of any one of Embodiments 1-28, wherein the antibody or antigen-binding fragment is capable of binding to a sarbecovirus.

Embodiment 30. The antibody or antigen-binding fragment of Embodiment 29, wherein the sarbecovirus is a Clade 1b sarbecovirus.

Embodiment 31. The antibody or antigen-binding fragment of Embodiment 29 or Embodiment 30, wherein the sarbecovirus is SARS-CoV-2 WT, SARS-CoV-2 B.1.1.529, SARS-CoV-2 BA.1, SARS-CoV-2 BA.2, SARS-CoV-2 BA.2.12, SARS-CoV-2 BA2.75.2, SARS-CoV-2 BA.2.86, SARS-CoV-2 BA.3, SARS-CoV-2 BA.4, SARS-CoV-2 BA.5, SARS-CoV-2 XBB.1, SARS-CoV-2 BQ.1.1, SARS-CoV-2 XBB.1.5, SARS-CoV-2 CH.1.1, SARS-CoV-2 XBB.2.3, SARS-CoV-2 EG.5, SARS-CoV-2 EG.5.1, SARS-CoV-2 XBB.1.16.1, SARS-CoV-2 XBB1.16.6, SARS-CpV-2 FL.1.5.1, SARS-CoV-2 HK.3, SARS-CoV-2 HV.1, SARS-CoV-2 JD.1.1, SARS-CoV-2 JN.1, SARS-CoV, RATG13, PANG/GD, or PAND/GX.

Embodiment 32. The antibody or antigen-binding fragment of any one of Embodiments 1-31, wherein the antibody or antigen-binding fragment is capable of binding to a surface glycoprotein, when the surface glycoprotein is expressed on a cell surface of a host cell and/or is comprised on a virion.

Embodiment 33. The antibody or antigen-binding fragment of any one of Embodiments 1-32 which is capable of binding to a surface glycoprotein from two or more (e.g., two, three, four, five, or more) sarbecoviruses.

Embodiment 34. The antibody or antigen-binding fragment of any one of Embodiments 1-33, which is capable of neutralizing an infection by one or more sarbecoviruses in an in vitro model of infection and/or in an in vivo animal model of infection and/or in a human.

Embodiment 35. The antibody or antigen-binding fragment of any one of Embodiments 1-34, which is capable of neutralizing an infection by two or more sarbecoviruses in an in vitro model of infection and/or in an in vivo animal model of infection and/or in a human.

Embodiment 36. The antibody or antigen-binding fragment of any one of Embodiments 1-35, which: (i) recognizes an epitope in the Spike protein of two or more, three or more, four or more, or five or more sarbecoviruses; (ii) is capable of blocking an interaction between the Spike protein of two or more, three or more, four or more, or five or more sarbecoviruses and their respective cell surface receptor(s), wherein, optionally, a cell surface receptor comprises a human ACE2; (iii) recognizes an epitope that is conserved in the Spike protein of two or more, three or more, four or more, or five or more sarbecoviruses; (iv) is cross-reactive against two or more, three or more, four or more, or five or more sarbecoviruses, optionally comprising one or more clade 1b sarbecoviruses; or (v) any combination of (i)-(iv).

Embodiment 37. The antibody or antigen-binding fragment of any one of Embodiments 1-36, wherein the antibody or antigen-binding fragment comprises a heavy chain constant domain (CH) or a Fc polypeptide or a fragment thereof.

Embodiment 38. The antibody or antigen-binding fragment of Embodiment 37, wherein the CH or Fc polypeptide or fragment thereof comprises an amino acid sequence according to any one of SEQ ID NOs: 30-66.

Embodiment 39. The antibody or antigen-binding fragment of any one of Embodiments 1-38, which is an IgG, IgA, IgM, IgE, or IgD isotype.

Embodiment 40. The antibody or antigen-binding fragment of Embodiment 39, which is an IgG isotype selected from IgG1, IgG2, IgG3, and IgG4, and is preferably an IgG1 isotype.

Embodiment 41. The antibody or antigen-binding fragment of Embodiment 40, wherein the antibody comprises a heavy chain constant domain (CH) comprising an amino acid sequence according to SEQ ID NO: 49.

Embodiment 42. The antibody or antigen-binding fragment of any one of Embodiments 1-41, wherein the antibody or antigen-binding fragment comprises a light chain constant domain (CL) comprising an amino acid sequence according to SEQ ID NO: 186.

Embodiment 43. An antibody or antigen-binding fragment comprising:
a heavy chain (HC) comprising a heavy chain variable domain (VH) comprising an amino acid sequence according to SEQ ID NO: 184 and a heavy chain constant domain (CH) comprising an amino acid sequence according to SEQ ID NO: 49; and
a light chain (LC) comprising a light chain variable domain (VL) comprising an amino acid sequence according to SEQ ID NO: 113 and a light chain constant domain (CL) comprising an amino acid sequence according to SEQ ID NO: 186.

Embodiment 44. An antibody or antigen-binding fragment comprising:
a heavy chain (HC) comprising a heavy chain variable domain (VH) according to any one of Embodiments 1-27 and a heavy chain constant domain (CH) comprising an amino acid sequence according to SEQ ID NO: 49; and
a light chain (LC) comprising a light chain variable domain (VL) according to any one of Embodiments 1-27 and a light chain constant domain (CL) comprising an amino acid sequence according to SEQ ID NO: 186.

Embodiment 45. The antibody or antigen-binding fragment of Embodiment 43 or Embodiment 44, wherein the HC comprises an amino acid sequence according to SEQ ID NO: 203 or SEQ ID NO: 203 without the C-terminal lysine, and the LC comprises an amino acid sequence according to SEQ ID NO: 204, wherein, optionally, the antibody or antigen-binding fragment comprises two heavy chains that each comprise the amino acid sequence according to SEQ ID NO:203 or SEQ ID NO:203 without the C-terminal lysine and two light chains that each comprise the amino acid sequence according to SEQ ID NO:204, wherein, further optionally, the antibody or antigen-binding fragment comprises two heavy chains that each consist of the amino acid sequence according to SEQ ID NO:203 or SEQ ID NO:203 without the C-terminal lysine and two light chains that each consist of the amino acid sequence according to SEQ ID NO:204.

Embodiment 46. The antibody or antigen-binding fragment of Embodiment 45, wherein the HC consists of an amino acid sequence according to SEQ ID NO: 203 or SEQ ID NO: 203 without the C-terminal lysine, and the LC consists of an amino acid sequence according to SEQ ID NO: 204.

Embodiment 47. The antibody or antigen-binding fragment of any one of Embodiments 1-46, which is human, humanized, or chimeric.

Embodiment 48. The antibody or antigen-binding fragment of any one of Embodiments 1-47, wherein the antibody, or the antigen-binding fragment, comprises a human antibody, a monoclonal antibody, a purified antibody, a single chain antibody, a Fab, a Fab′, a F(ab′)2, a Fv, a scFv, or a scFab.

Embodiment 49. The antibody or antigen-binding fragment of any one of Embodiments 1-48, wherein the antibody or antigen-binding fragment comprises two identical VH domains and two identical VL domains, wherein each VH and VL together form an antigen-binding site.

Embodiment 50. The antibody or antigen-binding fragment of any one of Embodiments 1-49, wherein the antibody or antigen-binding fragment is a multi-specific antibody or antigen-binding fragment.

Embodiment 51. The antibody or antigen-binding fragment of Embodiment 50, wherein the antibody or antigen-binding fragment is a bispecific antibody or antigen-binding fragment.

Embodiment 52. An antibody or antigen-binding fragment comprising: a first VH and a first VL; and a second VH and a second VL, wherein the first VH and VL comprise an amino acid sequence having at least 85% identity to the amino acid sequence according to SEQ ID NO: 184, and an amino acid sequence having at least 85% identity to the amino acid sequence according to SEQ ID NO: 113, respectively; wherein the first VH and the second VL are different from the second VH and VL; wherein the first VH and the first VL together form a first antigen-binding site; and wherein the second VH and the second VL together form a second antigen-binding site.

Embodiment 53. An antibody or antigen-binding fragment comprising: a first VH and a first VL, both according to at any one of Embodiments 1-27; and a second VH and VL, wherein the first VH and the first VL together form a first antigen-binding site; and wherein the second VH and the second VL together form a second antigen-binding site.

Embodiment 54. The antibody or antigen-binding fragment of Embodiment 52, wherein the second VH and VL comprise an amino acid sequence having at least 85% identity to the amino acid sequence according to SEQ ID NOs: 67, 81, 84, 87, 90, 93, 97, 101, 105, 109, 137, 139, 142, 166, 170, 174, 179, 182, or 184, and an amino acid sequence having at least 85% identity to the amino acid sequence according to SEQ ID NOs: 71, 77, 113, 116, 119, 122, 125, 128, 131, 133, 145, 150, 152, 155, 159, or 163, respectively; wherein the first VH and the second VL are different from the second VH and VL; wherein the first VH and the first VL together form a first antigen-binding site; and wherein the second VH and the second VL together form a second antigen-binding site.

Embodiment 55. An antibody drug conjugate (ADC) comprising an antibody according to any one of Embodiments 1-54 conjugated to a drug, wherein the drug is an anti-viral or anti-inflammatory small molecule.

Embodiment 56. An isolated polynucleotide encoding the antibody or antigen-binding fragment of any one of Embodiments 1-54.

Embodiment 57. The polynucleotide of Embodiment 56, wherein the polynucleotide comprises or consists of a nucleic acid sequence having at least 85% identity to the nucleic acid sequence according to one or more of SEQ ID NOs: 115 and 183.

Embodiment 58. The polynucleotide of Embodiment 57, wherein the polynucleotide further comprise a nucleic acid sequence according to one or more of SEQ ID NOs: 225-248.

Embodiment 59. The polynucleotide of any one of Embodiments 56-58, wherein the polynucleotide comprises deoxyribonucleic acid (DNA) or ribonucleic acid (RNA), wherein the RNA optionally comprises messenger RNA (mRNA), optionally self-amplifying RNA (saRNA), trans-amplifying RNA (taRNA), or circular RNA (circRNA).

Embodiment 60. The polynucleotide of Embodiment 59, wherein the mRNA further comprises at a polynucleotide encoding a replication protein having at least 85% identity to the amino acid sequence according to SEQ ID NO: 206, or to a portion thereof corresponding to one or more of non-structural proteins 1-4 (nsP1-4).

Embodiment 61. The polynucleotide of any one of Embodiments 56-60, wherein the polynucleotide is codon-optimized for expression in a host cell.

Embodiment 62. The polynucleotide of Embodiment 61, wherein the host cell is a production host cell.

Embodiment 63. The polynucleotide of Embodiment 61, wherein the host cell is an in vivo human host cell.

Embodiment 64. A recombinant vector, DNA therapeutic, or RNA therapeutic comprising the polynucleotide of any one of Embodiments 56-63.

Embodiment 65. A DNA therapeutic construct or RNA therapeutic construct comprising the polynucleotide of any one of Embodiments 56-63 encapsulated in a carrier, wherein the carrier optionally comprises a lipid, a lipid-derived delivery vehicle, such as a liposome, a solid lipid nanoparticle, an oily suspension, a submicron lipid emulsion, a lipid microbubble, an inverse lipid micelle, a cochlear liposome, a lipid microtubule, a lipid microcylinder, lipid nanoparticle (LNP), or a nanoscale platform.

Embodiment 66. A host cell comprising the polynucleotide of any one of Embodiments 56-63 and/or the vector, DNA therapeutic, or RNA therapeutic of Embodiment 64 or 65, wherein the polynucleotide is heterologous to the host cell.

Embodiment 67. A human B cell comprising the polynucleotide of any one of Embodiments 56-63 and/or the vector, DNA therapeutic, or RNA therapeutic of Embodiment 64 or 65, wherein polynucleotide is heterologous to the human B cell and/or wherein the human B cell is immortalized.

Embodiment 68. A composition comprising: (i) the antibody or antigen-binding fragment of any one of Embodiments 1-54; (ii) the ADC of Embodiment 55; (iii) the polynucleotide of any one of Embodiments 56-63; (iv) the recombinant vector, DNA therapeutic, or RNA therapeutic of Embodiment 64 or 65; (v) the host cell of Embodiment 66; and/or (vi) the human B cell of Embodiment 67, and a pharmaceutically acceptable excipient, carrier, or diluent.

Embodiment 69. The composition of Embodiment 68, comprising two or more antibodies or antigen-binding fragments, wherein the first antibody or antigen-binding fragment is according to any one of Embodiments 1-54.

Embodiment 70. The composition of Embodiment 68, comprising a first polynucleotide according to any one of Embodiments 56-63 or a recombinant vector, DNA therapeutic, or RNA therapeutic of Embodiment 64 or 65; and a second polynucleotide.

Embodiment 71. The composition of Embodiment 70, wherein the first polypeptide comprises or consists of a nucleic acid sequence having at least 85% identity to the nucleic acid sequence according to SEQ ID NO: 184, and the second polypeptide comprises or consists of a nucleic acid sequence having at least 85% identity to the nucleic acid sequence according to SEQ ID NO: 115.

Embodiment 72. A method of treating a sarbecovirus infection (e.g. infection by a SARS-CoV-2) in a subject, the method comprising administering to the subject an effective amount of (i) the antibody or antigen-binding fragment of any one of Embodiments 1-54; (ii) the ADC of Embodiment 55; (iii) the polynucleotide of any one of Embodiments 56-63; (iv) the recombinant vector, DNA therapeutic, or RNA therapeutic of Embodiment 64 or 65; (v) the host cell of Embodiment 66; and/or (vi) the human B cell of Embodiment 67, and/or (vii) the composition of any one of Embodiments 68-71.

Embodiment 73. The antibody or antigen-binding fragment of any one of Embodiments 1-54, the ADC of Embodiment 55, the polynucleotide of any one of Embodiments 56-63, the recombinant vector, DNA therapeutic, or RNA therapeutic of Embodiment 64 or 65, the host cell of Embodiment 66, the human B cell of Embodiment 67, and/or the composition of any one of Embodiments 68-71 for use in a method of treating a sarbecovirus infection (e.g. infection by a SARS-CoV-2) in a subject.

Embodiment 74. The antibody or antigen-binding fragment of any one of Embodiments 1-54, the ADC of Embodiment 55, the polynucleotide of any one of Embodiments 56-63, the recombinant vector, DNA therapeutic, or RNA therapeutic of Embodiment 64 or 65, the host cell of Embodiment 66, the human B cell of Embodiment 67, and/or the composition of any one of Embodiments 68-71 for use in the preparation of a medicament for the treatment of a sarbecovirus infection (e.g. infection by a SARS-CoV-2) in a subject.

Embodiment 75. The antibody or antigen-binding fragment of any one of Embodiments 1-54, the ADC of Embodiment 55, the polynucleotide of any one of Embodiments 56-63, the recombinant vector, DNA therapeutic, or RNA therapeutic of Embodiment 64 or 65, the host cell of Embodiment 66, the human B cell of Embodiment 67, and/or the composition of any one of Embodiments 68-71, wherein the antibody or antigen-binding fragment binds to two or more sarbecovirus S proteins, as measured using biolayer interferometry.

Embodiment 76. A kit comprising a liquid composition comprising (i) the antibody or antigen-binding fragment of any one of Embodiments 1-54; (ii) the ADC of Embodiment 55; (iii) the polynucleotide of any one of Embodiments 56-63; (iv) the recombinant vector, DNA therapeutic, or RNA therapeutic of Embodiment 64 or 65; (v) the host cell of Embodiment 66; and/or (vi) the human B cell of Embodiment 67, and/or (vii) the composition of any one of Embodiments 68-71, and instructions for use thereof in treating a SARS-CoV-2 infection in a subject.

Embodiment 77. The kit of Embodiment 76, wherein the instructions for use are for the method of Embodiment 72 or the use according to any one of Embodiments 73-76.

Embodiment 78. A method for in vitro diagnosis of a sarbecovirus infection (e.g. infection by a SARS-CoV-2), the method comprising: (i) contacting a sample from a subject with an antibody or antigen-binding fragment of any one of Embodiments 1-54; and (ii) detecting a complex comprising an antigen and the antibody, or comprising an antigen and the antigen-binding fragment.

Embodiment 79. A method for producing an antibody or antigen-binding fragment of any one of Embodiments 1-54, wherein the method comprises culturing a production host cell expressing the antibody or antigen-binding fragment under conditions and for a time sufficient to produce the antibody, or the antigen-binding fragment.

Embodiment 80. The method for producing an antibody or antigen-binding fragment of Embodiment 79, wherein the host cell comprises a recombinant vector, DNA therapeutic, or RNA therapeutic of any one of Embodiments 64 or 65.

Embodiment 81. The method for producing an antibody or antigen-binding fragment of Embodiment 80, wherein the host cell is a mammalian cell.

TABLE 2
Sequences

| Sequence | SEQ ID | |
|---|---|---|
| Description | NO | Sequence |
| SARS-CoV-2 | 1 | MFVFLVLLPLVSSQCVNLTTRTQLPPAYTNSFTRGVYYPDKVFRSSVL |
| surface | | HSTQDLFLPFFSNVTWFHAIHVSGTNGTKRFDNPVLPFNDGVYFASTE |
| glycoprotein | | KSNIIRGWIFGTTLDSKTQSLLIVNNATNVVIKVCEFQFCNDPFLGVYY |
| GenBank: | | HKNNKSWMESEFRVYSSANNCTFEYGKQGNFKNLREFVFKNIDGYF |
| QHD43416.1; | | KIYSKHTPINLVRDLPQGFSALEPLVDLPIGINITRFQTLLALHRSYLTP |
| Jan. 23, 2020 | | GDSSSGWTAGAAAYYVGYLQPRTFLLKYNENGTITDAVDCALDPLSE |
| | | TKCTLKSFTVEKGIYQTSNFRVQPTESIVRFPNITNLCPFGEVFNATRF |
| | | ASVYAWNRKRISNCVADYSVLYNSASFSTFKCYGVSPTKLNDLCFTN |
| | | VYADSFVIRGDEVRQIAPGQTGKIADYNYKLPDDFTGCVIAWNSNNL |
| | | DSKVGGNYNYLYRLFRKSNLKPFERDISTEIYQAGSTPCNGVEGENCY |
| | | FPLQSYGFQPTNGVGYQPYRVVVLSFELLHAPATVCGPKKSTNLVKN |
| | | KCVNFNFNGLTGTGVLTESNKKFLPFQQFGRDIADTTDAVRDPQTLEI |
| | | LDITPCSFGGVSVITPGTNTSNQVAVLYQDVNCTEVPVAIHADQLTPT |
| | | WRVYSTGSNVFQTRAGCLIGAEHVNNSYECDIPIGAGICASYQTQTNS |
| | | PRRARSVASQSIIAYTMSLGAENSVAYSNNSIAIPTNFTISVTTEILPVS |
| | | MTKTSVDCTMYICGDSTECSNLLLQYGSFCTQLNRALTGIAVEQDKN |
| | | TQEVFAQVKQIYKTPPIKDFGGFNFSQILPDPSKPSKRSFIEDLLFNKVT |
| | | LADAGFIKQYGDCLGDIAARDLICAQKFNGLTVLPPLLTDEMIAQYTS |
| | | ALLAGTITSGWTFGAGAALQIPFAMQMAYRFNGIGVTQNVLYENQK |
| | | LIANQFNSAIGKIQDSLSSTASALGKLQDVVNQNAQALNTLVKQLSSN |
| | | FGAISSVLNDILSRLDKVEAEVQIDRLITGRLQSLQTYVTQQLIRAAEIR |
| | | ASANLAATKMSECVLGQSKRVDFCGKGYHLMSFPQSAPHGVVFLHV |
| | | TYVPAQEKNFTTAPAICHDGKAHFPREGVFVSNGTHWFVTQRNFYEP |
| | | QIITTDNTFVSGNCDVVIGIVNNTVYDPLQPELDSFKEELDKYFKNHTS |
| | | PDVDLGDISGINASVVNIQKEIDRLNEVAKNLNESLIDLQELGKYEQYI |
| | | KWPWYIWLGFIAGLIAIVMVTIMLCCMTSCCSCLKGCCSCGSCCKFD |
| | | EDDSEPVLKGVKLHYT |
| SARS-CoV-2 | 2 | NITNLCPFGEVFNATRFASVYAWNRKRISNCVADYSVLYNSASFSTFK |
| surface | | CYGVSPTKLNDLCFTNVYADSFVIRGDEVRQIAPGQTGKIADYNYKL |
| glycoprotein | | PDDFTGCVIAWNSNNLDSKVGGNYNYLYRLFRKSNLKPFERDISTEIY |
| RBD | | QAGSTPCNGVEGFNCYFPLQSYGFQPTNGVGYQPYRVVVLSFELLHA |
| GenBank: | | PATVCGPKKSTNLVKNKCVNFNFNGLTGTG |
| QHD43416.1; | | |
| Jan. 23, 2020 | | |
| SARS-CoV-2 | 3 | NSNNLDSKVGGNYNYLYRLFRKSNLKPFERDISTEIYQAGSTPCNGVE |
| RBM | | GFNCYFPLQSYGFQPTNGVGYQPY |
| GenBank: | | |
| QHD43416.1; | | |
| Jan. 23, 2020 | | |
| Linker (aa) | 4 | GSTSGSGKPGSGEGSTKG |
| Linker (aa) | 5 | GSGKPGSGEG |
| Linker (aa) | 6 | GKPGSGEG |
| Linker (aa) | 7 | SGKPGSGE |
| Linker (aa) | 8 | GGGGSGGGGSGGGGS |
| Linker (aa) | 9 | GGGGSGGGGSGGGGSGGGGSGGGGS |
| | | GGGGSGGGGSGGGGSGGGGSGGGGS |
| Linker (aa) | 10 | GSTSGGGSGGGSGGGGSS |
| Linker (aa) | 11 | EGKSSGSGSESKVD |
| Linker (aa) | 12 | KESGSVSSEQLAQFRSLD |
| Linker (aa) | 13 | GGGGS |
| S2X259 VH | 14 | QVQLVQSGAEVKKPGSSVKVSCKASGGIFNTYTISWVRQAPGQGLE |
| | | WMGRIILMSGMANYAQKIQGRVTITADKSTSTAYMELTSLRSDDTA |
| | | VYYCARGFNGNYYGWGDDDAFDISGQGTLVTVYS |
| S2X259 VL | 15 | QTVLTQPPSVSGAPGQRVTISCTGSNSNIGAGYDVHWYQQLPGTAPK |
| | | LLICGNSNRPSGVPDRFSGSKSGTSASLAITGLQAEDEADYYCQSYDS |
| | | SLSGPNWVFGGGTKLTVL |
| S309 VH (aa) | 16 | QVQLVQSGAEVKKPGASVKVSCKASGYPFTSYGISWVRQAPGQGLE |
| | | WMGWISTYNGNTNYAQKFQGRVTMTTDTSTTTGYMELRRLRSDDT |
| | | AVYYCARDYTRGAWFGESLIGGFDNWGQGTLVTVSS |
| S309 VL (VK) | 17 | EIVLTQSPGTLSLSPGERATLSCRASQTVSSTSLAWYQQKPGQAPRLLI |
| (aa) (identical to | | YGASSRATGIPDRFSGSGSGTDFTLTISRLEPEDFAVYYCQQHDTSLT |
| Sotrovimab VL | | FGGGTKVEIK |
| (VK) (aa)) | | |
| S2K146 VH (aa) | 18 | QVQLVESGGVVVQPGGSLRLSCAASGFTFHDHTMHWVRQAPGKGL |
| | | EWVSLITWNGGTIHYSDSVKGRFTISRDNSKNSLYLQMNSLRTEDTA |
| | | LYYCAKDLGRGGWYLPSDAWGQGTLVTVSS |
| S2K146 VL (aa) | 19 | QSVLTQPPSASGTPGQRVTISCSGSSANIGSNTVNWYQHLPGTAPKLL |
| | | IYSNNQRPSGVPDRFSGSKSGTSASLAISGLQSEDEADYYCAAWDDSL |
| | | KGVFGGGTKLTVL |
| S2X324-VH (aa) | 20 | QITLKESGPTLVKPTQTLTLTCTFSGFSVTTSGVGVGWIRQPPGKALE |
| | | YLALIYWDDDKRYSTSLKSRLTITKDTSKNQVVLTMTNMDPVDTAT |
| | | YYCARHTIPSIFDYWGQGILVTVSS |
| S2X324 VL (aa) | 21 | QPVLTQPASVSGSPGQSITISCTATSSDVGNYNYVSWYQHHPGKAPKL |
| | | MIYEVSNRPSGVSNRFSGSKSGNTASLTISGLQAEDEADYYCSSYTSS |
| | | SLLFGGGTKLTVL |
| Sotrovimab-VH | 22 | QVQLVQSGAEVKKPGASVKVSCKASGYPFTSYGISWVRQAPGQGLE |
| (aa) | | WMGWISTYQGNTNYAQKFQGRVTMTTDTSTTTGYMELRRLRSDDT |
| | | AVYYCARDYTRGAWFGESLIGGFDNWGQGTLVTVSS |
| Sotrovimab-VL | 23 | EIVLTQSPGTLSLSPGERATLSCRASQTVSSTSLAWYQQKPGQAPRLLI |
| (aa) (identical to | | YGASSRATGIPDRFSGSGSGTDFTLTISRLEPEDFAVYYCQQHDTSLT |
| S309 VL (VK)) | | FGGGTKVEIK |
| S312-VH (aa) | 24 | EVQLVETGGGLIQPGGSLTLSCAASGLVVSYNYMTWVRQAPGKGLE |
| | | WLSVIYAGGSTFYADSVKGRFTVSRDNSNNTLYLQMNNLRAEDTAV |
| | | YFCARWGQVRFTSEEAYYKYGLDVWGQGTTVTVSS |
| S312-VL (VK) | 25 | DIQLTQSPSFLSASVGDRVTITCRASQGISNYLAWYQQRPGKAPKLLIF |
| (aa) | | TASTLQSGVSSRFSGSGSGTEFTLTISSLQPEDLATYYCQQLNSNLQG |
| | | TFGQGTRVEIK |
| S3O13-VH (aa) | 26 | QVQLVQSGAEVKKPGASVKVSCKASGYTFTMSAMHWVRQAPGQRP |
| | | EWMGWITAGTGDTKYSQKFQGRVTITSDTSATTAYMELSSLRSEDT |
| | | AVYYCAKDGDYAAEYFQHWGQGTLVTVSS |
| S3O13-VL | 27 | DIVMTQSPSTLSASVGDRVTITCRASQSINSWLAWYQQKPGKAPNLLI |
| (VK) (aa) | | SDASNLESGVPSRFSGSGSGTEFTLTISSLQPDDFATYYCQQYNTYPSF |
| | | GQGTKVEIK |
| S3L17-VH (aa) | 28 | EVQLVESGGGLVQPGGSLRLSCAASEIIVSSNYMNWVRQAPGKGLE |
| | | WVSVIYAGGSTFYADSVRGRFTISRDNSKNTLYLQMNSLRPDDTAIY |
| | | YCARDLGPWGMDVWGQGTTVTVSS |
| S3L17-VL | 29 | DIQMTQSPSSLSASVGDRVAITCRASHGINNDLAWYQQKPGKVPKLL |
| (VK) (aa) | | IYAASTLQSGVPSRFSGSGSGTDFTLTISSLQPEDVATYYCQHYSSDPR |
| | | FTFGPGTKVDIK |
| Wild-type human | 30 | ASTKGPSVFPLAPSSKSTSGGTAALGCLVKDYFPEPVTVSWNSGALTS |
| IgG1 CH1-CH3 | | GVHTFPAVLQSSGLYSLSSVVTVPSSSLGTQTYICNVNHKPSNTKVDK |
| (UniProtKB | | KVEPKSCDKTHTCPPCPAPELLGGPSVFLFPPKPKDTLMISRTPEVTCVV |
| P01857); hinge | | VDVSHEDPEVKFNWYVDGVEVHNAKTKPREEQYNSTYRVVSVLTVLHQD |
| underlined, CH2 | | WLNGKEYKCKVSNKALPAPIEKTISKAKGQPREPQVYTLPPSRDELTKNQ |
| italicized | | VSLTCLVKGFYPSDIAVEWESNGQPENNYKTTPPVLDSDGSFFLYSKL |
| | | TVDKSRWQQGNVFSCSVMHEALHNHYTQKSLSLSPGK |
| Wild-type human | 31 | EPKSCDKTHTCPPCPAPELLGGPSVFLFPPKPKDTLMISRTPEVTCVVVD |
| IgG1 hinge-CH2- | | VSHEDPEVKFNWYVDGVEVHNAKTKPREEQYNSTYRVVSVLTVLHQDWL |
| CH3; hinge | | NGKEYKCKVSNKALPAPIEKTISKAKGQPREPQVYTLPPSRDELTKNQVS |
| underlined, CH2 | | LTCLVKGFYPSDIAVEWESNGQPENNYKTTPPVLDSDGSFFLYSKLTV |
| italicized | | DKSRWQQGNVFSCSVMHEALHNHYTQKSLSLSPGK |
| Wild-type human | 32 | APELLGGPSVFLFPPKPKDTLMISRTPEVTCVVVDVSHEDPEVKFNWY |
| IgG1 CH2 | | VDGVEVHNAKTKPREEQYNSTYRVVSVLTVLHQDWLNGKEYKCKV |
| | | SNKALPAPIEKTISKAK |
| Wild-type human | 33 | GQPREPQVYTLPPSRDELTKNQVSLTCLVKGFYPSDIAVEWESNGQPE |
| IgG1 CH3 | | NNYKTTPPVLDSDGSFFLYSKLTVDKSRWQQGNVFSCSVMHEALHN |
| | | HYTQKSLSLSPGK |
| Wild-type human | 34 | EPKSCDKTHTCPPCPAPELLGGPSVFLFPPKPKDTLMISRTPEVTCVVVD |
| IgG1 hinge-CH2; | | VSHEDPEVKFNWYVDGVEVHNAKTKPREEQYNSTYRVVSVLTVLHQDWL |
| hinge underlined, | | NGKEYKCKVSNKALPAPIEKTISKAK |
| CH2 italicized | | |
| IgG1m3 CH1- | 35 | ASTKGPSVFPLAPSSKSTSGGTAALGCLVKDYFPEPVTVSWNSGALTS |
| CH3 | | GVHTFPAVLQSSGLYSLSSVVTVPSSSLGTQTYICNVNHKPSNTKVDK |
| | | RVEPKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPS |
| | | REEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| IgG1m17,1 CH1- | 36 | ASTKGPSVFPLAPSSKSTSGGTAALGCLVKDYFPEPVTVSWNSGALTS |
| CH3 | | GVHTFPAVLQSSGLYSLSSVVTVPSSSLGTQTYICNVNHKPSNTKVDK |
| | | KVEPKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| IgG1m3,1 CH1- | 37 | ASTKGPSVFPLAPSSKSTSGGTAALGCLVKDYFPEPVTVSWNSGALTS |
| CH3 | | GVHTFPAVLQSSGLYSLSSVVTVPSSSLGTQTYICNVNHKPSNTKVDK |
| | | RVEPKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| Human IgG1 | 38 | ASTKGPSVFPLAPSSKSTSGGTAALGCLVKDYFPEPVTVSWNSGALTS |
| CH1-CH3- | | GVHTFPAVLQSSGLYSLSSVVTVPSSSLGTQTYICNVNHKPSNTKVDK |
| GAALIE | | KVEPKSCDKTHTCPPCPAPELLAGPSVFLFPPKPKDTLMISRTPEVTCV |
| mutation | | VVDVSHEDPEVKFNWYVDGVEVHNAKTKPREEQYNSTYRVVSVLT |
| | | VLHQDWLNGKEYKCKVSNKALPLPEEKTISKAKGQPREPQVYTLPPS |
| | | RDELTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| Human IgG1 | 39 | EPKSCDKTHTCPPCPAPELLAGPSVFLFPPKPKDTLMISRTPEVTCVVV |
| hinge-CH2-CH3- | | DVSHEDPEVKFNWYVDGVEVHNAKTKPREEQYNSTYRVVSVLTVLH |
| GAALIE | | QDWLNGKEYKCKVSNKALPLPEEKTISKAKGQPREPQVYTLPPSRDE |
| mutation | | LTKNQVSLTCLVKGFYPSDIAVEWESNGQPENNYKTTPPVLDSDGSFF |
| | | LYSKLTVDKSRWQQGNVFSCSVMHEALHNHYTQKSLSLSPGK |
| Human IgG1 | 40 | APELLAGPSVFLFPPKPKDTLMISRTPEVTCVVVDVSHEDPEVKFNWY |
| CH2-GAALIE | | VDGVEVHNAKTKPREEQYNSTYRVVSVLTVLHQDWLNGKEYKCKV |
| mutation | | SNKALPLPEEKTISKAK |
| Human IgG1 | 41 | EPKSCDKTHTCPPCPAPELLAGPSVFLFPPKPKDTLMISRTPEVTCVVV |
| hinge-CH2; | | DVSHEDPEVKFNWYVDGVEVHNAKTKPREEQYNSTYRVVSVLTVLH |
| -GAALIE | | QDWLNGKEYKCKVSNKALPLPEEKTISKAK |
| mutation | | |
| IgG1m3 CH1- | 42 | ASTKGPSVFPLAPSSKSTSGGTAALGCLVKDYFPEPVTVSWNSGALTS |
| CH3-GAALIE | | GVHTFPAVLQSSGLYSLSSVVTVPSSSLGTQTYICNVNHKPSNTKVDK |
| mutation | | RVEPKSCDKTHTCPPCPAPELLAGPSVFLFPPKPKDTLMISRTPEVTCV |
| | | VVDVSHEDPEVKFNWYVDGVEVHNAKTKPREEQYNSTYRVVSVLT |
| | | VLHQDWLNGKEYKCKVSNKALPLPEEKTISKAKGQPREPQVYTLPPS |
| | | REEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| IgG1m17,1 CH1- | 43 | ASTKGPSVFPLAPSSKSTSGGTAALGCLVKDYFPEPVTVSWNSGALTS |
| CH3-GAALIE | | GVHTFPAVLQSSGLYSLSSVVTVPSSSLGTQTYICNVNHKPSNTKVDK |
| mutation | | KVEPKSCDKTHTCPPCPAPELLAGPSVFLFPPKPKDTLMISRTPEVTCV |
| | | VVDVSHEDPEVKFNWYVDGVEVHNAKTKPREEQYNSTYRVVSVLT |
| | | VLHQDWLNGKEYKCKVSNKALPLPEEKTISKAKGQPREPQVYTLPPS |
| | | RDELTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| IgG1m3,1 CH1- | 44 | ASTKGPSVFPLAPSSKSTSGGTAALGCLVKDYFPEPVTVSWNSGALTS |
| CH3-GAALIE | | GVHTFPAVLQSSGLYSLSSVVTVPSSSLGTQTYICNVNHKPSNTKVDK |
| mutation | | RVEPKSCDKTHTCPPCPAPELLAGPSVFLFPPKPKDTLMISRTPEVTCV |
| | | VVDVSHEDPEVKFNWYVDGVEVHNAKTKPREEQYNSTYRVVSVLT |
| | | VLHQDWLNGKEYKCKVSNKALPLPEEKTISKAKGQPREPQVYTLPPS |
| | | RDELTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| Human IgG1 | 45 | ASTKGPSVFPLAPSSKSTSGGTAALGCLVKDYFPEPVTVSWNSGALTS |
| CH1-CH3-LS | | GVHTFPAVLQSSGLYSLSSVVTVPSSSLGTQTYICNVNHKPSNTKVDK |
| mutation | | KVEPKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSD |
| | | GSFFLYSKLTVDKSRWQQGNVFSCSVLHEALHSHYTQKSLSLSPGK |
| Human IgG1 | 46 | EPKSCDKTHTCPPCPAPELLGGPSVFLFPPKPKDTLMISRTPEVTCVVV |
| hinge-CH2-CH3- | | DVSHEDPEVKFNWYVDGVEVHNAKTKPREEQYNSTYRVVSVLTVLH |
| LS mutation | | QDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFF |
| | | LYSKLTVDKSRWQQGNVFSCSVLHEALHSHYTQKSLSLSPGK |
| Human IgG1 | 47 | GQPREPQVYTLPPSRDELTKNQVSLTCLVKGFYPSDIAVEWESNGQPE |
| CH3-LS | | NNYKTTPPVLDSDGSFFLYSKLTVDKSRWQQGNVFSCSVLHEALHSH |
| mutation | | YTQKSLSLSPGK |
| IgG1m3 CH1- | 48 | ASTKGPSVFPLAPSSKSTSGGTAALGCLVKDYFPEPVTVSWNSGALTS |
| CH3-LS | | GVHTFPAVLQSSGLYSLSSVVTVPSSSLGTQTYICNVNHKPSNTKVDK |
| mutation | | RVEPKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPS |
| | | REEMTKNQVSLTCLVKGFYPSDIAVEWESNGQPENNYKTTPPVLDSD |
| | | GSFFLYSKLTVDKSRWQQGNVFSCSVLHEALHSHYTQKSLSLSPGK |
| IgG1m17,1 | 49 | ASTKGPSVFPLAPSSKSTSGGTAALGCLVKDYFPEPVTVSWNSGALTS |
| IGHG1*01 CH1- | | GVHTFPAVLQSSGLYSLSSVVTVPSSSLGTQTYICNVNHKPSNTKVDK |
| CH3-LS | | KVEPKSCDKTHTCPPCPAPELLGGPSVFLFPPKPKDTLMISRTPEVTCV |
| mutation | | VVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSD |
| | | GSFFLYSKLTVDKSRWQQGNVFSCSVLHEALHSHYTQKSLSLSPGK |
| IgG1m3,1 CH1- | 50 | ASTKGPSVFPLAPSSKSTSGGTAALGCLVKDYFPEPVTVSWNSGALTS |
| CH3-LS | | GVHTFPAVLQSSGLYSLSSVVTVPSSSLGTQTYICNVNHKPSNTKVDK |
| mutation | | RVEPKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSD |
| | | GSFFLYSKLTVDKSRWQQGNVFSCSVLHEALHSHYTQKSLSLSPGK |
| Human IgG1 | 51 | ASTKGPSVFPLAPSSKSTSGGTAALGCLVKDYFPEPVTVSWNSGALTS |
| CH1-CH3-LA | | GVHTFPAVLQSSGLYSLSSVVTVPSSSLGTQTYICNVNHKPSNTKVDK |
| mutation | | KVEPKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSD |
| | | GSFFLYSKLTVDKSRWQQGNVFSCSVLHEALHAHYTQKSLSLSPGK |
| Human IgG1 | 52 | EPKSCDKTHTCPPCPAPELLGGPSVFLFPPKPKDTLMISRTPEVTCVVV |
| hinge-CH2-CH3- | | DVSHEDPEVKFNWYVDGVEVHNAKTKPREEQYNSTYRVVSVLTVLH |
| LA mutation | | QDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFF |
| | | LYSKLTVDKSRWQQGNVFSCSVLHEALHAHYTQKSLSLSPGK |
| Human IgG1 | 53 | GQPREPQVYTLPPSRDELTKNQVSLTCLVKGFYPSDIAVEWESNGQPE |
| CH3-LA | | NNYKTTPPVLDSDGSFFLYSKLTVDKSRWQQGNVFSCSVLHEALHAH |
| mutation | | YTQKSLSLSPGK |
| IgG1m3 CH1- | 54 | ASTKGPSVFPLAPSSKSTSGGTAALGCLVKDYFPEPVTVSWNSGALTS |
| CH3-LA | | GVHTFPAVLQSSGLYSLSSVVTVPSSSLGTQTYICNVNHKPSNTKVDK |
| mutation | | RVEPKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPS |
| | | REEMTKNQVSLTCLVKGFYPSDIAVEWESNGQPENNYKTTPPVLDSD |
| | | GSFFLYSKLTVDKSRWQQGNVFSCSVLHEALHAHYTQKSLSLSPGK |
| IgG1m17,1 CH1- | 55 | ASTKGPSVFPLAPSSKSTSGGTAALGCLVKDYFPEPVTVSWNSGALTS |
| CH3-LA | | GVHTFPAVLQSSGLYSLSSVVTVPSSSLGTQTYICNVNHKPSNTKVDK |
| mutation | | KVEPKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSD |
| | | GSFFLYSKLTVDKSRWQQGNVFSCSVLHEALHAHYTQKSLSLSPGK |
| IgG1m3,1 CH1- | 56 | ASTKGPSVFPLAPSSKSTSGGTAALGCLVKDYFPEPVTVSWNSGALTS |
| CH3-LA | | GVHTFPAVLQSSGLYSLSSVVTVPSSSLGTQTYICNVNHKPSNTKVDK |
| mutation | | RVEPKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSD |
| | | GSFFLYSKLTVDKSRWQQGNVFSCSVLHEALHAHYTQKSLSLSPGK |
| Human IgG1 | 57 | ASTKGPSVFPLAPSSKSTSGGTAALGCLVKDYFPEPVTVSWNSGALTS |
| CH1-CH3- | | GVHTFPAVLQSSGLYSLSSVVTVPSSSLGTQTYICNVNHKPSNTKVDK |
| GAALIE and LS | | KVEPKSCDKTHTCPPCPAPELLAGPSVFLFPPKPKDTLMISRTPEVTCV |
| mutations | | VVDVSHEDPEVKFNWYVDGVEVHNAKTKPREEQYNSTYRVVSVLT |
| | | VLHQDWLNGKEYKCKVSNKALPLPEEKTISKAKGQPREPQVYTLPPS |
| | | RDELTKNQVSLTCLVKGFYPSDIAVEWESNGQPENNYKTTPPVLDSD |
| | | GSFFLYSKLTVDKSRWQQGNVFSCSVLHEALHSHYTQKSLSLSPGK |
| Human IgG1 | 58 | EPKSCDKTHTCPPCPAPELLAGPSVFLFPPKPKDTLMISRTPEVTCVVV |
| hinge-CH2-CH3- | | DVSHEDPEVKFNWYVDGVEVHNAKTKPREEQYNSTYRVVSVLTVLH |
| GAALIE and | | QDWLNGKEYKCKVSNKALPLPEEKTISKAKGQPREPQVYTLPPSRDE |
| LS mutations | | LTKNQVSLTCLVKGFYPSDIAVEWESNGQPENNYKTTPPVLDSDGSFF |
| | | LYSKLTVDKSRWQQGNVFSCSVLHEALHSHYTQKSLSLSPGK |
| IgG1m3 CH1- | 59 | ASTKGPSVFPLAPSSKSTSGGTAALGCLVKDYFPEPVTVSWNSGALTS |
| CH3-GAALIE | | GVHTFPAVLQSSGLYSLSSVVTVPSSSLGTQTYICNVNHKPSNTKVDK |
| and LS mutations | | RVEPKSCDKTHTCPPCPAPELLAGPSVFLFPPKPKDTLMISRTPEVTCV |
| | | VVDVSHEDPEVKFNWYVDGVEVHNAKTKPREEQYNSTYRVVSVLT |
| | | VLHQDWLNGKEYKCKVSNKALPLPEEKTISKAKGQPREPQVYTLPPS |
| | | REEMTKNQVSLTCLVKGFYPSDIAVEWESNGQPENNYKTTPPVLDSD |
| | | GSFFLYSKLTVDKSRWQQGNVFSCSVLHEALHSHYTQKSLSLSPGK |
| IgG1m17,1 CH1- | 60 | ASTKGPSVFPLAPSSKSTSGGTAALGCLVKDYFPEPVTVSWNSGALTS |
| CH3-GAALIE | | GVHTFPAVLQSSGLYSLSSVVTVPSSSLGTQTYICNVNHKPSNTKVDK |
| and LS mutations | | KVEPKSCDKTHTCPPCPAPELLAGPSVFLFPPKPKDTLMISRTPEVTCV |
| | | VVDVSHEDPEVKFNWYVDGVEVHNAKTKPREEQYNSTYRVVSVLT |
| | | VLHQDWLNGKEYKCKVSNKALPLPEEKTISKAKGQPREPQVYTLPPS |
| | | RDELTKNQVSLTCLVKGFYPSDIAVEWESNGQPENNYKTTPPVLDSD |
| | | GSFFLYSKLTVDKSRWQQGNVFSCSVLHEALHSHYTQKSLSLSPGK |
| IgG1m3,1 CH1- | 61 | ASTKGPSVFPLAPSSKSTSGGTAALGCLVKDYFPEPVTVSWNSGALTS |
| CH3-GAALIE | | GVHTFPAVLQSSGLYSLSSVVTVPSSSLGTQTYICNVNHKPSNTKVDK |
| and LS mutations | | RVEPKSCDKTHTCPPCPAPELLAGPSVFLFPPKPKDTLMISRTPEVTCV |
| | | VVDVSHEDPEVKFNWYVDGVEVHNAKTKPREEQYNSTYRVVSVLT |
| | | VLHQDWLNGKEYKCKVSNKALPLPEEKTISKAKGQPREPQVYTLPPS |
| | | RDELTKNQVSLTCLVKGFYPSDIAVEWESNGQPENNYKTTPPVLDSD |
| | | GSFFLYSKLTVDKSRWQQGNVFSCSVLHEALHSHYTQKSLSLSPGK |
| Human IgG1 | 62 | ASTKGPSVFPLAPSSKSTSGGTAALGCLVKDYFPEPVTVSWNSGALTS |
| CH1-CH3- | | GVHTFPAVLQSSGLYSLSSVVTVPSSSLGTQTYICNVNHKPSNTKVDK |
| GAALIE and LA | | KVEPKSCDKTHTCPPCPAPELLAGPSVFLFPPKPKDTLMISRTPEVTCV |
| mutations | | VVDVSHEDPEVKFNWYVDGVEVHNAKTKPREEQYNSTYRVVSVLT |
| | | VLHQDWLNGKEYKCKVSNKALPLPEEKTISKAKGQPREPQVYTLPPS |
| | | RDELTKNQVSLTCLVKGFYPSDIAVEWESNGQPENNYKTTPPVLDSD |
| | | GSFFLYSKLTVDKSRWQQGNVFSCSVLHEALHAHYTQKSLSLSPGK |
| Human IgG1 | 63 | EPKSCDKTHTCPPCPAPELLAGPSVFLFPPKPKDTLMISRTPEVTCVVV |
| hinge-CH2-CH3- | | DVSHEDPEVKFNWYVDGVEVHNAKTKPREEQYNSTYRVVSVLTVLH |
| GAALIE and | | QDWLNGKEYKCKVSNKALPLPEEKTISKAKGQPREPQVYTLPPSRDE |
| LA mutations | | LTKNQVSLTCLVKGFYPSDIAVEWESNGQPENNYKTTPPVLDSDGSFF |
| | | LYSKLTVDKSRWQQGNVFSCSVLHEALHAHYTQKSLSLSPGK |
| IgG1m3 CH1- | 64 | ASTKGPSVFPLAPSSKSTSGGTAALGCLVKDYFPEPVTVSWNSGALTS |
| CH3-GAALIE | | GVHTFPAVLQSSGLYSLSSVVTVPSSSLGTQTYICNVNHKPSNTKVDK |
| and LA mutations | | RVEPKSCDKTHTCPPCPAPELLAGPSVFLFPPKPKDTLMISRTPEVTCV |
| | | VVDVSHEDPEVKFNWYVDGVEVHNAKTKPREEQYNSTYRVVSVLT |
| | | VLHQDWLNGKEYKCKVSNKALPLPEEKTISKAKGQPREPQVYTLPPS |
| | | REEMTKNQVSLTCLVKGFYPSDIAVEWESNGQPENNYKTTPPVLDSD |
| | | GSFFLYSKLTVDKSRWQQGNVFSCSVLHEALHAHYTQKSLSLSPGK |
| IgG1m17,1 CH1- | 65 | ASTKGPSVFPLAPSSKSTSGGTAALGCLVKDYFPEPVTVSWNSGALTS |
| CH3-GAALIE | | GVHTFPAVLQSSGLYSLSSVVTVPSSSLGTQTYICNVNHKPSNTKVDK |
| and LA mutations | | KVEPKSCDKTHTCPPCPAPELLAGPSVFLFPPKPKDTLMISRTPEVTCV |
| | | VVDVSHEDPEVKFNWYVDGVEVHNAKTKPREEQYNSTYRVVSVLT |
| | | VLHQDWLNGKEYKCKVSNKALPLPEEKTISKAKGQPREPQVYTLPPS |
| | | RDELTKNQVSLTCLVKGFYPSDIAVEWESNGQPENNYKTTPPVLDSD |
| | | GSFFLYSKLTVDKSRWQQGNVFSCSVLHEALHAHYTQKSLSLSPGK |
| IgG1m3,1 CH1- | 66 | ASTKGPSVFPLAPSSKSTSGGTAALGCLVKDYFPEPVTVSWNSGALTS |
| CH3-GAALIE | | GVHTFPAVLQSSGLYSLSSVVTVPSSSLGTQTYICNVNHKPSNTKVDK |
| and LA mutations | | RVEPKSCDKTHTCPPCPAPELLAGPSVFLFPPKPKDTLMISRTPEVTCV |
| | | VVDVSHEDPEVKFNWYVDGVEVHNAKTKPREEQYNSTYRVVSVLT |
| | | VLHQDWLNGKEYKCKVSNKALPLPEEKTISKAKGQPREPQVYTLPPS |
| | | RDELTKNQVSLTCLVKGFYPSDIAVEWESNGQPENNYKTTPPVLDSD |
| | | GSFFLYSKLTVDKSRWQQGNVFSCSVLHEALHAHYTQKSLSLSPGK |
| S2V29-VH | 67 | EVQLVESGGSVVQPGRALRLSCAASGITFSSFGMYWVRQAPGKGLE |
| (also S2V29- | | WLGVIAYDGSNTYYADSVKGRFTISRDNSKNTLYLQMNSLRIEDTAV |
| VH.1) (aa) | | YYCAKDYYPLLSYYYGLDVWGQGTTVTVSS |
| S2V29-CDRH1 | 68 | GITFSSFG |
| also S2V29-v37.2 | | |
| CDRH1 | | |
| S2V29-CDRH2 | 69 | IAYDGSNT |
| S2V29-CDRH3 | 70 | AKDYYPLLSYYYGLDV |
| also S2V29-v37.2 | | |
| CDRH3 | | |
| S2V29-VL-11a | 71 | QSVLTQPRSVSGSPGQSVTISCTGTSSDVGAYNYVSWYQQHPGKAPK |
| (also S2V29- | | FMIYDVDQRPSGVPDRFSGSKSGNTASLIISGLQAEDEADYYCCSYAG |
| VL.1) (aa) | | SYIWVFGGGTQLTVL |
| S2V29-VL-11a | 72 | SSDVGAYNY |
| CDRL1 | | |
| also S2V29-v37.2 | | |
| CDRL1 | | |
| S2V29-VL-11a | 73 | DVD |
| CDRL2 | | |
| also S2V29-v37.2 | | |
| CDRL2 | | |
| S2V29-VL-11a | 74 | CSYAGSYIWV |
| CDRL3 | | |
| S2V29-VH (nt) | 75 | GAGGTGCAGCTGGTGGAGTCTGGGGGAAGTGTGGTCCAGCCTGGG |
| | | AGGGCCCTGAGACTCTCCTGTGCAGCCTCTGGAATCACCTTCAGT |
| | | AGCTTTGGCATGTACTGGGTCCGCCAGGCTCCAGGCAAGGGGCTG |
| | | GAGTGGCTGGGAGTTATAGCGTATGATGGAAGTAATACATACTA |
| | | TGCAGACTCCGTGAAGGGCCGATTCACCATCTCCAGAGACAATTC |
| | | CAAGAACACGCTGTATTTGCAAATGAACAGCCTGAGAATTGAGGA |
| | | CACGGCTGTCTATTATTGTGCGAAAGATTATTACCCGCTGCTATC |
| | | GTACTACTACGGTTTGGACGTCTGGGGCCAAGGGACCACGGTCA |
| | | CCGTATCCTCA |
| S2V29-VL-11a | 76 | CAGTCTGTGCTGACTCAGCCTCGCTCAGTGTCCGGGTCTCCTGGAC |
| (nt) | | AGTCAGTCACCATTTCCTGCACTGGAACCAGCAGTGATGTCGGT |
| | | GCTTATAACTATGTCTCCTGGTACCAACAACACCCAGGCAAAGCC |
| | | CCCAAATTCATGATTTATGATGTCGATCAGCGGCCCTCAGGGGTC |
| | | CCTGATCGCTTCTCTGGCTCCAAGTCTGGCAACACGGCCTCCCTGA |
| | | TCATCTCTGGGCTCCAGGCTGAGGATGAGGCTGATTATTACTGCTG |
| | | CTCATATGCTGGCAGCTACATTTGGGTGTTCGGCGGAGGGACCC |
| | | AACTGACCGTCCTAG |
| S2V29-VL-11b | 77 | QSVLTQPRSVSGSPGQSVTISCTGTSSDVGAYNYVSWYQQHPGKAPK |
| (aa) | | FMIYDVDQRPSGVPDRFSGSKSGN |
| | | TASLIISGLQAEDEADYYCSSYAGSYIWVFGGGTQLTVL |
| S2V29-VL-11b | 78 | SSYAGSYIWV |
| CDRL3 | | |
| S2V29-VH.1 (nt) | 79 | GAAGTGCAACTAGTGGAAAGTGGTGGTAGTGTCGTGCAGCCTGGC |
| | | AGAGCCCTCAGACTGAGCTGTGCTGCTTCTGGCATCACCTTCTCCT |
| | | CTTTTGGCATGTACTGGGTGCGGCAAGCTCCCGGAAAAGGACTGG |
| | | AATGGCTGGGCGTGATCGCCTACGACGGCTCTAATACCTACTACG |
| | | CCGATTCCGTGAAGGGCCGCTTCACAATCTCCAGAGATAACTCCA |
| | | AGAACACCCTGTACCTGCAGATGAACTCTCTGCGGATCGAGGACA |
| | | CCGCCGTGTACTACTGCGCCAAGGACTACTATCCTCTGCTGTCCTA |
| | | CTACTACGGCCTGGACGTGTGGGGCCAGGGCACCACAGTGACCGT |
| | | GTCCAGC |
| S2V29-VL.1 (nt) | 80 | CAAAGTGTGCTAACTCAACCACGAAGTGTGTCTGGCTCTCCAGGA |
| | | CAATCCGTGACCATCTCCTGCACCGGCACCTCCAGCGATGTGGGC |
| | | GCCTACAACTACGTGTCCTGGTACCAGCAGCACCCTGGCAAGGCC |
| | | CCTAAGTTCATGATCTACGACGTGGACCAGCGGCCTTCTGGCGTGC |
| | | CCGATAGATTCTCCGGCAGCAAATCCGGCAACACCGCTTCTCTGAT |
| | | CATCAGCGGCCTGCAGGCCGAGGACGAAGCCGACTACTACTGCTG |
| | | TTCTTATGCTGGCTCCTACATCTGGGTCTTTGGCGGCGGAACACAG |
| | | CTGACCGTGCTG |
| S2V29-VH.2 (aa) | 81 | EVQLVESGGSVVQPGRALRLSCAASGITFSSFGMYWVRQAPGKGLE |
| | | WLGVIAYKGSNTYYADSVKGRFTISRDNSKNTLYLQMNSLRIEDTAV |
| | | YYCAKDYYPLLSYYYGLDVWGQGTTVTVSS |
| S2V29-VH.2 | 82 | IAYKGSNT |
| CDRH2 | | |
| S2V29-VH.2 (nt) | 83 | GAAGTGCAACTAGTGGAAAGTGGTGGTAGTGTCGTGCAGCCTGGC |
| | | AGAGCCCTCAGACTGAGCTGTGCTGCTTCTGGCATCACCTTCTCCT |
| | | CTTTTGGCATGTACTGGGTGCGGCAAGCTCCCGGAAAAGGACTGG |
| | | AATGGCTGGGCGTGATCGCCTACAAGGGCTCTAATACCTACTACG |
| | | CCGATTCCGTGAAGGGCCGCTTCACAATCTCCAGAGATAACTCCA |
| | | AGAACACCCTGTACCTGCAGATGAACTCTCTGCGGATCGAGGACA |
| | | CCGCCGTGTACTACTGCGCCAAGGACTACTATCCTCTGCTGTCCTA |
| | | CTACTACGGCCTGGACGTGTGGGGCCAGGGCACCACAGTGACCGT |
| | | GTCCAGC |
| S2V29-VH.3 (aa) | 84 | EVQLVESGGSVVQPGRALRLSCAASGITFSSFGMYWVRQAPGKGLE |
| | | WLGVIAYSGSNTYYADSVKQRFTISRDNSKNTLYLQMNSLRIEDTAV |
| | | YYCAKDYYPLLSYYYGLDVWGQGTTVTVSS |
| S2V29-VH.3 | 85 | IAYSGSNT |
| CDRH2 | | |
| S2V29-VH.3 (nt) | 86 | GAAGTGCAACTAGTGGAAAGTGGTGGTAGTGTCGTGCAGCCTGGC |
| | | AGAGCCCTCAGACTGAGCTGTGCTGCTTCTGGCATCACCTTCTCCT |
| | | CTTTTGGCATGTACTGGGTGCGGCAAGCTCCCGGAAAAGGACTGG |
| | | AATGGCTGGGCGTGATCGCCTACTCCGGCTCTAATACCTACTACGC |
| | | CGATTCCGTGAAGGGCCGCTTCACAATCTCCAGAGATAACTCCAA |
| | | GAACACCCTGTACCTGCAGATGAACTCTCTGCGGATCGAGGACAC |
| | | CGCCGTGTACTACTGCGCCAAGGACTACTATCCTCTGCTGTCCTAC |
| | | TACTACGGCCTGGACGTGTGGGGCCAGGGCACCACAGTGACCGTG |
| | | TCCAGC |
| S2V29-VH.4 | 87 | EVQLVESGGSVVQPGRALRLSCAASGITFSSFGMYWVRQAPGKGLE |
| | | WLGVIAYEGSNTYYADSVKQRFTISRDNSKNTLYLQMNSLRIEDTAV |
| | | YYCAKDYYPLLSYYYGLDVWGQGTTVTVSS |
| S2V29-VH.4 | 88 | IAYEGSNT |
| CDRH2 | | |
| S2V29-VH.4 (nt) | 89 | GAAGTGCAACTAGTGGAAAGTGGTGGTAGTGTCGTGCAGCCTGGC |
| | | AGAGCCCTCAGACTGAGCTGTGCTGCTTCTGGCATCACCTTCTCCT |
| | | CTTTTGGCATGTACTGGGTGCGGCAAGCTCCCGGAAAAGGACTGG |
| | | AATGGCTGGGCGTGATCGCCTACGAGGGCTCTAATACCTACTACG |
| | | CCGATTCCGTGAAGGGCCGCTTCACAATCTCCAGAGATAACTCCA |
| | | AGAACACCCTGTACCTGCAGATGAACTCTCTGCGGATCGAGGACA |
| | | CCGCCGTGTACTACTGCGCCAAGGACTACTATCCTCTGCTGTCCTA |
| | | CTACTACGGCCTGGACGTGTGGGGCCAGGGCACCACAGTGACCGT |
| | | GTCCAGC |
| S2V29-VH.5 | 90 | EVQLVESGGSVVQPGRALRLSCAASGITFSSFGMYWVRQAPGKGLE |
| | | WLGVIAYDASNTYYADSVKGRFTISRDNSKNTLYLQMNSLRIEDTAV |
| | | YYCAKDYYPLLSYYYGLDVWGQGTTVTVSS |
| S2V29-VH.5 | 91 | IAYDASNT |
| CDRH2 | | |
| S2V29-VH.5 (nt) | 92 | GAAGTGCAACTAGTGGAAAGTGGTGGTAGTGTCGTGCAGCCTGGC |
| | | AGAGCCCTCAGACTGAGCTGTGCTGCTTCTGGCATCACCTTCTCCT |
| | | CTTTTGGCATGTACTGGGTGCGGCAAGCTCCCGGAAAAGGACTGG |
| | | AATGGCTGGGCGTGATCGCCTACGACGCCTCTAATACCTACTACGC |
| | | CGATTCCGTGAAGGGCCGCTTCACAATCTCCAGAGATAACTCCAA |
| | | GAACACCCTGTACCTGCAGATGAACTCTCTGCGGATCGAGGACAC |
| | | CGCCGTGTACTACTGCGCCAAGGACTACTATCCTCTGCTGTCCTAC |
| | | TACTACGGCCTGGACGTGTGGGGCCAGGGCACCACAGTGACCGTG |
| | | TCCAGC |
| S2V29-VH.6 | 93 | EVQLVESGGSVVQPGRALRLSCAASGITFSSFGMYWVRQAPGKGLE |
| | | WLGVIAYSGSNTYYADSVKGRFTISRDNSKNTLYLQMNSLRIEDTAV |
| | | YYCAKDYYPLLSYYYGIDVWGQGTTVTVSS |
| S2V29-VH.6 | 94 | IAYSGSNT |
| CDRH2 | | |
| S2V29-VH.6 | 95 | AKDYYPLLSYYYGIDV |
| CDRH3 | | |
| S2V29-VH.6 (nt) | 96 | GAAGTGCAACTAGTGGAAAGTGGTGGTAGTGTCGTGCAGCCTGGC |
| | | AGAGCCCTCAGACTGAGCTGTGCTGCTTCTGGCATCACCTTCTCCT |
| | | CTTTTGGCATGTACTGGGTGCGGCAAGCTCCCGGAAAAGGACTGG |
| | | AATGGCTGGGCGTGATCGCCTACTCCGGCTCTAATACCTACTACGC |
| | | CGATTCCGTGAAGGGCCGCTTCACAATCTCCAGAGATAACTCCAA |
| | | GAACACCCTGTACCTGCAGATGAACTCTCTGCGGATCGAGGACAC |
| | | CGCCGTGTACTACTGCGCCAAGGACTACTATCCTCTGCTGTCCTAC |
| | | TACTACGGCATCGACGTGTGGGGCCAGGGCACCACAGTGACCGTG |
| | | TCCAGC |
| S2V29-VH.7 | 97 | EVQLVESGGSVVQPGRALRLSCAASGITFSSFGMYWVRQAPGKGLE |
| | | WLGVIAYEGSNTYYADSVKGRFTISRDNSKNTLYLQMNSLRIEDTAV |
| | | YYCAKDYYPLLSYYYGIDVWGQGTTVTVSS |
| S2V29-VH.7 | 98 | IAYEGSNT |
| CDRH2 | | |
| S2V29-VH.7 | 99 | AKDYYPLLSYYYGIDV |
| CDRH3 | | |
| S2V29-VH.7 (nt) | 100 | GAAGTGCAACTAGTGGAAAGTGGTGGTAGTGTCGTGCAGCCTGGC |
| | | AGAGCCCTCAGACTGAGCTGTGCTGCTTCTGGCATCACCTTCTCCT |
| | | CTTTTGGCATGTACTGGGTGCGGCAAGCTCCCGGAAAAGGACTGG |
| | | AATGGCTGGGCGTGATCGCCTACGAGGGCTCTAATACCTACTACG |
| | | CCGATTCCGTGAAGGGCCGCTTCACAATCTCCAGAGATAACTCCA |
| | | AGAACACCCTGTACCTGCAGATGAACTCTCTGCGGATCGAGGACA |
| | | CCGCCGTGTACTACTGCGCCAAGGACTACTATCCTCTGCTGTCCTA |
| | | CTACTACGGCATCGACGTGTGGGGCCAGGGCACCACAGTGACCGT |
| | | GTCCAGC |
| S2V29-VH.8 | 101 | EVQLVESGGSVVQPGRALRLSCAASGITFSSFGMYWVRQAPGKGLE |
| | | WLGVIAYKGSNTYYADSVKGRFTISRDNSKNTLYLQMNSLRIEDTAV |
| | | YYCAKDYYPLLSYYYGIDVWGQGTTVTVSS |
| S2V29-VH.8 | 102 | IAYKGSNT |
| CDRH2 | | |
| S2V29-VH.8 | 103 | AKDYYPLLSYYYGIDV |
| CDRH3 | | |
| S2V29-VH.8 (nt) | 104 | GAAGTGCAACTAGTGGAAAGTGGTGGTAGTGTCGTGCAGCCTGGC |
| | | AGAGCCCTCAGACTGAGCTGTGCTGCTTCTGGCATCACCTTCTCCT |
| | | CTTTTGGCATGTACTGGGTGCGGCAAGCTCCCGGAAAAGGACTGG |
| | | AATGGCTGGGCGTGATCGCCTACAAGGGCTCTAATACCTACTACG |
| | | CCGATTCCGTGAAGGGCCGCTTCACAATCTCCAGAGATAACTCCA |
| | | AGAACACCCTGTACCTGCAGATGAACTCTCTGCGGATCGAGGACA |
| | | CCGCCGTGTACTACTGCGCCAAGGACTACTATCCTCTGCTGTCCTA |
| | | CTACTACGGCATCGACGTGTGGGGCCAGGGCACCACAGTGACCGT |
| | | GTCCAGC |
| S2V29-VH.9 | 105 | EVQLVESGGSVVQPGRALRLSCAASGITFSSFGMYWVRQAPGKGLE |
| | | WLGVIAYDASNTYYADSVKGRFTISRDNSKNTLYLQMNSLRIEDTAV |
| | | YYCAKDYYPLLSYYYGIDVWGQGTTVTVSS |
| S2V29-VH.9 | 106 | IAYDASNT |
| CDRH2 | | |
| S2V29-VH.9 | 107 | AKDYYPLLSYYYGIDV |
| CDRH3 | | |
| S2V29-VH.9 (nt) | 108 | GAAGTGCAACTAGTGGAAAGTGGTGGTAGTGTCGTGCAGCCTGGC |
| | | AGAGCCCTCAGACTGAGCTGTGCTGCTTCTGGCATCACCTTCTCCT |
| | | CTTTTGGCATGTACTGGGTGCGGCAAGCTCCCGGAAAAGGACTGG |
| | | AATGGCTGGGCGTGATCGCCTACGACGCCTCTAATACCTACTACGC |
| | | CGATTCCGTGAAGGGCCGCTTCACAATCTCCAGAGATAACTCCAA |
| | | GAACACCCTGTACCTGCAGATGAACTCTCTGCGGATCGAGGACAC |
| | | CGCCGTGTACTACTGCGCCAAGGACTACTATCCTCTGCTGTCCTAC |
| | | TACTACGGCATCGACGTGTGGGGCCAGGGCACCACAGTGACCGTG |
| | | TCCAGC |
| S2V29-VH.10 | 109 | EVQLVESGGSVVQPGRALRLSCAASGITFSSFGMYWVRQAPGKGLE |
| | | WLGVIAYDDSNTYYADSVKGRFTISRDNSKNTLYLQMNSLRIEDTAV |
| | | YYCAKDYYPLLSYYYGIDVWGQGTTVTVSS |
| S2V29-VH.10 | 110 | IAYDDSNT |
| CDRH2 | | |
| S2V29-VH.10 | 111 | AKDYYPLLSYYYGIDV |
| CDRH3 | | |
| S2V29-VH.10 | 112 | GAAGTGCAACTAGTGGAAAGTGGTGGTAGTGTCGTGCAGCCTGGC |
| (nt) | | AGAGCCCTCAGACTGAGCTGTGCTGCTTCTGGCATCACCTTCTCCT |
| | | CTTTTGGCATGTACTGGGTGCGGCAAGCTCCCGGAAAAGGACTGG |
| | | AATGGCTGGGCGTGATCGCCTACGACGACTCTAATACCTACTACG |
| | | CCGATTCCGTGAAGGGCCGCTTCACAATCTCCAGAGATAACTCCA |
| | | AGAACACCCTGTACCTGCAGATGAACTCTCTGCGGATCGAGGACA |
| | | CCGCCGTGTACTACTGCGCCAAGGACTACTATCCTCTGCTGTCCTA |
| | | CTACTACGGCATCGACGTGTGGGGCCAGGGCACCACAGTGACCGT |
| | | GTCCAGC |
| S2V29-VL.2 | 113 | QSVLTQPRSVSGSPGQSVTISCTGTSSDVGAYNYVSWYQQHPGKAPK |
| (C91S) (aa) | | FMIYDVDQRPSGVPDRFSGSKSGNTASLIISGLQAEDEADYYCSSYAG |
| | | SYIWVFGGGTQLTVL |
| S2V29-VL.2 | 114 | SSYAGSYIWV |
| CDRL3 | | |
| S2V29-VL.2 (nt) | 115 | CAAAGTGTGCTAACTCAACCACGAAGTGTGAGCGGCTCTCCTGGC |
| | | CAGTCCGTGACCATCTCCTGTACCGGCACCTCTAGCGATGTGGGCG |
| | | CTTACAACTACGTGTCCTGGTATCAACAGCACCCCGGCAAGGCCC |
| | | CTAAGTTCATGATCTACGACGTGGACCAGCGGCCATCTGGCGTGC |
| | | CTGATAGATTCTCCGGCTCCAAATCCGGCAACACCGCTTCTCTGAT |
| | | CATCTCTGGCCTGCAGGCCGAGGACGAAGCCGACTACTACTGCTC |
| | | CTCCTACGCCGGATCTTACATCTGGGTCTTTGGCGGCGGAACACAG |
| | | CTGACCGTGCTG |
| S2V29-VL.3 (aa) | 116 | QSVLTQPRSVSGSPGQSVTISCTGTSSDVGAYNYVSWYQQHPGKAPK |
| | | FMIYDVDQRPSGVPDRFSGSKSGNTASLIISGLQAEDEADYYCASYAG |
| | | SYIWVFGGGTQLTVL |
| S2V29-VL.3 | 117 | ASYAGSYIWV |
| CDRL3 | | |
| S2V29-VL.3 (nt) | 118 | CAAAGTGTGCTAACTCAACCACGAAGTGTGTCTGGCTCTCCAGGA |
| | | CAATCCGTGACCATCTCCTGCACCGGCACCTCCAGCGATGTGGGC |
| | | GCCTACAACTACGTGTCCTGGTACCAGCAGCACCCTGGCAAGGCC |
| | | CCTAAGTTCATGATCTACGACGTGGACCAGCGGCCTTCTGGCGTGC |
| | | CCGATAGATTCTCCGGCAGCAAATCCGGCAACACCGCTTCTCTGAT |
| | | CATCAGCGGCCTGCAGGCCGAGGACGAAGCCGACTACTACTGCGC |
| | | CTCTTATGCTGGCTCCTACATCTGGGTCTTTGGCGGCGGAACACAG |
| | | CTGACCGTGCTG |
| S2V29-VL.4 (aa) | 119 | QSVLTQPRSVSGSPGQSVTISCTGTSSDVGAYNYVSWYQQHPGKAPK |
| | | FMIYDVDQRPSGVPDRFSGSKSQNTASLIISGLQAEDEADYYCVSYAG |
| | | SYIWVFGGGTQLTVL |
| S2V29-VL.4 | 120 | VSYAGSYIWV |
| CDRL3 | | |
| S2V29-VL.4 (nt) | 121 | CAAAGTGTGCTAACTCAACCACGAAGTGTGTCTGGCTCTCCAGGA |
| | | CAATCCGTGACCATCTCCTGCACCGGCACCTCCAGCGATGTGGGC |
| | | GCCTACAACTACGTGTCCTGGTACCAGCAGCACCCTGGCAAGGCC |
| | | CCTAAGTTCATGATCTACGACGTGGACCAGCGGCCTTCTGGCGTGC |
| | | CCGATAGATTCTCCGGCAGCAAATCCGGCAACACCGCTTCTCTGAT |
| | | CATCAGCGGCCTGCAGGCCGAGGACGAAGCCGACTACTACTGCGT |
| | | GTCTTATGCTGGCTCCTACATCTGGGTCTTTGGCGGCGGAACACAG |
| | | CTGACCGTGCTG |
| S2V29-VL.5 (aa) | 122 | QSVLTQPRSVSGSPGQSVTISCTGTSSDVGAYNYVSWYQQHPGKAPK |
| | | FMIYDVDQRPSGVPDRFSGSKSQNTASLIISGLQAEDEADYYCYSYAG |
| | | SYIWVFGGGTQLTVL |
| S2V29-VL.5 | 123 | YSYAGSYIWV |
| CDRL3 | | |
| S2V29-VL.5 (nt) | 124 | CAAAGTGTGCTAACTCAACCACGAAGTGTGTCTGGCTCTCCAGGA |
| | | CAATCCGTGACCATCTCCTGCACCGGCACCTCCAGCGATGTGGGC |
| | | GCCTACAACTACGTGTCCTGGTACCAGCAGCACCCTGGCAAGGCC |
| | | CCTAAGTTCATGATCTACGACGTGGACCAGCGGCCTTCTGGCGTGC |
| | | CCGATAGATTCTCCGGCAGCAAATCCGGCAACACCGCTTCTCTGAT |
| | | CATCAGCGGCCTGCAGGCCGAGGACGAAGCCGACTACTACTGCTA |
| | | CTCTTATGCTGGCTCCTACATCTGGGTCTTTGGCGGCGGAACACAG |
| | | CTGACCGTGCTG |
| S2V29-VL.6 (aa) | 125 | QSVLTQPRSVSGSPGQSVTISCTGTSSDVGAYNYVSWYQQHPGKAPK |
| | | FMIYDVDQRPSGVPDRFSGSKSGNTASLIISGLQAEDEADYYCGSYAG |
| | | SYIWVFGGGTQLTVL |
| S2V29-VL.6 | 126 | GSYAGSYIWV |
| CDRL3 | | |
| S2V29-VL.6 (nt) | 127 | CAAAGTGTGCTAACTCAACCACGAAGTGTGTCTGGCTCTCCAGGA |
| | | CAATCCGTGACCATCTCCTGCACCGGCACCTCCAGCGATGTGGGC |
| | | GCCTACAACTACGTGTCCTGGTACCAGCAGCACCCTGGCAAGGCC |
| | | CCTAAGTTCATGATCTACGACGTGGACCAGCGGCCTTCTGGCGTGC |
| | | CCGATAGATTCTCCGGCAGCAAATCCGGCAACACCGCTTCTCTGAT |
| | | CATCAGCGGCCTGCAGGCCGAGGACGAAGCCGACTACTACTGCGG |
| | | CTCTTATGCTGGCTCCTACATCTGGGTCTTTGGCGGCGGAACACAG |
| | | CTGACCGTGCTG |
| S2V29-VL.7 (aa) | 128 | QSVLTQPRSVSGSPGQSVTISCTGTSSDVGAYNYVSWYQQHPGKAPK |
| | | FMIYDVDQRPSGVPDRFSGSKSGNTASLIISGLQAEDEADYYCSYAGS |
| | | YIWVFGGGTQLTVL |
| S2V29-VL.7 | 129 | SYAGSYIWV |
| CDRL3 | | |
| S2V29-VL.7 (nt) | 130 | CAAAGTGTGCTAACTCAACCACGAAGTGTGTCTGGCTCTCCAGGA |
| | | CAATCCGTGACCATCTCCTGCACCGGCACCTCCAGCGATGTGGGC |
| | | GCCTACAACTACGTGTCCTGGTACCAGCAGCACCCTGGCAAGGCC |
| | | CCTAAGTTCATGATCTACGACGTGGACCAGCGGCCTTCTGGCGTGC |
| | | CCGATAGATTCTCCGGCAGCAAATCCGGCAACACCGCTTCTCTGAT |
| | | CATCAGCGGCCTGCAGGCCGAGGACGAAGCCGACTACTACTGTTC |
| | | TTATGCTGGCTCCTACATCTGGGTCTTTGGCGGCGGAACACAGCTG |
| | | ACCGTGCTG |
| S2V29-VL.8 (aa) | 131 | QSVLTQPRSVSGSPGQSVTISCTGTSSDVGAYNYVSWYQQHPGKAPK |
| | | FMIYDVDQRPSGVPDRFSGSKSGNTASLIISGLQAEDEADYYSCSYAG |
| | | SYIWVFGGGTQLTVL |
| S2V29-VL.8 (nt) | 132 | CAAAGTGTGCTAACTCAACCACGAAGTGTGTCTGGCTCTCCAGGA |
| | | CAATCCGTGACCATCTCCTGCACCGGCACCTCCAGCGATGTGGGC |
| | | GCCTACAACTACGTGTCCTGGTACCAGCAGCACCCTGGCAAGGCC |
| | | CCTAAGTTCATGATCTACGACGTGGACCAGCGGCCTTCTGGCGTGC |
| | | CCGATAGATTCTCCGGCAGCAAATCCGGCAACACCGCTTCTCTGAT |
| | | CATCAGCGGCCTGCAGGCCGAGGACGAAGCCGACTACTACTCCTG |
| | | TTCTTATGCTGGCTCCTACATCTGGGTCTTTGGCGGCGGAACACAG |
| | | CTGACCGTGCTG |
| S2V29-VL.9 (aa) | 133 | QSVLTQPRSVSGSPGQSVTISCTGTSSDVGAYNYVSWYQQHPGKAPK |
| | | FMIYDVDQRPSGVPDRFSGSKSGNTASLIISGLQAEDEADYYACSYAG |
| | | SYIWVFGGGTQLTVL |
| S2V29-VL.9 (nt) | 134 | CAAAGTGTGCTAACTCAACCACGAAGTGTGTCTGGCTCTCCAGGA |
| | | CAATCCGTGACCATCTCCTGCACCGGCACCTCCAGCGATGTGGGC |
| | | GCCTACAACTACGTGTCCTGGTACCAGCAGCACCCTGGCAAGGCC |
| | | CCTAAGTTCATGATCTACGACGTGGACCAGCGGCCTTCTGGCGTGC |
| | | CCGATAGATTCTCCGGCAGCAAATCCGGCAACACCGCTTCTCTGAT |
| | | CATCAGCGGCCTGCAGGCCGAGGACGAAGCCGACTACTACGCCTG |
| | | TTCTTATGCTGGCTCCTACATCTGGGTCTTTGGCGGCGGAACACAG |
| | | CTGACCGTGCTG |
| S2V29-VH.1 (nt) | 135 | GAAGTGCAACTAGTGGAAAGTGGTGGTAGTGTCGTGCAGCCTGGC |
| (alternative | | AGAGCCCTCAGACTGAGCTGTGCTGCTTCTGGCATCACCTTCTCCT |
| codon) | | CTTTTGGCATGTACTGGGTGCGGCAAGCTCCCGGAAAAGGACTGG |
| | | AATGGCTGGGCGTGATCGCCTACGACGGCTCTAATACCTACTACG |
| | | CCGATTCCGTGAAGGGCCGCTTCACAATCTCCAGAGATAACTCCA |
| | | AGAACACCCTGTACCTGCAGATGAACTCTCTGCGGATCGAGGACA |
| | | CCGCCGTGTACTACTGCGCCAAGGACTACTATCCTCTGCTGTCCTA |
| | | CTACTACGGCCTGGACGTGTGGGGCCAGGGCACCACAGTGACCGT |
| | | GTCCAGC |
| S2V29-VH.11 | 136 | GAAGTGCAACTAGTGGAAAGTGGTGGTAGTGTCGTGCAGCCTGGC |
| (nt) (V50F) | | AGAGCCCTGAGACTGTCTTGTGCTGCTTCTGGCATCACCTTCAGCA |
| | | GCTTTGGCATGTACTGGGTGCGGCAAGCTCCCGGAAAAGGCCTGG |
| | | AATGGCTGGGCTTCATCGCCTACGACGGCTCCAACACCTACTACGC |
| | | CGACTCCGTGAAGGGCCGCTTCACCATCTCCAGAGATAATTCCAA |
| | | GAACACCCTGTACCTGCAGATGAACTCTCTGCGGATCGAGGACAC |
| | | CGCCGTGTACTACTGCGCCAAGGACTACTACCCTCTGCTGTCCTAC |
| | | TACTATGGACTCGATGTGTGGGGCCAGGGCACAACCGTGACCGTG |
| | | TCCTCT |
| S2V29-VH.11 | 137 | EVQLVESGGSVVQPGRALRLSCAASGITFSSFGMYWVRQAPGKGLE |
| (aa) (V50F) | | WLGFIAYDGSNTYYADSVKGRFTISRDNSKNTLYLQMNSLRIEDTAV |
| | | YYCAKDYYPLLSYYYGLDVWGQGTTVTVSS |
| S2V29-VH.12 | 138 | GAAGTGCAACTAGTGGAAAGTGGTGGTAGTGTGGTGCAGCCTGGC |
| (nt) (D54N) | | AGAGCTCTGAGACTGTCTTGTGCTGCTTCTGGCATCACCTTTTCTTC |
| | | TTTCGGCATGTACTGGGTCAGACAGGCCCCCGGCAAGGGCCTGGA |
| | | ATGGCTGGGAGTGATCGCCTACAACGGCTCCAACACCTACTACGC |
| | | CGACTCCGTGAAAGGACGGTTCACCATCTCCCGGGACAACTCCAA |
| | | GAATACACTGTACCTGCAGATGAACAGCCTGCGCATCGAGGACAC |
| | | CGCCGTGTACTACTGCGCCAAGGACTACTATCCTCTGCTGTCCTAC |
| | | TACTACGGCCTCGATGTGTGGGGCCAAGGCACCACCGTGACCGTG |
| | | TCCAGC |
| S2V29-VH.12 | 139 | EVQLVESGGSVVQPGRALRLSCAASGITFSSFGMYWVRQAPGKGLE |
| (aa) (D54N) | | WLGVIAYNGSNTYYADSVKGRFTISRDNSKNTLYLQMNSLRIEDTAV |
| | | YYCAKDYYPLLSYYYGLDVWGQGTTVTVSS |
| S2V29-VH.12 | 140 | IAYNGSNT |
| CDRH2 | | |
| S2V29-VH.13 | 141 | GAAGTGCAACTAGTGGAAAGTGGTGGTAGTGTCGTGCAGCCTGGA |
| (nt) (I51D-A52K) | | CGCGCCCTGAGACTCTCTTGTGCTGCTTCTGGCATCACCTTCAGCA |
| | | GCTTCGGCATGTACTGGGTGCGGCAAGCTCCTGGAAAAGGCCTGG |
| | | AATGGCTGGGCGTGGACAAGTACGACGGCTCCAACACCTACTACG |
| | | CCGATTCCGTGAAGGGCAGATTTACAATCTCCAGAGATAATTCCA |
| | | AGAACACCCTGTACCTGCAGATGAACTCTCTGCGGATCGAGGACA |
| | | CCGCCGTGTACTACTGCGCCAAGGACTACTACCCCCTGCTGTCCTA |
| | | CTATTACGGCCTGGACGTGTGGGGCCAGGGCACCACCGTGACCGT |
| | | GTCCTCT |
| S2V29-VH.13 | 142 | EVQLVESGGSVVQPGRALRLSCAASGITFSSFGMYWVRQAPGKGLE |
| (aa)) (I51D- | | WLGVDKYDGSNTYYADSVKGRFTISRDNSKNTLYLQMNSLRIEDTA |
| A52K) | | VYYCAKDYYPLLSYYYGLDVWGQGTTVTVSS |
| S2V29-VH.13 | 143 | DKYDGSNT |
| CDRH2 | | |
| S2V29-VL.10 | 144 | CAAAGTGTGCTAACTCAACCACGAAGTGTGTCTGCCTCTCCAGGC |
| (nt) (Y32H- | | CAGTCCGTGACCATCTCTTGTACCGGCACATCCTCCGACGTGGGCG |
| I50M-D52N- | | CTCACAACTACGTGTCCTGGTACCAGCAGCACCCTGGCAAGGCCC |
| C91S-S92V- | | CTAAGTTCATGATGTACAACGTGGATCAACGGCCTTCTGGCGTGCC |
| Y93D-198R) | | CGACAGATTTAGCGGAAGCAAAAGCGGCAACACCGCCTCTCTGAT |
| | | CATCTCCGGCCTGCAGGCCGAGGACGAAGCCGACTACTACTGCTC |
| | | CGTCGATGCTGGCTCCTATAGATGGGTGTTCGGCGGCGGAACCCA |
| | | GCTGACCGTGCTG |
| S2V29-VL.10 | 145 | QSVLTQPRSVSGSPGQSVTISCTGTSSDVGAHNYVSWYQQHPGKAPK |
| (aa) (Y32H- | | FMMYNVDQRPSGVPDRFSGSKSGNTASLIISGLQAEDEADYYCSVDA |
| I50M-D52N- | | GSYRWVFGGGTQLTV |
| C91S-S92V- | | |
| Y93D-198R) | | |
| S2V29-VL.10 | 146 | SSDVGAHNY |
| CDRL1 | | |
| S2V29-VL.10 | 147 | NVD |
| CDRL2 | | |
| S2V29-VL.10 | 148 | SVDAGSYRWV |
| CDRL3 | | |
| S2V29-VL.11 | 149 | CAAAGTGTGCTAACTCAACCACGAAGTGTGTCTGGCTCTCCTGGCC |
| (nt) (Y32H- | | AGTCCGTGACCATCAGCTGTACCGGCACCTCCTCCGACGTGGGCG |
| D52N-C91S- | | CTCACAACTACGTGTCCTGGTACCAGCAGCACCCCGGCAAGGCCC |
| S92V-Y93D- | | CTAAGTTCATGATCTACAACGTGGATCAACGGCCATCTGGCGTGCC |
| I98R) | | TGACAGATTTAGCGGATCTAAAAGCGGCAACACCGCCTCTCTGAT |
| | | CATCTCCGGCCTGCAGGCCGAGGACGAAGCCGACTACTACTGCTC |
| | | CGTCGATGCTGGCTCCTATAGATGGGTGTTCGGCGGCGGAACACA |
| | | GCTGACCGTGCTG |
| S2V29-VL.11 | 150 | QSVLTQPRSVSGSPGQSVTISCTGTSSDVGAHNYVSWYQQHPGKAPK |
| (aa) (Y32H- | | FMIYNVDQRPSGVPDRFSGSKSGNTASLIISGLQAEDEADYYCSVDAG |
| D52N-C91S- | | SYRWVFGGGTQLTVL |
| S92V-Y93D- | | |
| I98R) | | |
| S2V29-VL.12 | 151 | CAAAGTGTGCTAACTCAACCACGAAGTGTGTCTGGCTCTCCTGGCC |
| (nt) (Y32H- | | AATCCGTGACCATCTCCTGTACCGGCACAAGCTCCGACGTGGGCG |
| D52N-C91S- | | CCTATAACTACGTGTCCTGGTACCAGCAGCACCCCGGCAAGGCCC |
| S92V-Y93D- | | CTAAATTCATGATGTACAACGTGGACCACCGGCCTTCTGGCGTGCC |
| I98R) | | AGATAGATTCAGCGGATCCAAGTCCGGCAACACCGCTTCTCTGAT |
| | | CATCTCTGGCCTGCAGGCCGAGGACGAAGCCGACTACTACTGCTC |
| | | CGGCAATGCTGGCAGCTACAGATGGGTCTTTGGTGGAGGCACCCA |
| | | GCTGACCGTGCTG |
| S2V29-VL.12 | 152 | QSVLTQPRSVSGSPGQSVTISCTGTSSDVGAYNYVSWYQQHPGKAPK |
| (aa) (Y32H- | | FMMYNVDHRPSGVPDRFSGSKSGNTASLIISGLQAEDEADYYCSGNA |
| D52N-C91S- | | GSYRWVFGGGTQLTVL |
| S92V-Y93D- | | |
| I98R) | | |
| S2V29-VL.12 | 153 | SGNAGSYRWV |
| CDRL3 | | |
| S2V29-VL.13 | 154 | CAAAGTGTGCTAACTCAACCACGAAGTGTGAGCGGCTCTCCAGGA |
| (nt) (Y32N- | | CAATCTGTGACCATCTCCTGTACCGGCACCTCCTCCGACGTGGGCG |
| I50M-D52N- | | CCAACAACTACGTGTCCTGGTACCAGCAGCACCCTGGCAAAGCTC |
| Q55K-C91S- | | CTAAGTTCATGATGTACAACGTGGACAAGCGGCCTTCTGGCGTGC |
| S92V-Y93D- | | CCGATAGATTTAGCGGCTCCAAGTCCGGCAATACCGCCTCTCTGAT |
| I98K) | | CATCTCTGGCCTGCAGGCCGAGGACGAAGCCGACTACTACTGCTC |
| | | CGTCGATGCTGGCAGCTATAAGTGGGTGTTCGGCGGCGGAACACA |
| | | GCTGACCGTGCTG |
| S2V29-VL.13 | 155 | QSVLTQPRSVSGSPGQSVTISCTGTSSDVGANNYVSWYQQHPGKAPK |
| (aa) (Y32N- | | FMMYNVDKRPSGVPDRFSGSKSGNTASLIISGLQAEDEADYYCSVDA |
| I50M-D52N- | | GSYKWVFGGGTQLTVL |
| Q55K-C91S- | | |
| S92V-Y93D- | | |
| I98K) | | |
| SV29-VL.13 | 156 | SSDVGANNY |
| CDRL1 | | |
| SV29-VL.13 | 157 | SVDAGSYKWV |
| CDRL3 | | |
| S2V29-VL.14 | 158 | CAAAGTGTGCTAACTCAACCACGAAGTGTGAGCGGCTCTCCTGGC |
| (nt) (Y32H- | | CAGTCCGTGACCATCTCTTGTACCGGCACAAGCTCCGACGTGGGC |
| I50M-D52G- | | GCTCACAACTACGTGTCCTGGTACCAGCAGCACCCCGGCAAGGCC |
| C91S-S92F- | | CCTAAGTTCATGATGTACGGCGTGGACCAACGGCCTTCTGGTGTCC |
| Y93D-I98R) | | CAGATAGATTCTCCGGATCTAAAAGCGGCAACACCGCCTCTCTGA |
| | | TCATCTCCGGCCTGCAGGCCGAGGACGAAGCCGACTACTACTGCT |
| | | CCTTTGATGCTGGCTCCTATAGATGGGTGTTCGGCGGAGGCACCCA |
| | | GCTGACCGTGCTG |
| S2V29-VL.14 | 159 | QSVLTQPRSVSGSPGQSVTISCTGTSSDVGAHNYVSWYQQHPGKAPK |
| (aa) (Y32H- | | FMMYGVDQRPSGVPDRFSGSKSGNTASLIISGLQAEDEADYYCSFDA |
| I50M-D52G- | | GSYRWVFQGGTQLTVL |
| C91S-S92F- | | |
| Y93D-I98R) | | |
| S2V29-VL.14 | 160 | GVD |
| CDRL2 | | |
| S2V29-VL.14 | 161 | SFDAGSYRWV |
| CDRL3 | | |
| S2V29-VL.15 | 162 | CAAAGTGTGCTAACTCAACCACGAAGTGTGTCCGGCTCTCCAGGC |
| (nt) (Y32H- | | CAGTCCGTGACCATCTCCTGTACCGGCACCAGCTCTGACGTGGGCG |
| I50M-D52N- | | CTCACAACTACGTGTCCTGGTACCAGCAGCACCCTGGCAAGGCCC |
| C91S-S92D- | | CTAAGTTCATGATGTACAACGTGGACCAACGGCCTTCTGGCGTGCC |
| Y93D-I98R) | | CGATAGATTCAGCGGATCTAAATCCGGCAACACCGCCTCTCTGATC |
| | | ATCAGCGGCCTGCAGGCCGAGGATGAAGCCGACTACTACTGCTCC |
| | | GACGACGCTGGCTCCTATAGATGGGTCTTTGGCCGCGGAACACAG |
| | | CTGACCGTGCTG |
| S2V29-VL.15 | 163 | QSVLTQPRSVSGSPGQSVTISCTGTSSDVGAHNYVSWYQQHPGKAPK |
| (aa) (Y32H- | | FMMYNVDQRPSGVPDRFSGSKSGNTASLIISGLQAEDEADYYCSDDA |
| I50M-D52N- | | GSYRWVFGGGTQLTVL |
| C91S-S92D- | | |
| Y93D-I98R) | | |
| SV29-VL.15 | 164 | SDDAGSYRWV |
| CDRL3 | | |
| S2V29-VH.22 | 165 | GAAGTGCAACTAGTGGAAAGTGGTGGTAGTGTCGTGCAGCCTGGC |
| (nt) (V50S, A52S, | | AGAGCCCTCAGACTGAGCTGTGCTGCTTCTGGCATCACCTTCTCCT |
| N57F, Y101F, | | CTTTTGGCATGTACTGGGTGCGGCAAGCTCCCGGAAAAGGACTGG |
| Y107F) | | AATGGCTGGGCAGCATCAGCTACGACGGCTCTTTCACCTACTACGC |
| | | CGATTCCGTGAAGGGCCGCTTCACAATCTCCAGAGATAACTCCAA |
| | | GAACACCCTGTACCTGCAGATGAACTCTCTGCGGATCGAGGACAC |
| | | CGCCGTGTACTACTGCGCCAAGGACTACTTCCCTCTGCTGTCCTAC |
| | | TTCTACGGCCTGGACGTGTGGGGCCAGGGCACCACAGTGACCGTG |
| | | TCCAGC |
| S2V29-VH.22 | 166 | EVQLVESGGSVVQPGRALRLSCAASGITFSSFGMYWVRQAPGKGLE |
| (aa) (V50S, | | WLGSISYDGSFTYYADSVKGRFTISRDNSKNTLYLQMNSLRIEDTAV |
| A52S, N57F, | | YYCAKDYFPLLSYFYGLDVWGQGTTVTVSS |
| Y101F, Y107F) | | |
| S2V29-VH.22 | 167 | ISYDGSFT |
| CDRH2 | | |
| S2V29-VH.22 | 168 | AKDYFPLLSYFYGLDV |
| CDRH3 | | |
| S2V29-VH.24 | 169 | GAAGTGCAACTAGTGGAAAGTGGTGGTAGTGTCGTGCAGCCTGGC |
| (nt) (V50A, | | AGAGCCCTCAGACTGAGCTGTGCTGCTTCTGGCATCACCTTCTCCT |
| N57D, Y107F) | | CTTTTGGCATGTACTGGGTGCGGCAAGCTCCCGGAAAAGGACTGG |
| | | AATGGCTGGGCGCCATCGCCTACGACGGCTCTGACACCTACTACG |
| | | CCGATTCCGTGAAGGGCCGCTTCACAATCTCCAGAGATAACTCCA |
| | | AGAACACCCTGTACCTGCAGATGAACTCTCTGCGGATCGAGGACA |
| | | CCGCCGTGTACTACTGCGCCAAGGACTACTATCCTCTGCTGTCCTA |
| | | CTTCTACGGCCTGGACGTGTGGGGCCAGGGCACCACAGTGACCGT |
| | | GTCCAGC |
| S2V29-VH.24 | 170 | EVQLVESGGSVVQPGRALRLSCAASGITFSSFGMYWVRQAPGKGLE |
| (aa) (V50A, | | WLGAIAYDGSDTYYADSVKGRFTISRDNSKNTLYLQMNSLRIEDTAV |
| N57D, Y107F) | | YYCAKDYYPLLSYFYGLDVWGQGTTVTVSS |
| S2V29-VH.24 | 171 | IAYDGSDT |
| CDRH2 | | |
| also S2V29-v37.2 | | |
| CDRH2 | | |
| S2V29-VH.24 | 172 | AKDYYPLLSYFYGLDV |
| CDRH3 | | |
| S2V29-VH.25 | 173 | GAAGTGCAACTAGTGGAAAGTGGTGGTAGTGTCGTGCAGCCTGGC |
| (nt) (V50A, | | AGAGCCCTCAGACTGAGCTGTGCTGCTTCTGGCATCACCTTCTCCT |
| A52T, N57D, | | CTTTTGGCATGTACTGGGTGCGGCAAGCTCCCGGAAAAGGACTGG |
| Y107F) | | AATGGCTGGGCGCCATCACCTACGACGGCTCTGACACCTACTACG |
| | | CCGATTCCGTGAAGGGCCGCTTCACAATCTCCAGAGATAACTCCA |
| | | AGAACACCCTGTACCTGCAGATGAACTCTCTGCGGATCGAGGACA |
| | | CCGCCGTGTACTACTGCGCCAAGGACTACTATCCTCTGCTGTCCTA |
| | | CTTCTACGGCCTGGACGTGTGGGGCCAGGGCACCACAGTGACCGT |
| | | GTCCAGC |
| S2V29-VH.25 | 174 | EVQLVESGGSVVQPGRALRLSCAASGITFSSFGMYWVRQAPGKGLE |
| (aa) (V50A, | | WLGAITYDGSDTYYADSVKGRFTISRDNSKNTLYLQMNSLRIEDTAV |
| A52T, N57D, | | YYCAKDYYPLLSYFYGLDVWGQGTTVTVSS |
| Y107F) | | |
| S2V29-VH.25 | 175 | ITYDGSDT |
| CDRH2 | | |
| S2V29-VH.30 | 176 | GAAGTGCAACTAGTGGAAAGTGGTGGTAGTGTCGTGCAGCCTGGC |
| (nt) (V50Y, | | AGAGCCCTCAGACTGAGCTGTGCTGCTTCTGGCATCACCTTCTCCT |
| N57D, Y107F) | | CTTTTGGCATGTACTGGGTGCGGCAAGCTCCCGGAAAAGGACTGG |
| | | AATGGCTGGGCTACATCGCCTACGACGGCTCTGACACCTACTACG |
| | | CCGATTCCGTGAAGGGCCGCTTCACAATCTCCAGAGATAACTCCA |
| | | AGAACACCCTGTACCTGCAGATGAACTCTCTGCGGATCGAGGACA |
| | | CCGCCGTGTACTACTGCGCCAAGGACTACTATCCTCTGCTGTCCTA |
| | | CTTCTACGGCCTGGACGTGTGGGGCCAGGGCACCACAGTGACCGT |
| | | GTCCAGC |
| S2V29-VH.30 | 177 | EVQLVESGGSVVQPGRALRLSCAASGITFSSFGMYWVRQAPGKGLE |
| (aa) (V50Y, | | WLGYIAYDGSDTYYADSVKGRFTISRDNSKNTLYLQMNSLRIEDTAV |
| N57D, Y107F) | | YYCAKDYYPLLSYFYGLDVWGQGTTVTVSS |
| S2V29-VH.31 | 178 | GAAGTGCAACTAGTGGAAAGTGGTGGTAGTGTCGTGCAGCCTGGC |
| (nt) (V50Y, | | AGAGCCCTCAGACTGAGCTGTGCTGCTTCTGGCATCACCTTCTCCT |
| A52S, N57D, | | CTTTTGGCATGTACTGGGTGCGGCAAGCTCCCGGAAAAGGACTGG |
| Y107F) | | AATGGCTGGGCTACATCAGCTACGACGGCTCTGACACCTACTACG |
| | | CCGATTCCGTGAAGGGCCGCTTCACAATCTCCAGAGATAACTCCA |
| | | AGAACACCCTGTACCTGCAGATGAACTCTCTGCGGATCGAGGACA |
| | | CCGCCGTGTACTACTGCGCCAAGGACTACTATCCTCTGCTGTCCTA |
| | | CTTCTACGGCCTGGACGTGTGGGGCCAGGGCACCACAGTGACCGT |
| | | GTCCAGC |
| S2V29-VH.31 | 179 | EVQLVESGGSVVQPGRALRLSCAASGITFSSFGMYWVRQAPGKGLE |
| (aa) (V50Y, | | WLGYISYDGSDTYYADSVKGRFTISRDNSKNTLYLQMNSLRIEDTAV |
| A52S, N57D, | | YYCAKDYYPLLSYFYGLDVWGQGTTVTVSS |
| Y107F) | | |
| S2V29-VH.31 | 180 | ISYDGSDT |
| CDRH2 | | |
| S2V29-VH.36 | 181 | GAAGTGCAACTAGTGGAAAGTGGTGGTAGTGTCGTGCAGCCTGGC |
| (nt) (V50Y, | | AGAGCCCTCAGACTGAGCTGTGCTGCTTCTGGCATCACCTTCTCCT |
| A52S, N57D) | | CTTTTGGCATGTACTGGGTGCGGCAAGCTCCCGGAAAAGGACTGG |
| | | AATGGCTGGGCTACATCAGCTACGACGGCTCTGACACCTACTACG |
| | | CCGATTCCGTGAAGGGCCGCTTCACAATCTCCAGAGATAACTCCA |
| | | AGAACACCCTGTACCTGCAGATGAACTCTCTGCGGATCGAGGACA |
| | | CCGCCGTGTACTACTGCGCCAAGGACTACTATCCTCTGCTGTCCTA |
| | | CTACTACGGCCTGGACGTGTGGGGCCAGGGCACCACAGTGACCGT |
| | | GTCCAGC |
| S2V29-VH.36 | 182 | EVQLVESGGSVVQPGRALRLSCAASGITFSSFGMYWVRQAPGKGLE |
| (aa) (V50Y, | | WLGYISYDGSDTYYADSVKGRFTISRDNSKNTLYLQMNSLRIEDTAV |
| A52S, N57D) | | YYCAKDYYPLLSYYYGLDVWGQGTTVTVSS |
| S2V29-VH.37 | 183 | GAAGTGCAACTAGTGGAAAGTGGTGGTAGTGTCGTGCAGCCTGGC |
| (nt) (V50Y, | | AGAGCCCTCAGACTGAGCTGTGCTGCTTCTGGCATCACCTTCTCCT |
| N57D) | | CTTTTGGCATGTACTGGGTGCGGCAAGCTCCCGGAAAAGGACTGG |
| | | AATGGCTGGGCTACATCGCCTACGACGGCTCTGACACCTACTACG |
| | | CCGATTCCGTGAAGGGCCGCTTCACAATCTCCAGAGATAACTCCA |
| | | AGAACACCCTGTACCTGCAGATGAACTCTCTGCGGATCGAGGACA |
| | | CCGCCGTGTACTACTGCGCCAAGGACTACTATCCTCTGCTGTCCTA |
| | | CTACTACGGCCTGGACGTGTGGGGCCAGGGCACCACAGTGACCGT |
| | | GTCCAGC |
| S2V29-VH.37 | 184 | EVQLVESGGSVVQPGRALRLSCAASGITFSSFGMYWVRQAPGKGLE |
| (aa) (V50Y, | | WLGYIAYDGSDTYYADSVKGRFTISRDNSKNTLYLQMNSLRIEDTAV |
| N57D) | | YYCAKDYYPLLSYYYGLDVWGQGTTVTVSS |
| Light Chain | 185 | GGTCAGCCCAAGGCTGCCCCCTCGGTCACTCTGTTCCCGCCCTCGA |
| Lambda (nt) | | GTGAGGAGCTcCAAGCCAACAAGGCCACCCTGGTCTGTCTGATTAG |
| | | CGATTTCTACCCTGGTGCTGTCACTGTCGCATGGAAGGCCGATTCA |
| | | TCCCCCGTCAAAGCTGGTGTCGAGACAACAACCCCCAGCAAGCAG |
| | | TCTAACAATAAGTACGCCGCTTCCAGCTATCTGTCCCTGACACCTG |
| | | AGCAGTGGAAGAGCCACAGGTCTTACTCCTGTCAGGTCACTCACG |
| | | AAGGATCAACTGTGGAAAAAACTGTGGCACCAACCGAATGTTCA |
| Light Chain | 186 | GQPKAAPSVTLFPPSSEELQANKATLVCLISDFYPGAVTVAWKADSSP |
| Lambda (aa) | | VKAGVETTTPSKQSNNKYAASSYLSLTPEQWKSHRSYSCQVTHEGST |
| | | VEKTVAPTECS |
| S2V29-VH.A | 187 | EVQLVESGGSVVQPGRALRLSCAASGITFSSFGMYWVRQAPGKGLE |
| (V50Y) (aa) | | WLGYIAYDGSNTYYADSVKGRFTISRDNSKNTLYLQMNSLRIEDTAV |
| | | YYCAKDYYPLLSYYYGLDVWGQGTTVTVSS |
| S2V29-VH.B | 188 | EVQLVESGGSVVQPGRALRLSCAASGITFSSFGMYWVRQAPGKGLE |
| (V50*) (aa) | | WLG*IAYDGSNTYYADSVKGRFTISRDNSKNTLYLQMNSLRIEDTAV |
| | | YYCAKDYYPLLSYYYGLDVWGQGTTVTVSS |
| | | * may be any amino acid other than V |
| S2V29-VH.C | 189 | EVQLVESGGSVVQPGRALRLSCAASGITFSSFGMYWVRQAPGKGLE |
| (V50 aromatic) | | WLG*IAYDGSNTYYADSVKGRFTISRDNSKNTLYLQMNSLRIEDTAV |
| (aa) | | YYCAKDYYPLLSYYYGLDVWGQGTTVTVSS, * may be any amino |
| | | acid containing an aromatic ring, in particular F, W, |
| | | or T |
| S2V29-VH.D | 190 | EVQLVESGGSVVQPGRALRLSCAASGITFSSFGMYWVRQAPGKGLE |
| (V50 polar) (aa) | | WLG*IAYDGSNTYYADSVKGRFTISRDNSKNTLYLQMNSLRIEDTAV |
| | | YYCAKDYYPLLSYYYGLDVWGQGTTVTVSS, * may be any amino |
| | | acid having a polar side chain, in particular S, T, |
| | | C, N, Q, or Y |
| S2V29-VH.E | 191 | EVQLVESGGSVVQPGRALRLSCAASGITFSSFGMYWVRQAPGKGLE |
| (V50 | | WLG*IAYDGSNTYYADSVKGRFTISRDNSKNTLYLQMNSLRIEDTAV |
| amphipathic) (aa) | | YYCAKDYYPLLSYYYGLDVWGQGTTVTVSS, * may be any |
| | | amphipathic amino acid, in particular K, W, Y, or M |
| S2V29-VH.F | 192 | EVQLVESGGSVVQPGRALRLSCAASGITFSSFGMYWVRQAPGKGLE |
| (N57D) (aa) | | WLGVIAYDGSDTYYADSVKGRFTISRDNSKNTLYLQMNSLRIEDTAV |
| | | YYCAKDYYPLLSYYYGLDVWGQGTTVTVSS |
| S2V29-VH.G | 193 | EVQLVESGGSVVQPGRALRLSCAASGITFSSFGMYWVRQAPGKGLE |
| (N57*) (aa) | | WLGVIAYDGS*TYYADSVKGRFTISRDNSKNTLYLQMNSLRIEDTAV |
| | | YYCAKDYYPLLSYYYGLDVWGQGTTVTVSS, * may be any amino |
| | | acid other than N |
| S2V29-VH.H | 194 | EVQLVESGGSVVQPGRALRLSCAASGITFSSFGMYWVRQAPGKGLE |
| (N57 acidic) (aa) | | WLGVIAYDGS*TYYADSVKGRFTISRDNSKNTLYLQMNSLRIEDTAV |
| | | YYCAKDYYPLLSYYYGLDVWGQGTTVTVSS, * may be any acidic |
| | | amino acid, in particular D or E |
| S2V29-VH.I | 195 | EVQLVESGGSVVQPGRALRLSCAASGITFSSFGMYWVRQAPGKGLE |
| (N57 hydrophilic) | | WLGVIAYDGS*TYYADSVKGRFTISRDNSKNTLYLQMNSLRIEDTAV |
| (aa) | | YYCAKDYYPLLSYYYGLDVWGQGTTVTVSS, * may be any |
| | | hydrophilic amino acid other than N, in particular D, |
| | | E, S, T, C, Q, R, or H |
| S2V29-VH.J | 196 | EVQLVESGGSVVQPGRALRLSCAASGITFSSFGMYWVRQAPGKGLE |
| (V50Y, N57*) | | WLGYIAYDGS*TYYADSVKGRFTISRDNSKNTLYLQMNSLRIEDTAV |
| (aa) | | YYCAKDYYPLLSYYYGLDVWGQGTTVTVSS, * may be any amino |
| | | acid other than N |
| S2V29-VH.K | 197 | EVQLVESGGSVVQPGRALRLSCAASGITFSSFGMYWVRQAPGKGLE |
| (V50Y, N57 | | WLGYIAYDGS*TYYADSVKGRFTISRDNSKNTLYLQMNSLRIEDTAV |
| hydrophilic) (aa) | | YYCAKDYYPLLSYYYGLDVWGQGTTVTVSS, * may be any |
| | | hydrophilic amino acid other than N, in particular D, |
| | | E, S, T, C, Q, R, or H |
| S2V29-VH.K | 198 | EVQLVESGGSVVQPGRALRLSCAASGITFSSFGMYWVRQAPGKGLE |
| (V50Y, N57 | | WLGYIAYDGS*TYYADSVKGRFTISRDNSKNTLYLQMNSLRIEDTAV |
| acidic) (aa) | | YYCAKDYYPLLSYYYGLDVWGQGTTVTVSS, * may be any acidic |
| | | amino acid, in particular D or E |
| S2V29-VH.L | 199 | EVQLVESGGSVVQPGRALRLSCAASGITFSSFGMYWVRQAPGKGLE |
| (V50*, N57D) | | WLG*IAYDGSDTYYADSVKGRFTISRDNSKNTLYLQMNSLRIEDTAV |
| (aa) | | YYCAKDYYPLLSYYYGLDVWGQGTTVTVSS |
| | | * may be any amino acid other than V |
| S2V29-VH.M | 200 | EVQLVESGGSVVQPGRALRLSCAASGITFSSFGMYWVRQAPGKGLE |
| (V50 aromatic, | | WLG*IAYDGSDTYYADSVKGRFTISRDNSKNTLYLQMNSLRIEDTAV |
| N57D) (aa) | | YYCAKDYYPLLSYYYGLDVWGQGTTVTVSS, * may be any amino |
| | | acid containing an aromatic ring, in particular F, W, |
| | | or T |
| S2V29-VH.N | 201 | EVQLVESGGSVVQPGRALRLSCAASGITFSSFGMYWVRQAPGKGLE |
| (V50 polar, | | WLG*IAYDGSDTYYADSVKGRFTISRDNSKNTLYLQMNSLRIEDTAV |
| N57D) (aa) | | YYCAKDYYPLLSYYYGLDVWGQGTTVTVSS, * may be any amino |
| | | acid having a polar side chain, in particular S, T, C, |
| | | N, Q, or Y |
| S2V29-VH.O | 202 | EVQLVESGGSVVQPGRALRLSCAASGITFSSFGMYWVRQAPGKGLE |
| (V50 | | WLG*IAYDGSDTYYADSVKGRFTISRDNSKNTLYLQMNSLRIEDTAV |
| amphipathic, | | YYCAKDYYPLLSYYYGLDVWGQGTTVTVSS, * may be any |
| N57D) (aa) | | amphipathic amino acid, in particular K, W, Y, or M |
| S2V29-v37.2 HC | 203 | EVQLVESGGSVVQPGRALRLSCAASGITFSSFGMYWVRQAPGKGLE |
| (VH.37 + | | WLGYIAYDGSDTYYADSVKGRFTISRDNSKNTLYLQMNSLRIEDTAV |
| G1m17,1 CH1- | | YYCAKDYYPLLSYYYGLDVWGQGTTVTVSSASTKGPSVFPLAPSSK |
| CH3 + LS | | STSGGTAALGCLVKDYFPEPVTVSWNSGALTSGVHTFPAVLQSSGLY |
| mutation) (aa) | | SLSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSRDELTKNQVSLTCLV |
| | | KGFYPSDIAVEWESNGQPENNYKTTPPVLDSDGSFFLYSKLTVDKSR |
| | | WQQGNVFSCSVLHEALHSHYTQKSLSLSPGK |
| S2V29-v37.2 LC | 204 | QSVLTQPRSVSGSPGQSVTISCTGTSSDVGAYNYVSWYQQHPGKAPK |
| (VL.2 + Light | | FMIYDVDQRPSGVPDRFSGSKSGNTASLIISGLQAEDEADYYCSSYAG |
| chain Lambda) | | SYIWVFGGGTQLTVLGQPKAAPSVTLFPPSSEELQANKATLVCLISDF |
| (aa) | | YPGAVTVAWKADSSPVKAGVETTTPSKQSNNKYAASSYLSLTPEQW |
| | | KSHRSYSCQVTHEGSTVEKTVAPTECS |
| saRNA promoter | 205 | UAACCUGAAUGGACUACGACAUAGUCUAGUCCGCCAAGUCUAGC |
| (RNA nt) | | AUAUGGCCACCAUG |
| VEEV nsP1-4 | 206 | ILDIGSAPARRMYSKHKYHCICPMRCAEDPDRLYKYATKLKKNCKEI |
| (aa) | | TDKELDKKMKELAAVMSDPDLETETMCLHDDESCRYEGQVAVYQD |
| | | VYAVDGPTSLYHQANKGVRVAYWIGFDTTPFMFKNLAGAYPSYSTN |
| | | WADETVLTARNIGLCSSDVMERSRRGMSILRKKYLKPSNNVLFSVGS |
| | | TIYHEKRDLLRSWHLPSVFHLRGKQNYTCRCETIVSCDGYVVKRIAIS |
| | | PGLYGKPSGYAATMHREGFLCCKVTDTLNGERVSFPVCTYVPATLCD |
| | | QMTGILATDVSADDAQKLLVGLNQRIVVNGRTQRNTNTMKNYLLPV |
| | | VAQAFARWAKEYKEDQEDERPLGLRDRQLVMGCCWAFRRHKITSIY |
| | | KRPDTQTIIKVNSDFHSFVLPRIGSNTLEIGLRTRIRKMLEEHKEPSPLIT |
| | | AEDVQEAKCAADEAKEVREAEELRAALPPLAADVEEPTLEADVDLM |
| | | LQEAGAGSVETPRGLIKVTSYDGEDKIGSYAVLSPQAVLKSEKLSCIH |
| | | PLAEQVIVITHSGRKGRYAVEPYHGKVVVPEGHAIPVQDFQALSESAT |
| | | IVYNEREFVNRYLHHIATHGGALNTDEEYYKTVKPSEHDGEYLYDID |
| | | RKQCVKKELVTGLGLTGELVDPPFHEFAYESLRTRPAAPYQVPTIGVY |
| | | GVPGSGKSGIIKSAVTKKDLVVSAKKENCAEIIRDVKKMKGLDVNAR |
| | | TVDSVLLNGCKHPVETLYIDEAFACHAGTLRALIAIIRPKKAVLCGDP |
| | | KQCGFFNMMCLKVHFNHEICTQVFHKSISRRCTKSVTSVVSTLFYDK |
| | | KMRTTNPKETKIVIDTTGSTKPKQDDLILTCFRGWVKQLQIDYKGNEI |
| | | MTAAASQGLTRKGVYAVRYKVNENPLYAPTSEHVNVLLTRTEDRIV |
| | | WKTLAGDPWIKTLTAKYPGNFTATIEEWQAEHDAIMRHILERPDPTD |
| | | VFQNKANVCWAKALVPVLKTAGIDMTTEQWNTVDYFETDKAHSAEI |
| | | VLNQLCVRFFGLDLDSGLFSAPTVPLSIRNNHWDNSPSPNMYGLNKE |
| | | VVRQLSRRYPQLPRAVATGRVYDMNTGTLRNYDPRINLVPVNRRLP |
| | | HALVLHHNEHPQSDFSSFVSKLKGRTVLVVGEKLSVPGKMVDWLSD |
| | | RPEATFRARLDLGIPGDVPKYDIIFVNVRTPYKYHHYQQCEDHAIKLS |
| | | MLTKKACLHLNPGGTCVSIGYGYADRASESIIGAIARQFKFSRVCKPK |
| | | SSLEETEVLFVFIGYDRKARTHNSYKLSSTLTNIYTGSRLHEAGCAPSY |
| | | HVVRGDIATATEGVIINAANSKGQPGGGVCGALYKKFPESFDLQPIEV |
| | | GKARLVKGAAKHIIHAVGPNFNKVSEVEGDKQLAEAYESIAKIVNDN |
| | | NYKSVAIPLLSTGIFSGNKDRLTQSLNHLLTALDTTDADVAIYCRDKK |
| | | WEMTLKEAVARREAVEEICISDDSSVTEPDAELVRVHPKSSLAGRKG |
| | | YSTSDGKTFSYLEGTKFHQAAKDIAEINAMWPVATEANEQVCMYILG |
| | | ESMSSIRSKCPVEESEASTPPSTLPCLCIHAMTPERVQRLKASRPEQITV |
| | | CSSFPLPKYRITGVQKIQCSQPILFSPKVPAYIHPRKYLVETPPVDETPE |
| | | PSAENQSTEGTPEQPPLITEDETRTRTPEPIIIEEEEEDSISLLSDGPTHQV |
| | | LQVEADIHGPPSVSSSSWSIPHASDEDVDSLSILDTLEGASVTSGATSA |
| | | ETNSYFAKSMEFLARPVPAPRTVFRNPPHPAPRTRTPSLAPSRACSRTS |
| | | LVSTPPGVNRVITREELEALTPSRTPSRSVSRTSLVSNPPGVNRVITREE |
| | | FEAFVAQQQ*RFDAGAYIFSSDTGQGHLQQKSVRQTVLSEVVLERTE |
| | | LEISYAPRLDQEKEELLRKKLQLNPTPANRSRYQSRKVENMKAITARR |
| | | ILQGLGHYLKAEGKVECYRTLHPVPLYSSSVNRAFSSPKVAVEACNA |
| | | MLKENFPTVASYCIIPEYDAYLDMVDGASCCLDTASFCPAKLRSFPKK |
| | | HSYLEPTIRSAVPSAIQNTLQNVLAAATKRNCNVTQMRELPVLDSAAF |
| | | NVECFKKYACNNEYWETFKENPIRLTEENVVNYITKLKGPKAAALFA |
| | | KTHNLNMLQDIPMDRFVMDLKRDVKVTPGTKHTEERPKVQVIQAAD |
| | | PLATAYLCGIHRELVRRLNAVLLPNIHTLFDMSAEDFDAIIAEHFQPGD |
| | | CVLETDIASFDKSEDDAMALTALMILEDLGVDAELLTLIEAAFGEISSI |
| | | HLPTKTKFKFGAMMKSGMFLTLFVNTVINIVIASRVLRERLTGSPCAA |
| | | FIGDDNIVKGVKSDKLMADRCATWLNMEVKIIDAVVGEKAPYFCGG |
| | | FILCDSVTGTACRVADPLKRLFKLGKPLAADDEHDDDRRRALHEEST |
| | | RWNRVGILSELCKAVESRYETVGTSIIVMAMTTLASSVKSFSYLRGAP |
| | | ITLYG |
| S2V29-VH.37 | 207 | EVQLVESGGSVVQPGRALRLSCAAS |
| Framework 1 (aa) | | |
| S2V29-VH.37 | 208 | MYWVRQAPGKGLEWLGY |
| Framework 2 (aa) | | |
| S2V29-VH.37 | 209 | YYADSVKGRFTISRDNSKNTLYLQMNSLRIEDTAVYYC |
| Framework 3 (aa) | | |
| S2V29-VH.37 | 210 | WGQGTTVTVSS |
| Framework 4 (aa) | | |
| S2V29-VL.2 | 211 | QSVLTQPRSVSGSPGQSVTISCTGT |
| Framework 1 (aa) | | |
| S2V29-VL.2 | 212 | VSWYQQHPGKAPKFMIY |
| Framework 2 (aa) | | |
| S2V29-VL.2 | 213 | QRPSGVPDRFSGSKSGNTASLIISGLQAEDEADYYC |
| Framework 3 (aa) | | |
| S2V29-VL.2 | 214 | FGGGTQLTVL |
| Framework 4 (aa) | | |
| S2V29-VL.37 | 215 | YIAYDGSDT |
| CDRH2+Fr.A | | |
| (aa) | | |
| S2V29-VL.37 | 216 | WLGYIAYDGSDTYY |
| CDRH2+Fr.B | | |
| (aa) | | |
| S2V29-VH.37 | 217 | GITFSSFGMYWVRQAPGKGLEWLGYIAYDGSDTAKDYYPLLSYYY |
| H1-H3 (aa) | | GLDV |
| S2V29-VH.37 | 218 | GITFSSFGMYWVRQAPGKGLEWLGYIAYDGSDT |
| H1-H2 (aa) | | |
| S2V29-VH.37 | 219 | IAYDGSDTYYADSVKGRFTISRDNSKNTLYLQMNSLRIEDTAVYYCA |
| H2-H3 (aa) | | KDYYPLLSYYYGLDV |
| S2V29-VH.37 | 220 | YIAYDGSDTYYADSVKGRFTISRDNSKNTLYLQMNSLRIEDTAVYYC |
| H2 + Fr.A-H3 (aa) | | AKDYYPLLSYYYGLDV |
| S2V29.VH.37 | 221 | WLGYIAYDGSDTYYADSVKGRFTISRDNSKNTLYLQMNSLRIEDTAV |
| H2 + Fr.B-H3 (aa) | | YYCAKDYYPLLSYYYGLDV |
| S2V29-VL.2 L1- | 222 | SSDVGAYNYVSWYQQHPGKAPKFMIYDVDQRPSGVPDRFSGSKSGN |
| L3 (aa) | | TASLIISGLQAEDEADYYCSSYAGSYIWV |
| S2V29-VL.2 L1- | 223 | SSDVGAYNYVSWYQQHPGKAPKFMIYDVD |
| L2 (aa) | | |
| S2V29-VL.2 L2- | 224 | DVDQRPSGVPDRFSGSKSGNTASLIISGLQAEDEADYYCSSYAGSYIW |
| L3 (aa) | | V |
| S2V29-VH.37 | 225 | GGCATCACCTTCTCCTCTTTTGGC |
| CDRH1 (nt) | | |
| S2V29-VH.37 | 226 | ATCGCCTACGACGGCTCTGAC |
| CDRH2 (nt) | | |
| S2V29-VH.37 | 227 | GCCAAGGACTACTATCCTCTGCTGTCCTACTACTACGGCCTGGACGT |
| CDRH3 (nt) | | G |
| S2V29-VH.2 | 228 | TCTAGCGATGTGGGCGCTTACAACTAC |
| CDRL1 (nt) | | |
| S2V29-VH.2 | 229 | GACGTGGAC |
| CDRL2 (nt) | | |
| S2V29-VH.2 | 230 | TCCTCCTACGCCGGATCTTACATCTGGGTC |
| CDRL3 (nt) | | |
| S2V29-VH.37 | 231 | GAAGTGCAACTAGTGGAAAGTGGTGGTAGTGTCGTGCAGCCTGGCAG |
| Framework 1 (nt) | | AGCCCTCAGACTGAGCTGTGCTGCTTCT |
| S2V29-VH.37 | 232 | ATGTACTGGGTGCGGCAAGCTCCCGGAAAAGGACTGGAATGGCTGGG |
| Framework 2 (nt) | | CTAC |
| S2V29-VH.37 | 233 | CCTACTACGCCGATTCCGTGAAGGGCCGCTTCACAATCTCCAGAGAT |
| Framework 3 (nt) | | AACTCCAAGAACACCCTGTACCTGCAGATGAACTCTCTGCGGATCGA |
| | | GGACACCGCCGTGTACTACTGC |
| S2V29-VH.37 | 234 | TGGGGCCAGGGCACCACAGTGACCGTGTCCAGC |
| Framework 4 (nt) | | |
| S2V29-VL.2 | 235 | CAAAGTGTGCTAACTCAACCACGAAGTGTGAGCGGCTCTCCTGGC |
| Framework 1 (nt) | | CAGTCCGTGACCATCTCCTGTACCGGCACC |
| S2V29-VL.2 | 236 | GTGTCCTGGTATCAACAGCACCCCGGCAAGGCCCCTAAGTTCATGAT |
| Framework 2 (nt) | | CTAC |
| S2V29-VL.2 | 237 | CAGCGGCCATCTGGCGTGCCTGATAGATTCTCCGGCTCCAAATCCGG |
| Framework 3 (nt) | | CAACACCGCTTCTCTGATCATCTCTGGCCTGCAGGCCGAGGACGAAG |
| | | CCGACTACTACTGC |
| S2V29-VL.2 | 238 | TTTGGCGGCGGAACACAGCTGACCGTGCTG |
| Framework 4 (nt) | | |
| S2V29-VL.37 | 239 | CTACATCGCCTACGACGGCTCTGAC |
| CDRH2 + Fr.A | | |
| (nt) | | |
| S2V29-VL.37 | 240 | TGGCTGGGCTACATCGCCTACGACGGCTCTGACACCTACTAC |
| CDRH2 + Fr.B (nt) | | |
| S2V29-VH.37 | 241 | GGCATCACCTTCTCCTCTTTTGGCATGTACTGGGTGCGGCAAGCTCC |
| H1-H3 (nt) | | CGGAAAAGGACTGGAATGGCTGGGCTACATCGCCTACGACGGCTCTG |
| | | ACACCTACTACGCCGATTCCGTGAAGGGCCGCTTCACAATCTCCAGA |
| | | GATAACTCCAAGAACACCCTGTACCTGCAGATGAACTCTCTGCGGAT |
| | | CGAGGACACCGCCGTGTACTACTGCGCCAAGGACTACTATCCTCTGC |
| | | TGTCCTACTACTACGGCCTGGACGTG |
| S2V29-VH.37 | 242 | GGCATCACCTTCTCCTCTTTTGGCATGTACTGGGTGCGGCAAGCTCC |
| H1-H2 (nt) | | CGGAAAAGGACTGGAATGGCTGGGCTACATCGCCTACGACGGCTCTG |
| | | AC |
| S2V29-VH.37 | 243 | ATCGCCTACGACGGCTCTGACACCTACTACGCCGATTCCGTGAAGGG |
| H2-H3 (nt) | | CCGCTTCACAATCTCCAGAGATAACTCCAAGAACACCCTGTACCTGC |
| | | AGATGAACTCTCTGCGGATCGAGGACACCGCCGTGTACTACTGCGCC |
| | | AAGGACTACTATCCTCTGCTGTCCTACTACTACGGCCTGGACGTG |
| S2V29-VH.37 | 244 | TACATCGCCTACGACGGCTCTGACACCTACTACGCCGATTCCGTGAA |
| H2+Fr.A-H3 (nt) | | GGGCCGCTTCACAATCTCCAGAGATAACTCCAAGAACACCCTGTACC |
| | | TGCAGATGAACTCTCTGCGGATCGAGGACACCGCCGTGTACTACTGC |
| | | GCCAAGGACTACTATCCTCTGCTGTCCTACTACTACGGCCTGGACGT |
| | | G |
| S2V29.VH.37 | 245 | TGGCTGGGCTACATCGCCTACGACGGCTCTGACACCTACTACGCCGA |
| H2 + Fr.B-H3 (nt) | | TTCCGTGAAGGGCCGCTTCACAATCTCCAGAGATAACTCCAAGAACA |
| | | CCCTGTACCTGCAGATGAACTCTCTGCGGATCGAGGACACCGCCGTG |
| | | TACTACTGCGCCAAGGACTACTATCCTCTGCTGTCCTACTACTACGG |
| | | CCTGGACGTG |
| S2V29-VL.2 L1- | 246 | TCTAGCGATGTGGGCGCTTACAACTACGTGTCCTGGTATCAACAGCA |
| L3 (nt) | | CCCCGGCAAGGCCCCTAAGTTCATGATCTACGACGTGGACCAGCGGC |
| | | CATCTGGCGTGCCTGATAGATTCTCCGGCTCCAAATCCGGCAACACC |
| | | GCTTCTCTGATCATCTCTGGCCTGCAGGCCGAGGACGAAGCCGACTA |
| | | CTACTGCTCCTCCTACGCCGGATCTTACATCTGGGTC |
| S2V29-VL.2 L1- | 247 | TCTAGCGATGTGGGCGCTTACAACTACGTGTCCTGGTATCAACAGCA |
| L2 (nt) | | CCCCGGCAAGGCCCCTAAGTTCATGATCTACGACGTGGA |
| S2V29-VL.2 L2- | 248 | GACGTGGACCAGCGGCCATCTGGCGTGCCTGATAGATTCTCCGGCTC |
| L3 (nt) | | CAAATCCGGCAACACCGCTTCTCTGATCATCTCTGGCCTGCAGGCCG |
| | | AGGACGAAGCCGACTACTACTGCTCCTCCTACGCCGGATCTTACATC |
| | | TGGGTC |
| S2V29-VH.37 | 249 | GAAGUGCAACUAGUGGAAAGUGGUGGUAGUGUCGUGCAGCCUG |
| (RNA nt) | | GCAGAGCCCUCAGACUGAGCUGUGCUGCUUCUGGCAUCACCUUC |
| | | UCCUCUUUUGGCAUGUACUGGGUGCGGCAAGCUCCCGGAAAAGG |
| | | ACUGGAAUGGCUGGGCUACAUCGCCUACGACGGCUCUGACACCU |
| | | ACUACGCCGAUUCCGUGAAGGGCCGCUUCACAAUCUCCAGAGAU |
| | | AACUCCAAGAACACCCUGUACCUGCAGAUGAACUCUCUGCGGAU |
| | | CGAGGACACCGCCGUGUACUACUGCGCCAAGGACUACUAUCCUC |
| | | UGCUGUCCUACUACUACGGCCUGGACGUGUGGGGCCAGGGCACC |
| | | ACAGUGACCGUGUCCAGC |
| S2V29-VL.2 | 250 | CAAAGUGUGCUAACUCAACCACGAAGUGUGAGCGGCUCUCCUGG |
| (RNA nt) | | CCAGUCCGUGACCAUCUCCUGUACCGGCACCUCUAGCGAUGUGG |
| | | GCGCUUACAACUACGUGUCCUGGUAUCAACAGCACCCCGGCAAG |
| | | GCCCCUAAGUUCAUGAUCUACGACGUGGACCAGCGGCCAUCUGG |
| | | CGUGCCUGAUAGAUUCUCCGGCUCCAAAUCCGGCAACACCGCUU |
| | | CUCUGAUCAUCUCUGGCCUGCAGGCCGAGGACGAAGCCGACUAC |
| | | UACUGCUCCUCCUACGCCGGAUCUUACAUCUGGGUCUUUGGCGG |
| | | CGGAACACAGCUGACCGUGCUG |
| Linker (aa) | 251 | BPXXXZ, wherein each X is independently a glycine (G) |
| | | or serine (S), B is a positively charged amino acid |
| | | and Z is glycine (G) or a negatively charged amino acid |
| Linker (aa) | 252 | (GxS)y, wherein x is 1-10 and y is 1-10 |

*Table 2 indicates CDRH1, CDRH2 and CDRH3 (IMGT definition), in that order, in bold in each corresponding VH sequence; and CDRLI, CDRL2, CDRL3, in that order, in bold in each corresponding VL sequence.

TABLE 3
S2V29 and Variants (amino acid SEQ ID NOs)

| | | | | | | | | | VH | VL |
|---|---|---|---|---|---|---|---|---|---|---|
| Antibody | VH | CDRH1 | CDRH2 | CDRH3 | VL | CDRL1 | CDRL2 | CDRL3 | (nt) | (nt) |
| S2V29-VH | 67 | 68 | 69 | 70 | | | | | 75, 79, | |
| (also VH.1) | | | | | | | | | 135 | |
| S2V29-VH.2 | 81 | 68 | 82 | 70 | | | | | 83 | |
| S2V29-VH.3 | 84 | 68 | 85 | 70 | | | | | 86 | |
| S2V29-VH.4 | 87 | 68 | 88 | 70 | | | | | 89 | |
| S2V29-VH.5 | 90 | 68 | 91 | 70 | | | | | 92 | |
| S2V29-VH.6 | 93 | 68 | 94 | 95 | | | | | 96 | |
| S2V29-VH.7 | 97 | 68 | 98 | 99 | | | | | 100 | |
| S2V29-VH.8 | 101 | 68 | 102 | 103 | | | | | 104 | |
| S2V29-VH.9 | 105 | 68 | 106 | 107 | | | | | 108 | |
| S2V29-VH.10 | 109 | 68 | 110 | 111 | | | | | 112 | |
| S2V29-VH.11 | 137 | 68 | 69 | 70 | | | | | 136 | |
| S2V29-VH.12 | 139 | 68 | 140 | 70 | | | | | 138 | |
| S2V29-VH.13 | 142 | 68 | 143 | 70 | | | | | 141 | |
| S2V29-VH.22 | 166 | 68 | 167 | 168 | | | | | 165 | |
| S2V29-VH.24 | 170 | 68 | 171 | 172 | | | | | 169 | |
| S2V29-VH.25 | 174 | 68 | 175 | 172 | | | | | 173 | |
| S2V29-VH.30 | 177 | 68 | 171 | 172 | | | | | 176 | |
| S2V29-VH.31 | 179 | 68 | 180 | 172 | | | | | 178 | |
| S2V29-VH.36 | 182 | 68 | 180 | 70 | | | | | 181 | |
| S2V29-VH.37 | 184 | 68 | 171 | 70 | | | | | 183 | |
| S2V29-VL11a | | | | | 71 | 72 | 73 | 74 | | 76, 80 |
| (also VL.1) | | | | | | | | | | |
| S2V29-VL11b | | | | | 77 | 72 | 73 | 78 | | |
| S2V29-VL.2 | | | | | 113 | 72 | 73 | 114 | | 115 |
| S2V29-VL.3 | | | | | 116 | 72 | 73 | 117 | | 118 |
| S2V29-VL.4 | | | | | 119 | 72 | 73 | 120 | | 121 |
| S2V29-VL.5 | | | | | 122 | 72 | 73 | 123 | | 124 |
| S2V29-VL.6 | | | | | 125 | 72 | 73 | 126 | | 127 |
| S2V29-VL.7 | | | | | 128 | 72 | 73 | 129 | | 130 |
| S2V29-VL.8 | | | | | 131 | 72 | 73 | 74 | | 132 |
| S2V29-VL.9 | | | | | 133 | 72 | 73 | 74 | | 134 |
| S2V29-VL.10 | | | | | 145 | 146 | 147 | 148 | | 144 |
| S2V29-VL.11 | | | | | 150 | 146 | 147 | 148 | | 149 |
| S2V29-VL.12 | | | | | 152 | 72 | 147 | 153 | | 151 |
| S2V29-VL.13 | | | | | 155 | 156 | 147 | 157 | | 154 |
| S2V29-VL.14 | | | | | 159 | 146 | 160 | 161 | | 158 |
| S2V29-VL.15 | | | | | 163 | 146 | 147 | 164 | | 162 |

TABLE 4
Summary of Amino Acid Substitutions in S2V29 VH Variants

| | FR1 | | | | | FR2 | CDR2 | | | |
|---|---|---|---|---|---|---|---|---|---|---|
| Position | S10 | V11 | R16 | A17 | L18 | V50 | I51 | A52 | D54 | N57 |
| VH.1 | — | — | — | — | — | — | — | — | — | — |
| VH.11 | — | — | — | — | — | F | — | — | — | — |
| VH.12 | — | — | — | — | — | — | — | — | N | — |
| VH.13 | — | — | — | — | — | — | D | K | — | — |
| VH.22 | — | — | — | — | — | S | — | S | — | F |
| VH.24 | — | — | — | — | — | A | — | — | — | D |
| VH.25 | — | — | — | — | — | A | — | T | — | D |
| VH.30 | — | — | — | — | — | Y | — | — | — | D |
| VH.31 | — | — | — | — | — | Y | — | S | — | D |
| VH.36 | — | — | — | — | — | Y | — | S | — | D |
| VH.37 | — | — | — | — | — | Y | — | — | — | D |

Summary of Amino Acid Substitutions in S2V29 VH Variants

| | FR3 | CDR3 | | |
|---|---|---|---|---|
| Position | I88 | Y101 | L103 | Y107 |
| VH.1 | — | — | — | — |
| VH.11 | — | — | — | — |
| VH.12 | — | — | — | — |
| VH.13 | — | — | — | — |
| VH.22 | — | F | — | F |
| VH.24 | — | — | — | F |
| VH.25 | — | — | — | F |
| VH.30 | — | — | — | F |
| VH.31 | — | — | — | F |
| VH.36 | — | — | — | F |
| VH.37 | — | — | — | — |

### EXAMPLES

### Example 1

### Antibody Production and Materials

The following materials and methods were used these Examples 1-4 unless otherwise noted.

### Cell Lines

Cell lines used in Example 1 were obtained from ATCC (HEK293T, Vero and Vero-E6), ThermoFisher Scientific (Expi293F™ cells).

### Recombinant Protein Production

Wild-type SARS-CoV-2 RBD (with N-terminal signal peptide and ‘ETGT’, and C-terminal 8xHis-tag) was expressed in Expi293F cells at 37° C. and 8% CO₂ in the presence of 10 μM kifunensine. Transfection was performed using the ExpiFectamine 293 Transfection Kit (Thermo Fisher Scientific). Cell culture supernatant was collected four days after transfection and supplemented with 10×PBS to a final concentration of 2.5×PBS (342.5 mM NaCl, 6.75 mM KCl and 29.75 mM phosphates). SARS-CoV-2 S hexapro protein, used for cryo-EM single particle studies, was expressed and purified using known methods.

### Example 2

### Testing Methods

### Binding and Neutralization Testing

The ability of S2V29 antibodies to block ACE2 receptor binding to the SARS-CoV-2 RBD was evaluated by competition ELISA. SARS-CoV-2 mouse/rabbit Fc-tagged RBDs (final concentration 20 ng/ml) were incubated with serially diluted recombinant mAbs (from 25 μg/ml) and incubated for 1 h 37° C. The complex RBD: mAbs was then added to a pre-coated hACE2 (2 μg/ml in PBS) 96-well plate MaxiSorp (Nunc) and incubated 1 hour at room temperature. Subsequently, the plates were washed and a goat anti-mouse/rabbit IgG (Southern Biotech) coupled to alkaline phosphatase (Jackson Immunoresearch) added to detect mouse Fc-tagged RBDs binding. After further washing, the substrate (p-NPP, Sigma) was added, and plates read at 405 nm using a microplate reader (Biotek). The percentage of inhibition was calculated as follows: (1-((OD sample-OD neg ctr)/(OD pos. ctr-OD neg. ctr))*100.

To evaluate neutralization potency, dose-response inhibition assays were carried out using a vesicular stomatitis virus (VSV) pseudotyping platform.

Pseudotyped viruses were prepared using Lenti-X 293 cells seeded in 15-cm dishes. Briefly, cells in culture medium (DMEM supplemented with 10% heat-inactivated FBS, 1% PenStrep) were transfected with 25 μg of plasmid encoding for the corresponding S glycoprotein using TransIT-Lenti (Mirus) as transfectant reagent. One day post-transfection, cells were infected with vesicular stomatitis virus (VSV) (G*ΔG-luciferase) for 1 h, washed 3 times in PBS with Ca²⁺/Mg²⁺ (Thermo Fisher) before adding 25 ml of culture medium/dish. Particles were harvested after 18-24 h, clarified from cellular debris by centrifugation at 2,000×g for 20 min at 4° C., aliquoted and stored at −80° C. until use in neutralization experiments.

For neutralization experiments, Vero E6 cells were seeded at 20,000 cells/well in culture medium into white 96-well plates (PerkinElmer, 6005688) and cultured overnight at 37° C. 5% CO2. Ten-point 3-fold mAb serial dilutions were prepared in culture medium and mixed 1:1 with pseudotyped VSV prepared in culture medium in order to infect cells with the desired MOI. After 60 min incubation at 37° C., cell culture medium was aspirated and 50 μl of PVs/mAb mixture was added onto cells and incubated 60 min at 37° C. 5% CO₂. After 60 min, 100 μl of culture medium was added to the cells and incubation at 37° C. 5% CO₂ followed in the next 16-24 h. At the end of the incubation time, culture medium was removed from the cells and 50 μl/well of Steadylite (PerkinElmer) diluted 1:2 with PBS with Ca²⁺Mg²⁺ was added to the cells and incubated in the dark for 10 min.

Luminescence signals were read using a Synergy H1 Hybrid Multi-Mode plate reader (Biotek). Measurements were done in duplicate and at least six wells per plate contained untreated infected cells (defining the 0% of neutralization, “MAX RLU” value) and infected cells in the presence of controls defining the 100% of neutralization, “MIN RLU” value. Average of Relative light units (RLUs) of untreated infected wells (MAX RLUave) was subtracted by the average of MIN RLU (MIN RLUave) and used to normalize percentage of neutralization of individual RLU values of experimental data according to the following formula: (1-(RLUX-MIN RLUave)/(MAX RLUave−MIN RLUave))×100. Data were analyzed and visualized with Prism (Version 9.1.1). IC50 values were calculated from the interpolated value from the log(inhibitor) versus response, using variable slope (four parameters) nonlinear regression with an upper constraint of ≤100, and a lower constraint equal to 0.

### Example 3

### Comparison Antibodies

Various comparison anti-SARS-CoV-2 antibodies are used in these Examples.

S2H97 is described in Starr, T. N. et al. SARS-CoV-2 RBD antibodies that maximize breadth and resistance to escape. Nature 597, 97-102 (2021),

S2X259 is described in Tortorici, M. A., et al. Broad sarbecovirus neutralization by a human monoclonal antibody. Nature 597, 103-108 (2021).

S2K146 is described in Park, Y-J., et al. Antibody-mediated broad sarbecovirus neutralization through ACE2 molecular mimicry. Science 375 (6579), 449-454 (2022).

S2X324 is described in WO2021/226560 and Cameroni, E. et al. Broadly neutralizing antibodies overcome SARS-CoV-2 Omicron antigenic shift. Nature 602, 664-670 (2022).

S309 is described in Pinto, D., et al. Cross-neutralization of SARS-CoV-2 by a human monoclonal SARS-CoV antibody. Nature 583, 290-295 (2020). Sotrovimab (VIR-7831) was developed from S309 and is described in Cathcart, A., et al. The dual function monoclonal antibodies VIR-7831 and VIR-7832 demonstrate potent in vitro and in vivo activity against SARS-CoV-2; bioRxiv 2021.03.09.434607. Sotrovimab, in addition to having the VH and VL sequences noted in Table 2, is an IgG1 antibody with a LS Fc mutation.

S3L17, S312, and S3013, in addition to having the sequences noted in Table 2, are described in WO2024/006472.

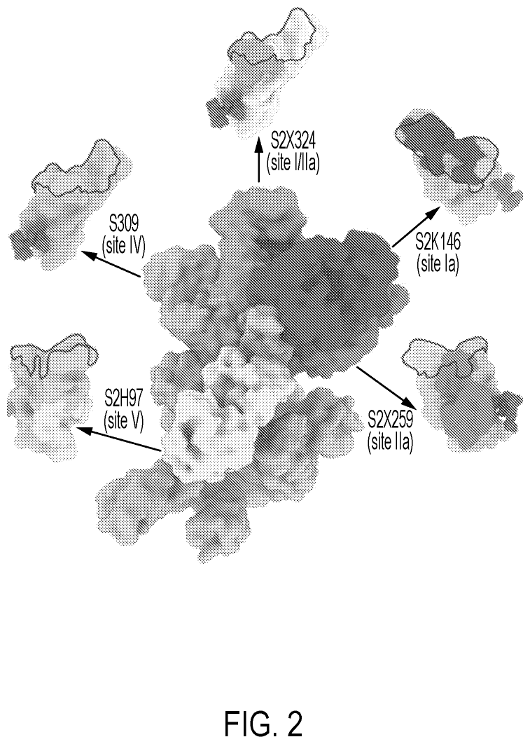

The antigen-binding site of certain comparison antibodies used in indicated in Table 5 and, for certain comparison antibodies, is also illustrated in FIG. 2.

TABLE 5
Characteristics of certain comparison antibodies

| | | | | VH/VL | AG |
|---|---|---|---|---|---|
| mAb | VH/VL SEQS | AG SITE | mAb | SEQS | SITE |
| S2X259 | 14, 15 | SITE IIa | S2K146 | 18, 19 | Ia |
| S309 | 16, 17 | SITE IV | S2X324 | 20, 21 | I/IIa |
| Sotrovimab | 22, 23 | SITE IV | S312 | 24, 25 | |
| S3013 | 26, 27 | | S3L17 | 28, 29 | |

Variant antibodies of S2V29 are summarized in Table 3 and Table 4.

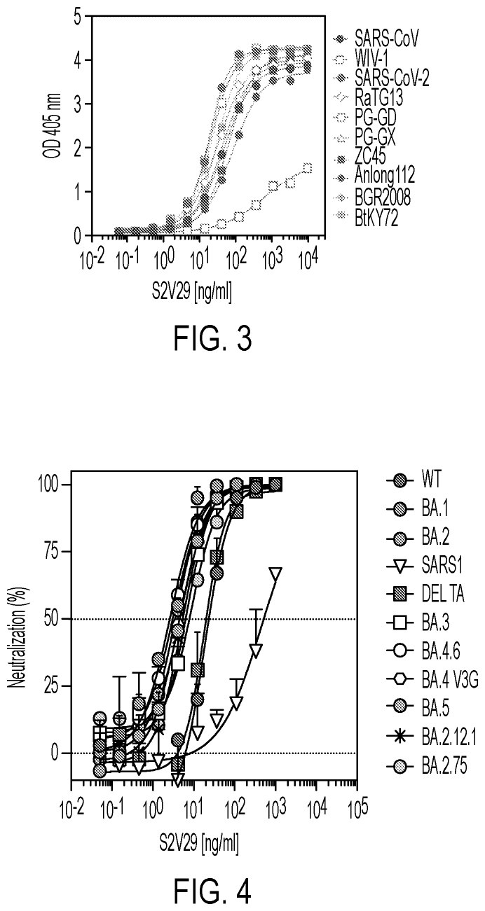

FIG. 3 shows binding by S2V29-v1.1 to various sarbecoviruses and FIG. 4 shows neutralization against SARS-CoV-2 variant lineages by S2V29-v1.1, demonstrating the ability of S2V29-v1.1, the parental antibody of S2V29-v37.2 (see Example 5), to bind and neutralize a wide array of SARS-CoV-2 variant lineages, including multiple variants lineages that have been variants of concern.

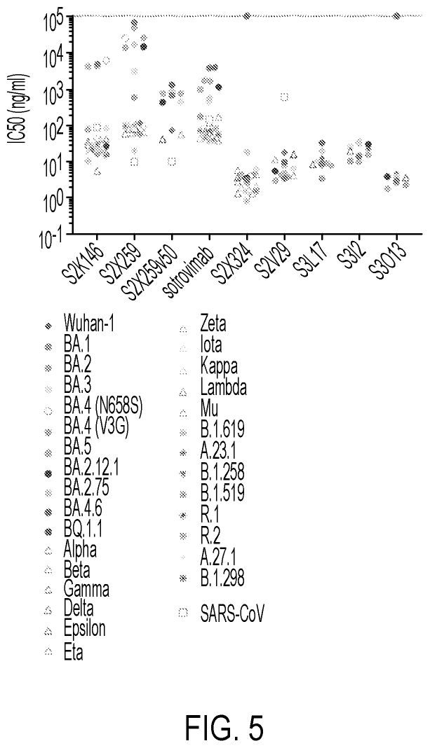

FIG. 5 summarizes neutralizing activity against SARS-CoV-2 Omicron variant lineages for S2V29-v1.1 as compared to comparison antibodies S2K146, S2X259, S2X259v50, sotrovimab, S2X324, S3L17, S312, and S3013. FIG. 5 also shows improved SARS-CoV neutralization activity for S2V29-v1.1 as compared to the comparison antibodies.

Table 6 shows a summary of results from the binding competition assays. The antibodies listed across the top row were tested for competitive (“C”), partial competition (“PC”) or non-competitive (“NC”) binding against the antibodies listed in the left-most column.

TABLE 6
Binding Competition Assay Results

| | | II mAb | | | |
|---|---|---|---|---|---|
| | | S2V29 | S3013 | S3L17 | S312 |
| ImAb | S2V29-v1.1 | C | NC | C | C |
| | S3O13 | NC | C | NC | NC |
| | S3L17 | PC | NC | C | C |
| | S3I2 | PC | NC | C | C |

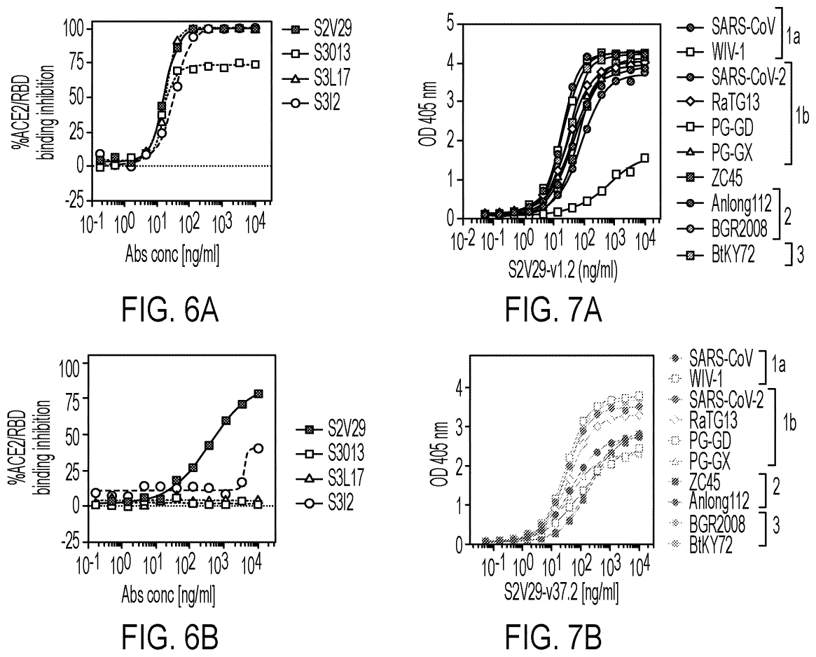

FIG. 6 shows the effects of parental S2V29-v1.1 antibody and comparison antibodies on inhibition of ACE2 binding to RBDs of SARS-CoV-2 (FIG. 6A) and SARS-CoV (FIG. 6B), demonstrating that S2V29-v1.1 has an unusual capability of strongly inhibiting ACE2/RBD binding of both SARS-CoV-2 and SARS-CoV.

Binding affinity tests for S2V29-v1.2 and S2V29-v37.2 and comparison antibody S3L17 for SARS-Co-V-2 variant lineages RBDs (see Example 5) showed picomolar binding activity and dissociation at the limit of detection for many variant lineages (Table 7). A connection between high affinity and a high barrier to escape mutants was observed.

TABLE 7
Binding Affinity (K_{D} (nM))

| RBD | S3L17 | S2V29-v1.2 | S2V29-v37.2 |
|---|---|---|---|
| Wuhan-Hu-1 | 0.07 | <0.02 | 0.16 |
| BA.1 | 0.08 | 0.05 | 0.39 |
| BA.2 | 0.18 | 0.02 | 0.37 |
| BA.4/5 | 0.08 | 0.04 | 0.20 |
| BA.2.75 | 0.27 | 0.03 | |
| BA.2.75.2 | 1.29 | <0.02 | |
| BA.4.6 | 0.05 | <0.02 | |
| BQ.1.1 | 1.26 | <0.02 | 0.08 |
| CH1.1 | | | 0.09 |
| XBB.1.5 | | | 0.07 |

Evolutionary breadth binding testing demonstrated that S2V29-v1.2 binds to all clades 1-3 RBDs (FIG. 7A) and S2V29-v37.2 has a similar broad binding profile (FIG. 7B). This is unexpectedly high breadth for ACE2-competing antibodies.

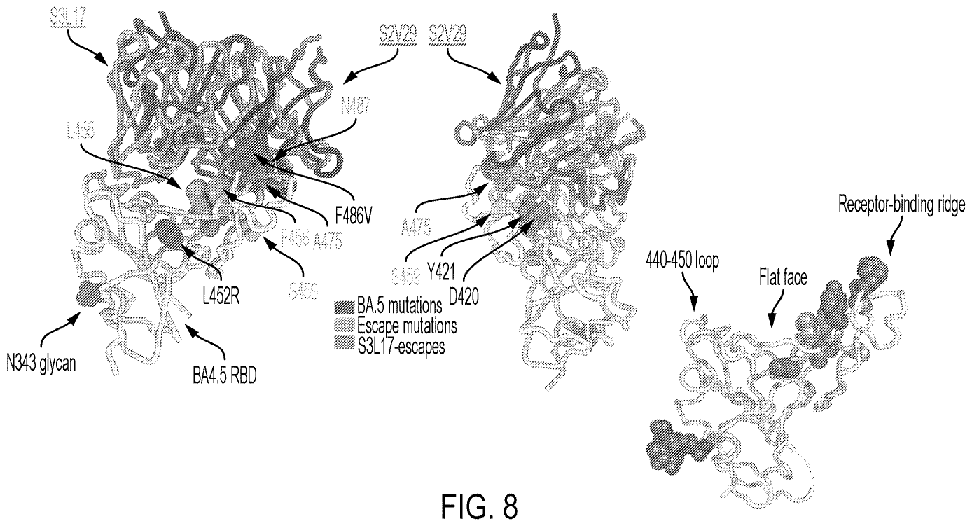

Likely binding configurations for S2V29-v1.2 and S3L17 on the RBD were determined (FIG. 8). The epitopes were partially overlapping, but the antibodies showed different escape profiles and no functional antagonism, such that there is likely a benefit to combining these two receptor-mimic antibodies. S2V29-v37.2 is expected to bind to the SARS-CoV-2 RBD in a manner similar to S2V29-v1.2.

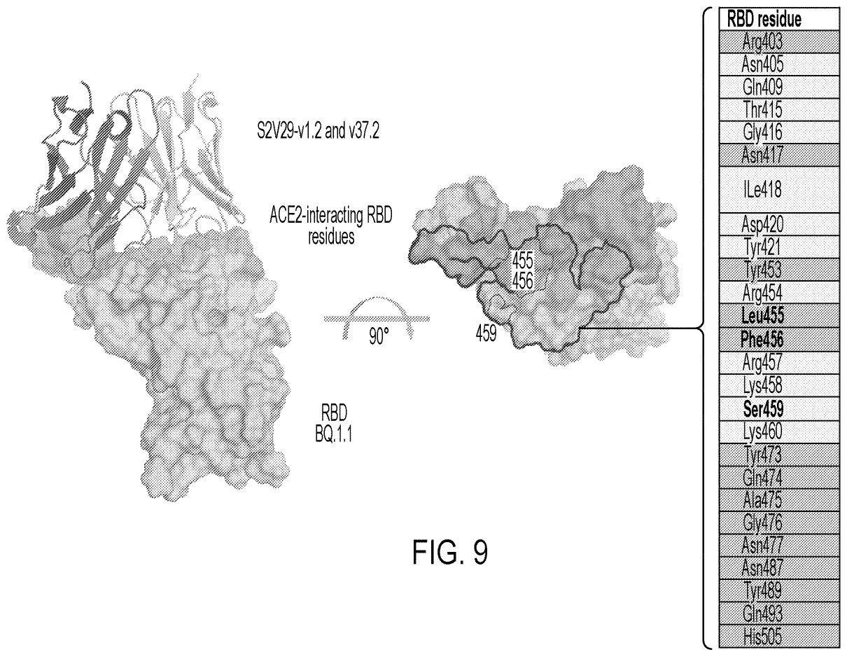

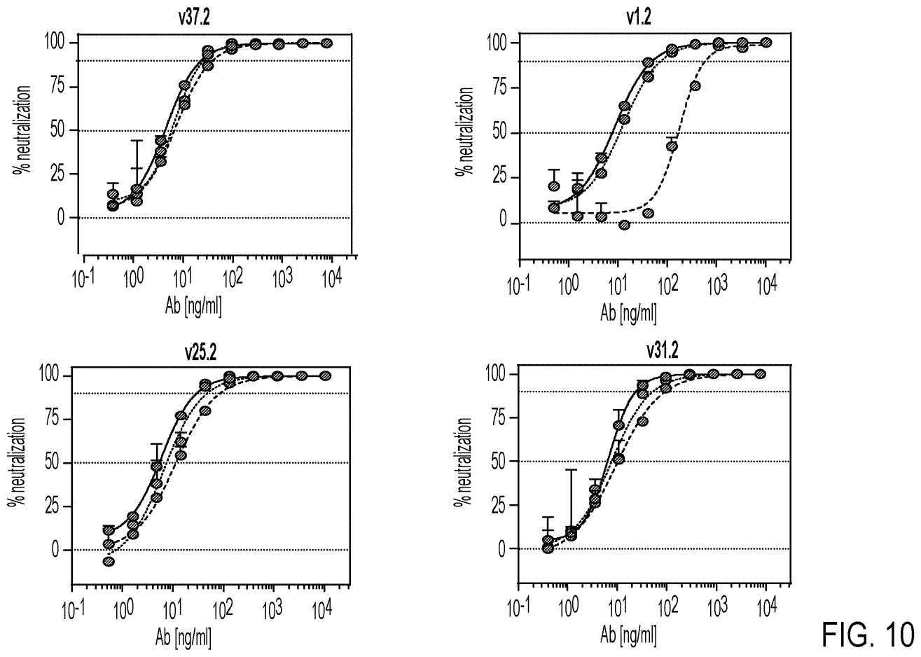

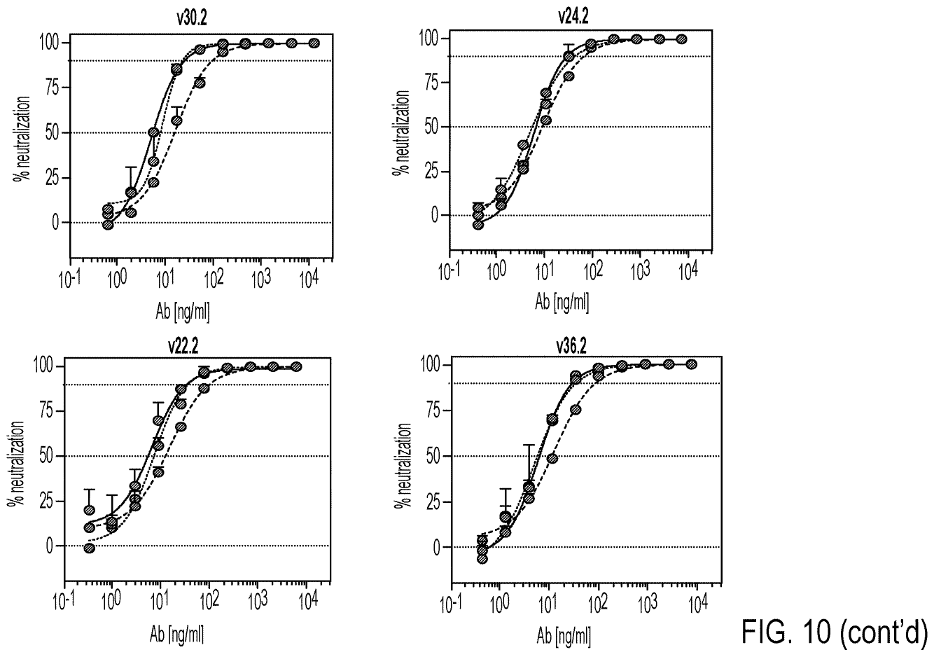

Likely binding of S2V29-v1.2 on the RBD of SARS-CoV-2 BQ.1.1 was further mapped to determine how S2V29-v1.2 competes with ACE for binding to the RBD (FIG. 9) and key contact residues, Leu455, Phe456, and Ser459, were identified. ACE 2 interacting RBD residues are also illustrated in FIG. 9. The S2V29-v1.2 epitope on the RBD is also illustrated with a black outline in FIG. 9. S2V29-v37.2 is expected to bind to the same epitope with the same key contact residues.

### Example 4

### S2V29 Variant Antibody Testing

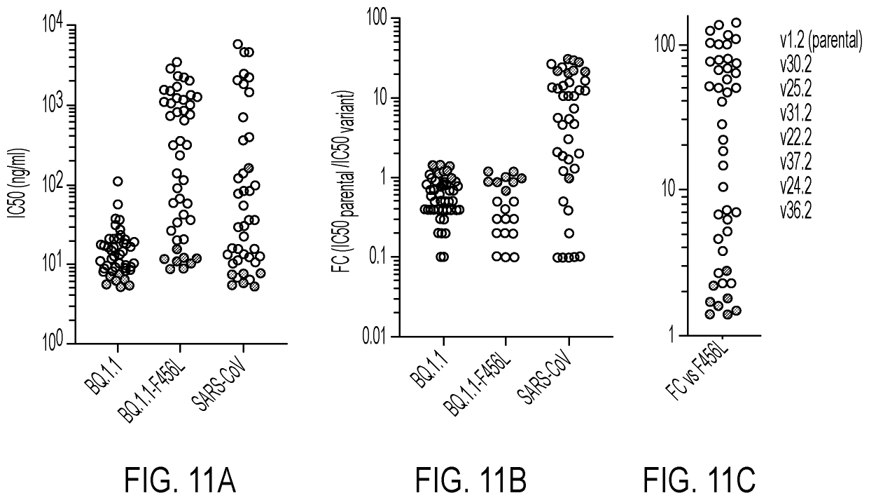

Further testing of specific S2V29 variant antibody neutralization against SARS-CoV-2 variants and SARS-CoV was performed. FIG. 10 provides neutralization assay results for a number of S2V29 variant antibodies and shows that seven of these variant antibodies with VL.2, including S2V29-v37.2, have a neutralization potency for SARS-CoV that is similar to their neutralization potencies for SARS-CoV-2 BQ.1.1. Other S2V29 variant antibodies performed less well (data not shown). SARS-CoV-2 and SARS-CoV neutralization data for S2V29 variant antibodies is also summarized in FIG. 11 (particularly FIG. 11A, FIG. 11B, and FIG. 11C) and Table 8, which provides data illustrated in FIG. 11, as well as in Table 9 (presenting results from a separate experiment than that of Table 8) with fold change (FC) of variant antibodies as compared to S2V29-v1.2 provided.

TABLE 8
SARS-CoV-2 Variant Neutralization by S2V29 Variant Antibodies

| | S2V29- | | | | | | | |
|---|---|---|---|---|---|---|---|---|
| | [x] | v30.2 | v25.2 | v31.2 | v22.2 | v37.2 | v24.2 | v36.2 |
| IC50 | BQ.1.1 | 5.5 | 5.3 | 6.4 | 7.0 | 5.6 | 6.6 | 6.5 |
| (ng/ | BQ.1.1- | 15.6 | 11.6 | 10.4 | 12.0 | 8.7 | 9.1 | 11.8 |
| ml) | F456L | | | | | | | |
| | SARS- | 8.0 | 7.5 | 7.7 | 5.3 | 7.7 | 5.5 | 5.9 |
| | CoV | | | | | | | |
| FC | BQ.1.1 | 1.4 | 1.4 | 1.2 | 1.1 | 1.4 | 1.2 | 1.2 |
| IC50 | BQ.1.1- | 0.7 | 0.9 | 1.0 | 0.9 | 1.2 | 1.2 | 0.9 |
| par/ | F456L | | | | | | | |
| IC50 | SARS- | 20.7 | 22.0 | 21.4 | 31.4 | 21.7 | 29.9 | 28.1 |
| Var | CoV | | | | | | | |

| | | | | | | | |
|---|---|---|---|---|---|---|---|
| Fold Loss vs. | 2.8 | 2.2 | 1.6 | 1.7 | 1.5 | 1.4 | 1.8 |
| F456L | | | | | | | |

TABLE 9
SARS-CoV Neutralization by S2V29 Variant Antibodies

| Variant | IC50 (ng · ml) | FC vs v1.2 |
|---|---|---|
| S2V29-v1.2 | 688.8 | |
| S2V29-v24.2 | 4.2 | 165.4 |
| S2V29-v25.2 | 4.7 | 147.7 |
| S2V29-v37.2 | 6.0 | 115.7 |

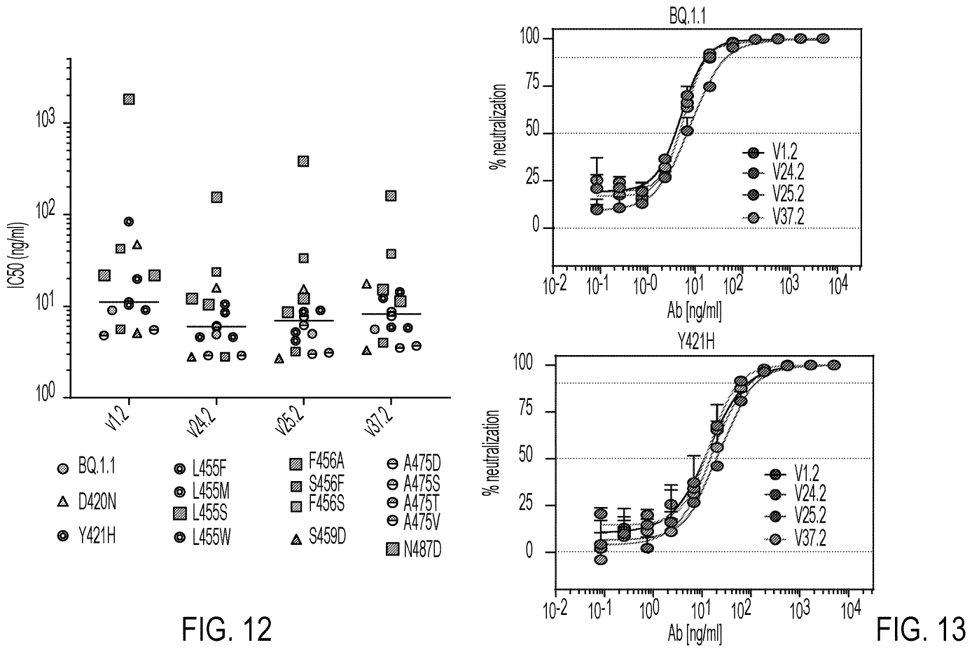

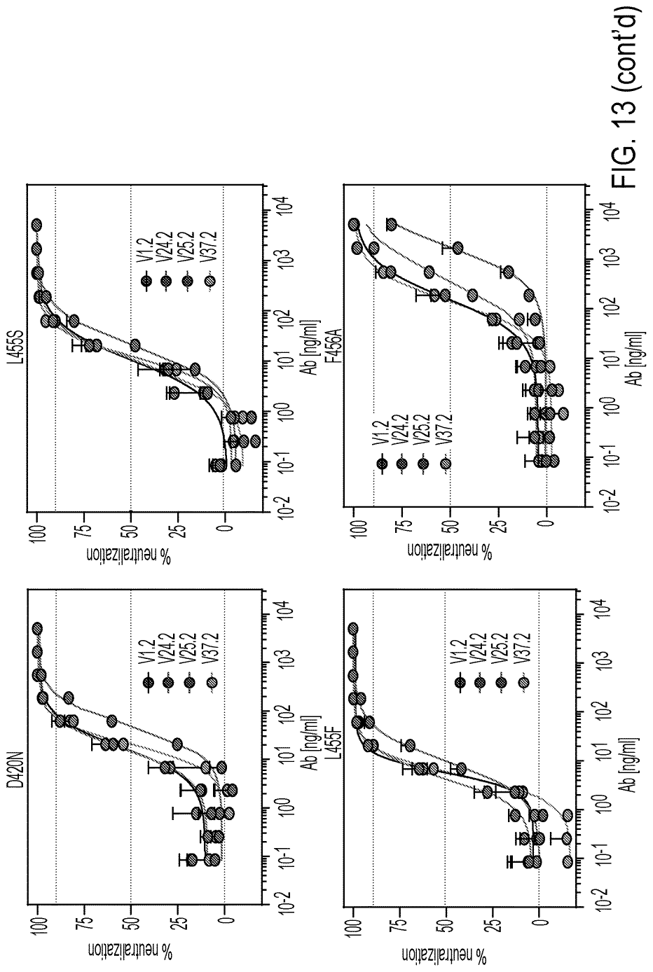

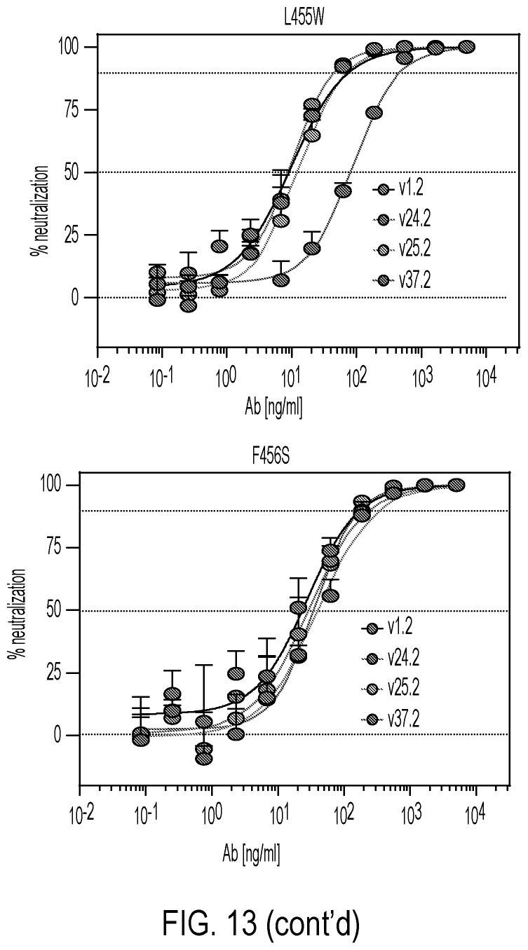

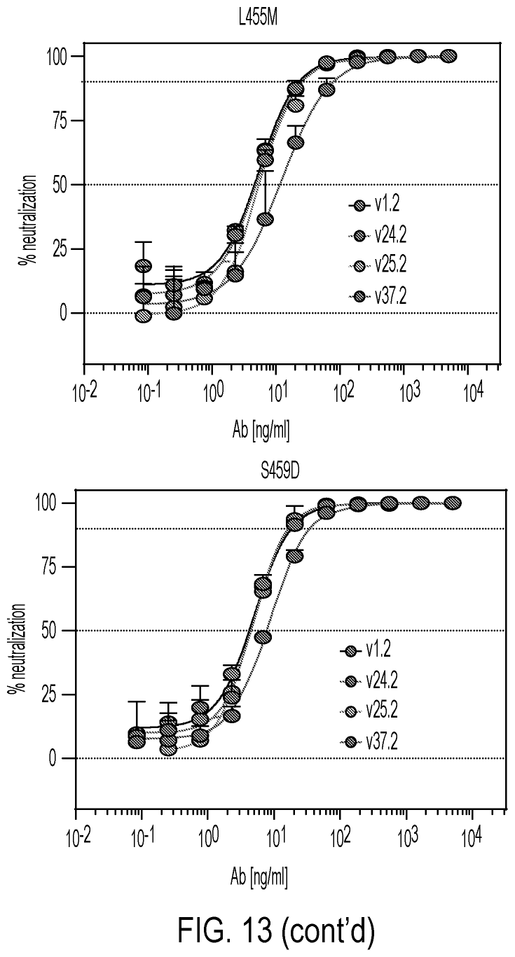

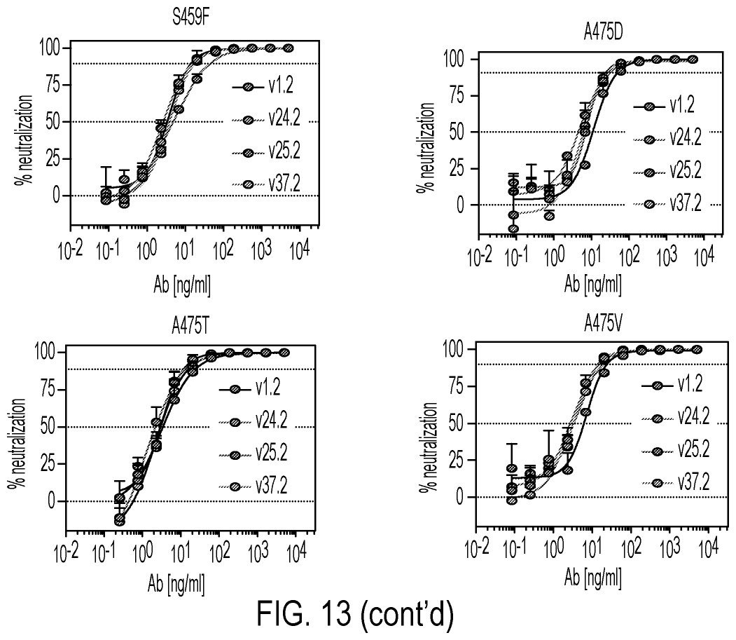

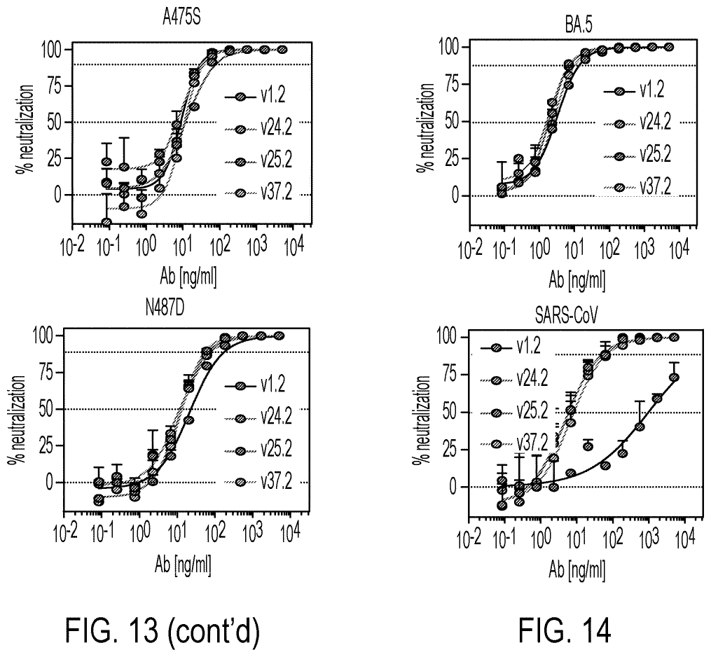

Selected S2V29 variant antibodies were also tested for neutralization against a panel of VSV-pp mutants of the SARS-CoV-2 BQ.1.1 variant lineage, and results are presented in FIG. 12, FIG. 13, Table 20, and Table 21.

TABLE 20
SARS-CoV-2 BQ.1.1 Mutant Neutralization by S2V29 Variant Antibodies

| | | IC50 (ng/mL) | | | | | | | |
|---|---|---|---|---|---|---|---|---|---|
| Mutation* | FC** | v1.2 | v30.2 | v25.2 | v31.2 | v22.2 | v37.2 | v24.2 | v36.2 |
| BQ1.1. | | | 5.1 | 5.6 | 5.3 | 5.9 | 5.0 | 5.4 | 6.1 |
| D420N | 4.4 | 87 | 11.4 | 10.3 | 11.1 | 26.5 | 13.3 | 13.1 | 13.0 |
| Y421R | 7.3 | 224 | 38.6 | 27.8 | 50.6 | 83.8 | 27.2 | 20.5 | 46.8 |
| L455Q | 3 | 59 | 13.9 | 16.1 | 17.0 | 18.9 | 9.2 | 10.6 | 11.6 |
| L455W | 8.8 | 81 | 11.4 | 10.8 | 13.7 | 9.2 | 6.8 | 8.4 | 12.2 |
| F456A | 56.5 | 868 | 115.8 | 224.7 | 764.9 | 299.2 | 103.7 | 92.2 | 431.7 |
| F456L | 1.3 | 11 | 14.7 | 13.7 | 19.3 | 18.0 | 9.2 | 15.2 | 18.6 |
| F456Q | 17.5 | 317 | 286.3 | 264.2 | 1055.4 | 380.1 | 199.2 | 223.1 | 784.6 |
| A475K | 15.1 | 233 | 61.7 | 82.6 | 115.6 | 145.4 | 79.8 | 74.5 | 125.9 |
| A475R | 7 | 111 | 34.2 | 33.7 | 46.2 | 67.5 | 19.3 | 16.3 | 27.1 |
| F456R | >100 | 10000 | >5000 | NN | NN | >5000 | 1854.4 | 2826.5 | NN |

*Mutation is as compared to BQ.1.1
**v1.2-FC vs. BQ.1.1

TABLE 21
SARS-CoV-2 BQ.1.1 Mutant Neutralization by S2V29 Variant Antibodies

| | IC50 (ng/ml) | | | | FC Compared to BQ.1.1 | | | | FC vs S2V29-v1.2 | | |
|---|---|---|---|---|---|---|---|---|---|---|---|
| Variant | v1.2 | v24.2 | v25.2 | v37.2 | v1.2 | v24.2 | v25.2 | v37.2 | v24.2 | v25.2 | v37.2 |
| BQ.1.1 | 9.0 | 4.9 | 5.0 | 5.6 | 1.0 | 1.0 | 1.0 | 1.0 | 1.8 | 1.8 | 1.6 |
| D420N | 47.2 | 15.9 | 15.4 | 17.6 | 5.5 | 3.3 | 3.1 | 3.3 | 3.0 | 3.1 | 2.7 |
| Y421H | 19.8 | 10.5 | 8.7 | 14.3 | 2.3 | 2.3 | 1.8 | 2.6 | 1.9 | 2.4 | 1.4 |
| L455F | 10.3 | 4.6 | 5.2 | 5.9 | 1.2 | 1.0 | 1.0 | 1.1 | 2.2 | 2.0 | 1.7 |
| L455M | 9.1 | 4.6 | 4.2 | 5.8 | 1.2 | 1.0 | 0.9 | 1.0 | 20. | 2.2 | 1.6 |
| L455S | 22.0 | 10.4 | 8.7 | 11.5 | 2.5 | 2.2 | 1.8 | 2.1 | 2.1 | 2.6 | 1.9 |
| L455W | 84.0 | 8.5 | 9.0 | 12.2 | 9.6 | 1.8 | 1.8 | 2.2 | 9.9 | 9.3 | 6.9 |
| F456A | 1834.8 | 154.4 | 381.7 | 160.2 | 209.9 | 32.3 | 76.4 | 28.6 | 11.9 | 4.8 | 11.5 |
| S456F | 5.6 | 2.8 | 3.2 | 4.0 | 0.6 | 0.6 | 0.6 | 0.7 | 2.0 | 1.8 | 1.4 |
| F456S | 42.5 | 23.6 | 33.5 | 37.3 | 4.9 | 5.0 | 6.7 | 6.7 | 1.8 | 1.3 | 1.1 |
| S459D | 5.1 | 2.8 | 2.7 | 3.3 | 0.7 | 0.7 | 0.6 | 0.7 | 1.8 | 1.9 | 1.5 |
| A475D | 11.0 | 5.9 | 7.7 | 8.7 | 1.3 | 1.2 | 1.5 | 1.6 | 1.9 | 1.4 | 1.3 |
| A475S | 11.1 | 6.1 | 6.2 | 7.8 | 1.3 | 1.4 | 1.4 | 1.6 | 1.8 | 1.9 | 1.5 |
| A475T | 4.8 | 2.9 | 3.1 | 3.7 | 0.5 | 0.6 | 0.6 | 0.7 | 1.6 | 1.6 | 1.3 |
| A475V | 5.5 | 2.9 | 3.0 | 3.5 | 0.7 | 0.6 | 0.6 | 0.6 | 1.9 | 1.8 | 1.6 |
| N587D | 21.9 | 12.2 | 12.1 | 15.1 | 2.6 | 2.6 | 2.4 | 2.7 | 1.8 | 1.8 | 1.5 |

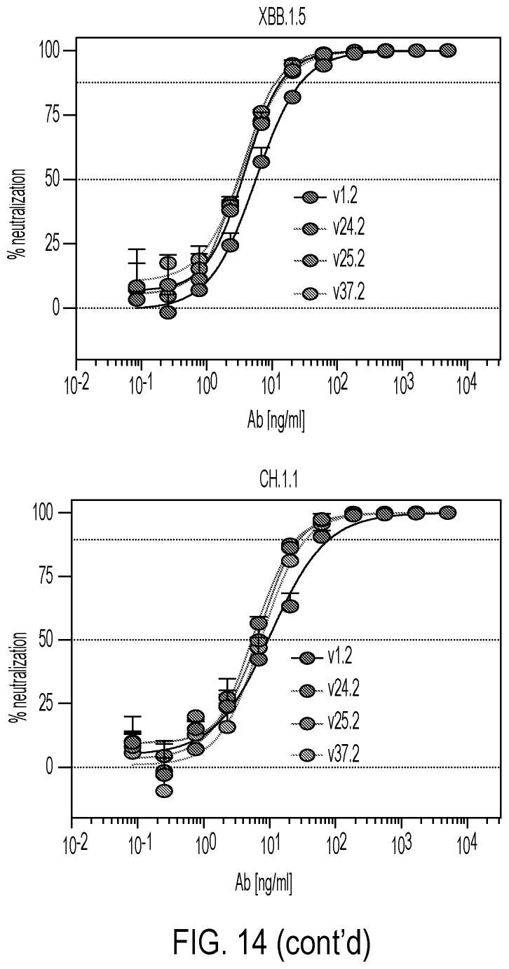

S2V29 variant antibodies demonstrated efficacy versus circulating SARS-CoV-2 variant lineage mutants at a level similar to that of the S2V29-v1.2 antibody (FIG. 14 and Table 22).

TABLE 22
SARS-CoV-2 Neutralization by S2V29 Variant Antibodies

| | IC50 (ng/mL) | | | | FC vs S2V29-v1.2 | | |
|---|---|---|---|---|---|---|---|
| Variant | v1.2 | v24.2 | v25.2 | v37.2 | v24.2 | v25.2 | v37.2 |
| XBB.1.5 | 5.2 | 3.3 | 2.6 | 3.4 | 1.6 | 2.0 | 1.6 |
| CH.1.1 | 11.2 | 6.6 | 7.4 | 8.9 | 1.7 | 1.5 | 1.3 |
| BA.5 | 2.6 | 1.6 | 1.5 | 2.1 | 1.6 | 1.7 | 1.2 |

S2V29-v37.2 was selected for further testing. As illustrated in FIG. 7A and Table 7, S2V29-v37.2 demonstrated a K_{D} (nM) in subnanomolar ranges for all SARS-CoV-2 variant lineages (representing a wide array of viral variants). In addition, this data, along with escape mutant data, established that there is a connection between high affinity and a high barrier to escape for S2V29 antibodies, including S2V29-v37.2.

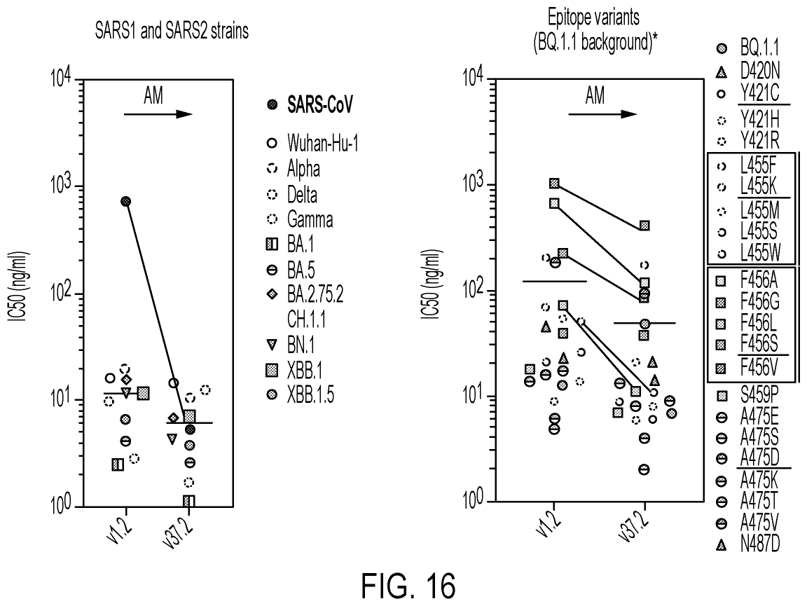

S2V29-v37.2, as illustrated in FIG. 15, contains two Fab amino acid variations in the VH as compared to S2V29-v1.2, and demonstrates SARS-CoV-2 potency similar to that of S2V29-v1.2, but also demonstrated a 100 fold improvement in neutralization against SARS-CoV as compared to S2V29-v37.2 (FIG. 16). SARS-CoV and SARS-CoV-2 differ at residues 455 and 456, and S2V29-v37.2 demonstrated improved potency against key escape variants (such as SARS-CoV-2 F456L variants) at these RBD positions, and generally demonstrated neutralization of a wide variety of SARS-CoV-2 variants (FIG. 16). For some SARS-CoV-2 variants, S2V29-v37.2 demonstrated higher neutralization than S2V29-v1.2. For example, S2V29-v37.2 neutralizes BG.1.1-F456L at a 6.8 fold higher potency and XBB.1.5-F456L at a 2.7 fold higher potency than S2V29-v1.2.

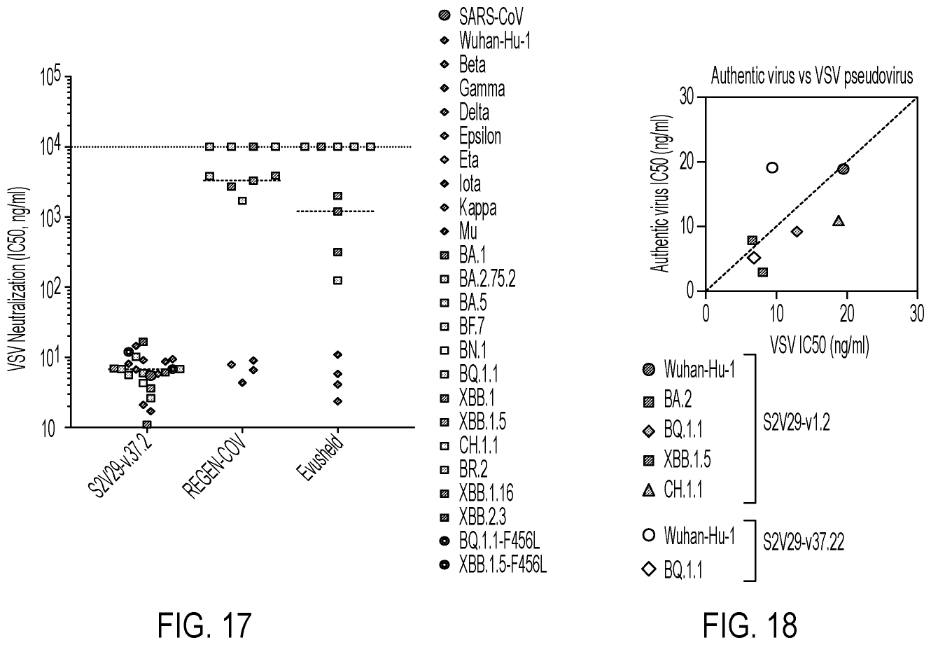

Neutralization of various SARS-CoV-2 variant lineages by S2V29-v37.2 and comparison antibodies comparison antibodies REGEN-CoV and Evushield was determined by VSV pseudovirus assay (FIG. 17). S2V29-v37.2 exhibited a similar potency as the comparison antibodies and a significantly wider breadth of neutralization capabilities across viral variant lineages.

FIG. 18 shows a comparison of results obtained with authentic virus vs. VSV pseudovirus for S2V29-v1.2 and S2V29-v37.2. Authentic virus data are consistent with VSV pseudovirus data for both variant antibodies.

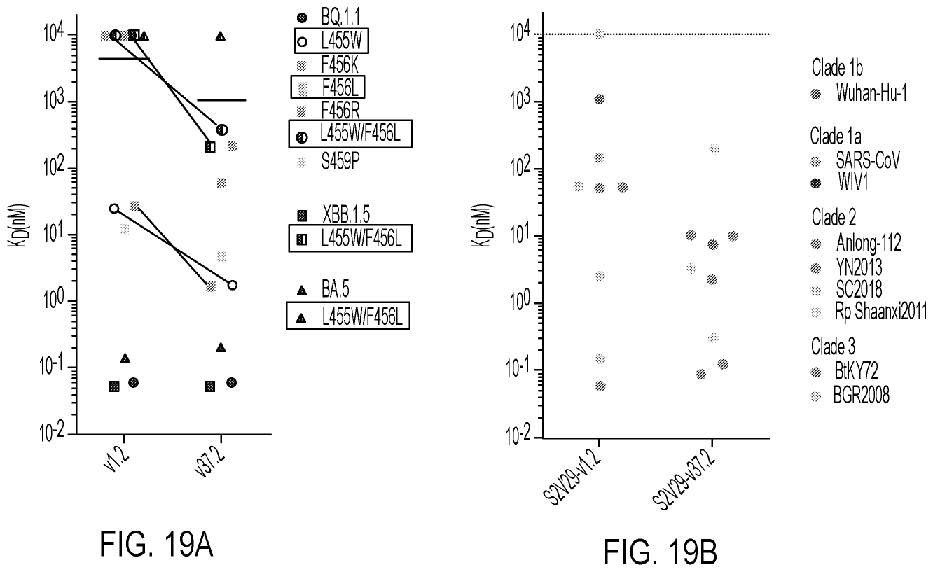

Binding affinity of S2V29-v1.2 and S2V29-v37.2 Fabs to recombinant RBDs from various sarbecoviruses (FIG. 19A) and RBDs with escape mutations (FIG. 19B) was measured. S2V29-v37.2 showed similar or improved (as compared to S2V29-v1.2) binding affinity for a wide array of sarbecoviruses and escape mutations.

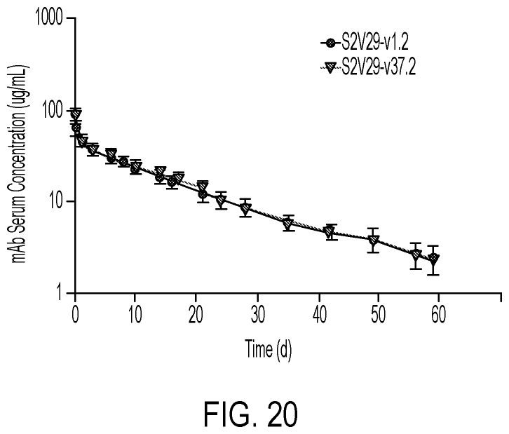

FIG. 20 shows the results of pharmokinetic studies in Tg32 SCID mice through 3 weeks post injection. Based on this data, it is expected that S2V29-v1.2 would provide both suitable therapeutic effect and six months of prophylactic effect following a single IM injection.

### Example 5

### Development and Testing of S2V29-v37.2 Vs. S2V29-v1.2

S2V29-v1.2 as compared to the parental S2V29-v1.1 antibody contains a mutation of a germline encoded cysteine residue to serine to reduce the risk of improper cysteine bridges.

S2V29-v37.2 was developed by affinity-maturing S2V29-v1.2 against the SARS-CoV-1 RBD by machine learning-assisted computational design combined with a yeast display selection system. This yielded S2V29-v37.2, which differs from S2V29-v1.2 at two residues, heavy chain V50Y and N57D.

S2V29-v1.2 or S2V29-v37.2 VH and VL sequences were obtained by RT-PCR and subcloned in IgG1, G1m3-LS expression vectors (G1m3 allotype with M428L/N434S mutations in the Fc moiety).

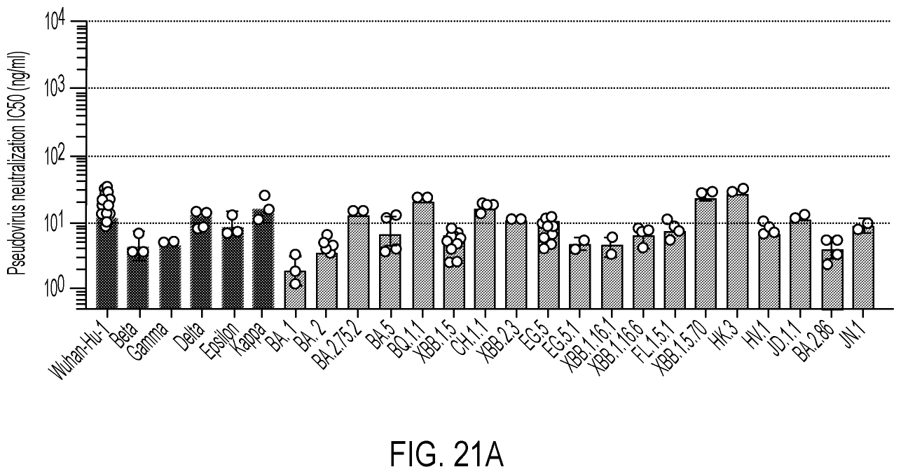

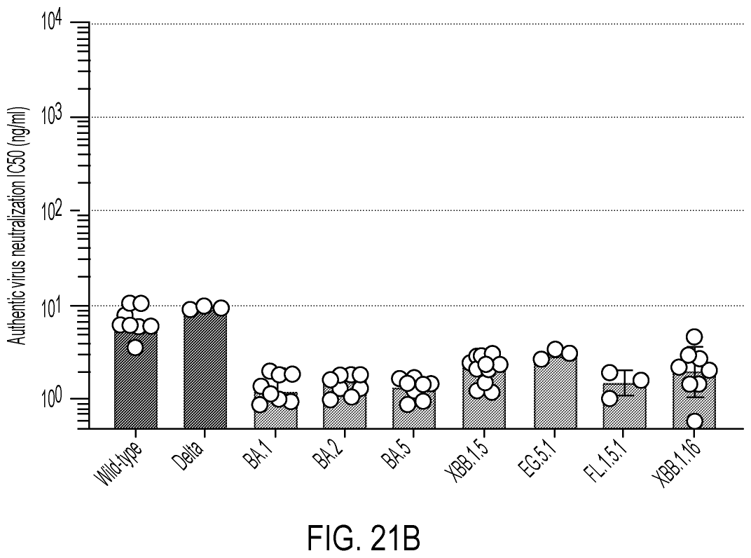

S2V29-v37.2 was tested in both pseudovirus and live virus assays. Pseudovirus assays were conducted using VeroE6 cells and a number of SARS-CoV-2 variant lineages (FIG. 21A). S2V29-v37.2 neutralized a large panel of pre- and post-Omicron vesicular stomatitis virus (VSV)-based SARS-CoV-2 S pseudotyped viruses (pseudoviruses) with high potency (IC50 ranging between 1.9 and 31.2 ng/ml; median 8.2 ng/ml for a 25-strain panel. Authentic live virus neutralization assays were conduced with VeroE6 cells using S2V29-v37.2 and wild-type virus (same S haplotype as Wuhan-Hu-1) and a number of variant lineages of interest (FIG. 21B). S2V29-v37.2 also showed high viral neutralization of authentic SARS-CoV-2 viral isolates, with an IC50 of 1.3-9.6 ng/ml and a median IC50 of 2.0 ng/ml for a 9-strain panel.

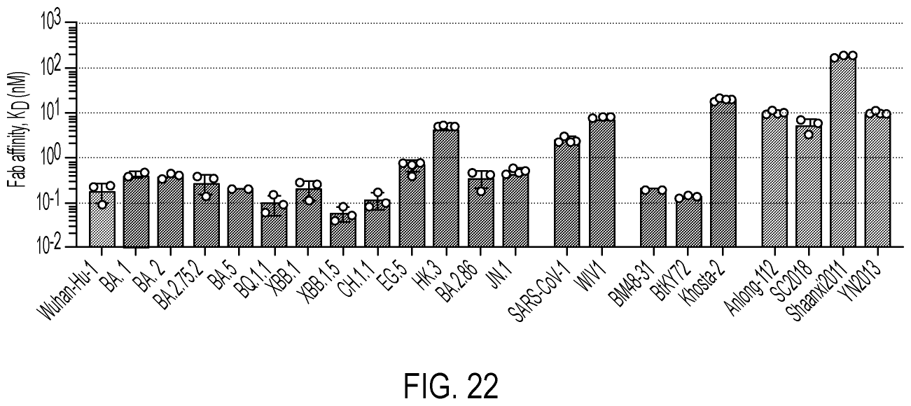

S2V29-v37.2 Fab fragment binding affinity for a number of SARS-CoV-2 variant lineages, as well as the SARS-CoV-1 Urbani strain and a number of other sarbecoviruses representing a variety of viral clades (WIV1, BM48-31, BtKY72, Khosta-2, Anlong-112, SC2018, Shaanxi2011, and YN2013) was measured by SPR (FIG. 22). The S2V29-v37.2 Fab fragment bound with sub-nanomolar affinity to most sarbecovirus clade 1b and clade 3 viruses, with a KD 0.05-5.0 nM, and a median KD of 0.26 nM for the 13-strain panel of SARS-CoV-2 variant lineages, and with nanomolar affinity to other sarbecovirus RBD.

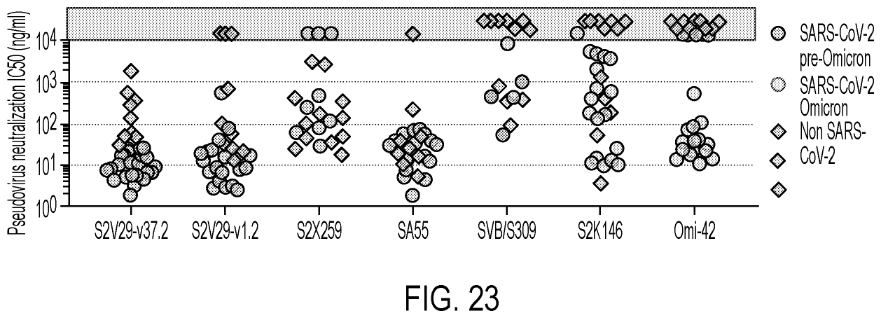

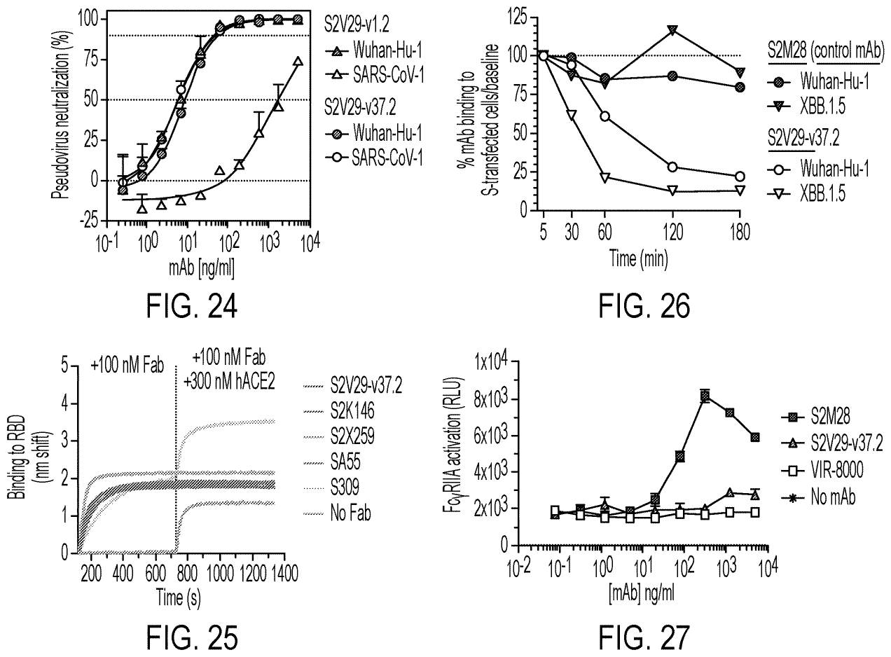

Pseudovirus neutralization was performed in VeroE6 cells using S2V29-v37.2 and a number of comparison antibodies, including S2V29-v1.2, and SARS-CoV-2 pre-Omicron and Omicron variant lineages. S2V29-v.37.2 showed improved performance as compared to comparison antibodies in neutralization of SARS-COVS-2 pre-Omicron and Omicron while also exhibiting lower IC50s for representatives of a number of other sarbecovirus clades, including Clade 1a SARS-CoV (FIG. 23). Neutralization in a pseudovirus assay using VeroE6 cells versus mAb concentration for S2V29-v37.2 and S2V29-v1.2 was plotted for the Wuhan-Hu-1 SARVS-CoV-2 lineage and SARS-CoV-1 (FIG. 24). S2V29-v37.2 demonstrated two orders of magnitude improved neutralization potency against SARS-CoV-1 as compared to S2V29-v1.2.

With respect to non-S2V29 comparison antibodies (FIG. 23), similar to S2V29-v37.2, S2X259 (which binds to SARS-CoV-2 antigenic site IIa, non-RBM) cross-reacted with members of all four sarbecovirus RBD clades although it did not recognize as many clade 2 RBDs. Moreover, S2X259 demonstrated lower neutralization potency against SARS-CoV-2 Wuhan-Hu-1, relative to S2V29-v37.2, and lost activity against recent Omicron variant lineages. S309/sotrovimab (which binds to SARS-CoV-2 antigenic site IV, non-RBM) cross-reacted with clade 1b and 1a RBDs, including the divergent bat ACE2-utilizing Rc-0319 viruses, but not with clade 2 and 3 RBDs. The potent RBM-targeting mAbs S2K146 (which binds to SARS-CoV-2 antigenic site Ia), Omi-42 (which binds to SARS-CoV-2 antigenic site Ia) and SA55 (which binds to SARS-CoV-2 antigenic site Ib) revealed different patterns of cross-reactivity against sarbecoviruses. S2K146 is a unique example of an RBM-specific mAb isolated in the pre-Omicron era that retains activity, albeit reduced, against the majority of Omicron strains to date, a finding that may be attributed to its receptor molecular mimicry. S2K146 cross-reacted with SARS-CoV-1 and some related clade 1a sarbecoviruses, as well as selected clade 3 sarbecoviruses. Omi-42, which is in clinical development in the United States, bound to human ACE2-utilizing clade 1b RBDs and did not neutralize recently-dominant F456L-harboring SARS-CoV-2 XBB variant lineages. Finally, SA55, in clinical development in China, broadly reacted with most ACE2-utilizing RBDs (clades 1b, 1a, and 3; lacking binding to a few clade 1b and 3 RBDs), but did not cross-react with any of the clade 2 RBDs tested.

Bio-layer interferometry (BLI) was also performed using S2V29-v37.2 and a number of comparison antibody Fab fragments to examine competition with monomeric ACE2 for Wuhan-Hu-1 RRC binding (FIG. 25). S2V29-v37.2 competed with ACE2 for binding to the SARS-CoV-2 RBD. All comparison mAbs also competed with ACE2, with the exception of S309.

The ability of S2V29-v37.2 to induce S1 shedding was also examined (FIG. 26). As has been observed for other RBM-targeting mAbs, S2V29-v37.2 efficiently triggered S1 shedding, a mechanism that can contribute to viral neutralization.37

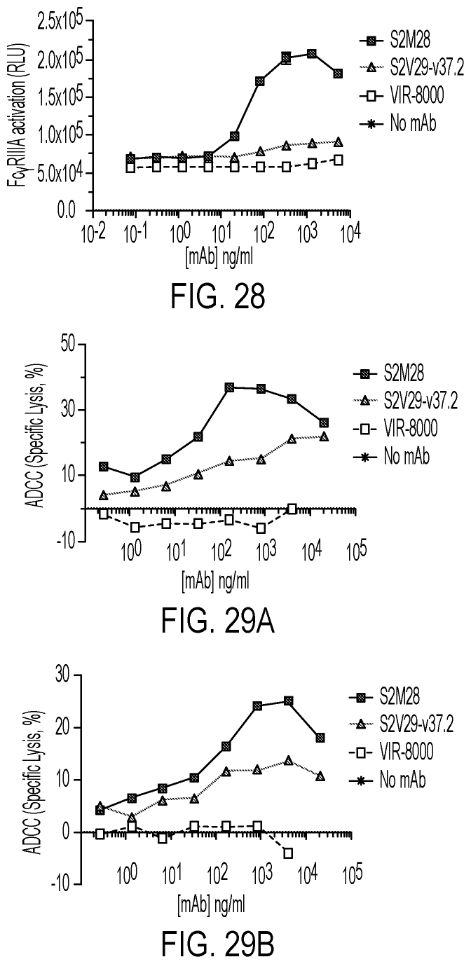

Effector functions of S2V29-v37.2, particularly FcγRIIa activation (FIG. 27) and FcγRIIIa activation (FIG. 28) were also examined in further detail in vitro using a Jurkat cell line-based reporter assay. Possibly due to S1 shedding kinetics, S2V29-v37.2 weakly activated FcγRIIa and FcγRIIIa in vitro. However, using primary human cells to evaluate NK-mediated antibody-dependent cell cytotoxicity (ADCC; measured using NanoLuc HiBiT Extracellular Detection Reagent) in freshly isolated cells from two previously-FcγRIIIa genotyped donors (heterozygous F/V158 upper graph, or homozygous high-affinity V/V158, lower graph) having surface expression of SARS-CoV-2 S and intracellular expression of HiBiT (FIG. 29A and FIG. 29B), moderate antibody-dependent cell cytotoxicity (ADCC) was observed. These findings suggest that S2V29-v37.2 antiviral activity may employ secondary mechanisms beyond direct viral neutralization.

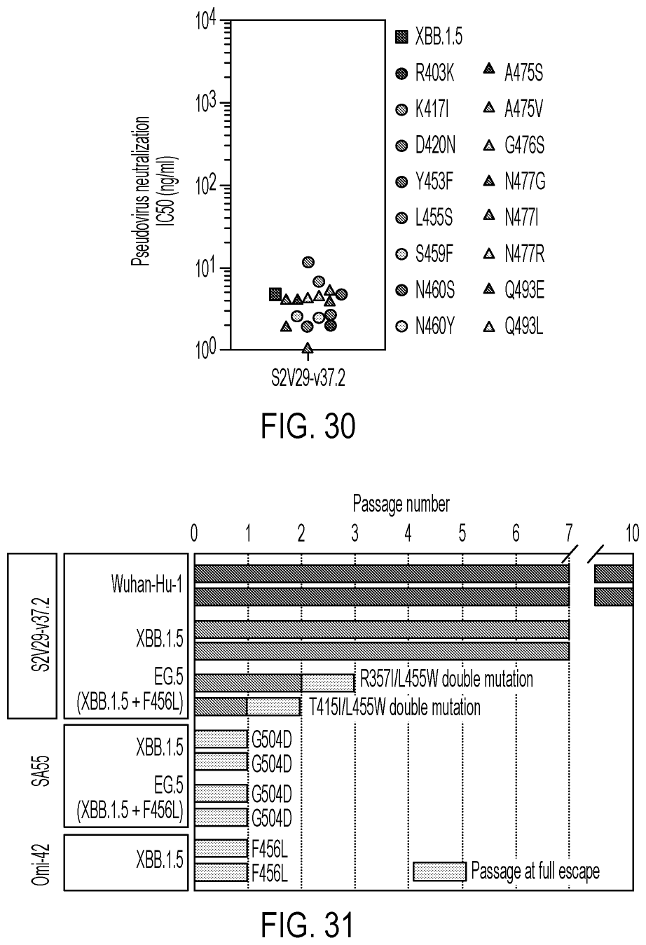

To evaluate the resilience of S2V29-v37.2 to epitope mutations found in circulating viruses, pseudovirus neutralization studies were performed on the XBB.1.5 background using all epitope variants which appear in the GISAID database with >0.005% prevalence, up to 27 Dec. 2023 (FIG. 30). All were potently neutralized by S2V29-v37.2.

To directly evaluate viral escape from S2V29-v37.2, replicating VSV (rVSV) chimeras harboring SARS-CoV-2 S variant glycoproteins instead of the endogenous VSV G were used in experiments performed with S2V29-v37.2 alongside SA55 and Omi-42 mAbs as benchmarks. (FIG. 31). A single round of passaging was sufficient to select viral escapes from the SA55 mAb (G504D in the XBB.1.5 and EG.5 S backgrounds) and Omi-42 (F456L in the XBB.1.5 S background). In contrast, no S2V29-v37.2 escape mutants were isolated for the Wuhan-Hu-1 and XBB.1.5 S backgrounds after ten and seven rounds of serial passaging, respectively. S2V29-v37.2 escape was only observed in the EG.5 S background after two or three rounds of serial passaging which led to the emergence of the L455W mutation combined with either R357I or T415I.

To validate the DMS and serial passaging results, S2V29-v37.2-mediated neutralization of a large panel of SARS-CoV-2 S pseudovirus variant lineages was evaluated (FIG. 30 and FIGS. 33-35). Specifically, FIG. 32 and Table 23 show results for S2V29-v37.2-mediated neutralization of SARS-CoV-2 pseudoviruses carrying mutations that were observed as DMS binding escapes in one or more strain background, plus F456L.

In Table 23, mutations are annotated by: FL=fold-change reduction in ACE2 affinity in the XBB.1.5 background (evaluated by DMS45, available from tstarrlab.github.io/SARS-CoV-2-RBD_DMS_Omicron-XBB-BQ/RBD-heatmaps/; except F456L is from an SPR measurement from the present work); PsV=impact on pseudovirus infectivity (- is typical range, ⬇ is on the low end of typical, ⬇⬇ is a large decrease); NC=minimum number of nucleotide changes required for the mutation to occur, and CT=total counts in the GISAID database as of Jan. 17, 2024. ND: Not determined; nt: not tested. The ND mutations were challenging to test for neutralization because of a strong defect in pseudovirus infectivity.

TABLE 23
S2V29-v37.2-mediated neutralization of SARS-CoV-2

| | F456D | F456E | F456P | F456K | F456R | F456A | F456G | F456L | F456V | F459P | WT |
|---|---|---|---|---|---|---|---|---|---|---|---|
| FL | 1700 | 480 | 66 | 15 | 18 | 15 | 22 | 1.6 | 5 | 3 | na |
| PsV | ↓↓ | nt | ↓↓ | ↓↓ | ↓↓ | ↓↓ | ↓↓ | — | — | — | — |
| NC | 2 | 3 | 2 | 3 | 2 | 2 | 2 | 1 | 1 | 1 | na |
| CT | 0 | 1 | 1 | 0 | 2 | 0 | 0 | 291K | 142 | 57 | 16.0M |

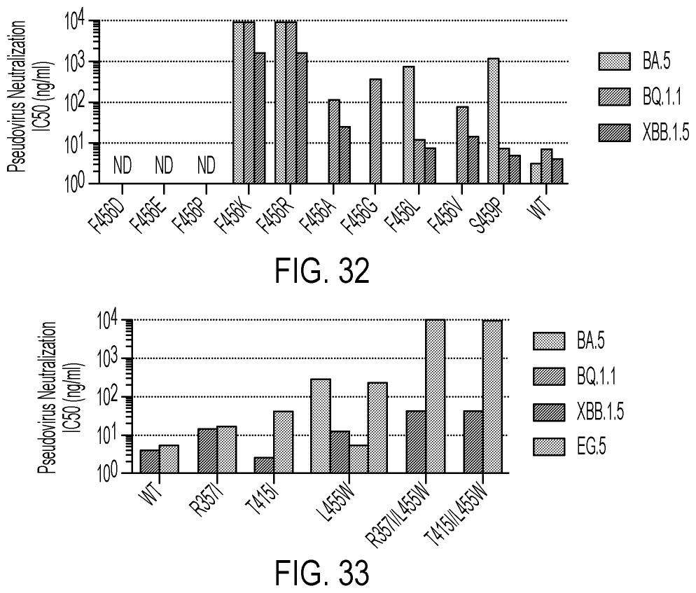

FIG. 33 and Table 24 show results for S2V29-v37.2-mediated neutralization of SARS-CoV-2 pseudoviruses carrying mutations that were observed during EG.5 rVSV resistance selection experiment. In Table 24, mutations are annotated by total GSAID counts (GSAID) as well as counts in the +F456L background (i.e. EG.5, +F456L) in the GISAID database as of Jan. 17, 2024.

TABLE 24
S2V29-v37.2-mediated neutralization of SARS-CoV-2
from rVSV resistance selection

| | | | | | R357I/ | T415I/ |
|---|---|---|---|---|---|---|
| | WT | R357I | T415I | L455W | L455W | L455W |
| GISAID | 16.4M | 175 | 63 | 540 | 0 | 0 |
| +F456L | 291K | 6 | 2 | 462 | 0 | 0 |

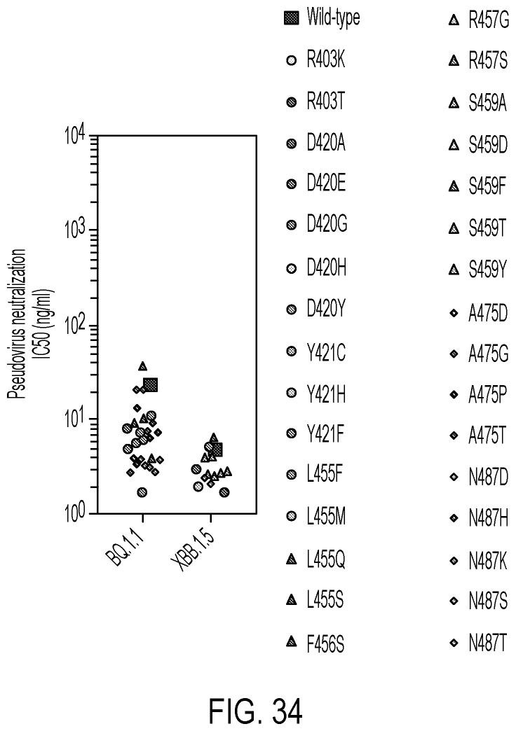

FIG. 34 shows results for S2V29-v37.2-mediated neutralization of SARS-CoV-2 pseudoviruses carrying additional epitope mutations, which are all below 0.005% frequency in GISAID, but are all accessible by a single nucleotide change from wild-type sequences. Mutations were tested in either the BQ.1.1 or XBB.1.5 background; some mutations were tested in both backgrounds.

The impact of mutations identified by DMS, and that retained sufficient pseudovirus infectivity, on S2V29-v37.2-mediated neutralization (F456K, F456R, F456A, F456G and S459P, showed complete loss of neutralization with the F456K and F456R mutations (0 and 2 occurrences in GISAID respectively), which reduced ACE2 binding affinity and pseudovirus infectivity (FIG. 32). The effect of mutations on neutralizing activity was dependent on the S background. For instance, S459P promoted escape from S2V29-v37.2-mediated neutralization in an early-Omicron (BA.5) but not in later-Omicron (BQ.1.1 and XBB.1.5) backgrounds (FIG. 32). These results might be explained by remodeling of the putative BA.5 RBD-N460/VL-Y97 hydrogen bond to an RBD-K460/VH-D57 salt bridge (the latter interaction is observed in the S2V29-v37.2-bound EG.5 structure). L455W did not promote neutralization escape in the BQ.1.1 or XBB.1.5 S backgrounds, but did so in the BA.5 and EG.5 S backgrounds, likely due to the presence of the N460 or F456L mutations, respectively (FIG. 33). Although S2V29-v37.2 potently neutralized currently circulating strains harboring F456L, this mutation reduced neutralization potency by two orders of magnitude in an N460-harboring (BA.5) RBD (FIG. 32).

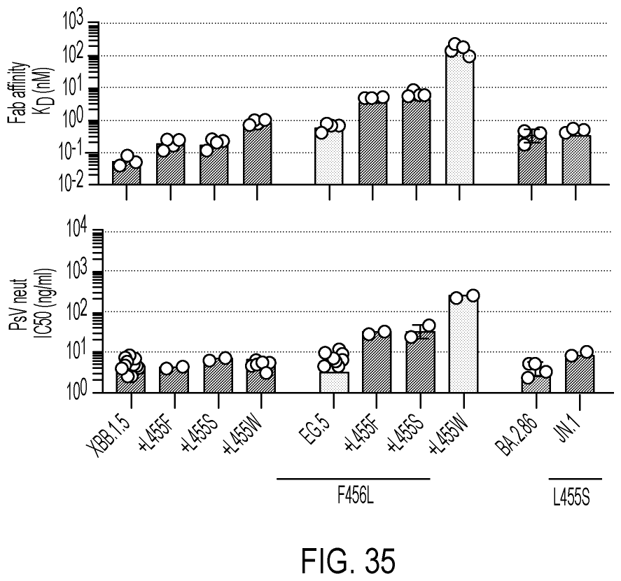

The above findings point to L455W as a potential key mutation for viral escape from S2V29-v37.2. Consistent with the serial passaging results, L455W in the background of EG.5 did not completely escape S2V29-v37.2-mediated neutralization or binding in the absence of either the R357I or the T415I mutation. Specifically, as shown in FIG. 35, the impact of mutations at RBD position 455 on S2V29-v37.2 Fab binding affinity measured by SPR (top) and on S2V29-v37.2-mediated pseudovirus neutralization (bottom) showed neutralization IC50: EG.5-L455W 236 ng/ml, EG.5-L455W/R357I>1250 ng/ml, EG.5-L455W/T415I>1250 ng/ml; and binding affinity: EG.5-L455W KD 160 nM (FIG. 33 and FIG. 35).

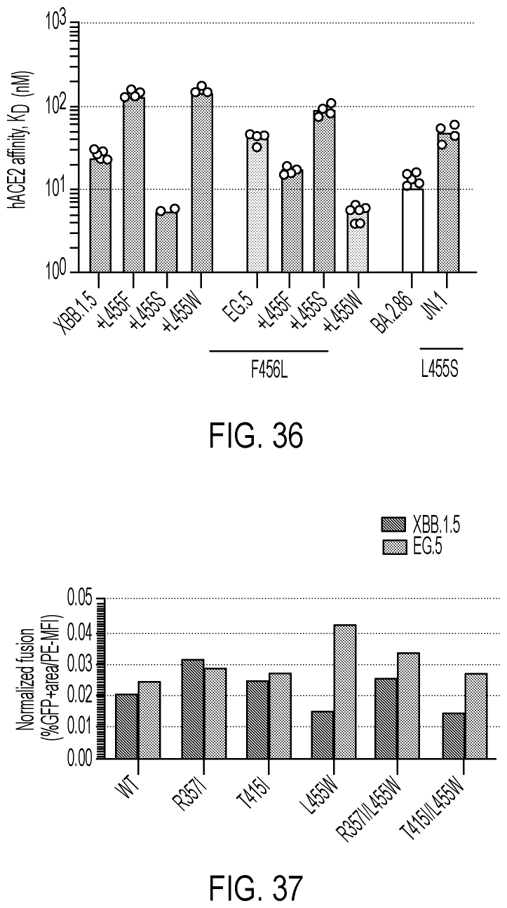

The impact of the L455W substitution on markers of viral fitness was also assessed. Whereas the L455W substitution reduced ACE2 binding affinity (5.9-fold) in the XBB.1.5 background, it enhanced ACE2 binding in the EG.5 (XBB.1.5+F456L) background as measured by SPR (FIG. 36), consistent with results from an S: ACE2-mediated cell-cell fusion assay using VeroE6/TMPRSS2 and SARS-CoV-2 S-expressing cells, normalized by SARS-CoV-2 S expression (FIG. 37). Additionally, L455W is anticipated to be equivalently or more immune evasive than L455F or L455S; 47 the latter two mutations have recently been associated with epidemic spread, likely driven by the strong convergent immune pressure at the RBD positions 455 and 456. These observations are at odds with the very low frequency of L455W in the GISAID database (0.0033% in all backgrounds; 0.17% in F456L background, FIG. 33).

The following materials and methods were used in this Example 5:

### Cell Lines

Cell lines were obtained from ATCC (HEK293T, Vero-E6), Thermo-Fisher Scientific (HEK293F), Invitrogen (Expi-CHO cells) and Takara (Lenti-X 293T cells). Vero-TMPRSS2 (Vero-T) cells were generated in-house. Expi-293 (Invitrogen) and Expi-CHO cells (Invitrogen) were maintained in Expi-293 Expression Medium (Invitrogen) and ExpiCHO-Expression Medium (Gibco), respectively. Vero E6 and Lenti-X cells were cultured in DMEM high glucose with GlutaMAX™ (Gibco) supplemented with 10% fetal bovine serum (FBS) (Integro) and 1% Penicillin-Streptomycin (Gibco). Vero-T cells were cultured in DMEM high glucose with GlutaMAX™ (Gibco) supplemented with 10% fetal bovine serum (FBS) (Integro), 1% Penicillin-Streptomycin (Gibco) and 8 μg/mL puromycin (Gibco). All cell lines used in this study, except HEK293F and Expi293 used for protein expression and HEK293T used for sarbecovirus neutralizations, were routinely tested for mycoplasma and found to be mycoplasma-free.

### Antibody Isolation and Recombinant Production

S2V29-v1.1 mAb was isolated from peripheral blood mononuclear cells (PBMC) of SARS-CoV-2 convalescent and vaccinated individuals under study protocols approved by local institutional review boards (Canton Ticino Ethics Committee, Switzerland, Comitato Etico Milano Areal). The donor provided written informed consent for the use of blood and blood derivatives for research.

PBMC were isolated by Ficoll density gradient centrifugation and B cells were enriched by staining with CD19 PE-Cy7 (BD Bioscience, cat. 341113) and incubation with anti-PE microbeads (Miltenyi Biotec, cat. 130-048-801), followed by positive selection using LS columns (Miltenyi Biotec, cat. 130-042-401). Enriched B cells were stained with anti-IgM, anti-IgD, anti-CD14 and anti-IgA, all PE labelled, and prefusion SARS-CoV-2 S with a biotinylated Avi tag conjugated to Streptavidin Alexa-Fluor 647 (Fisher scientific, cat. 10308062). SARS-CoV-2 S-specific IgG+ memory B cells were sorted by flow cytometry via gating for PE-4 negative and Alexa-Fluor 647 positive cells. Antigen-specific memory B cells were co-cultured with mesenchymal stromal cells (MSC) in the presence of a cocktail of stimuli that induces the proliferation and differentiation of B cells into antibody secreting cells. After 7 days of culture, B cell supernatants were screened for the presence of antibodies of interest.

S2V29-v1.1 VH and VL sequences were obtained by RT-PCR and subcloned in IgG1 expression vectors; the amino-acid sequence of the original S2V29-VL. 1 isolated from B cells carried a germline encoded cysteine residue which was mutated to serine in S2V29-VL.2 to reduce the risk of forming improper disulfide bonds (the mAb carrying the Cys to Ser mutation is variant antibody S2V29-v1.2). The VH and VL amino-acid sequences of the comparison IgGs were available from previous work or retrieved from publications and the DNA sequence was produced with codon optimization for expression in hamster cells, then subcloned into IgG1 expression plasmids. The antibodies were expressed as recombinant human IgG1 (G1m3 allotype) carrying the half-life extending M428L/N434S (LS) mutation in the Fc moiety. ExpiCHO cells were transiently transfected with heavy and light chain expression vectors as described previously. For in vivo experiments in Syrian hamsters, S2V29-v37.2 and a control mAb (specific to Plasmodium falciparum sporozoite) were produced with a Syrian hamster IgG2 Fc.

For binding and competition measurements, S2V29-v37.2 Fab and SA55 Fab were obtained by fragmentation of the corresponding IgG using the FabLACTICA Fab kit (Genovis, Cat #: A2-AFK-025) according to manufacturer's protocol. The Fab-containing fraction was concentrated and buffer-exchanged into filtered HBS buffer (10 mM HEPES pH 7.5, 150 mM NaCl) using an Amicon 10 kDa cutoff concentrator (Millipore Sigma, Cat #: ACS501024). The IgG digestion reactions were analyzed by SDS-PAGE. Recombinant S309 Fab and S2X259 Fab used for the ACE2 competition experiment were expressed in HEK293 suspension cells, purified using CaptureSelect IgG-CH1 resin and buffer exchanged into PBS (ATUM Bio). Recombinant S2K146 Fab used for the ACE2 competition experiment was expressed in ExpiCHO and purified using CaptureSelect CH1-XL MiniChrom columns (Thermo Fisher Scientific).

### Machine-Learning-Assisted Affinity Maturation

The coding sequences of the S2V29 VH and VL domains were subcloned into a yeast vector for cell surface Fab display, with DNA encoding a V5 epitope tag fused in-frame to the CL domain. Antibody libraries of 1×10⁷ DNA sequence variants were designed to diversify every CDR position, with the selection of possible mutations guided by homology modeling and structure-based design using TRIAD. The libraries were constructed by PCR-amplifying the CDRs with DNA oligonucleotides containing degenerate codons and reassembling the vector in vitro with either Golden Gate Assembly, Gibson Assembly, or overlap extension PCR, as appropriate for each library design. Each library was transformed into S. cerevisiae. After expressing Fabs, cells were washed with PBS containing 0.1% BSA and incubated in the same buffer containing mouse anti-V5 antibody (SV5-Pk1, Bio-Rad Laboratories, Inc.) and either 1 μM of biotinylated SARS-CoV-1 RBD or 100 nM of biotinylated SARS-CoV-2 BQ.1.1 RBD. Cells were washed and stained with PE-streptavidin (Jackson ImmunoResearch, Inc.) and goat anti-mouse StarBright Blue 700 (Bio-Rad Laboratories, Inc.), and then subject to FACS (WOLF Cell Sorter, NanoCellect Biomedical, Inc.). Cells were binned according to levels of Fab display and antigen binding, and antibody coding sequences were sequenced using a MiniSeq (Illumina, Inc.) or a MinION (Oxford Nanopore Technologies, plc). Antibody variant sequences were used to train AI models predicting binding affinity towards the RBDs of SARS-CoV-1 and SARS-CoV-2 BQ.1.1. These models were used to design an optimized 8×10⁶ library, which contained antibody variants expected to bind RBDs from both viruses with greater affinity than S2V29. (The S2V29: RBD BQ.1.1 structure had not yet been solved at the time of library design, so the models did not involve input from structure.) Using serial rounds of FACS, the optimized library was enriched for antibody variants that bind to the SARS-CoV-1 RBD, and the enriched populations were subsequently screened to ensure retention of binding to the SARS-CoV-2 BQ1.1 RBD. Enriched populations from each round were deep-sequenced, and antibody variant abundance and prediction scores were used to select candidate antibodies for expression in mammalian cells and downstream evaluation of purified mAbs.

### Recombinant RBD and ACE2 Production

SARS-CoV-2 RBD proteins (residues 328-531 of S protein from GenBank NC_045512.2 with N-terminal signal peptide from mouse Ig heavy chain and C-terminal 8xHis-AviTag or Thrombin-8xHis-AviTag) and other sarbecovirus RBD proteins for SPR binding assays were expressed in Expi293F cells at 37° C. and 8% CO2. Transfections were performed using the ExpiFectamine 293 Transfection Kit (Thermo Fisher Scientific). Cell culture supernatants were collected four to five days after transfection and supplemented with 10×PBS to a final concentration of 2.5×PBS (342.5 mM NaCl, 6.75 mM KCl and 29.75 mM phosphates). RBD proteins were purified by IMAC using Cobalt resin and buffer exchanged into PBS by size exclusion chromatography using a Superdex 200 Increase 10/300 GL column (Cytiva). For BLI experiments, recombinant SARS-CoV-2 RBD strain Wuhan-Hu-1 was purified by cobalt affinity chromatography and biotinylated using a BirA biotin-protein ligase bulk reaction kit (Avidity) followed by size-exclusion chromatography using a S200 Increase 10/300 GL column (Cytiva) into PBS.

XBB.1.5, BQ.1.1, and EG.5 RBD constructs used for crystallization included a C-terminal 8xHis-Avi, Thrombin-TwinStrep-8xHis, or Thrombin-8xHisTag, respectively. Proteins were expressed similarly as XBB.1.5, BQ.1.1, and EG.5 RBD constructs used for SPR binding assays, as described above, except with the addition of 10 μM kifunensine. Cell culture supernatant was collected four days after transfection and supplemented with 10×PBS to a final concentration of 2.5×PBS. Protein was purified using a HisTALON Superflow cartridge (Takara) followed by buffer exchange into PBS using a Superdex 200 Increase 10/300 GL column (Cytiva).

For SPR binding measurements, recombinant human ACE2 (residues 19-615 from Uniprot Q9BYF1 with a C-terminal thrombin cleavage site-TwinStrep-10xHis-GGG-tag, and N-terminal signal peptide) was expressed in Expi293F cells at 37° C. and 8% CO₂. Transfection was performed using the ExpiFectamine 293 Transfection Kit (Thermo Fisher Scientific). Cell culture supernatant was collected six days after transfection, adjusted to a final concentration of 80 mM Tris-HCl pH 8.0, 100 mM NaCl, and then incubated with BioLock solution (IBA GmbH). ACE2 was purified using a StrepTrap HP column (Cytiva) followed by isolation of monomeric ACE2 by size exclusion chromatography using a Superdex 200 Increase 10/300 GL column (Cytiva) pre-equilibrated in 20 mM Tris-HCl pH 7.5, 150 mM NaCl. Recombinant ACE2 used for BLI competition experiments (residues 19-615 from Uniprot Q9BYF1 with a C-terminal Avi-10xHis-GGG-tag, and N-terminal signal peptide) was expressed in Expi293 cells as described above and purified using a HisTrap excel column followed by buffer exchange using a Superdex 200 Increase 10/300 GL column (Cytiva) pre-equilibrated in PBS.

### Neutralization of Authentic SARS-CoV-2 Viruses

VeroE6 cells were seeded into flat bottom tissue culture 96-well plates at 20,000 cells/well and cultured overnight at 37° C. Twenty-four hours later, 9-point 1:4 serial dilutions of S2V29-v37.2 were prepared in infection media (DMEM+2% BSA) and each dilution was tested in triplicate per plate (range: 1,250 to 0.019 ng/mL final concentration). SARS-CoV-2 authentic virus stock was diluted in infection media for a final concentration of 200 plaque forming units per well (MOI 0.01). Antibody dilutions were added to virus and incubated for 30 minutes at 37° C. Media was removed from the cells, mAb-virus complexes were added, and cells were incubated at 37° C. At 18-30 hours post-infection (wild-type USA-WA1/2020 18 h, Delta 30 h, BA.1 24 h, BA.2 30 h, BA.5 18 h, XBB.1.5 24 h, XBB.1.6 30 h, EG.5.1 24 h, FL. 1.5.1 30 h), cells were fixed with 4% PFA for 30 minutes at RT, then washed 3 times with PBS to remove residual PFA. The cells were permeabilized with 100 μL of 0.25% Triton X-100 in PBS for 30 minutes at RT, followed by three washes with PBS. Cells were incubated with 50 μL of anti-SARS-CoV-2 nucleocapsid antibody (Sino Biologicals, 40143-R001) at 1:2000 for 1 hour at RT. Plates were washed three times with PBS and then incubated for 1 hour at RT with 50 μL/well of goat anti-rabbit IgG Alexa647 (Invitrogen, A-21245) secondary antibody at a final dilution of 1:1000 mixed with 2 μg/mL Hoechst dye. After washing 3 times with PBS, 200 μL of fresh PBS was added for imaging. Plates were imaged on a Cytation5 plate reader. Whole well images were acquired (12 images at 4× magnification per well) and nucleocapsid-positive cells were counted using the manufacturer's software.

### Affinity Determination by Surface Plasmon Resonance (SPR)

Measurements were performed using a Biacore 8K or Biacore T200 instrument. CM5 chips with covalently immobilized anti-AviTag polyclonal antibody (GenScript, Cat #: A00674-40) were used to capture His-AviTag containing RBDs. Running buffer was 1×HBS-EP+ pH 7.4 (10 mM HEPES, 150 mM NaCl, 3 mM EDTA and 0.05% v/v Surfactant P20) (Cytiva, Cat #: BR100669). Experiments were performed with a 3-point 4-fold dilution series of monomeric S2V29-v37.2 Fab starting at 50 nM (50, 12.5, 3.13 nM) or monomeric TwinStrep-His-tagged ACE2 starting at 300 nM (300, 75, 18.75 nM).

Experiments were run as single-cycle kinetics with n-2-4 replicates for each RBD ligand. Data were double reference-subtracted and fit to a binding model using the Biacore Insight software. The 1:1 binding model was used to determine the kinetic parameters. For S2V29-v37.2 Fab binding to Shaanxi2011, a constant Rmax calculated from the RBD capture level was applied to account for the low Rmax from the default analysis.

### Production of VSV-Based SARS-CoV-2 and SARS-CoV-1 Urbani S Pseudotyped Virus

To generate SARS-CoV-2 and SARS-CoV-1 Urbani S pseudotyped vesicular stomatitis virus (VSV) for use in neutralization assays employing VeroE6 cells, Lenti-X 293T cells were seeded in 15-cm dishes. The next day, cells were transfected with the plasmid encoding for the SARS-CoV-2 spike variant (or SARS-CoV-1 Urbani) using TransIT-Lenti (Mirus Bio) according to the manufacturer's instructions. One day post-transfection, cells were infected with VSV (G*ΔG-luciferase) (Kerafast) at an MOI of 3 infectious units/cell. Viral inoculum was washed off after one hour and cells were incubated for another day at 37° C. The cell supernatant containing S pseudotyped VSV was collected at day 2 post-transfection, centrifuged at 1,000×g for 5 minutes to remove cellular debris, aliquoted, and frozen at −80° C.

### VSV-Based Sarbecovirus (Clade 1b, Clade La, and Clade 2) S Pseudotyped Virus Production

To generate sarbecovirus S pseudotyped VSV for use in neutralization assays performed in HEK-293T-hACE2 and HEK-293T-R.alc.ACE2, constructs for membrane-anchored S glycoproteins from SARS-CoV-1 Urbani, BA.2.86 (WPL86459.1), GX-Pangolin (QIA48623.1), Khosta-1 (QVN46559.1), Khosta-2 (QVN46569.1), SARS-CoV-1 Civet007 (AAU04646.1), RaTG13delta21 (QHR63300.2), WIVI (AGZ48828.1), RsSHC014 (AGZ48806.1), PRD-0038 (QTJ30153.1), PRD-0038-dm (harboring mutations of the SARS-CoV-2 equivalent positions K493Y/T498W) (QTJ30153.1), and BtKY72 (APO40579.1) were codon optimized and synthesized by Genscript for mammalian cells expression, cloned in frame with a Kozak sequence to direct translation and harboring wild-type signal peptides. The last 21 residues were deleted, except for SARS-CoV-1 Urbani, Civet007, GX-Pangolin, and WIV1. Khosta-1, Khosta-2, RaTG13, RsSHC014, PRD-0038, PRD-0038-dm (K493Y/T498W) S genes were synthesized with a triple Flag tag while the rest of the genes were synthesized with no tag. All the S genes were cloned into the HDM vector except for WIV1 S and SARS-CoV-1 Urbani S which were cloned into pcDNA3.1 (−) and GX-Pangolin S which was cloned into phCMV1.

Sarbecovirus S pseudotyped VSV were generated as previously described. Briefly, HEK293T cells in DMEM supplemented with 10% FBS and 1% PenStrep and seeded in poly-D-lysine coated 10-cm dishes were transfected with a mixture of 24 μg of the corresponding plasmid encoding for: SARS-CoV-1 Urbani S, BA.2.86 S, WIVI S, RaTG13 S, SARS-CoV-1 Civet007 S, Khosta-1, Khosta-2 S, GX-Pangolin S, RsSHC014 S, PRD-0038 S S, PRD-0038-dm S or BtKY72 S and 60 μl Lipofectamine 2000 (Life Technologies) in 3 ml of Opti-MEM, following manufacturer's instructions. After 5 h at 37° C., DMEM supplemented with 20% FBS and 1% PenStrep was added. The next day, cells were washed three times with DMEM and were transduced with VSVΔG-luc. After 2 h, virus inoculum was removed and cells were washed five times with DMEM prior to the addition of DMEM supplemented with anti-VSV-G antibody [Il-mouse hybridoma supernatant diluted 1 to 25 (v/v), from CRL-2700, ATCC] to minimize parental background. After 18-24 h, supernatants containing pseudotyped VSV were harvested, centrifuged at 2,000×g for 5 minutes to remove cellular debris, filtered with a 0.45 μm membrane, concentrated 10 times using a 30 kDa cut off membrane (Amicon), aliquoted, and frozen at −80° C. until use.

### Neutralization of SARS-CoV-2 (and SARS-CoV-1 Urbani) Pseudoviruses

For neutralization of VSV-based SARS-CoV-2 (and SARS-CoV-1 Urbani) S pseudotyped viruses, Vero E6 cells were seeded into clear bottom white walled 96-well plates (PerkinElmer) at 20,000 cells/well and cultured overnight at 37° C. The next day, 9-point 3-fold serial dilutions of mAbs were prepared in media. SARS-CoV-2 (or SARS-CoV-1 Urbani) pseudotyped VSVs were diluted at 0.1 MOI in media and added 1:1 to each mAb dilution. Virus: mAb mixtures were incubated for 1 hour at 37° C. Media was removed from the Vero E6 cells and 50 μL of virus: mAb mixtures were added to the cells. One hour post-infection, 100 μL medium was added to all wells. After 20-24 hours incubation at 37° C., medium was removed and 100 μL Steadylite plus reagent (diluted 1:1 in DPBS) was added to each well. The plates were incubated at room temperature for 10 minutes and luminescence was read on a plate reader. Two to three technical replicates were measured and at least two biological repeats were performed. For most neutralizations performed with the S2V29-v1.2 mAb and for the majority of S2V29-v37.2 neutralizations against single-site point mutations (but not S2V29-v37.2 neutralizations against SARS-CoV-2 strains), experiments were performed with the addition of 100 ng/ml anti-VSV-G Ab. All data were normalized based on internal control RLU values (untreated cells for 100% neutralization and infected cells with or without anti-VSV-G antibody for 0% neutralization) and plotted with GraphPad Prism (version 10.1.2) using a nonlinear regression 4-parameters model.

### Neutralization of Sarbecovirus Pseudoviruses

For pseudotyped VSV sarbecovirus neutralizations, HEK293T cells were transiently transfected with plasmids encoding for full-length human ACE2 or R. Alcyone ACE2 following the protocol described. Briefly, HEK293T cells at 90% confluency and seeded in poly-D-lysine coated 10-cm dishes were transfected with a mixture of 8 μg of the corresponding plasmid encoding the ACE2 ortholog and 30 μl of Lipofectamine 2000 (Life Technologies) prepared in Opti-MEM according to the manufacturer's instructions. After 5 h at 37° C., cells were trypsinized, seeded into poly-D-lysine coated clear bottom white walled 96-well plates at 40,000 cells/well and cultured overnight at 37° C. For neutralizations, eleven 2-fold serial dilutions of SA55, S2K146, S2X259, Omi-42, S309, S2V29-v1.2 or S2V29-v37.2 IgGs were prepared in DMEM. 20 μl of the different sarbecovirus pseudotypes described above were added 1:1 (v/v) to each IgG and mixtures were incubated for 45-60 min at 37° C. After removing their media, transfected HEK293T cells were washed two times with DMEM and 40 μL of the mixture containing virus: IgG were added. Two hours later, 40 AL DMEM were added to the cells. After 17-20 h, 60 μL of One-Glo-EX substrate (Promega) were added to each well and incubated on a plate shaker in the dark. After 5-15 min incubation, plates were read on a Biotek plate reader. (S309 neutralization of SARS-CoV-1 Urbani was performed with Vero-TMPRSS2 cells, but otherwise was performed as described.) Measurements were done in duplicate with at least two biological replicates. Relative luciferase units were plotted and normalized in Prism (GraphPad): cells alone without pseudotyped virus were defined as 0% infection, and cells with virus only (no sera) were defined as 100% infection.

### Competition of mAb and ACE2 for Binding to RBD by Biolayer Interferometry (BLI)

Protein reagents were diluted in 10× Kinetics Buffer (Sartorius, Cat #: 18-1105). The experiment was performed on an Octet Red instrument. Streptavidin biosensors (Sartorius, Cat #: 18-5019) were hydrated in water for 15 min before the experiment. Biotinylated His-Avi-tagged Wuhan (Wu-WT) RBD was immobilized on the sensors at 10 μg/mL for 10 s. Wu-WT RBD-immobilized sensors were then dipped into Kinetics Buffer for 60 s to establish a baseline before being dipped into a 100 nM Fab solution for 600 s (association phase 1) and subsequently into a mixture of 100 nM of the same Fab plus 300 nM His-Avi-tagged ACE2 solution for 600 s (association phase 2).

Cell-Surface mAb-Mediated S1 Shedding

Expi-CHO cells were transiently transfected with plasmids encoding for Wuhan-Hu-1 and XBB.1.5 S. After 48 h of incubation at 37° C., cells were collected, counted, and 90,000 cells/well were dispensed into a round-bottom 96 well plate (Corning). After a washing step with wash buffer (PBS supplemented with 0.01% BSA), cells were incubated with 15 μg/ml mAbs (diluted in wash buffer) for 180, 120, 60, 30 or 5 min at 37° C. After the incubation for the allotted time, cells were washed with ice-cold wash buffer and stained with 1.5 μg/mL Alexa Fluor647-labeled Goat Anti Human IgG secondary Ab (Jackson Immunoresearch) for 20 min on ice in the dark. Cells were then washed twice with cold wash buffer and analyzed using a ZE5 cytometer (Biorad) with acquisition chamber T=4° C. Binding at each time point (MFI) was determined normalizing to the MFI at 5 minutes time point and data plotted using GraphPad Prism v. 9.1.1.

### Fc-Mediated Effector Function

Determination of mAb-Dependent Activation of Human FcγRIIa and FcγRIIIa

Activation of human FcγRIIa (allele H131) and FcγRIIIa (high-affinity binding allele V158) was tested using validated, commercially available bioreporter assays in which CHO cells stably expressing SARS-CoV-2 Wuhan-Hu-1 spike protein (CHO-CoV-2-Spike) were used as the target antigen. Nine-point serial dilutions of mAbs (5,000 ng/ml to 0.076 ng/ml) were incubated with 12,500 (for FcγRIIIa) or 10,000 (for FcγRIIa) CHO-CoV-2-Spike cells per 96-plate well in a white, flat-bottom plate for 25 minutes at room temperature. Jurkat effector cells (Promega; Cat. Nr.: G7018 and G9995) expressing indicated FcγRs and stably transfected with an NFAT-driven luciferase gene were thawed, diluted in assay buffer, and added to the plate at an effector to target cell ratio of 6:1 for FcRyIIIa or 5:1 for FcγIIa. Control wells were also included that were used to measure antibody-independent activation (containing target cells and effector cells but no antibody) and background luminescence of the plate (wells containing assay buffer only). Plates were incubated for 20 hours at 37° C. with 5% CO2. Activation of human FcγRs in this bioassay results in the NFAT-mediated expression of the luciferase reporter gene. Luminescence was measured with a luminometer (Bio-Tek) after adding the Bio-Glo™ Luciferase Assay Reagent according to the manufacturer's instructions.

Determination of NK-Cell Mediated Antibody-Dependent Cellular Cytotoxicity 3,000 SARS-CoV-2 Wuhan-Hu-1 S CHO-K1 (HaloTag-HiBiT) cells per well were thaw and seeded in a white polypropylene 96 well round-bottom plate and incubated overnight in a 37° C., 5% CO₂ humified incubator. The day after, serial dilutions of mAbs (serially diluted 5-fold in AIM-V Medium from 20,000 ng/ml to 0.26 ng/ml) were added to the plate already containing the cells (incubation for 10 min prior to mixing with NK cells). Wells without antibody or containing Digitonin (Sigma-Aldrich; Cat. Nr.: D141) at 100 ug/ml were used as negative and positive controls. Finally, also primary human NK cells freshly isolated from whole EDTA blood (Becton Dickinson A G, Cat. Nr.: 367525) from two previously genotyped donors expressing heterozygous affinity (F/V158) or high affinity (V/V158) FcγRIIIa using the MACSxpress NK isolation Kit (Miltenyi, Cat. Nr.: 130-127-695) following the manufacturer's instructions and resuspended in AIM-V medium were added as effectors at an effector-to-target ratio of 10:1 (30′000 cells/well) and the plate was incubated in a 37° C., 5% CO₂ humified incubator for 4 hours. ADCC results in the lysis of target cells which release HiBiT protein into the medium producing luminescence, which can be with a luminometer after addition of NanoLuc HiBiT Extracellular Detection reagent (Promega; Cat. Nr.: N2421) according to the manufacturer's instructions. In brief, after substrate reconstitution, 70 ul/well was added and incubated in the dark for 10 minutes and luminescence was then measured. The percent specific lysis was determined by applying the following formula: (specific RLU−negative control RLU)/(positive RLU−negative control RLU)×100.
Plaque-Based Selection of SARS-CoV-2 mAb Escape Mutants by rVSV

### Production of VSV-SARS-CoV-2 S Chimeras

Recovery of recombinant VSV was performed as described (Whelan et al., 1995). Briefly, BSRT7/5 cells (Buchholz et al., 1999) were inoculated with vaccinia virus vTF7-3 (Fuerst et al., 1986) and subsequently transfected with T7-expression plasmids encoding VSV N, P, L, and G, and an antigenomic copy of the viral genome. Cell culture supernatants were collected at 72 h, clarified by centrifugation (5 min at 1,000×g), and filtered through a 0.22 mm filter. Virus was plaque-purified on Vero CCL81 cells in the presence of 25 mg/mL of cytosine arabinoside (Sigma-Aldrich), and plaques in agarose plugs were amplified on Vero CCL81 cells. Viral stocks were amplified on MA104 cells at an MOI of 0.01 in Medium 199 containing 2% FBS and 20 mM HEPES pH 7.7 at 34° C. Viral supernatants were harvested upon extensive cytopathic effect and clarified of cell debris by centrifugation at 1,000×g for 5 min. Aliquots were maintained at 80° C.

### Selection of Monoclonal Antibody Resistant Mutants (MARMs)

Replicating VSV-SARS-CoV-2 S chimeras were used to select for MARMs as previously described. In brief, MARMs were recovered by plaque isolation on Vero E6 cells (ATCC, CRL-1586) with the indicated monoclonal antibody in the overlay. The concentration of monoclonal antibody in the overlay was determined by neutralization assays at a MOI of 100. Escape clones were plaque-purified on Vero cells (ATCC, CCL-81) in the presence of monoclonal antibody, and plaques in agarose plugs were amplified on MA104 cells (a gift from H. B. Greenberg (Stanford School of Medicine)) with the monoclonal antibody present in the medium. Viral stocks were amplified on MA104 cells at an MOI of 0.01 in Medium 199 containing 2% FBS and 20 mM HEPES pH 7.7 (Millipore Sigma) at 34° C. Viral supernatants were collected upon extensive cytopathic effect and clarified of cell debris by centrifugation at 1,000 g for 5 min. Aliquots were maintained at −80° C. Viral RNA was extracted from VSV-SARS-CoV-2 mutant viruses using RNeasy Mini kit (Qiagen), and S was amplified using OneStep RT-PCR Kit (Qiagen). The mutations were identified by Sanger sequencing (Genewiz). Their resistance was verified by subsequent virus infection in the presence or absence of antibody. In brief, Vero cells were seeded into 12-well plates for overnight. The virus was serially diluted using DMEM and cells were infected at 37° C. for 1 h. Cells were cultured with an agarose overlay in the presence or absence of monoclonal antibody at 34° C. for 2 days. Plates were scanned on a biomolecular imager and expression of eGFP is shown at 48 h after infection.

Selection of SARS-CoV-2 mAb Escape Mutants by rVSV Serial Passaging
Propagation of Replicating VSV-SARS-CoV-2 S Chimeras (rVSV)

Wuhan-Hu-1 rVSV (GFP) was produced as described earlier. All Omicron rVSV-spike constructs were designed in-house and purchased from VectorBuilder (en.vectorbuilder.com) and propagated in Vero-TMPRSS2 cells. For stock production, 100 mm dishes (Falcon Cat. 353003) of Vero-TMPRSS2 were infected at MOI=0.03 in infection medium (DMEM Gibco Cat. #11995-040, 1% Pen/strep Gibco Cat. #15140-122, 2% FCS VWR Cat. #97068-085, 20 mM HEPES pH 7.7 Gibco Cat. 15630080). Plates were incubated at 34° C., 5% CO₂ for 1 hour, after which inoculum was removed and fresh media was added (same formulation). Plates were incubated at 34° C., 5% CO₂ for 72 hours. CPE was assessed visually, and virus containing media was removed, clarified and stored at −80° C. for later use.

rVSV Titration

Vero-TMPRSS2 cells were plated in a 12-well format (CellTreat Cat. 229111). A five-point curve of 10-fold rVSV-spike dilutions were prepared (starting at 1:100 dilution) in infection medium. Diluted virus was incubated with the cells for 1 hour, after which inoculum was removed and plates washed once with PBS (Gibco Cat. 10010023). An overlay of 1% methylcellulose (Sigma Aldrich Cat. M7027-250G) was added to each well, and plates were incubated at 34° C. for 24 hours. Overlays were removed and plates fixed in 4% PFA for 30 minutes, and washed 3 times with PBS. Cells were permeabilized with 0.25% Triton X-100 (Sigma Aldrich Cat. X100-100ML) and stained with mouse anti-VSV-N primary antibody (clone 10G4, Kerafast Cat. EB0009). Plates were washed 3 times with PBS and stained with horseradish peroxidase-conjugated anti-mouse secondary (Jackson ImmunoResearch, Cat. 115-035-062) for 30 minutes at room temperature. Plates were washed 4 times and developed using True Blue substrate (SeraCare Cat. 5510-0030) for 15 minutes. A final wash step with ultrapure H₂O (Gibco Cat. 10977015) was used to inactivate the enzyme and foci were counted manually to calculate infectious titers.

rVSV Serial Passaging

To produce an even monolayer in a 12-well format, 2.5E5 Vero-TMPRSS2 cells were plated in 2 mL of complete DMEM (10% FCS). After 24 hours, rVSV-spike aliquots were thawed and diluted to infect cells at MOI=2 after neutralization. Antibody dilutions (7-point, 4-fold, final maximum concentration=20 mg/mL) were prepared in 2 mL deepwell plates (Nunc Cat. 260251) and diluted rVSV-spike was added. Antibody and virus mixture was incubated at 37° C. for 1 hour. After neutralization, virus was added to cells and incubated at 37° C. for 1 hour, with gentle rocking every 15 minutes. Inoculum was removed, wells were washed once with PBS, and fresh media was added, containing matched concentrations of antibody. Plates were incubated at 37° C. for 72 hours, and “percent CPE” was assessed via inverted light microscope. The algorithm used for selection was as follows: maximum antibody treatment at which CPE exceeds 20%. Supernatants from the selected well were collected, clarified and stored at −80° C. for later use. Supernatants from the ‘no antibody’ positive control well were also collected for passaging.

For subsequent passages, Vero-TMPRSS2 were plated as before. Passage 1 virus was diluted 1:5 in media containing antibody (diluted as before) and neutralized as above. Infection, selection, and collection were performed identically for each passage. Passaging was ceased after complete escape (CPE>20% at 20 mg/mL) or after 10 passages (7 passages for XBB.1.5-spike) elapsed. All passaging studies were performed in duplicate lineages.

rVSV Resistance Mutant Sequencing

Using Trizol (Invitrogen Cat. 15596026) phase extraction followed by column cleanup (Macherey-Nagel Cat. 740983.50), RNA was extracted from resistant rVSV-spike samples. RNA was reverse transcribed using Protoscript II reverse transcriptase (NEB Cat. M0368L) and cDNA for the SARS-CoV-2 spike gene was amplified in two fragments using specific primers (IDT) and KAPA HiFi HotStart ReadyMix (Roche Cat. 07958935001). PCR products were cleaned up using NucleoSpin Gel and PCR Clean-up (Macherey-Nagel Cat. 740609.250) and purity visualized via gel electrophoresis (Invitrogen Cat. A42100). Samples were submitted to MCLab (www.mclab.com) for Sanger sequencing, or Primordium (primordiumlabs.com) for Nanopore sequencing. Results were analyzed with Snapgene software (www.snapgene.com).

Plaque-Based Selection of SARS-CoV-2 mAb Escape Mutants by rVSV

Replicating VSV-SARS-CoV-2 S chimeras were used to select for monoclonal antibody resistant mutants (MARMs) as previously described. In brief, MARMs were recovered by plaque isolation on Vero E6 cells (ATCC, CRL-1586) with the indicated monoclonal antibody in the overlay. The concentration of monoclonal antibody in the overlay was determined by neutralization assays at a MOI of 100. Escape clones were plaque-purified on Vero cells (ATCC, CCL-81) in the presence of monoclonal antibody, and plaques in agarose plugs were amplified on MA104 cells (a gift from H. B. Greenberg (Stanford School of Medicine)) with the monoclonal antibody present in the medium. Viral stocks were amplified on MA104 cells at an MOI of 0.01 in Medium 199 containing 2% FBS and 20 mM HEPES pH 7.7 (Millipore Sigma) at 34° C. Viral supernatants were collected upon extensive cytopathic effect and clarified of cell debris by centrifugation at 1,000 g for 5 min. Aliquots were maintained at −80° C. Viral RNA was extracted from VSV-SARS-CoV-2 mutant viruses using RNeasy Mini kit (Qiagen), and S was amplified using OneStep RT-PCR Kit (Qiagen). The mutations were identified by Sanger sequencing (Genewiz). Their resistance was verified by subsequent virus infection in the presence or absence of antibody. In brief, Vero cells were seeded into 12-well plates for overnight. The virus was serially diluted using DMEM and cells were infected at 37° C. for 1 h. Cells were cultured with an agarose overlay in the presence or absence of monoclonal antibody at 34° C. for 2 days. Plates were scanned on a biomolecular imager and expression of eGFP is shown at 48 h after infection.

### Cell-Cell Fusion Between VeroE6/TMPRSS2 and SARS-CoV-2 Expressing Cells

### Split GFP Cell-Cell Fusion Assay

To evaluate the effect of point mutations in the XBB.1.5 and EG.5 S backgrounds in fusion, we used the split GFP cell-cell fusion assay. Briefly, BHK21 cells transfected with the different S point mutants and VeroE6/TMPRSS2 stably expressing GFP1-10 and GFP 11, respectively, were utilized to quantify cell-cell fusion over 18 hours. BHK21-GFP 1-10 cells were cultured in 6-well cell-culture grade plates and incubated overnight in DMEM containing 10% FBS and 8 μg/ml puromycin. Next day, BHK21-GFP1-10 cells were transfected with 4 ug of DNA encoding one S construct, Opti-MEM and lipofectamine2000 (Invitrogen Cat #11668027) following manufacture instructions and VeroE6/TMPRSS2 GFP11 cells in DMEM containing 10% FBS, 8 μg/ml puromycin and 4 μg/ml blasticidin were seeded in a black, glass bottom 96-well plate at ~36,000 cells per well and incubated overnight. After ~20 hr incubation, transfected BHK21-GFP1-10 cells were washed three times with FluoroBrite DMEM (Gibco Cat #A1896701) and incubated with 500 μL of Gibco cell dissociation buffer for 10 minutes at 37 C with agitation to facilitate resuspension of cells. Cells were then passed through cell-strainer capped tubes (Falcon Cat #08-771-23) to eliminate aggregates, and diluted using FluoroBrite DMEM to have ~60,000 cells per mL. VeroE6/TMPRSS2-GFP11 cells were carefully washed with FluoroBrite DMEM three times and 150 μL of transfected BHK21-GFP1-10 cells were added on top and co-cultured for a period of ~18 hours in the Cytation7 plate imager. Data were analyzed with GraphPad PRISM 10 to quantify GFP+ areas.

### Flow Cytometry for Normalization of Expression

Flow cytometry was utilized to quantify S expression levels on the cell surface of BHK21-GFP 1-10 cells. BHK21-GFP 1-10 cells under puromycin selection were cultured in 6 well cell-culture grade plates and incubated overnight. After ~20 hrs, the BHK21-GFP 1-10 cells were washed with unenriched DMEM and 2.5 mLs of fresh DMEM with 10% FBS was added to cells prior to transfection step. To match conditions with the cell-cell fusion assay, cells were transfected with 4 ug of S plasmid and lipofectamine2000 in Opti-MEM and incubated for ~38 hours. The BHK21-GFP 1-10 cells were then washed with Flow Stain buffer and 500 uL of Gibco cell dissociation buffer was added to remove cells from bottom of the 6 well plates. Cells were resuspended with Flow Stain buffer (eBioscience Cat #00-4222-26), filtered through a cell-strainer capped tube, diluted to ~1×10\hat{ }7 cells per mL. and added to a pyramid bottom 96-well plate. Cells in the plate were centrifuged and the supernatant discarded. Cells were resuspended in Flow Stain Buffer containing 5 μg/mL of S2L20 mAb and incubated at 4 C for ~25 minutes. Cells were washed 3 times with 100 μl of flow stain buffer before adding 0.5 μg of Anti-IgG Fc-PE Secondary Antibody per well (Thermo Fisher Scientific Cat #12-4998-82). After ~25 minutes at 4° C., cells were washed 3× with Flow Stain Buffer. To fix the cells, 100 μL of 2% paraformaldehyde was added to each well and incubated at 4 C for 15 minutes. Cells were then washed 2 times with Flow Stain Buffer prior to being resuspended in 50 μL of fresh Flow Stain Buffer. Cells were transferred into tubes with 450 μL of Flow Stain Buffer, kept on ice, shielded from direct light prior to being counted on BD Biosciences' FACSymphony. All results were analyzed on BD FlowJo 10.8.2. For normalization of expression and cell-cell fusion, total percentage of fusion after 18 hours was divided by mean fluorescence intensity (MFI).

### Prevalence Analysis in GISAID Database

The viral sequences and the corresponding metadata were obtained from GISAID EpiCoV project (www.gisaid.org/). Analysis was performed on sequences submitted to GISAID up to Jan. 17, 2024. S protein sequences were either obtained from GISAID download page or, for the most recently submitted sequences, from the genomic sequences with exonerate 2.4.0-haf93ef1_3 (quay.io/repository/biocontainers/exonerate?tab=tags) using protein to DNA alignment with parameters -m protein2dna -refine full -minintron 999999-percent 20 and using accession NC_045512.2 as a reference. Multiple sequence alignment of all spike proteins was performed with mafft 7.508—hec16e2b_0 (quay.io/repository/biocontainers/mafft?tab=tags&tag-7.508--hec16e2b_0) with parameters --mapout --auto--op 4.5 --reorder--keeplength --addfragments using the same reference as above. The --mapout parameter was used to retrieve insertions. S sequences that were <80% (1019/1273) of the canonical protein length were discarded. To identify each mutation prevalence, missingness (or ambiguous amino acids) was taken into account in both nominator and denominator. Per week prevalence of each mutation was then calculated to get the temporal trend.

The following references and any other references or patents or patent applications mentioned herein are incorporated by reference herein in their entireties, and specifically with respect to disclosure of any antibodies or experimental methods referenced herein: Antinori, A., and Bausch-Jurken, M. (2023). The burden of CoVID-19 in the immunocompromised patient: Implications for vaccination and needs for the future. J. Infect. Dis. 228, S4-S12; Evans, R. A., Dube, S., Lu, Y., Yates, M., Arnetorp, S., Barnes, E., Bell, S., Carty, L., Evans, K., Graham, S., et al. (2023). Impact of CoVID-19 on immunocompromised populations during the Omicron era: insights from the observational population-based INFORM study. Lancet Reg. Health Eur. 35, 100747; Taylor, P. C., Adams, A. C., Hufford, M. M., de la Torre, I., Winthrop, K., and Gottlieb, R. L. (2021). Neutralizing monoclonal antibodies for treatment of CoVID-19. Nat. Rev. Immunol. 21, 382-393; Corti, D., Purcell, L. A., Snell, G., and Veesler, D. (2021). Tackling CoVID-19 with neutralizing monoclonal antibodies. Cell 184, 3086-3108; Suribhatla, R., Starkey, T., Ionescu, M. C., Pagliuca, A., Richter, A., and Lee, L. Y. W. (2023). Systematic review and meta-analysis of the clinical effectiveness of tixagevimab/cilgavimab for prophylaxis of CoVID-19 in immunocompromised patients. Br. J. Haematol. 201, 813-823; Yang, M., Li, T., Wang, Y., Tran, C., Zhao, S., and Ao, G. (2022). Monoclonal antibody therapy improves severity and mortality of CoVID-19 in organ transplant recipients: A meta-analysis. J. Infect. 85, 436-480; Gentile, I., Foggia, M., Silvitelli, M., Sardanelli, A., Cattaneo, L., Viceconte, G., and Federico II CoVID team (2023). Early combination with remdesivir, nirmatrelvir/ritonavir and sotrovimab for the treatment of CoVID-19 in immunocompromised hosts. bioRxiv. 10.1101/2023.09.07.23295202; U.S. Department of Health and Human Services CoVID-19 Therapeutics Thresholds, Orders, and Replenishment. aspr.hhs.gov/CoVID-19/Therapeutics/Orders/; Cameroni, E., Bowen, J. E., Rosen, L. E., Saliba, C., Zepeda, S. K., Culap, K., Pinto, D., VanBlargan, L. A., De Marco, A., di Iulio, J., et al. (2022). Broadly neutralizing antibodies overcome SARS-CoV-2 Omicron antigenic shift. Nature 602, 664-670; Yang, S., Yu, Y., Jian, F., Song, W., Yisimayi, A., Chen, X., Xu, Y., Wang, P., Wang, J., Yu, L., et al. (2023). Antigenicity and infectivity characterisation of SARS-CoV-2 BA.2.86. Lancet Infect. Dis. 23, e457-e459; Wang, Q., Guo, Y., Liu, L., Schwanz, L. T., Li, Z., Nair, M. S., Ho, J., Zhang, R. M., Iketani, S., Yu, J., et al. (2023). Antigenicity and receptor affinity of SARS-CoV-2 BA.2.86 spike. Nature 624, 639-644; Arora, P., Kempf, A., Nehlmeier, I., Schulz, S. R., Jäck, H.-M., Pöhlmann, S., and Hoffmann, M. (2023). Omicron sublineage BQ.1.1 resistance to monoclonal antibodies. Lancet Infect. Dis. 23, 22-23; Addetia, A., Piccoli, L., Case, J. B., Park, Y.-J., Beltramello, M., Guarino, B., Dang, H., de Melo, G. D., Pinto, D., Sprouse, K., et al. (2023). Neutralization, effector function and immune imprinting of Omicron variants. Nature. 10.1038/s41586-023-06487-6; Stanford HVDB team (2023). Table 2: Virus variants and spike mutations vs monoclonal antibodies. Coronavirus Antiviral & Resistance Database. covdb.stanford.edu/susceptibility-data/table-mab-susc/; Pinto, D., Park, Y.-J., Beltramello, M., Walls, A. C., Tortorici, M. A., Bianchi, S., Jaconi, S., Culap, K., Zatta, F., De Marco, A., et al. (2020). Cross-neutralization of SARS-CoV-2 by a human monoclonal SARS-CoV antibody. Nature 583, 290-295; Starr, T. N., Czudnochowski, N., Liu, Z., Zatta, F., Park, Y.-J., Addetia, A., Pinto, D., Beltramello, M., Hernandez, P., Greaney, A. J., et al. (2021). SARS-CoV-2 RBD antibodies that maximize breadth and resistance to escape. Nature 597, 97-102; Tortorici, M. A., Czudnochowski, N., Starr, T. N., Marzi, R., Walls, A. C., Zatta, F., Bowen, J. E., Jaconi, S., Di Iulio, J., Wang, Z., et al. (2021). Broad sarbecovirus neutralization by a human monoclonal antibody. Nature 597, 103-108; Pinto, D., Sauer, M. M., Czudnochowski, N., Low, J. S., Tortorici, M. A., Housley, M. P., Noack, J., Walls, A. C., Bowen, J. E., Guarino, B., et al. (2021). Broad betacoronavirus neutralization by a stem helix-specific human antibody. Science 373, 1109-1116; Dacon, C., Peng, L., Lin, T.-H., Tucker, C., Lee, C.-C. D., Cong, Y., Wang, L., Purser, L., Cooper, A. J. R., Williams, J. K., et al. (2023). Rare, convergent antibodies targeting the stem helix broadly neutralize diverse betacoronaviruses. Cell Host Microbe 31, 97-111.e12; Low, J. S., Jerak, J., Tortorici, M. A., McCallum, M., Pinto, D., Cassotta, A., Foglierini, M., Mele, F., Abdelnabi, R., Weynand, B., et al. (2022). ACE2-binding exposes the SARS-CoV-2 fusion peptide to broadly neutralizing coronavirus antibodies. Science 377, 735-742; Dacon, C., Tucker, C., Peng, L., Lee, C.-C. D., Lin, T.-H., Yuan, M., Cong, Y., Wang, L., Purser, L., Williams, J. K., et al. (2022). Broadly neutralizing antibodies target the coronavirus fusion peptide. Science 377, 728-735; Tortorici, M. A., Beltramello, M., Lempp, F. A., Pinto, D., Dang, H. V., Rosen, L. E., McCallum, M., Bowen, J., Minola, A., Jaconi, S., et al. (2020). Ultrapotent human antibodies protect against SARS-CoV-2 challenge via multiple mechanisms. Science 370, 950-957; Nutalai, R., Zhou, D., Tuekprakhon, A., Ginn, H. M., Supasa, P., Liu, C., Huo, J., Mentzer, A. J., Duyvesteyn, H. M. E., Dijokaite-Guraliuc, A., et al. (2022). Potent cross-reactive antibodies following Omicron breakthrough in vaccinees. Cell 185, 2116-2131.e18; Starr, T. N., Greaney, A. J., Hilton, S. K., Ellis, D., Crawford, K. H. D., Dingens, A. S., Navarro, M. J., Bowen, J. E., Tortorici, M. A., Walls, A. C., et al. (2020). Deep Mutational Scanning of SARS-CoV-2 Receptor Binding Domain Reveals Constraints on Folding and ACE2 Binding. Cell 182, 1295-1310.e20; Taft, J. M., Weber, C. R., Gao, B., Ehling, R. A., Han, J., Frei, L., Metcalfe, S. W., Overath, M. D., Yermanos, A., Kelton, W., et al. (2022). Deep mutational learning predicts ACE2 binding and antibody escape to combinatorial mutations in the SARS-CoV-2 receptor-binding domain. Cell 185, 4008-4022.e14; Thomson, E. C., Rosen, L. E., Shepherd, J. G., Spreafico, R., da Silva Filipe, A., Wojcechowskyj, J. A., Davis, C., Piccoli, L., Pascall, D. J., Dillen, J., et al. (2021). Circulating SARS-CoV-2 spike N439K variants maintain fitness while evading antibody-mediated immunity. Cell 184, 1171-1187.e20; Park, Y.-J., Marco, A. D., Starr, T. N., Liu, Z., Pinto, D., Walls, A. C., Zatta, F., Zepeda, S. K., Bowen, J. E., Sprouse, K. R., et al. (2022). Antibody-mediated broad sarbecovirus neutralization through ACE2 molecular mimicry. Science 375, 449-454; Mykytyn, A. Z., Fouchier, R. A., and Haagmans, B. L. (2023). Antigenic evolution of SARS coronavirus 2. Curr. Opin. Virol. 62, 101349; Starr, T. N., Zepeda, S. K., Walls, A. C., Greaney, A. J., Alkhovsky, S., Veesler, D., and Bloom, J. D. (2022). ACE2 binding is an ancestral and evolvable trait of sarbecoviruses. Nature 603, 913-918; Lee, J., Zepeda, S. K., Park, Y.-J., Taylor, A. L., Quispe, J., Stewart, C., Leaf, E. M., Treichel, C., Corti, D., King, N. P., et al. (2023). Broad receptor tropism and immunogenicity of a clade 3 sarbecovirus. Cell Host Microbe 31, 1961-1973.e11; Murakami, S., Kitamura, T., Matsugo, H., Kamiki, H., Oyabu, K., Sekine, W., Takenaka-Uema, A., Sakai-Tagawa, Y., Kawaoka, Y., and Horimoto, T. (2022). Isolation of Bat Sarbecoviruses, Japan. Emerg. Infect. Dis. 28, 2500-2503; Cao, Y., Jian, F., Zhang, Z., Yisimayi, A., Hao, X., Bao, L., Yuan, F., Yu, Y., Du, S., Wang, J., et al. (2022). Rational identification of potent and broad sarbecovirus-neutralizing antibody cocktails from SARS convalescents. Cell Rep. 41, 111845; Cao, Y., Yisimayi, A., Jian, F., Song, W., Xiao, T., Wang, L., Du, S., Wang, J., Li, Q., Chen, X., et al. (2022). BA.2.12.1, BA.4 and BA.5 escape antibodies elicited by Omicron infection. Nature 608, 593-602; Zhou, D., Ren, J., Fry, E. E., and Stuart, D. I. (2023). Broadly neutralizing antibodies against CoVID-19. Curr. Opin. Virol. 61, 101332; Wang, Q., Guo, Y., Zhang, R. M., Ho, J., Mohri, H., Valdez, R., Manthei, D. M., Gordon, A., Liu, L., and Ho, D. D. (2023). Antibody neutralisation of emerging SARS-CoV-2 subvariants: EG.5.1 and XBC.1.6. Lancet Infect. Dis. 23, e397-e398; SA55 Injection Phase II Study in the Treatment of Mild/Moderate CoVID-19 Patients (2023). Clinicaltrials.gov. www.clinicaltrials.gov/study/NCT06042764; Walls, A. C., Xiong, X., Park, Y.-J., Tortorici, M. A., Snijder, J., Quispe, J., Cameroni, E., Gopal, R., Dai, M., Lanzavecchia, A., et al. (2019). Unexpected Receptor Functional Mimicry Elucidates Activation of Coronavirus Fusion. Cell 176, 1026-1039.e15; Piccoli, L., Park, Y.-J., Tortorici, M. A., Czudnochowski, N., Walls, A. C., Beltramello, M., Silacci-Fregni, C., Pinto, D., Rosen, L. E., Bowen, J. E., et al. (2020). Mapping Neutralizing and Immunodominant Sites on the SARS-CoV-2 Spike Receptor-Binding Domain by Structure-Guided High-Resolution Serology. Cell 183, 1024-1042.e21; Wrobel, A. G., Benton, D. J., Xu, P., Roustan, C., Martin, S. R., Rosenthal, P. B., Skehel, J. J., and Gamblin, S. J. (2020). SARS-CoV-2 and bat RaTG13 spike glycoprotein structures inform on virus evolution and furin-cleavage effects. Nat. Struct. Mol. Biol. 10.1038/s41594-020-0468-7; Lan, J., Ge, J., Yu, J., Shan, S., Zhou, H., Fan, S., Zhang, Q., Shi, X., Wang, Q., Zhang, L., et al. (2020). Structure of the SARS-CoV-2 spike receptor-binding domain bound to the ACE2 receptor. Nature 581, 215-220; Jian, F., Yang, S., Yu, Y., Song, W., Yisimayi, A., Chen, X., Xu, Y., Wang, P., Yu, L., Wang, J., et al. (2023). Convergent evolution of SARS-CoV-2 XBB lineages on receptor-binding domain 455-456 enhances antibody evasion and ACE2 binding. bioRxiv, 2023.08.30.555211. 10.1101/2023.08.30.555211; Yang, S., Yu, Y., Xu, Y., Jian, F., Song, W., Yisimayi, A., Wang, P., Wang, J., Liu, J., Yu, L., et al. (2023). Fast evolution of SARS-CoV-2 BA.2.86 to JN. 1 under heavy immune pressure. Lancet Infect. Dis. 10.1016/s1473-3099 (23) 00744-2; Cao, Y., Wang, J., Jian, F., Xiao, T., Song, W., Yisimayi, A., Huang, W., Li, Q., Wang, P., An, R., et al. (2022). Omicron escapes the majority of existing SARS-CoV-2 neutralizing antibodies. Nature 602, 657-663; Starr, T. N., Greaney, A. J., Stewart, C. M., Walls, A. C., Hannon, W. W., Veesler, D., and Bloom, J. D. (2022). Deep mutational scans for ACE2 binding, RBD expression, and antibody escape in the SARS-CoV-2 Omicron BA.1 and BA.2 receptor-binding domains. PLoS Pathog. 18, e1010951; Taylor, A. L., and Starr, T. N. (2023). Deep mutational scans of XBB.1.5 and BQ.1.1 reveal ongoing epistatic drift during SARS-CoV-2 evolution. PLoS Pathog. 19, e1011901; Qu, P., Evans, J. P., Zheng, Y.-M., Carlin, C., Saif, L. J., Oltz, E. M., Xu, K., Gumina, R. J., and Liu, S.-L. (2022). Evasion of neutralizing antibody responses by the SARS-CoV-2 BA.2.75 variant. Cell Host Microbe 30, 1518-1526.e4; Dadonaite, B., Brown, J., McMahon, T. E., Farrell, A. G., Asarnow, D., Stewart, C., Logue, J., Murrell, B., Chu, H. Y., Veesler, D., et al. (2023). Full-spike deep mutational scanning helps predict the evolutionary success of SARS-CoV-2 clades. bioRxiv. 10.1101/2023.11.13.566961; Tan, C. W., Chia, W. N., Zhu, F., Young, B. E., Chantasrisawad, N., Hwa, S.-H., Yeoh, A. Y.-Y., Lim, B. L., Yap, W. C., Pada, S. K. M. S., et al. (2022). SARS-CoV-2 Omicron variant emerged under immune selection. Nature Microbiology, 1-6; Regeneron Pharmaceuticals Third Quarter 2023 Earnings Call (2023). investor.regeneron.com/static-files/27bf1467-a410-414b-9bec-916fa90e7748; Park, Y.-J., Pinto, D., Walls, A. C., Liu, Z., De Marco, A., Benigni, F., Zatta, F., Silacci-Fregni, C., Bassi, J., Sprouse, K. R., et al. (2022). Imprinted antibody responses against SARS-CoV-2 Omicron sublineages. Science 378, 619-627; Tortorici, M. A., Addetia, A., Seo, A. J., Brown, J., Sprouse, K. R., Logue, J., Clark, E., Franko, N., Chu, H., and Veesler, D. (2023). Persistent immune imprinting after XBB.1.5 CoVID vaccination in humans. bioRxiv, 2023.11.28.569129. 10.1101/2023.11.28.569129; Fenwick, C., Turelli, P., Duhoo, Y., Lau, K., Herate, C., Marlin, R., Lamrayah, M., Campos, J., Esteves-Leuenberger, L., Farina, A., et al. (2023). Broadly potent anti-SARS-CoV-2 antibody shares 93% of epitope with ACE2 and provides full protection in monkeys. J. Infect. 87, 524-537; Lempp, F. A., Soriaga, L. B., Montiel-Ruiz, M., Benigni, F., Noack, J., Park, Y.-J., Bianchi, S., Walls, A. C., Bowen, J. E., Zhou, J., et al. (2021). Lectins enhance SARS-CoV-2 infection and influence neutralizing antibodies. Nature 598, 342-347; Bailey, L. J., Sheehy, K. M., Dominik, P. K., Liang, W. G., Rui, H., Clark, M., Jaskolowski, M., Kim, Y., Deneka, D., Tang, W.-J., et al. (2018). Locking the elbow: Improved antibody fab fragments as chaperones for structure determination. J. Mol. Biol. 430, 337-347; Lieu, R., Antonysamy, S., Druzina, Z., Ho, C., Kang, N. R., Pustilnik, A., Wang, J., and Atwell, S. (2020). Rapid and robust antibody Fab fragment crystallization utilizing edge-to-edge beta-sheet packing. PLoS One 15, e0232311; Lee, F. S., Anderson, A. G., and Olafson, B. D. (2023). Benchmarking TriadAb using targets from the second antibody modeling assessment. Protein Eng. Des. Sel. 36. 10.1093/protein/gzad013; Engler, C., Kandzia, R., and Marillonnet, S. (2008). A one pot, one step, precision cloning method with high throughput capability. PLoS One 3, e3647; Gibson, D. G., Young, L., Chuang, R.-Y., Venter, J. C., Hutchison, C. A., 3rd, and Smith, H. O. (2009). Enzymatic assembly of DNA molecules up to several hundred kilobases. Nat. Methods 6, 343-345; Benatuil, L., Perez, J. M., Belk, J., and Hsieh, C.-M. (2010). An improved yeast transformation method for the generation of very large human antibody libraries. Protein Eng. Des. Sel. 23, 155-159; Tortorici, M. A., Walls, A. C., Joshi, A., Park, Y.-J., Eguia, R. T., Miranda, M. C., Kepl, E., Dosey, A., Stevens-Ayers, T., Boeckh, M. J., et al. (2022). Structure, receptor recognition, and antigenicity of the human coronavirus CCoV-HuPn-2018 spike glycoprotein. Cell 185, 2279-2291.e17; Apaa, T., Withers, A. J., Staley, C., Blanchard, A., Bennett, M., Bremner-Harrison, S., Chadwick, E. A., Hailer, F., Harrison, S. W. R., Loose, M., et al. (2023). Sarbecoviruses of British horseshoe bats; sequence variation and epidemiology. J. Gen. Virol. 104. 10.1099/jgv.0.001859; Tan, C. C. S., Trew, J., Peacock, T. P., Mok, K. Y., Hart, C., Lau, K., Ni, D., Orme, C. D. L., Ransome, E., Pearse, W. D., et al. (2023). Genomic screening of 16 UK native bat species through conservationist networks uncovers coronaviruses with zoonotic potential. Nat. Commun. 14, 1-13; Wang, J., Pan, Y.-F., Yang, L.-F., Yang, W.-H., Luo, C.-M., Wang, J., Kuang, G.-P., Wu, W.-C., Gou, Q.-Y., Xin, G.-Y., et al. (2022). Individual bat viromes reveal the co-infection, spillover and emergence risk of potential zoonotic viruses. bioRxiv, 2022.11.23.517609. 10.1101/2022.11.23.517609; Starr, T. N., Greaney, A. J., Hannon, W. W., Loes, A. N., Hauser, K., Dillen, J. R., Ferri, E., Farrell, A. G., Dadonaite, B., McCallum, M., et al. (2022). Shifting mutational constraints in the SARS-CoV-2 receptor-binding domain during viral evolution. Science 377, 420-424; Gorrec, F. (2009). The MORPHEUS protein crystallization screen. J. Appl. Crystallogr. 42, 1035-1042; Kabsch, W. (2010). XDS. Acta Crystallogr. D Biol. Crystallogr. 66, 125-132; McCoy, A. J., Grosse-Kunstleve, R. W., Adams, P. D., Winn, M. D., Storoni, L. C., and Read, R. J. (2007). Phaser crystallographic software. J. Appl. Crystallogr. 40, 658-674; Emsley, P., Lohkamp, B., Scott, W. G., and Cowtan, K. (2010). Features and development of Coot. Acta Crystallogr. D Biol. Crystallogr. 66, 486-501; Murshudov, G. N., Skubák, P., Lebedev, A. A., Pannu, N. S., Steiner, R. A., Nicholls, R. A., Winn, M. D., Long, F., and Vagin, A. A. (2011). REFMAC5 for the refinement of macromolecular crystal structures. Acta Crystallogr. D Biol. Crystallogr. 67, 355-367; Liebschner, D., Afonine, P. V., Baker, M. L., Bunkóczi, G., Chen, V. B., Croll, T. I., Hintze, B., Hung, L.-W., Jain, S., McCoy, A. J., et al. (2019). Macromolecular structure determination using X-rays, neutrons and electrons: recent developments in Phenix. Acta Crystallogr. D Struct. Biol. 75, 861-877; Chen, V. B., Arendall, W. B., 3rd, Headd, J. J., Keedy, D. A., Immormino, R. M., Kapral, G. J., Murray, L. W., Richardson, J. S., and Richardson, D. C. (2010). MolProbity: all-atom structure validation for macromolecular crystallography. Acta Crystallogr. D Biol. Crystallogr. 66, 12-21; Addetia, A., Piccoli, L., Case, J. B., Park, Y.-J., Beltramello, M., Guarino, B., Dang, H., de Melo, G. D., Pinto, D., Sprouse, K., et al. (2023). Neutralization, effector function and immune imprinting of Omicron variants. Nature 621, 592-601; Case, D. A., Metin Aktulga, H., Belfon, K., Ben-Shalom, I., Brozell, S. R., Cerutti, D. S., Cheatham, T. E., III, Cruzeiro, V. W. D., Darden, T. A., Duke, R. E., et al. (2021). Amber 2021 (University of California, San Francisco); Maier, J. A., Martinez, C., Kasavajhala, K., Wickstrom, L., Hauser, K. E., and Simmerling, C. (2015). Ff14SB: Improving the accuracy of protein side chain and backbone parameters from ff99SB. J. Chem. Theory Comput. 11, 3696-3713; Kirschner, K. N., Yongye, A. B., Tschampel, S. M., González-Outeiriño, J., Daniels, C. R., Foley, B. L., and Woods, R. J. (2008). GLYCAM06: a generalizable biomolecular force field. Carbohydrates. J. Comput. Chem. 29, 622-655; Jorgensen, W. L., Chandrasekhar, J., Madura, J. D., Impey, R. W., and Klein, M. L. (1983). Comparison of simple potential functions for simulating liquid water. J. Chem. Phys. 79, 926-935; Joung, I. S., and Cheatham, T. E., 3rd (2008). Determination of alkali and halide monovalent ion parameters for use in explicitly solvated biomolecular simulations. J. Phys. Chem. B 112, 9020-9041; Momont, C., Dang, H. V., Zatta, F., Hauser, K., Wang, C., di Iulio, J., Minola, A., Czudnochowski, N., De Marco, A., Branch, K., et al. (2023). A pan-influenza antibody inhibiting neuraminidase via receptor mimicry. Nature 618, 590-597; Hauser, K., Essuman, B., He, Y., Coutsias, E., Garcia-Diaz, M., and Simmerling, C. (2016). A human transcription factor in search mode. Nucleic Acids Res. 44, 63-74; Eastman, P., Galvelis, R., Peláez, R. P., Abreu, C. R. A., Farr, S. E., Gallicchio, E., Gorenko, A., Henry, M. M., Hu, F., Huang, J., et al. (2023). OpenMM 8: Molecular dynamics simulation with machine learning potentials. 10.48550/ARXIV.2310.03121; Fass, J., Sivak, D., Crooks, G., Beauchamp, K., Leimkuhler, B., and Chodera, J. (2018) Quantifying configuration-sampling error in Langevin simulations of complex molecular systems. Entropy (Basel) 20, 318; Leimkuhler, B., and Matthews, C. (2013). Robust and efficient configurational molecular sampling via Langevin dynamics. J. Chem. Phys. 138, 174102; Leimkuhler, B., and Matthews, C. (2016). Efficient molecular dynamics using geodesic integration and solvent-solute splitting. Proc. Math. Phys. Eng. Sci. 472, 20160138; Darden, T., York, D., and Pedersen, L. (1993). Particle mesh Ewald: An N·log (N) method for Ewald sums in large systems. J. Chem. Phys. 98, 10089-10092; Shirts, M. R., Mobley, D. L., Chodera, J. D., and Pande, V. S. (2007). Accurate and efficient corrections for missing dispersion interactions in molecular simulations. J. Phys. Chem. B 111, 13052-13063; Case, J. B., Rothlauf, P. W., Chen, R. E., Liu, Z., Zhao, H., Kim, A. S., Bloyet, L.-M., Zeng, Q., Tahan, S., Droit, L., et al. (2020). Neutralizing Antibody and Soluble ACE2 Inhibition of a Replication-Competent VSV-SARS-CoV-2 and a Clinical Isolate of SARS-CoV-2. Cell Host Microbe 28, 475-485.e5; Liu, Z., VanBlargan, L. A., Bloyet, L.-M., Rothlauf, P. W., Chen, R. E., Stumpf, S., Zhao, H., Errico, J. M., Theel, E. S., Liebeskind, M. J., et al. (2021). Identification of SARS-CoV-2 spike mutations that attenuate monoclonal and serum antibody neutralization. Cell Host Microbe 29, 477-488.e4; Bolger, A. M., Lohse, M., and Usadel, B. (2014). Trimmomatic: a flexible trimmer for Illumina sequence data. Bioinformatics 30, 2114-2120; Li, H. (2011). A statistical framework for SNP calling, mutation discovery, association mapping and population genetical parameter estimation from sequencing data. Bioinformatics 27, 2987-2993; Wilm, A., Aw, P. P. K., Bertrand, D., Yeo, G. H. T., Ong, S. H., Wong, C. H., Khor, C. C., Petric, R., Hibberd, M. L., and Nagarajan, N. (2012). LoFreq: a sequence-quality aware, ultra-sensitive variant caller for uncovering cell-population heterogeneity from high-throughput sequencing datasets. Nucleic Acids Res. 40, 11189-11201; Danecek, P., Bonfield, J. K., Liddle, J., Marshall, J., Ohan, V., Pollard, M. O., Whitwham, A., Keane, T., McCarthy, S. A., Davies, R. M., et al. (2021). Twelve years of SAMtools and BCFtools. Gigascience 10. 10.1093/gigascience/giab008; di Iulio, J., Bartha, I., Wong, E. H. M., Yu, H.-C., Lavrenko, V., Yang, D., Jung, I., Hicks, M. A., Shah, N., Kirkness, E. F., et al. (2018). The human noncoding genome defined by genetic diversity. Nat. Genet. 50, 333-337.

The various embodiments described above can be combined to provide further embodiments. All of the U.S. patents, U.S. patent application publications, U.S. patent applications, foreign patents, foreign patent applications and non-patent publications referred to in this specification and/or listed in the Request, including U.S. Provisional Application No. 63/495,260, filed Apr. 10, 2023, U.S. Provisional Application No. 63/626,876, filed Jan. 30, 2024, and U.S. Provisional Application No. 63/557,275, filed Feb. 23, 2024, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An antibody, or an antigen-binding fragment,
comprising a heavy chain variable domain (VH) comprising a complementarity determining region (CDR) H1, CDRH2, and a CDRH3, and a light chain variable domain (VL) comprising a CDRL1, CDRL2, CDRL3, wherein;
(i) the CDRH1, CDRH2, CDRH3, CDRL1, CDRL2, and CDRL3 are according to SEQ ID NOs: 68, 171, 70, 72, 73, and 114, respectively; or
(ii) the CDRH1, CDRH2, CDRH3, CDRL1, CDRL2, and CDRL3 are according to SEQ ID NOs: 68, 215 or 216, 70, 72, 73, and 114, respectively.

2. (canceled)

3. The antibody, or an antigen-binding fragment according to claim 1, comprising a heavy chain variable domain (VH) and a light chain variable domain (VL), wherein:
(i) the VH is according to any one of SEQ ID NOs: 184 and 187-202, and the VL is according to SEQ ID NO: 113; or
(ii) the VH comprises an amino acid sequence according to one or more of SEQ ID NOs: 215-221, and the VL comprises an amino acid sequence according to any one of SEQ ID NOs: 113 and 222-224.

4.-9. (canceled)

10. The antibody or antigen-binding fragment of claim 1, wherein the antibody or antigen-binding fragment is capable of binding to:
(i) a sarbecovirus, optionally a Clade 1b sarbecovirus; and/or
(ii) a SARS-CoV-2 and a SARS-CoV.

11. (canceled)

12. The antibody or antigen-binding fragment of claim 1, wherein the antibody or antigen-binding fragment comprises a heavy chain constant domain (CH) or a Fc polypeptide or a fragment thereof comprising an amino acid sequence according to any one of SEQ ID NOs: 30-66.

13. The antibody or antigen-binding fragment of claim 12, wherein the antibody comprises a heavy chain constant domain (CH) comprising an amino acid sequence according to SEQ ID NO: 49 and/or the antibody or antigen-binding fragment comprises a light chain constant domain (CL) comprising an amino acid sequence according to SEQ ID NO: 186.

14. (canceled)

15. The antibody or antigen-binding fragment of claim 1, which is human, humanized, or chimeric and/or comprises a human antibody, a monoclonal antibody, a purified antibody, a single chain antibody, a Fab, a Fab′, a F(ab′)2, a Fv, a scFv, or a scFab.

16. The antibody or antigen-binding fragment of claim 1, wherein the antibody or antigen-binding fragment comprises two identical VH domains and two identical VL domains, wherein each VH and VL together form an antigen-binding site.

17. The antibody or antigen-binding fragment of claim 1, wherein the antibody or antigen-binding fragment is a bispecific antibody or antigen-binding fragment.

18. An antibody or antigen-binding fragment comprising: a first VH and a first VL; and a second VH and a second VL, wherein the first VH and first VL comprise an amino acid sequence having at least 85% identity to the amino acid sequence according to SEQ ID NO: 184, and an amino acid sequence having at least 85% identity to the amino acid sequence according to SEQ ID NO: 113, respectively; wherein the first VH and the first VL are different from the second VH and second VL; wherein the first VH and the first VL together form a first antigen-binding site; and wherein the second VH and the second VL together form a second antigen-binding site.

19. An antibody drug conjugate (ADC) comprising an antibody according to claim 1 conjugated to a drug, wherein the drug is an anti-viral or anti-inflammatory small molecule.

20. An isolated polynucleotide encoding the antibody or antigen-binding fragment of claim 1.

21. The polynucleotide of claim 20, wherein the polynucleotide comprises or consists of a nucleic acid sequence having at least 85% identity to the nucleic acid sequence according to one or more of SEQ ID NOs: 115 and 183.

22. The polynucleotide of claim 20, wherein the polynucleotide comprises deoxyribonucleic acid (DNA) or ribonucleic acid (RNA), wherein the RNA optionally comprises messenger RNA (mRNA), optionally self-amplifying RNA (saRNA), trans-amplifying RNA (taRNA), or circular RNA (circRNA).

23. A recombinant vector, DNA therapeutic, or RNA therapeutic comprising the polynucleotide of claim 20.

24. A DNA therapeutic construct or RNA therapeutic construct comprising the polynucleotide of claim 23, encapsulated in a carrier, wherein the carrier optionally comprises a lipid, a lipid-derived delivery vehicle, such as a liposome, a solid lipid nanoparticle, an oily suspension, a submicron lipid emulsion, a lipid microbubble, an inverse lipid micelle, a cochlear liposome, a lipid microtubule, a lipid microcylinder, lipid nanoparticle (LNP), or a nanoscale platform.

25. A host cell comprising the polynucleotide of claim 20, wherein the polynucleotide is heterologous to the host cell.

26. A host cell according to claim 25, wherein the host cell is a human B cell and the polynucleotide is heterologous to the human B cell and/or wherein the human B cell is immortalized.

27. A composition comprising: (i) the antibody or antigen-binding fragment of claim 1; and (ii) a pharmaceutically acceptable excipient, carrier, or diluent.

28. A method of treating a sarbecovirus infection (e.g. infection by a SARS-CoV-2) in a subject, the method comprising administering to the subject an effective amount of the antibody or antigen-binding fragment of claim 1.

29. (canceled)

30. A kit comprising a liquid composition comprising (i) the antibody or antigen-binding fragment of claim 1, and instructions for use thereof in treating a SARS-CoV-2 infection in a subject.


SEQUENCE LISTING (252 sequences)

SEQ ID NO: 1 - protein, 1263 aa, Severe acute respiratory syndrome-related coronavirus
  source 1..1263: note = SARS-CoV-2 surface glycoprotein GenBank: QHD43416.1; January 23, 2020
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 220 aa, Severe acute respiratory syndrome-related coronavirus
  source 1..220: note = SARS-CoV-2 surface glycoprotein RBD GenBank: QHD43416.1; January 23, 2020
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTG

SEQ ID NO: 3 - protein, 72 aa, Severe acute respiratory syndrome-related coronavirus
  source 1..72: note = SARS-CoV-2 RBM GenBank: QHD43416.1; January 23, 2020
NSNNLDSKVGGNYNYLYRLFRKSNLKPFERDISTEIYQAGSTPCNGVEGFNCYFPLQSYGFQPTNGVGYQPY

SEQ ID NO: 4 - protein, 18 aa, synthetic construct
  source 1..18: note = Linker (aa)
GSTSGSGKPGSGEGSTKG

SEQ ID NO: 5 - protein, 10 aa, synthetic construct
  source 1..10: note = Linker (aa)
GSGKPGSGEG

SEQ ID NO: 6 - protein, 8 aa, synthetic construct
  source 1..8: note = Linker (aa)
GKPGSGEG

SEQ ID NO: 7 - protein, 8 aa, synthetic construct
  source 1..8: note = Linker (aa)
SGKPGSGE

SEQ ID NO: 8 - protein, 15 aa, synthetic construct
  source 1..15: note = Linker (aa)
GGGGSGGGGSGGGGS

SEQ ID NO: 9 - protein, 50 aa, synthetic construct
  source 1..50: note = Linker (aa)
GGGGSGGGGSGGGGSGGGGSGGGGSGGGGSGGGGSGGGGSGGGGSGGGGS

SEQ ID NO: 10 - protein, 18 aa, synthetic construct
  source 1..18: note = Linker (aa)
GSTSGGGSGGGSGGGGSS

SEQ ID NO: 11 - protein, 14 aa, synthetic construct
  source 1..14: note = Linker (aa)
EGKSSGSGSESKVD

SEQ ID NO: 12 - protein, 18 aa, synthetic construct
  source 1..18: note = Linker (aa)
KESGSVSSEQLAQFRSLD

SEQ ID NO: 13 - protein, 5 aa, synthetic construct
  source 1..5: note = Linker (aa)
GGGGS

SEQ ID NO: 14 - protein, 126 aa, synthetic construct
  source 1..126: note = S2X259 VH
QVQLVQSGAEVKKPGSSVKVSCKASGGIFNTYTISWVRQAPGQGLEWMGRIILMSGMANYAQKIQGRVTITADKSTSTAYMELTSLRSDDTAVYYCARGFNGNYYGWGDDDAFDISGQGTLVTVYS

SEQ ID NO: 15 - protein, 113 aa, synthetic construct
  source 1..113: note = S2X259 VL
QTVLTQPPSVSGAPGQRVTISCTGSNSNIGAGYDVHWYQQLPGTAPKLLICGNSNRPSGVPDRFSGSKSGTSASLAITGLQAEDEADYYCQSYDSSLSGPNWVFGGGTKLTVL

SEQ ID NO: 16 - protein, 127 aa, synthetic construct [sequence omitted]

SEQ ID NO: 17 - protein, 107 aa, synthetic construct [sequence omitted]

SEQ ID NO: 18 - protein, 122 aa, synthetic construct
  source 1..122: note = S2K146 VH (aa)
QVQLVESGGVVVQPGGSLRLSCAASGFTFHDHTMHWVRQAPGKGLEWVSLITWNGGTIHYSDSVKGRFTISRDNSKNSLYLQMNSLRTEDTALYYCAKDLGRGGWYLPSDAWGQGTLVTVSS

SEQ ID NO: 19 - protein, 109 aa, synthetic construct
  source 1..109: note = S2K146 VL (aa)
QSVLTQPPSASGTPGQRVTISCSGSSANIGSNTVNWYQHLPGTAPKLLIYSNNQRPSGVPDRFSGSKSGTSASLAISGLQSEDEADYYCAAWDDSLKGVFGGGTKLTVL

SEQ ID NO: 20 - protein, 119 aa, synthetic construct
  source 1..119: note = S2X324-VH (aa)
QITLKESGPTLVKPTQTLTLTCTFSGFSVTTSGVGVGWIRQPPGKALEYLALIYWDDDKRYSTSLKSRLTITKDTSKNQVVLTMTNMDPVDTATYYCARHTIPSIFDYWGQGILVTVSS

SEQ ID NO: 21 - protein, 109 aa, synthetic construct
  source 1..109: note = S2X324 VL (aa)
QPVLTQPASVSGSPGQSITISCTATSSDVGNYNYVSWYQHHPGKAPKLMIYEVSNRPSGVSNRFSGSKSGNTASLTISGLQAEDEADYYCSSYTSSSLLFGGGTKLTVL

SEQ ID NO: 22 - protein, 127 aa, synthetic construct
  source 1..127: note = Sotrovimab - VH (aa)
QVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSS

SEQ ID NO: 23 - protein, 107 aa, synthetic construct [sequence omitted]

SEQ ID NO: 24 - protein, 127 aa, synthetic construct
  source 1..127: note = S3I2 - VH (aa)
EVQLVETGGGLIQPGGSLTLSCAASGLVVSYNYMTWVRQAPGKGLEWLSVIYAGGSTFYADSVKGRFTVSRDNSNNTLYLQMNNLRAEDTAVYFCARWGQVRFTSEEAYYKYGLDVWGQGTTVTVSS

SEQ ID NO: 25 - protein, 108 aa, synthetic construct
  source 1..108: note = S3I2 - VL (VK) (aa)
DIQLTQSPSFLSASVGDRVTITCRASQGISNYLAWYQQRPGKAPKLLIFTASTLQSGVSSRFSGSGSGTEFTLTISSLQPEDLATYYCQQLNSNLQGTFGQGTRVEIK

SEQ ID NO: 26 - protein, 120 aa, synthetic construct
  source 1..120: note = S3O13 - VH (aa)
QVQLVQSGAEVKKPGASVKVSCKASGYTFTMSAMHWVRQAPGQRPEWMGWITAGTGDTKYSQKFQGRVTITSDTSATTAYMELSSLRSEDTAVYYCAKDGDYAAEYFQHWGQGTLVTVSS

SEQ ID NO: 27 - protein, 106 aa, synthetic construct
  source 1..106: note = S3O13 - VL (VK) (aa)
DIVMTQSPSTLSASVGDRVTITCRASQSINSWLAWYQQKPGKAPNLLISDASNLESGVPSRFSGSGSGTEFTLTISSLQPDDFATYYCQQYNTYPSFGQGTKVEIK

SEQ ID NO: 28 - protein, 117 aa, synthetic construct
  source 1..117: note = S3L17 - VH (aa)
EVQLVESGGGLVQPGGSLRLSCAASEIIVSSNYMNWVRQAPGKGLEWVSVIYAGGSTFYADSVRGRFTISRDNSKNTLYLQMNSLRPDDTAIYYCARDLGPWGMDVWGQGTTVTVSS

SEQ ID NO: 29 - protein, 108 aa, synthetic construct
  source 1..108: note = S3L17 - VL (VK) (aa)
DIQMTQSPSSLSASVGDRVAITCRASHGINNDLAWYQQKPGKVPKLLIYAASTLQSGVPSRFSGSGSGTDFTLTISSLQPEDVATYYCQHYSSDPRFTFGPGTKVDIK

SEQ ID NO: 30 - protein, 330 aa, Homo sapiens [sequence omitted]

SEQ ID NO: 31 - protein, 232 aa, Homo sapiens
  source 1..232: note = Wild-type human IgG1 hinge-CH2-CH3
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 110 aa, Homo sapiens
  source 1..110: note = Wild-type human IgG1 CH2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 33 - protein, 107 aa, Homo sapiens
  source 1..107: note = Wild-type human IgG1 CH3
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 125 aa, Homo sapiens
  source 1..125: note = Wild-type human IgG1 hinge-CH2
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 35 - protein, 330 aa, synthetic construct
  source 1..330: note = IgG1m3 CH1-CH3
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 330 aa, synthetic construct [sequence omitted]

SEQ ID NO: 37 - protein, 330 aa, synthetic construct
  source 1..330: note = IgG1m3,1 CH1-CH3
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 330 aa, synthetic construct
  source 1..330: note = Human IgG1 CH1-CH3 - GAALIE mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 232 aa, synthetic construct
  source 1..232: note = Human IgG1 hinge-CH2-CH3 - GAALIE mutation
EPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 110 aa, synthetic construct
  source 1..110: note = Human IgG1 CH2 - GAALIE mutation
APELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAK

SEQ ID NO: 41 - protein, 125 aa, synthetic construct
  source 1..125: note = Human IgG1 hinge-CH2; - GAALIE mutation
EPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAK

SEQ ID NO: 42 - protein, 330 aa, synthetic construct
  source 1..330: note = IgG1m3 CH1-CH3 - GAALIE mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 330 aa, synthetic construct; identical to SEQ ID NO: 38
  source 1..330: note = IgG1m17,1 CH1-CH3- GAALIE mutation

SEQ ID NO: 44 - protein, 330 aa, synthetic construct
  source 1..330: note = IgG1m3,1 CH1-CH3 - GAALIE mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 330 aa, synthetic construct
  source 1..330: note = Human IgG1 CH1-CH3 - LS mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 46 - protein, 232 aa, synthetic construct
  source 1..232: note = Human IgG1 hinge-CH2-CH3- LS mutation
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 47 - protein, 107 aa, synthetic construct
  source 1..107: note = Human IgG1 CH3 - LS mutation
GQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 48 - protein, 330 aa, synthetic construct
  source 1..330: note = IgG1m3 CH1-CH3 - LS mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 49 - protein, 330 aa, synthetic construct; identical to SEQ ID NO: 45
  source 1..330: note = IgG1m17,1 IGHG1*01 CH1-CH3 - LS mutation

SEQ ID NO: 50 - protein, 330 aa, synthetic construct
  source 1..330: note = IgG1m3,1 CH1-CH3 - LS mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 51 - protein, 330 aa, synthetic construct
  source 1..330: note = Human IgG1 CH1-CH3 - LA mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 52 - protein, 232 aa, synthetic construct
  source 1..232: note = Human IgG1 hinge-CH2-CH3- LA mutation
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 53 - protein, 107 aa, synthetic construct
  source 1..107: note = Human IgG1 CH3 - LA mutation
GQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 54 - protein, 330 aa, synthetic construct
  source 1..330: note = IgG1m3 CH1-CH3 - LS mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 55 - protein, 330 aa, synthetic construct; identical to SEQ ID NO: 51
  source 1..330: note = IgG1m17,1 CH1-CH3 - LS mutation

SEQ ID NO: 56 - protein, 330 aa, synthetic construct
  source 1..330: note = IgG1m3,1 CH1-CH3 - LS mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 57 - protein, 330 aa, synthetic construct
  source 1..330: note = Human IgG1 CH1-CH3 - GAALIE and LS mutations
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 58 - protein, 232 aa, synthetic construct
  source 1..232: note = Human IgG1 hinge-CH2-CH3 - GAALIE and LS mutations
EPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 59 - protein, 330 aa, synthetic construct
  source 1..330: note = IgG1m3 CH1-CH3 - GAALIE and LS mutations
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 60 - protein, 330 aa, synthetic construct; identical to SEQ ID NO: 57
  source 1..330: note = IgG1m17,1 CH1-CH3 - GAALIE and LS mutations

SEQ ID NO: 61 - protein, 330 aa, synthetic construct
  source 1..330: note = IgG1m3,1 CH1-CH3 - GAALIE and LS mutations
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 62 - protein, 330 aa, synthetic construct
  source 1..330: note = Human IgG1 CH1-CH3 - GAALIE and LA mutations
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 63 - protein, 232 aa, synthetic construct
  source 1..232: note = Human IgG1 hinge-CH2-CH3 - GAALIE and LA mutations
EPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 64 - protein, 330 aa, synthetic construct
  source 1..330: note = IgG1m3 CH1-CH3 - GAALIE and LA mutations
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 65 - protein, 330 aa, synthetic construct; identical to SEQ ID NO: 62
  source 1..330: note = IgG1m17,1 CH1-CH3 - GAALIE and LA mutations

SEQ ID NO: 66 - protein, 330 aa, synthetic construct
  source 1..330: note = IgG1m3,1 CH1-CH3 - GAALIE and LA mutations
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 67 - protein, 123 aa, synthetic construct
  source 1..123: note = S2V29 - VH (also S2V29-VH.1) (aa)
EVQLVESGGSVVQPGRALRLSCAASGITFSSFGMYWVRQAPGKGLEWLGVIAYDGSNTYYADSVKGRFTISRDNSKNTLYLQMNSLRIEDTAVYYCAKDYYPLLSYYYGLDVWGQGTTVTVSS

SEQ ID NO: 68 - protein, 8 aa, synthetic construct
  source 1..8: note = S2V29 - CDRH1
GITFSSFG

SEQ ID NO: 69 - protein, 8 aa, synthetic construct
  source 1..8: note = S2V29 - CDRH2
IAYDGSNT

SEQ ID NO: 70 - protein, 16 aa, synthetic construct
  source 1..16: note = S2V29 - CDRH3
AKDYYPLLSYYYGLDV

SEQ ID NO: 71 - protein, 110 aa, synthetic construct
  source 1..110: note = S2V29 - VL-11a (also S2V29-VL.1) (aa)
QSVLTQPRSVSGSPGQSVTISCTGTSSDVGAYNYVSWYQQHPGKAPKFMIYDVDQRPSGVPDRFSGSKSGNTASLIISGLQAEDEADYYCCSYAGSYIWVFGGGTQLTVL

SEQ ID NO: 72 - protein, 9 aa, synthetic construct
  source 1..9: note = S2V29 - VL-11a CDRL1
SSDVGAYNY

SEQ ID NO: 73 - skipped

SEQ ID NO: 74 - protein, 10 aa, synthetic construct
  source 1..10: note = S2V29 - VL-11a CDRL3
CSYAGSYIWV

SEQ ID NO: 75 - DNA, 369 bp, synthetic construct
  source 1..369: mol_type = other DNA; note = S2V29 - VH (nt)
gaggtgcagctggtggagtctgggggaagtgtggtccagcctgggagggccctgagactctcctgtgcagcctctggaatcaccttcagtagctttggcatgtactgggtccgccaggctccaggcaaggggctggagtggctgggagttatagcgtatgatggaagtaatacatactatgcagactccgtgaagggccgattcaccatctccagagacaattccaagaacacgctgtatttgcaaatgaacagcctgagaattgaggacacggctgtctattattgtgcgaaagattattacccgctgctatcgtactactacggtttggacgtctggggccaagggaccacggtcaccgtatcctca

SEQ ID NO: 76 - DNA, 331 bp, synthetic construct
  source 1..331: mol_type = other DNA; note = S2V29 - VL-11a (nt)
cagtctgtgctgactcagcctcgctcagtgtccgggtctcctggacagtcagtcaccatttcctgcactggaaccagcagtgatgtcggtgcttataactatgtctcctggtaccaacaacacccaggcaaagcccccaaattcatgatttatgatgtcgatcagcggccctcaggggtccctgatcgcttctctggctccaagtctggcaacacggcctccctgatcatctctgggctccaggctgaggatgaggctgattattactgctgctcatatgctggcagctacatttgggtgttcggcggagggacccaactgaccgtcctag

SEQ ID NO: 77 - protein, 110 aa, synthetic construct
  source 1..110: note = S2V29 - VL-11b (aa)
QSVLTQPRSVSGSPGQSVTISCTGTSSDVGAYNYVSWYQQHPGKAPKFMIYDVDQRPSGVPDRFSGSKSGNTASLIISGLQAEDEADYYCSSYAGSYIWVFGGGTQLTVL

SEQ ID NO: 78 - protein, 10 aa, synthetic construct
  source 1..10: note = S2V29 - VL-11b CDRL3
SSYAGSYIWV

SEQ ID NO: 79 - DNA, 369 bp, synthetic construct
  source 1..369: mol_type = other DNA; note = S2V29-VH.1 (nt)
gaagtgcaactagtggaaagtggtggtagtgtcgtgcagcctggcagagccctcagactgagctgtgctgcttctggcatcaccttctcctcttttggcatgtactgggtgcggcaagctcccggaaaaggactggaatggctgggcgtgatcgcctacgacggctctaatacctactacgccgattccgtgaagggccgcttcacaatctccagagataactccaagaacaccctgtacctgcagatgaactctctgcggatcgaggacaccgccgtgtactactgcgccaaggactactatcctctgctgtcctactactacggcctggacgtgtggggccagggcaccacagtgaccgtgtccagc

SEQ ID NO: 80 - DNA, 330 bp, synthetic construct
  source 1..330: mol_type = other DNA; note = S2V29-VL.1 (nt)
caaagtgtgctaactcaaccacgaagtgtgtctggctctccaggacaatccgtgaccatctcctgcaccggcacctccagcgatgtgggcgcctacaactacgtgtcctggtaccagcagcaccctggcaaggcccctaagttcatgatctacgacgtggaccagcggccttctggcgtgcccgatagattctccggcagcaaatccggcaacaccgcttctctgatcatcagcggcctgcaggccgaggacgaagccgactactactgctgttcttatgctggctcctacatctgggtctttggcggcggaacacagctgaccgtgctg

SEQ ID NO: 81 - protein, 123 aa, synthetic construct
  source 1..123: note = S2V29-VH.2 (aa)
EVQLVESGGSVVQPGRALRLSCAASGITFSSFGMYWVRQAPGKGLEWLGVIAYKGSNTYYADSVKGRFTISRDNSKNTLYLQMNSLRIEDTAVYYCAKDYYPLLSYYYGLDVWGQGTTVTVSS

SEQ ID NO: 82 - protein, 8 aa, synthetic construct
  source 1..8: note = S2V29-VH.2 CDRH2
IAYKGSNT

SEQ ID NO: 83 - DNA, 369 bp, synthetic construct
  source 1..369: mol_type = other DNA; note = S2V29-VH.2 (nt)
gaagtgcaactagtggaaagtggtggtagtgtcgtgcagcctggcagagccctcagactgagctgtgctgcttctggcatcaccttctcctcttttggcatgtactgggtgcggcaagctcccggaaaaggactggaatggctgggcgtgatcgcctacaagggctctaatacctactacgccgattccgtgaagggccgcttcacaatctccagagataactccaagaacaccctgtacctgcagatgaactctctgcggatcgaggacaccgccgtgtactactgcgccaaggactactatcctctgctgtcctactactacggcctggacgtgtggggccagggcaccacagtgaccgtgtccagc

SEQ ID NO: 84 - protein, 123 aa, synthetic construct
  source 1..123: note = S2V29-VH.3 (aa)
EVQLVESGGSVVQPGRALRLSCAASGITFSSFGMYWVRQAPGKGLEWLGVIAYSGSNTYYADSVKGRFTISRDNSKNTLYLQMNSLRIEDTAVYYCAKDYYPLLSYYYGLDVWGQGTTVTVSS

SEQ ID NO: 85 - protein, 8 aa, synthetic construct
  source 1..8: note = S2V29-VH.3 CDRH2
IAYSGSNT

SEQ ID NO: 86 - DNA, 369 bp, synthetic construct
  source 1..369: mol_type = other DNA; note = S2V29-VH.3 (nt)
gaagtgcaactagtggaaagtggtggtagtgtcgtgcagcctggcagagccctcagactgagctgtgctgcttctggcatcaccttctcctcttttggcatgtactgggtgcggcaagctcccggaaaaggactggaatggctgggcgtgatcgcctactccggctctaatacctactacgccgattccgtgaagggccgcttcacaatctccagagataactccaagaacaccctgtacctgcagatgaactctctgcggatcgaggacaccgccgtgtactactgcgccaaggactactatcctctgctgtcctactactacggcctggacgtgtggggccagggcaccacagtgaccgtgtccagc

SEQ ID NO: 87 - protein, 123 aa, synthetic construct
  source 1..123: note = S2V29-VH.4
EVQLVESGGSVVQPGRALRLSCAASGITFSSFGMYWVRQAPGKGLEWLGVIAYEGSNTYYADSVKGRFTISRDNSKNTLYLQMNSLRIEDTAVYYCAKDYYPLLSYYYGLDVWGQGTTVTVSS

SEQ ID NO: 88 - protein, 8 aa, synthetic construct
  source 1..8: note = S2V29-VH.4 CDRH2
IAYEGSNT

SEQ ID NO: 89 - DNA, 369 bp, synthetic construct
  source 1..369: mol_type = other DNA; note = S2V29-VH.4 (nt)
gaagtgcaactagtggaaagtggtggtagtgtcgtgcagcctggcagagccctcagactgagctgtgctgcttctggcatcaccttctcctcttttggcatgtactgggtgcggcaagctcccggaaaaggactggaatggctgggcgtgatcgcctacgagggctctaatacctactacgccgattccgtgaagggccgcttcacaatctccagagataactccaagaacaccctgtacctgcagatgaactctctgcggatcgaggacaccgccgtgtactactgcgccaaggactactatcctctgctgtcctactactacggcctggacgtgtggggccagggcaccacagtgaccgtgtccagc

SEQ ID NO: 90 - protein, 123 aa, synthetic construct
  source 1..123: note = S2V29-VH.5
EVQLVESGGSVVQPGRALRLSCAASGITFSSFGMYWVRQAPGKGLEWLGVIAYDASNTYYADSVKGRFTISRDNSKNTLYLQMNSLRIEDTAVYYCAKDYYPLLSYYYGLDVWGQGTTVTVSS

SEQ ID NO: 91 - protein, 8 aa, synthetic construct
  source 1..8: note = S2V29-VH.5 CDRH2
IAYDASNT

SEQ ID NO: 92 - DNA, 369 bp, synthetic construct
  source 1..369: mol_type = other DNA; note = S2V29-VH.5 (nt)
gaagtgcaactagtggaaagtggtggtagtgtcgtgcagcctggcagagccctcagactgagctgtgctgcttctggcatcaccttctcctcttttggcatgtactgggtgcggcaagctcccggaaaaggactggaatggctgggcgtgatcgcctacgacgcctctaatacctactacgccgattccgtgaagggccgcttcacaatctccagagataactccaagaacaccctgtacctgcagatgaactctctgcggatcgaggacaccgccgtgtactactgcgccaaggactactatcctctgctgtcctactactacggcctggacgtgtggggccagggcaccacagtgaccgtgtccagc

SEQ ID NO: 93 - protein, 123 aa, synthetic construct
  source 1..123: note = S2V29-VH.6
EVQLVESGGSVVQPGRALRLSCAASGITFSSFGMYWVRQAPGKGLEWLGVIAYSGSNTYYADSVKGRFTISRDNSKNTLYLQMNSLRIEDTAVYYCAKDYYPLLSYYYGIDVWGQGTTVTVSS

SEQ ID NO: 94 - protein, 8 aa, synthetic construct
  source 1..8: note = S2V29-VH.6 CDRH2
IAYSGSNT

SEQ ID NO: 95 - protein, 16 aa, synthetic construct
  source 1..16: note = S2V29-VH.6 CDRH3
AKDYYPLLSYYYGIDV

SEQ ID NO: 96 - DNA, 369 bp, synthetic construct
  source 1..369: mol_type = other DNA; note = S2V29-VH.6 (nt)
gaagtgcaactagtggaaagtggtggtagtgtcgtgcagcctggcagagccctcagactgagctgtgctgcttctggcatcaccttctcctcttttggcatgtactgggtgcggcaagctcccggaaaaggactggaatggctgggcgtgatcgcctactccggctctaatacctactacgccgattccgtgaagggccgcttcacaatctccagagataactccaagaacaccctgtacctgcagatgaactctctgcggatcgaggacaccgccgtgtactactgcgccaaggactactatcctctgctgtcctactactacggcatcgacgtgtggggccagggcaccacagtgaccgtgtccagc

SEQ ID NO: 97 - protein, 123 aa, synthetic construct
  source 1..123: note = S2V29-VH.7
EVQLVESGGSVVQPGRALRLSCAASGITFSSFGMYWVRQAPGKGLEWLGVIAYEGSNTYYADSVKGRFTISRDNSKNTLYLQMNSLRIEDTAVYYCAKDYYPLLSYYYGIDVWGQGTTVTVSS

SEQ ID NO: 98 - protein, 8 aa, synthetic construct
  source 1..8: note = S2V29-VH.7 CDRH2
IAYEGSNT

SEQ ID NO: 99 - protein, 16 aa, synthetic construct
  source 1..16: note = S2V29-VH.7 CDRH3
AKDYYPLLSYYYGIDV

SEQ ID NO: 100 - DNA, 369 bp, synthetic construct
  source 1..369: mol_type = other DNA; note = S2V29-VH.7 (nt)
gaagtgcaactagtggaaagtggtggtagtgtcgtgcagcctggcagagccctcagactgagctgtgctgcttctggcatcaccttctcctcttttggcatgtactgggtgcggcaagctcccggaaaaggactggaatggctgggcgtgatcgcctacgagggctctaatacctactacgccgattccgtgaagggccgcttcacaatctccagagataactccaagaacaccctgtacctgcagatgaactctctgcggatcgaggacaccgccgtgtactactgcgccaaggactactatcctctgctgtcctactactacggcatcgacgtgtggggccagggcaccacagtgaccgtgtccagc

SEQ ID NO: 101 - protein, 123 aa, synthetic construct
  source 1..123: note = S2V29-VH.8
EVQLVESGGSVVQPGRALRLSCAASGITFSSFGMYWVRQAPGKGLEWLGVIAYKGSNTYYADSVKGRFTISRDNSKNTLYLQMNSLRIEDTAVYYCAKDYYPLLSYYYGIDVWGQGTTVTVSS

SEQ ID NO: 102 - protein, 8 aa, synthetic construct
  source 1..8: note = S2V29-VH.8 CDRH2
IAYKGSNT

SEQ ID NO: 103 - protein, 16 aa, synthetic construct
  source 1..16: note = S2V29-VH.8 CDRH3
AKDYYPLLSYYYGIDV

SEQ ID NO: 104 - DNA, 369 bp, synthetic construct
  source 1..369: mol_type = other DNA; note = S2V29-VH.8 (nt)
gaagtgcaactagtggaaagtggtggtagtgtcgtgcagcctggcagagccctcagactgagctgtgctgcttctggcatcaccttctcctcttttggcatgtactgggtgcggcaagctcccggaaaaggactggaatggctgggcgtgatcgcctacaagggctctaatacctactacgccgattccgtgaagggccgcttcacaatctccagagataactccaagaacaccctgtacctgcagatgaactctctgcggatcgaggacaccgccgtgtactactgcgccaaggactactatcctctgctgtcctactactacggcatcgacgtgtggggccagggcaccacagtgaccgtgtccagc

SEQ ID NO: 105 - protein, 123 aa, synthetic construct
  source 1..123: note = S2V29-VH.9
EVQLVESGGSVVQPGRALRLSCAASGITFSSFGMYWVRQAPGKGLEWLGVIAYDASNTYYADSVKGRFTISRDNSKNTLYLQMNSLRIEDTAVYYCAKDYYPLLSYYYGIDVWGQGTTVTVSS

SEQ ID NO: 106 - protein, 8 aa, synthetic construct
  source 1..8: note = S2V29-VH.9 CDRH2
IAYDASNT

SEQ ID NO: 107 - protein, 16 aa, synthetic construct
  source 1..16: note = S2V29-VH.9 CDRH3
AKDYYPLLSYYYGIDV

SEQ ID NO: 108 - DNA, 369 bp, synthetic construct
  source 1..369: mol_type = other DNA; note = S2V29-VH.9 (nt)
gaagtgcaactagtggaaagtggtggtagtgtcgtgcagcctggcagagccctcagactgagctgtgctgcttctggcatcaccttctcctcttttggcatgtactgggtgcggcaagctcccggaaaaggactggaatggctgggcgtgatcgcctacgacgcctctaatacctactacgccgattccgtgaagggccgcttcacaatctccagagataactccaagaacaccctgtacctgcagatgaactctctgcggatcgaggacaccgccgtgtactactgcgccaaggactactatcctctgctgtcctactactacggcatcgacgtgtggggccagggcaccacagtgaccgtgtccagc

SEQ ID NO: 109 - protein, 123 aa, synthetic construct
  source 1..123: note = S2V29-VH.10
EVQLVESGGSVVQPGRALRLSCAASGITFSSFGMYWVRQAPGKGLEWLGVIAYDDSNTYYADSVKGRFTISRDNSKNTLYLQMNSLRIEDTAVYYCAKDYYPLLSYYYGIDVWGQGTTVTVSS

SEQ ID NO: 110 - protein, 8 aa, synthetic construct
  source 1..8: note = S2V29-VH.10 CDRH2
IAYDDSNT

SEQ ID NO: 111 - protein, 16 aa, synthetic construct
  source 1..16: note = S2V29-VH.10 CDRH3
AKDYYPLLSYYYGIDV

SEQ ID NO: 112 - DNA, 369 bp, synthetic construct
  source 1..369: mol_type = other DNA; note = S2V29-VH.10 (nt)
gaagtgcaactagtggaaagtggtggtagtgtcgtgcagcctggcagagccctcagactgagctgtgctgcttctggcatcaccttctcctcttttggcatgtactgggtgcggcaagctcccggaaaaggactggaatggctgggcgtgatcgcctacgacgactctaatacctactacgccgattccgtgaagggccgcttcacaatctccagagataactccaagaacaccctgtacctgcagatgaactctctgcggatcgaggacaccgccgtgtactactgcgccaaggactactatcctctgctgtcctactactacggcatcgacgtgtggggccagggcaccacagtgaccgtgtccagc

SEQ ID NO: 113 - protein, 110 aa, synthetic construct; identical to SEQ ID NO: 77
  source 1..110: note = S2V29-VL.2 (C91S) (aa)

SEQ ID NO: 114 - protein, 10 aa, synthetic construct
  source 1..10: note = S2V29-VL.2 CDRL3
SSYAGSYIWV

SEQ ID NO: 115 - DNA, 330 bp, synthetic construct
  source 1..330: mol_type = other DNA; note = S2V29-VL.2 (nt)
caaagtgtgctaactcaaccacgaagtgtgagcggctctcctggccagtccgtgaccatctcctgtaccggcacctctagcgatgtgggcgcttacaactacgtgtcctggtatcaacagcaccccggcaaggcccctaagttcatgatctacgacgtggaccagcggccatctggcgtgcctgatagattctccggctccaaatccggcaacaccgcttctctgatcatctctggcctgcaggccgaggacgaagccgactactactgctcctcctacgccggatcttacatctgggtctttggcggcggaacacagctgaccgtgctg

SEQ ID NO: 116 - protein, 110 aa, synthetic construct
  source 1..110: note = S2V29-VL.3 (aa)
QSVLTQPRSVSGSPGQSVTISCTGTSSDVGAYNYVSWYQQHPGKAPKFMIYDVDQRPSGVPDRFSGSKSGNTASLIISGLQAEDEADYYCASYAGSYIWVFGGGTQLTVL

SEQ ID NO: 117 - protein, 10 aa, synthetic construct
  source 1..10: note = S2V29-VL.3 CDRL3
ASYAGSYIWV

SEQ ID NO: 118 - DNA, 330 bp, synthetic construct
  source 1..330: mol_type = other DNA; note = S2V29-VL.3 (nt)
caaagtgtgctaactcaaccacgaagtgtgtctggctctccaggacaatccgtgaccatctcctgcaccggcacctccagcgatgtgggcgcctacaactacgtgtcctggtaccagcagcaccctggcaaggcccctaagttcatgatctacgacgtggaccagcggccttctggcgtgcccgatagattctccggcagcaaatccggcaacaccgcttctctgatcatcagcggcctgcaggccgaggacgaagccgactactactgcgcctcttatgctggctcctacatctgggtctttggcggcggaacacagctgaccgtgctg

SEQ ID NO: 119 - protein, 110 aa, synthetic construct
  source 1..110: note = S2V29-VL.4 (aa)
QSVLTQPRSVSGSPGQSVTISCTGTSSDVGAYNYVSWYQQHPGKAPKFMIYDVDQRPSGVPDRFSGSKSGNTASLIISGLQAEDEADYYCVSYAGSYIWVFGGGTQLTVL

SEQ ID NO: 120 - protein, 10 aa, synthetic construct
  source 1..10: note = S2V29-VL.4 CDRL3
VSYAGSYIWV

SEQ ID NO: 121 - DNA, 330 bp, synthetic construct
  source 1..330: mol_type = other DNA; note = S2V29-VL.4 (nt)
caaagtgtgctaactcaaccacgaagtgtgtctggctctccaggacaatccgtgaccatctcctgcaccggcacctccagcgatgtgggcgcctacaactacgtgtcctggtaccagcagcaccctggcaaggcccctaagttcatgatctacgacgtggaccagcggccttctggcgtgcccgatagattctccggcagcaaatccggcaacaccgcttctctgatcatcagcggcctgcaggccgaggacgaagccgactactactgcgtgtcttatgctggctcctacatctgggtctttggcggcggaacacagctgaccgtgctg

SEQ ID NO: 122 - protein, 110 aa, synthetic construct
  source 1..110: note = S2V29-VL.5 (aa)
QSVLTQPRSVSGSPGQSVTISCTGTSSDVGAYNYVSWYQQHPGKAPKFMIYDVDQRPSGVPDRFSGSKSGNTASLIISGLQAEDEADYYCYSYAGSYIWVFGGGTQLTVL

SEQ ID NO: 123 - protein, 10 aa, synthetic construct
  source 1..10: note = S2V29-VL.5 CDRL3
YSYAGSYIWV

SEQ ID NO: 124 - DNA, 330 bp, synthetic construct
  source 1..330: mol_type = other DNA; note = S2V29-VL.5 (nt)
caaagtgtgctaactcaaccacgaagtgtgtctggctctccaggacaatccgtgaccatctcctgcaccggcacctccagcgatgtgggcgcctacaactacgtgtcctggtaccagcagcaccctggcaaggcccctaagttcatgatctacgacgtggaccagcggccttctggcgtgcccgatagattctccggcagcaaatccggcaacaccgcttctctgatcatcagcggcctgcaggccgaggacgaagccgactactactgctactcttatgctggctcctacatctgggtctttggcggcggaacacagctgaccgtgctg

SEQ ID NO: 125 - protein, 110 aa, synthetic construct
  source 1..110: note = S2V29-VL.6 (aa)
QSVLTQPRSVSGSPGQSVTISCTGTSSDVGAYNYVSWYQQHPGKAPKFMIYDVDQRPSGVPDRFSGSKSGNTASLIISGLQAEDEADYYCGSYAGSYIWVFGGGTQLTVL

SEQ ID NO: 126 - protein, 10 aa, synthetic construct
  source 1..10: note = S2V29-VL.6 CDRL3
GSYAGSYIWV

SEQ ID NO: 127 - DNA, 330 bp, synthetic construct
  source 1..330: mol_type = other DNA; note = S2V29-VL.6 (nt)
caaagtgtgctaactcaaccacgaagtgtgtctggctctccaggacaatccgtgaccatctcctgcaccggcacctccagcgatgtgggcgcctacaactacgtgtcctggtaccagcagcaccctggcaaggcccctaagttcatgatctacgacgtggaccagcggccttctggcgtgcccgatagattctccggcagcaaatccggcaacaccgcttctctgatcatcagcggcctgcaggccgaggacgaagccgactactactgcggctcttatgctggctcctacatctgggtctttggcggcggaacacagctgaccgtgctg

SEQ ID NO: 128 - protein, 109 aa, synthetic construct
  source 1..109: note = S2V29-VL.7 (aa)
QSVLTQPRSVSGSPGQSVTISCTGTSSDVGAYNYVSWYQQHPGKAPKFMIYDVDQRPSGVPDRFSGSKSGNTASLIISGLQAEDEADYYCSYAGSYIWVFGGGTQLTVL

SEQ ID NO: 129 - protein, 9 aa, synthetic construct
  source 1..9: note = S2V29-VL.7 CDRL3
SYAGSYIWV

SEQ ID NO: 130 - DNA, 327 bp, synthetic construct
  source 1..327: mol_type = other DNA; note = S2V29-VL.7 (nt)
caaagtgtgctaactcaaccacgaagtgtgtctggctctccaggacaatccgtgaccatctcctgcaccggcacctccagcgatgtgggcgcctacaactacgtgtcctggtaccagcagcaccctggcaaggcccctaagttcatgatctacgacgtggaccagcggccttctggcgtgcccgatagattctccggcagcaaatccggcaacaccgcttctctgatcatcagcggcctgcaggccgaggacgaagccgactactactgttcttatgctggctcctacatctgggtctttggcggcggaacacagctgaccgtgctg

SEQ ID NO: 131 - protein, 110 aa, synthetic construct
  source 1..110: note = S2V29-VL.8 (aa)
QSVLTQPRSVSGSPGQSVTISCTGTSSDVGAYNYVSWYQQHPGKAPKFMIYDVDQRPSGVPDRFSGSKSGNTASLIISGLQAEDEADYYSCSYAGSYIWVFGGGTQLTVL

SEQ ID NO: 132 - DNA, 330 bp, synthetic construct
  source 1..330: mol_type = other DNA; note = S2V29-VL.8 (nt)
caaagtgtgctaactcaaccacgaagtgtgtctggctctccaggacaatccgtgaccatctcctgcaccggcacctccagcgatgtgggcgcctacaactacgtgtcctggtaccagcagcaccctggcaaggcccctaagttcatgatctacgacgtggaccagcggccttctggcgtgcccgatagattctccggcagcaaatccggcaacaccgcttctctgatcatcagcggcctgcaggccgaggacgaagccgactactactcctgttcttatgctggctcctacatctgggtctttggcggcggaacacagctgaccgtgctg

SEQ ID NO: 133 - protein, 110 aa, synthetic construct
  source 1..110: note = S2V29-VL.9 (aa)
QSVLTQPRSVSGSPGQSVTISCTGTSSDVGAYNYVSWYQQHPGKAPKFMIYDVDQRPSGVPDRFSGSKSGNTASLIISGLQAEDEADYYACSYAGSYIWVFGGGTQLTVL

SEQ ID NO: 134 - DNA, 330 bp, synthetic construct
  source 1..330: mol_type = other DNA; note = S2V29-VL.9 (nt)
caaagtgtgctaactcaaccacgaagtgtgtctggctctccaggacaatccgtgaccatctcctgcaccggcacctccagcgatgtgggcgcctacaactacgtgtcctggtaccagcagcaccctggcaaggcccctaagttcatgatctacgacgtggaccagcggccttctggcgtgcccgatagattctccggcagcaaatccggcaacaccgcttctctgatcatcagcggcctgcaggccgaggacgaagccgactactacgcctgttcttatgctggctcctacatctgggtctttggcggcggaacacagctgaccgtgctg

SEQ ID NO: 135 - DNA, 369 bp, synthetic construct; identical to SEQ ID NO: 79
  source 1..369: mol_type = other DNA; note = S2V29-VH.1 (nt) (alternative codon)

SEQ ID NO: 136 - DNA, 369 bp, synthetic construct
  source 1..369: mol_type = other DNA; note = S2V29-VH.11 (nt) (V50F)
gaagtgcaactagtggaaagtggtggtagtgtcgtgcagcctggcagagccctgagactgtcttgtgctgcttctggcatcaccttcagcagctttggcatgtactgggtgcggcaagctcccggaaaaggcctggaatggctgggcttcatcgcctacgacggctccaacacctactacgccgactccgtgaagggccgcttcaccatctccagagataattccaagaacaccctgtacctgcagatgaactctctgcggatcgaggacaccgccgtgtactactgcgccaaggactactaccctctgctgtcctactactatggactcgatgtgtggggccagggcacaaccgtgaccgtgtcctct

SEQ ID NO: 137 - protein, 123 aa, synthetic construct
  source 1..123: note = S2V29-VH.11 (aa) (V50F)
EVQLVESGGSVVQPGRALRLSCAASGITFSSFGMYWVRQAPGKGLEWLGFIAYDGSNTYYADSVKGRFTISRDNSKNTLYLQMNSLRIEDTAVYYCAKDYYPLLSYYYGLDVWGQGTTVTVSS

SEQ ID NO: 138 - DNA, 369 bp, synthetic construct
  source 1..369: mol_type = other DNA; note = S2V29-VH.12 (nt) (D54N)
gaagtgcaactagtggaaagtggtggtagtgtggtgcagcctggcagagctctgagactgtcttgtgctgcttctggcatcaccttttcttctttcggcatgtactgggtcagacaggcccccggcaagggcctggaatggctgggagtgatcgcctacaacggctccaacacctactacgccgactccgtgaaaggacggttcaccatctcccgggacaactccaagaatacactgtacctgcagatgaacagcctgcgcatcgaggacaccgccgtgtactactgcgccaaggactactatcctctgctgtcctactactacggcctcgatgtgtggggccaaggcaccaccgtgaccgtgtccagc

SEQ ID NO: 139 - protein, 123 aa, synthetic construct
  source 1..123: note = S2V29-VH.12 (aa) (D54N)
EVQLVESGGSVVQPGRALRLSCAASGITFSSFGMYWVRQAPGKGLEWLGVIAYNGSNTYYADSVKGRFTISRDNSKNTLYLQMNSLRIEDTAVYYCAKDYYPLLSYYYGLDVWGQGTTVTVSS

SEQ ID NO: 140 - protein, 8 aa, synthetic construct
  source 1..8: note = S2V29-VH.12 CDRH2
IAYNGSNT

SEQ ID NO: 141 - DNA, 369 bp, synthetic construct
  source 1..369: mol_type = other DNA; note = S2V29-VH.13 (nt) (I51D-A52K)
gaagtgcaactagtggaaagtggtggtagtgtcgtgcagcctggacgcgccctgagactctcttgtgctgcttctggcatcaccttcagcagcttcggcatgtactgggtgcggcaagctcctggaaaaggcctggaatggctgggcgtggacaagtacgacggctccaacacctactacgccgattccgtgaagggcagatttacaatctccagagataattccaagaacaccctgtacctgcagatgaactctctgcggatcgaggacaccgccgtgtactactgcgccaaggactactaccccctgctgtcctactattacggcctggacgtgtggggccagggcaccaccgtgaccgtgtcctct

SEQ ID NO: 142 - protein, 123 aa, synthetic construct
  source 1..123: note = S2V29-VH.13 (aa) ) (I51D-A52K)
EVQLVESGGSVVQPGRALRLSCAASGITFSSFGMYWVRQAPGKGLEWLGVDKYDGSNTYYADSVKGRFTISRDNSKNTLYLQMNSLRIEDTAVYYCAKDYYPLLSYYYGLDVWGQGTTVTVSS

SEQ ID NO: 143 - protein, 8 aa, synthetic construct
  source 1..8: note = S2V29-VH.13 CDRH2
DKYDGSNT

SEQ ID NO: 144 - DNA, 330 bp, synthetic construct
  source 1..330: mol_type = other DNA; note = S2V29-VL.10 (nt) (Y32H-I50M-D52N-C91S-S92V-Y93D-I98R)
caaagtgtgctaactcaaccacgaagtgtgtctggctctccaggccagtccgtgaccatctcttgtaccggcacatcctccgacgtgggcgctcacaactacgtgtcctggtaccagcagcaccctggcaaggcccctaagttcatgatgtacaacgtggatcaacggccttctggcgtgcccgacagatttagcggaagcaaaagcggcaacaccgcctctctgatcatctccggcctgcaggccgaggacgaagccgactactactgctccgtcgatgctggctcctatagatgggtgttcggcggcggaacccagctgaccgtgctg

SEQ ID NO: 145 - protein, 109 aa, synthetic construct
  source 1..109: note = S2V29-VL.10 (aa) (Y32H-I50M-D52N-C91S-S92V-Y93D-I98R)
QSVLTQPRSVSGSPGQSVTISCTGTSSDVGAHNYVSWYQQHPGKAPKFMMYNVDQRPSGVPDRFSGSKSGNTASLIISGLQAEDEADYYCSVDAGSYRWVFGGGTQLTV

SEQ ID NO: 146 - protein, 9 aa, synthetic construct
  source 1..9: note = S2V29-VL.10 CDRL1
SSDVGAHNY

SEQ ID NO: 147 - skipped

SEQ ID NO: 148 - protein, 10 aa, synthetic construct
  source 1..10: note = S2V29-VL.10 CDRL3
SVDAGSYRWV

SEQ ID NO: 149 - DNA, 330 bp, synthetic construct
  source 1..330: mol_type = other DNA; note = S2V29-VL.11 (nt) (Y32H-D52N-C91S-S92V-Y93D-I98R)
caaagtgtgctaactcaaccacgaagtgtgtctggctctcctggccagtccgtgaccatcagctgtaccggcacctcctccgacgtgggcgctcacaactacgtgtcctggtaccagcagcaccccggcaaggcccctaagttcatgatctacaacgtggatcaacggccatctggcgtgcctgacagatttagcggatctaaaagcggcaacaccgcctctctgatcatctccggcctgcaggccgaggacgaagccgactactactgctccgtcgatgctggctcctatagatgggtgttcggcggcggaacacagctgaccgtgctg

SEQ ID NO: 150 - protein, 110 aa, synthetic construct
  source 1..110: note = S2V29-VL.11 (aa) (Y32H-D52N-C91S-S92V-Y93D-I98R)
QSVLTQPRSVSGSPGQSVTISCTGTSSDVGAHNYVSWYQQHPGKAPKFMIYNVDQRPSGVPDRFSGSKSGNTASLIISGLQAEDEADYYCSVDAGSYRWVFGGGTQLTVL

SEQ ID NO: 151 - DNA, 330 bp, synthetic construct
  source 1..330: mol_type = other DNA; note = S2V29-VL.12 (nt) (Y32H-D52N-C91S-S92V-Y93D-I98R)
caaagtgtgctaactcaaccacgaagtgtgtctggctctcctggccaatccgtgaccatctcctgtaccggcacaagctccgacgtgggcgcctataactacgtgtcctggtaccagcagcaccccggcaaggcccctaaattcatgatgtacaacgtggaccaccggccttctggcgtgccagatagattcagcggatccaagtccggcaacaccgcttctctgatcatctctggcctgcaggccgaggacgaagccgactactactgctccggcaatgctggcagctacagatgggtctttggtggaggcacccagctgaccgtgctg

SEQ ID NO: 152 - protein, 110 aa, synthetic construct
  source 1..110: note = S2V29-VL.12 (aa) (Y32H-D52N-C91S-S92V-Y93D-I98R)
QSVLTQPRSVSGSPGQSVTISCTGTSSDVGAYNYVSWYQQHPGKAPKFMMYNVDHRPSGVPDRFSGSKSGNTASLIISGLQAEDEADYYCSGNAGSYRWVFGGGTQLTVL

SEQ ID NO: 153 - protein, 10 aa, synthetic construct
  source 1..10: note = S2V29-VL.12 CDRL3
SGNAGSYRWV

SEQ ID NO: 154 - DNA, 330 bp, synthetic construct
  source 1..330: mol_type = other DNA; note = S2V29-VL.13 (nt) (Y32N-I50M-D52N-Q55K-C91S-S92V-Y93D-I98K)
caaagtgtgctaactcaaccacgaagtgtgagcggctctccaggacaatctgtgaccatctcctgtaccggcacctcctccgacgtgggcgccaacaactacgtgtcctggtaccagcagcaccctggcaaagctcctaagttcatgatgtacaacgtggacaagcggccttctggcgtgcccgatagatttagcggctccaagtccggcaataccgcctctctgatcatctctggcctgcaggccgaggacgaagccgactactactgctccgtcgatgctggcagctataagtgggtgttcggcggcggaacacagctgaccgtgctg

SEQ ID NO: 155 - protein, 110 aa, synthetic construct
  source 1..110: note = S2V29-VL.13 (aa) (Y32N-I50M-D52N-Q55K-C91S-S92V-Y93D-I98K)
QSVLTQPRSVSGSPGQSVTISCTGTSSDVGANNYVSWYQQHPGKAPKFMMYNVDKRPSGVPDRFSGSKSGNTASLIISGLQAEDEADYYCSVDAGSYKWVFGGGTQLTVL

SEQ ID NO: 156 - protein, 9 aa, synthetic construct
  source 1..9: note = SV29-VL.13 CDRL1
SSDVGANNY

SEQ ID NO: 157 - protein, 10 aa, synthetic construct
  source 1..10: note = SV29-VL.13 CDRL3
SVDAGSYKWV

SEQ ID NO: 158 - DNA, 330 bp, synthetic construct
  source 1..330: mol_type = other DNA; note = S2V29-VL.14 (nt) (Y32H-I50M-D52G-C91S-S92F-Y93D-I98R)
caaagtgtgctaactcaaccacgaagtgtgagcggctctcctggccagtccgtgaccatctcttgtaccggcacaagctccgacgtgggcgctcacaactacgtgtcctggtaccagcagcaccccggcaaggcccctaagttcatgatgtacggcgtggaccaacggccttctggtgtcccagatagattctccggatctaaaagcggcaacaccgcctctctgatcatctccggcctgcaggccgaggacgaagccgactactactgctcctttgatgctggctcctatagatgggtgttcggcggaggcacccagctgaccgtgctg

SEQ ID NO: 159 - protein, 110 aa, synthetic construct
  source 1..110: note = S2V29-VL.14 (aa) (Y32H-I50M-D52G-C91S-S92F-Y93D-I98R)
QSVLTQPRSVSGSPGQSVTISCTGTSSDVGAHNYVSWYQQHPGKAPKFMMYGVDQRPSGVPDRFSGSKSGNTASLIISGLQAEDEADYYCSFDAGSYRWVFGGGTQLTVL

SEQ ID NO: 160 - skipped

SEQ ID NO: 161 - protein, 10 aa, synthetic construct
  source 1..10: note = S2V29-VL.14 CDRL3
SFDAGSYRWV

SEQ ID NO: 162 - DNA, 330 bp, synthetic construct
  source 1..330: mol_type = other DNA; note = S2V29-VL.15 (nt) (Y32H-I50M-D52N-C91S-S92D-Y93D-I98R)
caaagtgtgctaactcaaccacgaagtgtgtccggctctccaggccagtccgtgaccatctcctgtaccggcaccagctctgacgtgggcgctcacaactacgtgtcctggtaccagcagcaccctggcaaggcccctaagttcatgatgtacaacgtggaccaacggccttctggcgtgcccgatagattcagcggatctaaatccggcaacaccgcctctctgatcatcagcggcctgcaggccgaggatgaagccgactactactgctccgacgacgctggctcctatagatgggtctttggcggcggaacacagctgaccgtgctg

SEQ ID NO: 163 - protein, 110 aa, synthetic construct
  source 1..110: note = S2V29-VL.15 (aa) (Y32H-I50M-D52N-C91S-S92D-Y93D-I98R)
QSVLTQPRSVSGSPGQSVTISCTGTSSDVGAHNYVSWYQQHPGKAPKFMMYNVDQRPSGVPDRFSGSKSGNTASLIISGLQAEDEADYYCSDDAGSYRWVFGGGTQLTVL

SEQ ID NO: 164 - protein, 10 aa, synthetic construct
  source 1..10: note = SV29-VL.15 CDRL3
SDDAGSYRWV

SEQ ID NO: 165 - DNA, 369 bp, synthetic construct
  source 1..369: mol_type = other DNA; note = S2V29-VH.22 (nt) (V50S, A52S, N57F, Y101F, Y107F)
gaagtgcaactagtggaaagtggtggtagtgtcgtgcagcctggcagagccctcagactgagctgtgctgcttctggcatcaccttctcctcttttggcatgtactgggtgcggcaagctcccggaaaaggactggaatggctgggcagcatcagctacgacggctctttcacctactacgccgattccgtgaagggccgcttcacaatctccagagataactccaagaacaccctgtacctgcagatgaactctctgcggatcgaggacaccgccgtgtactactgcgccaaggactacttccctctgctgtcctacttctacggcctggacgtgtggggccagggcaccacagtgaccgtgtccagc

SEQ ID NO: 166 - protein, 123 aa, synthetic construct
  source 1..123: note = S2V29-VH.22 (aa) (V50S, A52S, N57F, Y101F, Y107F)
EVQLVESGGSVVQPGRALRLSCAASGITFSSFGMYWVRQAPGKGLEWLGSISYDGSFTYYADSVKGRFTISRDNSKNTLYLQMNSLRIEDTAVYYCAKDYFPLLSYFYGLDVWGQGTTVTVSS

SEQ ID NO: 167 - protein, 8 aa, synthetic construct
  source 1..8: note = S2V29-VH.22 CDRH2
ISYDGSFT

SEQ ID NO: 168 - protein, 16 aa, synthetic construct
  source 1..16: note = S2V29-VH.22 CDRH3
AKDYFPLLSYFYGLDV

SEQ ID NO: 169 - DNA, 369 bp, synthetic construct
  source 1..369: mol_type = other DNA; note = S2V29-VH.24 (nt) (V50A, N57D, Y107F)
gaagtgcaactagtggaaagtggtggtagtgtcgtgcagcctggcagagccctcagactgagctgtgctgcttctggcatcaccttctcctcttttggcatgtactgggtgcggcaagctcccggaaaaggactggaatggctgggcgccatcgcctacgacggctctgacacctactacgccgattccgtgaagggccgcttcacaatctccagagataactccaagaacaccctgtacctgcagatgaactctctgcggatcgaggacaccgccgtgtactactgcgccaaggactactatcctctgctgtcctacttctacggcctggacgtgtggggccagggcaccacagtgaccgtgtccagc

SEQ ID NO: 170 - protein, 123 aa, synthetic construct
  source 1..123: note = S2V29-VH.24 (aa) (V50A, N57D, Y107F)
EVQLVESGGSVVQPGRALRLSCAASGITFSSFGMYWVRQAPGKGLEWLGAIAYDGSDTYYADSVKGRFTISRDNSKNTLYLQMNSLRIEDTAVYYCAKDYYPLLSYFYGLDVWGQGTTVTVSS

SEQ ID NO: 171 - protein, 8 aa, synthetic construct
  source 1..8: note = S2V29-VH.24 CDRH2
IAYDGSDT

SEQ ID NO: 172 - protein, 16 aa, synthetic construct
  source 1..16: note = S2V29-VH.24 CDRH3
AKDYYPLLSYFYGLDV

SEQ ID NO: 173 - DNA, 369 bp, synthetic construct
  source 1..369: mol_type = other DNA; note = S2V29-VH.25 (nt) (V50A, A52T, N57D, Y107F)
gaagtgcaactagtggaaagtggtggtagtgtcgtgcagcctggcagagccctcagactgagctgtgctgcttctggcatcaccttctcctcttttggcatgtactgggtgcggcaagctcccggaaaaggactggaatggctgggcgccatcacctacgacggctctgacacctactacgccgattccgtgaagggccgcttcacaatctccagagataactccaagaacaccctgtacctgcagatgaactctctgcggatcgaggacaccgccgtgtactactgcgccaaggactactatcctctgctgtcctacttctacggcctggacgtgtggggccagggcaccacagtgaccgtgtccagc

SEQ ID NO: 174 - protein, 123 aa, synthetic construct
  source 1..123: note = S2V29-VH.25 (aa) (V50A, A52T, N57D, Y107F)
EVQLVESGGSVVQPGRALRLSCAASGITFSSFGMYWVRQAPGKGLEWLGAITYDGSDTYYADSVKGRFTISRDNSKNTLYLQMNSLRIEDTAVYYCAKDYYPLLSYFYGLDVWGQGTTVTVSS

SEQ ID NO: 175 - protein, 8 aa, synthetic construct
  source 1..8: note = S2V29-VH.25 CDRH2
ITYDGSDT

SEQ ID NO: 176 - DNA, 369 bp, synthetic construct
  source 1..369: mol_type = other DNA; note = S2V29-VH.30 (nt) (V50Y, N57D, Y107F)
gaagtgcaactagtggaaagtggtggtagtgtcgtgcagcctggcagagccctcagactgagctgtgctgcttctggcatcaccttctcctcttttggcatgtactgggtgcggcaagctcccggaaaaggactggaatggctgggctacatcgcctacgacggctctgacacctactacgccgattccgtgaagggccgcttcacaatctccagagataactccaagaacaccctgtacctgcagatgaactctctgcggatcgaggacaccgccgtgtactactgcgccaaggactactatcctctgctgtcctacttctacggcctggacgtgtggggccagggcaccacagtgaccgtgtccagc

SEQ ID NO: 177 - protein, 123 aa, synthetic construct
  source 1..123: note = S2V29-VH.30 (aa) (V50Y, N57D, Y107F)
EVQLVESGGSVVQPGRALRLSCAASGITFSSFGMYWVRQAPGKGLEWLGYIAYDGSDTYYADSVKGRFTISRDNSKNTLYLQMNSLRIEDTAVYYCAKDYYPLLSYFYGLDVWGQGTTVTVSS

SEQ ID NO: 178 - DNA, 369 bp, synthetic construct
  source 1..369: mol_type = other DNA; note = S2V29-VH.31 (nt) (V50Y, A52S, N57D, Y107F)
gaagtgcaactagtggaaagtggtggtagtgtcgtgcagcctggcagagccctcagactgagctgtgctgcttctggcatcaccttctcctcttttggcatgtactgggtgcggcaagctcccggaaaaggactggaatggctgggctacatcagctacgacggctctgacacctactacgccgattccgtgaagggccgcttcacaatctccagagataactccaagaacaccctgtacctgcagatgaactctctgcggatcgaggacaccgccgtgtactactgcgccaaggactactatcctctgctgtcctacttctacggcctggacgtgtggggccagggcaccacagtgaccgtgtccagc

SEQ ID NO: 179 - protein, 123 aa, synthetic construct
  source 1..123: note = S2V29-VH.31 (aa) (V50Y, A52S, N57D, Y107F)
EVQLVESGGSVVQPGRALRLSCAASGITFSSFGMYWVRQAPGKGLEWLGYISYDGSDTYYADSVKGRFTISRDNSKNTLYLQMNSLRIEDTAVYYCAKDYYPLLSYFYGLDVWGQGTTVTVSS

SEQ ID NO: 180 - protein, 8 aa, synthetic construct
  source 1..8: note = S2V29-VH.31 CDRH2
ISYDGSDT

SEQ ID NO: 181 - DNA, 369 bp, synthetic construct
  source 1..369: mol_type = other DNA; note = S2V29-VH.36 (nt) (V50Y, A52S, N57D)
gaagtgcaactagtggaaagtggtggtagtgtcgtgcagcctggcagagccctcagactgagctgtgctgcttctggcatcaccttctcctcttttggcatgtactgggtgcggcaagctcccggaaaaggactggaatggctgggctacatcagctacgacggctctgacacctactacgccgattccgtgaagggccgcttcacaatctccagagataactccaagaacaccctgtacctgcagatgaactctctgcggatcgaggacaccgccgtgtactactgcgccaaggactactatcctctgctgtcctactactacggcctggacgtgtggggccagggcaccacagtgaccgtgtccagc

SEQ ID NO: 182 - protein, 123 aa, synthetic construct
  source 1..123: note = S2V29-VH.36 (aa) (V50Y, A52S, N57D)
EVQLVESGGSVVQPGRALRLSCAASGITFSSFGMYWVRQAPGKGLEWLGYISYDGSDTYYADSVKGRFTISRDNSKNTLYLQMNSLRIEDTAVYYCAKDYYPLLSYYYGLDVWGQGTTVTVSS

SEQ ID NO: 183 - DNA, 369 bp, synthetic construct
  source 1..369: mol_type = other DNA; note = S2V29-VH.37 (nt) (V50Y, N57D)
gaagtgcaactagtggaaagtggtggtagtgtcgtgcagcctggcagagccctcagactgagctgtgctgcttctggcatcaccttctcctcttttggcatgtactgggtgcggcaagctcccggaaaaggactggaatggctgggctacatcgcctacgacggctctgacacctactacgccgattccgtgaagggccgcttcacaatctccagagataactccaagaacaccctgtacctgcagatgaactctctgcggatcgaggacaccgccgtgtactactgcgccaaggactactatcctctgctgtcctactactacggcctggacgtgtggggccagggcaccacagtgaccgtgtccagc

SEQ ID NO: 184 - protein, 123 aa, synthetic construct
  source 1..123: note = S2V29-VH.37 (aa) (V50Y, N57D)
EVQLVESGGSVVQPGRALRLSCAASGITFSSFGMYWVRQAPGKGLEWLGYIAYDGSDTYYADSVKGRFTISRDNSKNTLYLQMNSLRIEDTAVYYCAKDYYPLLSYYYGLDVWGQGTTVTVSS

SEQ ID NO: 185 - DNA, 318 bp, synthetic construct
  source 1..318: mol_type = other DNA; note = IgG1 Light Chain Lambda (nt)
ggtcagcccaaggctgccccctcggtcactctgttcccgccctcgagtgaggagctccaagccaacaaggccaccctggtctgtctgattagcgatttctaccctggtgctgtcactgtcgcatggaaggccgattcatcccccgtcaaagctggtgtcgagacaacaacccccagcaagcagtctaacaataagtacgccgcttccagctatctgtccctgacacctgagcagtggaagagccacaggtcttactcctgtcaggtcactcacgaaggatcaactgtggaaaaaactgtggcaccaaccgaatgttca

SEQ ID NO: 186 - protein, 106 aa, synthetic construct
  source 1..106: note = IgG1 Light Chain Lambda (aa)
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 187 - protein, 123 aa, synthetic construct
  source 1..123: note = S2V29-VH.A (V50Y)
EVQLVESGGSVVQPGRALRLSCAASGITFSSFGMYWVRQAPGKGLEWLGYIAYDGSNTYYADSVKGRFTISRDNSKNTLYLQMNSLRIEDTAVYYCAKDYYPLLSYYYGLDVWGQGTTVTVSS

SEQ ID NO: 188 - protein, 123 aa, synthetic construct
  source 1..123: note = S2V29-VH.B (V50*)
  VARIANT 50: note = may be any amino acid other than V
EVQLVESGGSVVQPGRALRLSCAASGITFSSFGMYWVRQAPGKGLEWLGXIAYDGSNTYYADSVKGRFTISRDNSKNTLYLQMNSLRIEDTAVYYCAKDYYPLLSYYYGLDVWGQGTTVTVSS

SEQ ID NO: 189 - protein, 123 aa, synthetic construct; identical to SEQ ID NO: 188
  source 1..123: note = S2V29-VH.C (V50 aromatic)
  VARIANT 50: note = may be any amino acid containing an aromatic ring, in particular F, W, or T

SEQ ID NO: 190 - protein, 123 aa, synthetic construct; identical to SEQ ID NO: 188
  source 1..123: note = S2V29-VH.D (V50 polar)
  VARIANT 50: note = may be any amino acid having a polar side chain, in particular S, T, C, N, Q, or Y

SEQ ID NO: 191 - protein, 123 aa, synthetic construct; identical to SEQ ID NO: 188
  source 1..123: note = S2V29-VH.E (V50 amphipathic)
  VARIANT 50: note = may be any amphipathic amino acid, in particular K, W, Y, or M

SEQ ID NO: 192 - protein, 123 aa, synthetic construct
  source 1..123: note = S2V29-VH.F (N57D)
EVQLVESGGSVVQPGRALRLSCAASGITFSSFGMYWVRQAPGKGLEWLGVIAYDGSDTYYADSVKGRFTISRDNSKNTLYLQMNSLRIEDTAVYYCAKDYYPLLSYYYGLDVWGQGTTVTVSS

SEQ ID NO: 193 - protein, 123 aa, synthetic construct
  source 1..123: note = S2V29-VH.G (N57*)
  VARIANT 57: note = may be any amino acid other than N
EVQLVESGGSVVQPGRALRLSCAASGITFSSFGMYWVRQAPGKGLEWLGVIAYDGSXTYYADSVKGRFTISRDNSKNTLYLQMNSLRIEDTAVYYCAKDYYPLLSYYYGLDVWGQGTTVTVSS

SEQ ID NO: 194 - protein, 123 aa, synthetic construct; identical to SEQ ID NO: 193
  source 1..123: note = S2V29-VH.H (N57 acidic)
  VARIANT 57: note = may be any acidic amino acid, in particular D or E

SEQ ID NO: 195 - protein, 123 aa, synthetic construct; identical to SEQ ID NO: 193
  source 1..123: note = S2V29-VH.I (N57 hydrophilic)
  VARIANT 57: note = may be any hydrophilic amino acid other than N, in particular D, E, S, T, C, Q, R, or H

SEQ ID NO: 196 - protein, 123 aa, synthetic construct
  source 1..123: note = S2V29-VH.J (V50Y, N57*)
  VARIANT 57: note = may be any amino acid other than N
EVQLVESGGSVVQPGRALRLSCAASGITFSSFGMYWVRQAPGKGLEWLGYIAYDGSXTYYADSVKGRFTISRDNSKNTLYLQMNSLRIEDTAVYYCAKDYYPLLSYYYGLDVWGQGTTVTVSS

SEQ ID NO: 197 - protein, 123 aa, synthetic construct; identical to SEQ ID NO: 196
  source 1..123: note = S2V29-VH.K (V50Y, N57 hydrophilic)
  VARIANT 57: note = may be any hydrophilic amino acid other than N, in particular D, E, S, T, C, Q, R, or H

SEQ ID NO: 198 - protein, 123 aa, synthetic construct; identical to SEQ ID NO: 196
  source 1..123: note = S2V29-VH.K (V50Y, N57 acidic)
  VARIANT 57: note = may be any acidic amino acid, in particular D or E

SEQ ID NO: 199 - protein, 123 aa, synthetic construct
  source 1..123: note = S2V29-VH.L (V50*, N57D)
  VARIANT 50: note = may be any amino acid other than V
EVQLVESGGSVVQPGRALRLSCAASGITFSSFGMYWVRQAPGKGLEWLGXIAYDGSDTYYADSVKGRFTISRDNSKNTLYLQMNSLRIEDTAVYYCAKDYYPLLSYYYGLDVWGQGTTVTVSS

SEQ ID NO: 200 - protein, 123 aa, synthetic construct; identical to SEQ ID NO: 199
  source 1..123: note = S2V29-VH.M (V50 aromatic, N57D)
  VARIANT 50: note = may be any amino acid containing an aromatic ring, in particular F, W, or T

SEQ ID NO: 201 - protein, 123 aa, synthetic construct; identical to SEQ ID NO: 199
  source 1..123: note = S2V29-VH.N (V50 polar, N57D)
  VARIANT 50: note = may be any amino acid having a polar side chain, in particular S, T, C, N, Q, or Y

SEQ ID NO: 202 - protein, 123 aa, synthetic construct; identical to SEQ ID NO: 199
  source 1..123: note = S2V29-VH.O (V50 amphipathic, N57D)
  VARIANT 50: note = may be any amphipathic amino acid, in particular K, W, Y, or M

SEQ ID NO: 203 - protein, 453 aa, synthetic construct
  source 1..453: note = S2V29-v37.2 HC (VH.37 + G1m17,1 CH1-CH3 + LS mutation)
EVQLVESGGSVVQPGRALRLSCAASGITFSSFGMYWVRQAPGKGLEWLGYIAYDGSDTYYADSVKGRFTISRDNSKNTLYLQMNSLRIEDTAVYYCAKDYYPLLSYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 204 - protein, 216 aa, synthetic construct
  source 1..216: note = S2V29-v37.2 LC (VL.2 + Light chain Lambda)
QSVLTQPRSVSGSPGQSVTISCTGTSSDVGAYNYVSWYQQHPGKAPKFMIYDVDQRPSGVPDRFSGSKSGNTASLIISGLQAEDEADYYCSSYAGSYIWVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 205 - RNA, 58 nt, synthetic construct
  source 1..58: mol_type = other RNA; note = saRNA promoter
taacctgaatggactacgacatagtctagtccgccaagtctagcatatggccaccatg

SEQ ID NO: 206 - protein, 2433 aa, synthetic construct
  source 1..2433: note = VEEV nsP1-4
ILDIGSAPARRMYSKHKYHCICPMRCAEDPDRLYKYATKLKKNCKEITDKELDKKMKELAAVMSDPDLETETMCLHDDESCRYEGQVAVYQDVYAVDGPTSLYHQANKGVRVAYWIGFDTTPFMFKNLAGAYPSYSTNWADETVLTARNIGLCSSDVMERSRRGMSILRKKYLKPSNNVLFSVGSTIYHEKRDLLRSWHLPSVFHLRGKQNYTCRCETIVSCDGYVVKRIAISPGLYGKPSGYAATMHREGFLCCKVTDTLNGERVSFPVCTYVPATLCDQMTGILATDVSADDAQKLLVGLNQRIVVNGRTQRNTNTMKNYLLPVVAQAFARWAKEYKEDQEDERPLGLRDRQLVMGCCWAFRRHKITSIYKRPDTQTIIKVNSDFHSFVLPRIGSNTLEIGLRTRIRKMLEEHKEPSPLITAEDVQEAKCAADEAKEVREAEELRAALPPLAADVEEPTLEADVDLMLQEAGAGSVETPRGLIKVTSYDGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKKMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKMVDWLSDRPEATFRARLDLGIPGDVPKYDIIFVNVRTPYKYHHYQQCEDHAIKLSMLTKKACLHLNPGGTCVSIGYGYADRASESIIGAIARQFKFSRVCKPKSSLEETEVLFVFIGYDRKARTHNSYKLSSTLTNIYTGSRLHEAGCAPSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVDETPEPSAENQSTEGTPEQPPLITEDETRTRTPEPIIIEEEEEDSISLLSDGPTHQVLQVEADIHGPPSVSSSSWSIPHASDFDVDSLSILDTLEGASVTSGATSAETNSYFAKSMEFLARPVPAPRTVFRNPPHPAPRTRTPSLAPSRACSRTSLVSTPPGVNRVITREELEALTPSRTPSRSVSRTSLVSNPPGVNRVITREEFEAFVAQQQXRFDAGAYIFSSDTGQGHLQQKSVRQTVLSEVVLERTELEISYAPRLDQEKEELLRKKLQLNPTPANRSRYQSRKVENMKAITARRILQGLGHYLKAEGKVECYRTLHPVPLYSSSVNRAFSSPKVAVEACNAMLKENFPTVASYCIIPEYDAYLDMVDGASCCLDTASFCPAKLRSFPKKHSYLEPTIRSAVPSAIQNTLQNVLAAATKRNCNVTQMRELPVLDSAAFNVECFKKYACNNEYWETFKENPIRLTEENVVNYITKLKGPKAAALFAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTALMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILSELCKAVESRYETVGTSIIVMAMTTLASSVKSFSYLRGAPITLYG

SEQ ID NO: 207 - protein, 25 aa, synthetic construct
  source 1..25: note = S2V29-VH.37 Framework 1 (aa)
EVQLVESGGSVVQPGRALRLSCAAS

SEQ ID NO: 208 - protein, 17 aa, synthetic construct
  source 1..17: note = S2V29-VH.37 Framework 2 (aa)
MYWVRQAPGKGLEWLGY

SEQ ID NO: 209 - protein, 38 aa, synthetic construct
  source 1..38: note = S2V29-VH.37 Framework 3 (aa)
YYADSVKGRFTISRDNSKNTLYLQMNSLRIEDTAVYYC

SEQ ID NO: 210 - protein, 11 aa, synthetic construct
  source 1..11: note = S2V29-VH.37 Framework 4 (aa)
WGQGTTVTVSS

SEQ ID NO: 211 - protein, 25 aa, synthetic construct
  source 1..25: note = S2V29-VL.2 Framework 1 (aa)
QSVLTQPRSVSGSPGQSVTISCTGT

SEQ ID NO: 212 - protein, 17 aa, synthetic construct
  source 1..17: note = S2V29-VL.2 Framework 2 (aa)
VSWYQQHPGKAPKFMIY

SEQ ID NO: 213 - protein, 36 aa, synthetic construct
  source 1..36: note = S2V29-VL.2 Framework 3 (aa)
QRPSGVPDRFSGSKSGNTASLIISGLQAEDEADYYC

SEQ ID NO: 214 - protein, 10 aa, synthetic construct
  source 1..10: note = S2V29-VL.2 Framework 4 (aa)
FGGGTQLTVL

SEQ ID NO: 215 - protein, 9 aa, synthetic construct
  source 1..9: note = S2V29-VL.37 CDRH2+Fr.A (aa)
YIAYDGSDT

SEQ ID NO: 216 - protein, 14 aa, synthetic construct
  source 1..14: note = S2V29-VL.37 CDRH2+Fr.B (aa)
WLGYIAYDGSDTYY

SEQ ID NO: 217 - protein, 49 aa, synthetic construct
  source 1..49: note = S2V29-VH.37 H1-H3 (aa)
GITFSSFGMYWVRQAPGKGLEWLGYIAYDGSDTAKDYYPLLSYYYGLDV

SEQ ID NO: 218 - protein, 33 aa, synthetic construct
  source 1..33: note = S2V29-VH.37 H1-H2 (aa)
GITFSSFGMYWVRQAPGKGLEWLGYIAYDGSDT

SEQ ID NO: 219 - protein, 62 aa, synthetic construct
  source 1..62: note = S2V29-VH.37 H2-H3 (aa)
IAYDGSDTYYADSVKGRFTISRDNSKNTLYLQMNSLRIEDTAVYYCAKDYYPLLSYYYGLDV

SEQ ID NO: 220 - protein, 63 aa, synthetic construct
  source 1..63: note = S2V29-VH.37 H2+Fr.A-H3 (aa)
YIAYDGSDTYYADSVKGRFTISRDNSKNTLYLQMNSLRIEDTAVYYCAKDYYPLLSYYYGLDV

SEQ ID NO: 221 - protein, 66 aa, synthetic construct
  source 1..66: note = S2V29.VH.37 H2+Fr.B-H3 (aa)
WLGYIAYDGSDTYYADSVKGRFTISRDNSKNTLYLQMNSLRIEDTAVYYCAKDYYPLLSYYYGLDV

SEQ ID NO: 222 - protein, 75 aa, synthetic construct
  source 1..75: note = S2V29-VL.2 L1-L3 (aa)
SSDVGAYNYVSWYQQHPGKAPKFMIYDVDQRPSGVPDRFSGSKSGNTASLIISGLQAEDEADYYCSSYAGSYIWV

SEQ ID NO: 223 - protein, 29 aa, synthetic construct
  source 1..29: note = S2V29-VL.2 L1-L2 (aa)
SSDVGAYNYVSWYQQHPGKAPKFMIYDVD

SEQ ID NO: 224 - protein, 49 aa, synthetic construct
  source 1..49: note = S2V29-VL.2 L2-L3 (aa)
DVDQRPSGVPDRFSGSKSGNTASLIISGLQAEDEADYYCSSYAGSYIWV

SEQ ID NO: 225 - DNA, 24 bp, synthetic construct
  source 1..24: mol_type = other DNA; note = S2V29-VH.37 CDRH1 (nt)
ggcatcaccttctcctcttttggc

SEQ ID NO: 226 - DNA, 21 bp, synthetic construct
  source 1..21: mol_type = other DNA; note = S2V29-VH.37 CDRH2 (nt)
atcgcctacgacggctctgac

SEQ ID NO: 227 - DNA, 48 bp, synthetic construct
  source 1..48: mol_type = other DNA; note = S2V29-VH.37 CDRH3 (nt)
gccaaggactactatcctctgctgtcctactactacggcctggacgtg

SEQ ID NO: 228 - DNA, 27 bp, synthetic construct
  source 1..27: mol_type = other DNA; note = S2V29-VH.2 CDRL1 (nt)
tctagcgatgtgggcgcttacaactac

SEQ ID NO: 229 - skipped

SEQ ID NO: 230 - DNA, 30 bp, synthetic construct
  source 1..30: mol_type = other DNA; note = S2V29-VH.2 CDRL3 (nt)
tcctcctacgccggatcttacatctgggtc

SEQ ID NO: 231 - DNA, 75 bp, synthetic construct
  source 1..75: mol_type = other DNA; note = S2V29-VH.37 Framework 1 (nt)
gaagtgcaactagtggaaagtggtggtagtgtcgtgcagcctggcagagccctcagactgagctgtgctgcttct

SEQ ID NO: 232 - DNA, 51 bp, synthetic construct
  source 1..51: mol_type = other DNA; note = S2V29-VH.37 Framework 2 (nt)
atgtactgggtgcggcaagctcccggaaaaggactggaatggctgggctac

SEQ ID NO: 233 - DNA, 116 bp, synthetic construct
  source 1..116: mol_type = other DNA; note = S2V29-VH.37 Framework 3 (nt)
cctactacgccgattccgtgaagggccgcttcacaatctccagagataactccaagaacaccctgtacctgcagatgaactctctgcggatcgaggacaccgccgtgtactactgc

SEQ ID NO: 234 - DNA, 33 bp, synthetic construct
  source 1..33: mol_type = other DNA; note = S2V29-VH.37 Framework 4 (nt)
tggggccagggcaccacagtgaccgtgtccagc

SEQ ID NO: 235 - DNA, 75 bp, synthetic construct
  source 1..75: mol_type = other DNA; note = S2V29-VL.2 Framework 1 (nt)
caaagtgtgctaactcaaccacgaagtgtgagcggctctcctggccagtccgtgaccatctcctgtaccggcacc

SEQ ID NO: 236 - DNA, 51 bp, synthetic construct
  source 1..51: mol_type = other DNA; note = S2V29-VL.2 Framework 2 (nt)
gtgtcctggtatcaacagcaccccggcaaggcccctaagttcatgatctac

SEQ ID NO: 237 - DNA, 108 bp, synthetic construct
  source 1..108: mol_type = other DNA; note = S2V29-VL.2 Framework 3 (nt)
cagcggccatctggcgtgcctgatagattctccggctccaaatccggcaacaccgcttctctgatcatctctggcctgcaggccgaggacgaagccgactactactgc

SEQ ID NO: 238 - DNA, 30 bp, synthetic construct
  source 1..30: mol_type = other DNA; note = S2V29-VL.2 Framework 4 (nt)
tttggcggcggaacacagctgaccgtgctg

SEQ ID NO: 239 - DNA, 25 bp, synthetic construct
  source 1..25: mol_type = other DNA; note = S2V29-VL.37 CDRH2+Fr.A (nt)
ctacatcgcctacgacggctctgac

SEQ ID NO: 240 - DNA, 42 bp, synthetic construct
  source 1..42: mol_type = other DNA; note = S2V29-VL.37 CDRH2+Fr.B (nt)
tggctgggctacatcgcctacgacggctctgacacctactac

SEQ ID NO: 241 - DNA, 261 bp, synthetic construct
  source 1..261: mol_type = other DNA; note = S2V29-VH.37 H1-H3 (nt)
ggcatcaccttctcctcttttggcatgtactgggtgcggcaagctcccggaaaaggactggaatggctgggctacatcgcctacgacggctctgacacctactacgccgattccgtgaagggccgcttcacaatctccagagataactccaagaacaccctgtacctgcagatgaactctctgcggatcgaggacaccgccgtgtactactgcgccaaggactactatcctctgctgtcctactactacggcctggacgtg

SEQ ID NO: 242 - DNA, 96 bp, synthetic construct
  source 1..96: mol_type = other DNA; note = S2V29-VH.37 H1-H2 (nt)
ggcatcaccttctcctcttttggcatgtactgggtgcggcaagctcccggaaaaggactggaatggctgggctacatcgcctacgacggctctgac

SEQ ID NO: 243 - DNA, 186 bp, synthetic construct
  source 1..186: mol_type = other DNA; note = S2V29-VH.37 H2-H3 (nt)
atcgcctacgacggctctgacacctactacgccgattccgtgaagggccgcttcacaatctccagagataactccaagaacaccctgtacctgcagatgaactctctgcggatcgaggacaccgccgtgtactactgcgccaaggactactatcctctgctgtcctactactacggcctggacgtg

SEQ ID NO: 244 - DNA, 189 bp, synthetic construct
  source 1..189: mol_type = other DNA; note = S2V29-VH.37 H2+Fr.A-H3 (nt)
tacatcgcctacgacggctctgacacctactacgccgattccgtgaagggccgcttcacaatctccagagataactccaagaacaccctgtacctgcagatgaactctctgcggatcgaggacaccgccgtgtactactgcgccaaggactactatcctctgctgtcctactactacggcctggacgtg

SEQ ID NO: 245 - DNA, 198 bp, synthetic construct
  source 1..198: mol_type = other DNA; note = S2V29.VH.37 H2+Fr.B-H3 (nt)
tggctgggctacatcgcctacgacggctctgacacctactacgccgattccgtgaagggccgcttcacaatctccagagataactccaagaacaccctgtacctgcagatgaactctctgcggatcgaggacaccgccgtgtactactgcgccaaggactactatcctctgctgtcctactactacggcctggacgtg

SEQ ID NO: 246 - DNA, 225 bp, synthetic construct
  source 1..225: mol_type = other DNA; note = S2V29-VL.2 L1-L3 (nt)
tctagcgatgtgggcgcttacaactacgtgtcctggtatcaacagcaccccggcaaggcccctaagttcatgatctacgacgtggaccagcggccatctggcgtgcctgatagattctccggctccaaatccggcaacaccgcttctctgatcatctctggcctgcaggccgaggacgaagccgactactactgctcctcctacgccggatcttacatctgggtc

SEQ ID NO: 247 - DNA, 86 bp, synthetic construct
  source 1..86: mol_type = other DNA; note = S2V29-VL.2 L1-L2 (nt)
tctagcgatgtgggcgcttacaactacgtgtcctggtatcaacagcaccccggcaaggcccctaagttcatgatctacgacgtgga

SEQ ID NO: 248 - DNA, 147 bp, synthetic construct
  source 1..147: mol_type = other DNA; note = S2V29-VL.2 L2-L3 (nt)
gacgtggaccagcggccatctggcgtgcctgatagattctccggctccaaatccggcaacaccgcttctctgatcatctctggcctgcaggccgaggacgaagccgactactactgctcctcctacgccggatcttacatctgggtc

SEQ ID NO: 249 - RNA, 369 nt, synthetic construct
  source 1..369: mol_type = other RNA; note = S2V29-VH.37 (RNA nt)
gaagtgcaactagtggaaagtggtggtagtgtcgtgcagcctggcagagccctcagactgagctgtgctgcttctggcatcaccttctcctcttttggcatgtactgggtgcggcaagctcccggaaaaggactggaatggctgggctacatcgcctacgacggctctgacacctactacgccgattccgtgaagggccgcttcacaatctccagagataactccaagaacaccctgtacctgcagatgaactctctgcggatcgaggacaccgccgtgtactactgcgccaaggactactatcctctgctgtcctactactacggcctggacgtgtggggccagggcaccacagtgaccgtgtccagc

SEQ ID NO: 250 - RNA, 330 nt, synthetic construct
  source 1..330: mol_type = other RNA; note = S2V29-VL.2 (RNA nt)
caaagtgtgctaactcaaccacgaagtgtgagcggctctcctggccagtccgtgaccatctcctgtaccggcacctctagcgatgtgggcgcttacaactacgtgtcctggtatcaacagcaccccggcaaggcccctaagttcatgatctacgacgtggaccagcggccatctggcgtgcctgatagattctccggctccaaatccggcaacaccgcttctctgatcatctctggcctgcaggccgaggacgaagccgactactactgctcctcctacgccggatcttacatctgggtctttggcggcggaacacagctgaccgtgctg

SEQ ID NO: 251 - protein, 6 aa, synthetic construct
  source 1..6: note = Linker (aa)
  VARIANT 3..5: note = or serine (S)
  VARIANT 1: note = or a positively charged amino acid
  VARIANT 6: note = or a negatively charged amino acid
BPGGGZ

SEQ ID NO: 252 - protein, 110 aa, synthetic construct
  source 1..110: note = Linker (aa) - (GxS)y, wherein x is 1-10 and y is 1-10
  VARIANT 12..110: note = or absent
  VARIANT 23..110: note = or absent
  VARIANT 34..110: note = or absent
  VARIANT 45..110: note = or absent
  VARIANT 56..110: note = or absent
  VARIANT 67..110: note = or absent
  VARIANT 78..110: note = or absent
  VARIANT 89..110: note = or absent
  VARIANT 100..110: note = or absent
GGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGS